SEQUENCE LISTING (26 sequences)

SEQ ID NO: 1 - protein, 5 aa, synthetic construct
NYGVH

SEQ ID NO: 2 - protein, 16 aa, synthetic construct
VIWSGGNTDYNTPFTS

SEQ ID NO: 3 - protein, 11 aa, synthetic construct
ALTYYDYEFAY

SEQ ID NO: 4 - protein, 11 aa, synthetic construct
RASQSIGTNIH

SEQ ID NO: 5 - protein, 7 aa, synthetic construct
YASESIS

SEQ ID NO: 6 - protein, 9 aa, synthetic construct
QQNNNWPTT

SEQ ID NO: 7 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 8 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 9 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, synthetic construct
SGDYYWS

SEQ ID NO: 12 - protein, 16 aa, synthetic construct
YIYYSGSTDYNPSLKS

SEQ ID NO: 13 - protein, 11 aa, synthetic construct
VSIFGVGTFDY

SEQ ID NO: 14 - protein, 11 aa, synthetic construct
RASQSVSSYLA

SEQ ID NO: 15 - protein, 7 aa, synthetic construct
DASNRAT

SEQ ID NO: 16 - protein, 9 aa, synthetic construct
HQYGSTPLT

SEQ ID NO: 17 - protein, 121 aa, synthetic construct [sequence omitted]

SEQ ID NO: 18 - protein, 107 aa, synthetic construct [sequence omitted]

SEQ ID NO: 19 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 4 aa, synthetic construct
GFLG

SEQ ID NO: 22 - protein, 4 aa, synthetic construct
ALAL

SEQ ID NO: 23 - protein, 4 aa, synthetic construct
GGFG

SEQ ID NO: 24 - protein, 4 aa, synthetic construct
KAAN

SEQ ID NO: 25 - protein, 4 aa, synthetic construct
KAAD

SEQ ID NO: 26 - protein, 4 aa, synthetic construct
VKGG

(19) United States
(12) Patent Application Publication
(10) Pub. No.: US 2026/0191975 A1
(43) Pub. Date: Jul. 9, 2026

(54) NOVEL ANTIBODY-DRUG CONJUGATE AND USE THEREOF

(71) Applicant: HUTCHMED LIMITED, Shanghai (CN)

(72) Inventors: Weihan ZHANG, Shanghai (CN); Haibin YANG, Shanghai (CN); Wei-Guo SU, Shanghai (CN)

(21) Appl. No.: 19/419,922

(22) Filed: Dec. 15, 2025

Related U.S. Application Data
(63) Continuation of application No. PCT/CN2025/131665, filed on Oct. 31, 2025.

(30) Foreign Application Priority Data
Oct. 31, 2024 (CN) 202411546713.8
Oct. 22, 2025 (CN) 202511517613.7

(51) Int. Cl. A61K 47/68; A61P 35/00; C07K 16/28

(52) U.S. Cl. CPC A61K 47/6803; A61K 47/6849; A61K 47/6889; A61P 35/00; C07K 16/2863

(57) ABSTRACT

The present invention relates to the field of biopharmaceuticals, and in particular to an antibody-drug conjugate (ADC) that specifically binds to EGFR, a preparation method therefor and the use thereof.

### CROSS-REFERENCE TO RELATED APPLICATIONS

This application is a continuation of International Application No. PCT/CN2025/131665 filed Oct. 31, 2025, which claims priority to Chinese Patent Application No. 202411546713.8 filed Oct. 31, 2024, and Chinese Patent Application No. 202511517613.7 filed Oct. 22, 2025, the contents of each of which are hereby incorporated by reference in their entireties.

### REFERENCE TO A SEQUENCE LISTING

The contents of the electronic sequence listing (2025-12-10-HML-17-PCTC-SequenceListing.xml; Size: 25,203 bytes; and Date of Creation: Dec. 10, 2025) is herein incorporated by reference in its entirety.

### TECHNICAL FIELD

The present invention relates to the field of biopharmaceuticals, and in particular to an antibody-drug conjugate (ADC) targeting EGFR, a preparation method therefor and the use thereof.

### BACKGROUND ART

Phosphatidylinositol kinases are a large class of kinases with phosphoinositol lipids as substrates, including several family subgroups such as phosphatidylinositol-3 kinases (PI3Ks) and phosphatidylinositol-3 kinase-related kinases (PIKKs) (Burke J E et al. Mol Cell. 2018; 71 (5): 653-673). PI3Ks can be divided into class I, class II, and class III PI3Ks in mammals. Among them, the class I PI3Ks are the most common and are more closely related to the occurrence and development of tumors, which can be further divided into class IA and class IB PI3Ks. The class IA PI3K consists of three genes encoding catalytic subunits (PIK3CA, PIK3CB, and PIK3CD) and three genes encoding regulatory subunits (PIK3R1, PIK3R2, and PIK3R3). The three catalytic subunit genes encode three highly homologous class IA catalytic isomers, respectively: p110α, p110β, and p110δ, while the three regulatory subunit genes produce 5 regulatory subunits collectively referred to as p85-type (p85α, p55α, p50α, p85β, and p55γ). The class IB PI3K is a heterodimer consisting of a p110γ catalytic subunit encoded by PIK3CG and a regulatory isomer p101 encoded by PIK3R5 or a p87 encoded by PIK3R6. The p110α and p110β are widely expressed, while the expressions of the p110δ and p110γ are generally restricted to leukocytes (Thorpe L M et al. 2015; 15 (1): 7-24). The activation of PI3Ks is initiated by the binding of extracellular growth factors, cytokines, and chemokines to a tyrosine kinase receptor (RTK) or a G protein coupled receptor (GPCR), which phosphorylates the regulatory subunits of PI3K and activates the catalytic subunit p110 of PI3K. Activation of PI3K phosphorylates the 3′ hydroxy group of phosphatidylinositol lipids (PtdIns), resulting in phosphatidylinositol 3,4,5-trisphosphate (PIP3), which further activates downstream effectors such as protein kinase B (AKT) and mammalian target of rapamycin (mTOR), thereby controlling physiological and cellular functions such as cell growth, proliferation, survival, movement, and metabolism. Phosphatase and tensin homolog (PTEN) inactivate the PI3K signaling pathway by dephosphorylating PIP3 (Millis S Z et al. JAMA Oncol. 2016; 2 (12): 1565-1573). PIKKs are a subgroup of phosphatidylinositol kinase family closely related to the kinase domain of PI3K protein family, which has family members including dysregulated telangiectasia mutant kinase (ATM), ATM- and Rad3-related kinase (ATR), DNA-dependent protein kinase catalytic subunit (DNA-PK), mammalian target of rapamycin (mTOR), etc. The family members of PIKKs are involved in sensing and repairing cellular DNA damages as well as regulating cell growth, proliferation, survival, and metabolism (Shaik A et al. 2020; 25 (8): 1510-1537).

The PI3K signaling pathway is one of the most common activation signals in many types of cancers. This pathway controls tumor cell growth, proliferation, survival, differentiation, protein synthesis, glucose metabolism, migration, and apoptosis. In cancers, the PI3K signaling pathway often leads to constitutive activation due to protein abnormalities in the signaling pathway. Common signal protein abnormalities include: activating mutation and/or amplification of the catalytic subunit a (PIK3CA); no expression, decreased expression or inactivating mutation of regulatory subunits, such as PIK3R1; no expression, decreased expression or inactivating mutation of PTENs; mutation and/or amplification of AKT; mutation and/or amplification of mTOR; activating mutation and/or amplification of the upstream RTKs (e.g., HER2, HER3, c-Met, FGFR1, FGFR2, FGFR3, IGF1R, RET, ROS1, ALK, etc.); activating mutation and/or amplification of Ras family proteins, and so forth. In pre-clinical animal models, these alterations are sufficient to induce tumorigenesis. Highly frequent abnormalities of the PI3K signaling pathway-related proteins and genes have been observed in various solid tumors, including but not limited to breast cancer, endometrial cancer, prostate cancer, anal cancer, hepatocellular cancer, colorectal cancer, renal cancer, bladder cancer, head and neck squamous cell carcinoma, gastric cancer, esophageal cancer, ovarian cancer, pancreatic cancer, non-small cell lung cancer, small cell lung cancer, sarcoma, thyroid cancer, and various squamous cell carcinomas, melanoma, and glioblastoma (Millis S Z et al. JAMA Oncol. 2016; 2 (12): 1565-1573).

The critical roles of PI3Ks in cancers make them become very attractive therapeutic targets, and various small molecule inhibitors of different types targeting the key components in this signaling pathway have entered clinical development, involving a range of patients with different cancers. However, currently only selective PI3Kα, AKT and mTOR inhibitors need to be in combination with other drugs for approval in the treatment of HR-positive, HER2-negative metastatic breast cancer. Moreover, pan-PI3K and PIKK targeting inhibitors should produce stronger anti-tumor activities because they can simultaneously inhibit various different signal proteins in the PI3K signaling pathway and can overcome the feedback activation produced by the selective PI3K inhibitors; it is possible to better inhibit DNA damage repair induced by radiotherapy or chemotherapy, induce tumor cell apoptosis, and is capable of further enhancing anti-tumor activity if PIKKs (such as ATM, ATR, DNA-PK, and mTOR) are simultaneously targeted. Nevertheless, pan-PI3K inhibitors, pan-PI3K and mTOR dual-target inhibitors, and pan-PI3K and PIKK targeting inhibitors progress slowly due to the relatively high clinical toxicity. If the clinical toxicity can be effectively controlled and the safety window can be improved in cancer treatment, it can produce better clinical benefits in the treatment of more types of cancers with abnormal activation of the PI3K signaling pathway. Currently, antibody-drug conjugates (ADCs) are considered to be the most effective technical means to achieve this goal. An antibody-drug conjugate is composed of an antibody, a payload, and a linker. A biologically active molecular payload (e.g., a pan-PI3K and PIKK targeting inhibitor) is covalently conjugated to an antibody via a linker; the antibody (such as monoclonal antibody) can specifically recognize a specific target on the surface of tumor cells, thereby targeting the ADC to the surface of cancer cells and enabling it to enter into cancer cells through endocytosis. The linker is then cleaved under the action of low pH values, lysosomal enzymatic reactions, etc. in the cells, and the payload is released, thereby achieving the effect of specifically killing cancer cells without damaging normal tissue cells, accordingly reducing the non-tumor tissue toxicity or systemic toxicity of the biologically active molecular payload. An ideal linker should ensure that the ADC drug maintains sufficient stability and integrity in the blood circulation, and upon reaching therapeutic targets (tumor cells), can efficiently release the payload (Chau C H et al. 2019; 394(10200): 793-804; Drago J Z et al. Nat Rev Clin Oncol. 2021; 18(6): 327-344).

The human epidermal growth factor receptor (EGFR) is a receptor tyrosine kinase that plays an important role in cell proliferation, differentiation and migration. Abnormal activation of EGFR, such as overexpression or mutation, is closely associated with the occurrence and development of a variety of tumors, such as non-small cell lung cancer, head and neck squamous cell carcinoma, esophageal squamous carcinoma and colorectal cancer (Karlsen E A et al., Cells. 2021; 10(5): 1206). Currently, a number of small molecule EGFR inhibitors and monoclonal antibodies have been approved by the FDA for use in tumor therapy, showing clinical benefits in some patients. However, not all of these drugs are capable of completely curing tumors, and most patients develop resistance after a certain period of treatment. In recent years, some EGFR-targeted ADC drugs have been also under development. For example, MRG003, which is progressing rapidly, has shown promising activity in clinical trials for nasopharyngeal cancer and head and neck cancer. One of the resistance mechanisms of EGFR monoclonal antibodies is the constitutive activation of downstream effector molecules of EGFR, including activation of the PI3K/AKT signaling pathway (Brand T M et al., Cancer Biol Ther. 2011 May 1; 11(9): 777-92). Multiple studies have also reported that the combination of EGFR monoclonal antibodies and PI3K inhibitors can overcome such resistance, showing better anti-tumor activity (Tsuchihashi H et al., Oncol Rep. 2020 September; 44(3): 863-872). Therefore, simultaneously targeting EGFR and PI3K/PIKK is a direction worthy of exploration.

Compared with the small molecule inhibitors, the ADCs loaded with pan-PI3K and PIKK targeting inhibitors conjugate the antibodies targeting tumor surface antigens with potent payloads through cleavable linkers, thereby achieving specifically killing cancer cells expressing such tumor surface antigens without damaging normal tissue cells, reducing the normal tissue toxicity and systematic toxicity of the payloads, and thus improving the safety window and enhancing anti-tumor activity. At present, no ADC loaded with pan-PI3K and PIKK targeting inhibitors has entered clinical research. The clinical medical needs have not been met obviously. The present invention is intended to develop a novel antibody-drug conjugate targeting EGFR, with pan-PI3K and PIKK targeting inhibitors as payloads.

### SUMMARY OF THE INVENTION

The present invention provides an antibody-drug conjugate having a structure represented by formula (I) or a pharmaceutically acceptable salt thereof, or a solvate, a racemic mixture, an enantiomer, a diastereomer or a tautomer thereof:

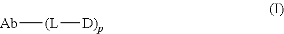

wherein
Ab is an antibody that can specifically bind to EGFR or an antigen-binding fragment thereof;
D is a PI3K and/or PIKK inhibitor;
L is a linker, which links Ab to D; and
p is an integer from 1 to 20.

In some embodiments, the PI3K and/or PIKK inhibitor has a structure represented by formula (II):

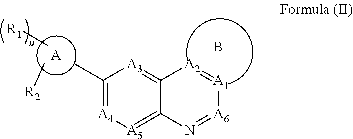

wherein
A₃, A₄, A₅, and A₆ are independently N or CR₅; R₅ is independently selected from: H, C₁₋₆ alkyl, C₃₋₉ cycloalkyl, —CN, —NH₂, —OH, —SH, —O(C₁₋₆ alkyl), halogen, or C₁₋₆ haloalkyl;

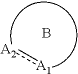

represents a 5- to 6-membered heteroaryl ring or a 3- to 8-membered heterocyclyl ring, and the 5- to 6-membered heteroaryl ring or 3- to 8-membered heterocyclyl ring is optionally substituted with one or more groups selected from oxo, ═NCN, or R₃;
ring A is 5- to 14-membered heteroaryl;
R₁, R₂, and R₃ are independently selected from: H, C₁₋₆ alkyl, C₂₋₈ alkenyl, C₂₋₈ alkynyl, C₆-C₁₀ aryl, C₃₋₉ cycloalkyl, —C(O)R^{a}, —C(O)OR^{b}, —CN, —C(O)NR^{c}R^{d}, —NR^{c}R^{d}, —NR^{c}C(O)R^{a}, —NR^{c}S(O)ₙR^{e}, —NR^{c}S(O)ₙNR^{f}R^{g}, —NR^{c}C(O)OR^{b}, —NR^{c}C(O)NR^{d}R^{e}, —NO₂, —OR^{b}, —SR^{b}, —S(O)ₙR^{e}, —S(O)ₙR^{c}R^{d}, halogen, C₁₋₆ haloalkyl, 5- to 6-membered heteroaryl, and 3- to 12-membered heterocyclyl;
R^{a}, R^{b}, R^{e}, R^{d}, R^{e}, R^{f}, and R^{g} are each independently selected from hydrogen, C₁₋₆ alkyl, C₂₋₈ alkenyl, C₂₋₈ alkynyl, C₆-C₁₀ aryl, C₃₋₉ cycloalkyl, C₁₋₆ haloalkyl, 5- to 6-membered heteroaryl, and 3- to 12-membered heterocyclyl;
or R^{a} and R^{c}, and/or R^{c} and R^{d}, and/or R^{c} and R^{e}, and/or R^{c} and R^{f}, and/or R^{d} and R^{e}, and/or R^{g} and R^{f} together with the atoms to which they are attached form 3- to 12-membered heterocyclyl;
wherein the C₁₋₆ alkyl, C₂₋₈ alkenyl, C₂₋₈ alkynyl, C₁₋₆ haloalkyl, C₆₋-C₁₀ aryl, C₃₋₉ cycloalkyl, 5- to 6-membered heteroaryl, and 3- to 12-membered heterocyclyl, at each occurrence, can each independently and optionally be substituted with one or more groups selected from: deuterium, C₁₋₆ alkyl, C₂₋₈ alkenyl, C₂₋₈ alkynyl, C₆-C₁₀ aryl, C₃₋₉ cycloalkyl, —C(O)R^{a}, —C(O)OR^{b}, —CN, —C(O)NR^{c}R^{d}, —NR^{c}R^{d}, —NR^{c}C(O)R^{a}, —NR^{c}S(O)ₙR^{e}, —NR^{c}S(O)ₙNR^{f}R^{g}, —NR^{c}C(O)OR^{b}, —NR^{c}C(O)NR^{d}R^{e}, —NO₂, —C₁₋₆ alkylene-OR^{b}, —OR^{b}, —S(O)ₙR^{e}, —S(O)ₙR^{c}R^{d}, halogen, C₁₋₆ haloalkyl, 5- to 6-membered heteroaryl, and 3- to 8-membered heterocyclyl;
n, at each occurrence, is independently 0, 1, or 2;
u, at each occurrence, is independently 0, 1, 2, or 3; and [symbol] represents a single bond or a double bond; and
wherein one of the R₁, R₂ or R₃, when present, is covalently linked to the L, and the valence of the atom at the linking site does not exceed the normal valence of the atom due to the one or more substituents to which it is linked.

The present invention further provides a pharmaceutical composition comprising the antibody-drug conjugate and/or at least one pharmaceutically acceptable salt thereof of the present invention, and at least one pharmaceutically acceptable carrier.

The present invention further provides a medicament for inhibiting the activity of PI3K and/or PIKK, the medicament comprising an effective amount of the at least one antibody-drug conjugate and/or at least one pharmaceutically acceptable salt thereof of the present invention.

The present invention further provides the use of the antibody-drug conjugate and/or at least one pharmaceutically acceptable salt thereof in the manufacture of a drug for the treatment of a disease or condition mediated at least in part by PI3K and/or PIKK. The disease or condition mediated at least in part by PI3K and/or PIKK is cancer or autoimmune disease. Preferably, the cancer is a cancer associated with the activity of the PI3K and/or PIKK kinases.

The present invention further provides a method of inhibiting the activity of PI3K and/or PIKK, comprising contacting an effective amount of the at least one antibody-drug conjugate and/or at least one pharmaceutically acceptable salt thereof with the kinase. In another preferred embodiment, the method of inhibition is in vitro and non-therapeutic.

The present invention further provides a method of treating disease or condition responsive to the inhibition of PI3K and/or PIKK, comprising administering an effective amount of the at least one antibody-drug conjugate and/or at least one pharmaceutically acceptable salt thereof to a subject in need of treatment of the disease or condition.

The present invention further provides a method of delivering a PI3K and/or PIKK inhibitor to a cell or tissue expressing EGFR, comprising conjugating the PI3K and/or PIKK inhibitor to an antibody that immunospecifically binds to an EGFR epitope to obtain an antibody-drug conjugate (abbreviated as “ADC”), and exposing the cell or tissue to the ADC.

### DETAILED DESCRIPTION OF THE INVENTION

### Definitions

As used in the specification, the following words, phrases and symbols are generally intended to have the meanings as set forth below, except to the extent that the context in which they are used indicates otherwise.

A dash (“−”) that is not between two letters or symbols is used to indicate a point of attachment for a substituent. For example, —O(C₁₋₆ alkyl) refers to the attachment of C₁₋₆ alkyl to the rest of the molecule through an oxygen atom.

The term “alkyl” as used herein refers to a straight or branched saturated hydrocarbon radical containing 1-18 carbon atoms (C₁₋₁₈), preferably 1-10 carbon atoms (C₁₋₁₀), more preferably 1-6 carbon atoms (C₁₋₆), and even more preferably 1-4 carbon atoms (C₁₋₄) or 1-3 carbon atoms (C₁₋₃). When the term “alkyl” is prefixed with “C_{a-b}”, it means the number of carbon atoms in the alkyl, wherein a is the minimum number of carbon atoms in the alkyl and b is the maximum number of carbon atoms in the alkyl. For example, “C₁₋₆ alkyl” means an alkyl containing 1-6 carbon atoms. “C₁₋₃ alkyl” means an alkyl containing 1-3 carbon atoms. Examples of C₁₋₆ alkyl include, but are not limited to, methyl, ethyl, propyl (e.g., n-propyl, and i-propyl), butyl (e.g., n-butyl, iso-butyl, sec-butyl, and tert-butyl), pentyl (e.g., n-pentyl, iso-pentyl, and neo-pentyl), hexyl, and the like. When located between two dashes (“−”) (e.g., —(C₁₋₆ alkyl)-OH), the alkyl represents an alkylene.

The term “alkylene” as used herein refers to a divalent radical of an alkyl as defined above that is attached to the rest of the molecule by two single bonds on the same carbon atom or different carbon atoms, respectively. In one embodiment, the alkylene is an alkylene of 1-6 carbon atoms (C₁₋₆ alkylene). In another embodiment, the alkylene is C₁₋₃ alkylene, C₁₋₂ alkylene or C₁ alkylene. Examples of C₁₋₆ alkylene include, but are not limited to, methylene (—CH₂—), 1,2-ethylene (—CH₂CH₂—), 1,1-ethylene (—CH(CH₃)—), 1,3-propylene (—CH₂—CH₂—CH₂—), 1,1-propylene (—CH(CH₂CH₃)—), 2,2-propylene (—C(CH₃)₂—), 1,2-propylene (—CH(CH₃)CH₂—), 1,4-butylene (—CH₂—CH₂—CH₂—CH₂—), and the like.

The term “alkenyl” as used herein refers to a straight or branched unsaturated hydrocarbon radical containing one or more, for example 1, 2, or 3 carbon-carbon double bonds (C═C) and 2-18 carbon atoms (C₂₋₁₈), preferably 2-10 carbon atoms (C₂₋₁₀), more preferably 2-6 carbon atoms (C₂₋₆), and even more preferably 2-4 carbon atoms (C₂₋₄). When the term “alkenyl” is prefixed with “C_{a-b}”, it means the number of carbon atoms in the alkenyl, wherein a is the minimum number of carbon atoms in the alkenyl and b is the maximum number of carbon atoms in the alkenyl. For example, “C₂₋₆ alkenyl” means an alkenyl containing 2-6 carbon atoms. “C₂₋₄ alkenyl” means an alkenyl containing 2-4 carbon atoms. Examples of C₂₋₆ alkenyl include, but are not limited to, ethenyl, propenyl (e.g., 2-propenyl), and butenyl (e.g., 2-butenyl), and the like. The point of attachment for the alkenyl can be on or not on the double bonds.

The term “alkynyl” as used herein refers to a straight or branched unsaturated hydrocarbon radical containing one or more, for example 1, 2, or 3 carbon-carbon triple bonds (C≡C) and 2-18 carbon atoms (C₂₋₁₈), preferably 2-10 carbon atoms (C₂₋₁₀), more preferably 2-6 carbon atoms (C₂₋₆), and even more preferably 2-4 carbon atoms (C₂₋₄). When the term “alkynyl” is prefixed with “C_{a-b}”, it means the number of carbon atoms in the alkynyl, wherein a is the minimum number of carbon atoms in the alkynyl and b is the maximum number of carbon atoms in the alkynyl. For example, “C₂₋₆ alkynyl” means an alkynyl containing 2-6 carbon atoms. “C₂₋₄ alkynyl” means an alkynyl containing 2-4 carbon atoms. Examples of C₂₋₆ alkynyl include, but are not limited to, ethynyl, propynyl (e.g., 2-propynyl), and butynyl (e.g., 2-butynyl), and the like. The point of attachment for the alkynyl can be on or not on the triple bonds.

The term “halogen” or “halo” as used herein refers to fluoro, chloro, bromo, and iodo, preferably fluoro, chloro, and bromo, more preferably fluoro and chloro.

The term “haloalkyl” as used herein refers to an alkyl as defined herein, in which one or more, for example 1, 2, 3, 4, or 5, or all hydrogen atoms are replaced with halogen atoms, and when more than one hydrogen atoms are replaced by halogen atoms, the halogen atoms can be the same or different from each other. In one embodiment, the term “haloalkyl” as used herein refers to an alkyl as defined herein, in which two or more, for example 2, 3, 4, or 5, or all hydrogen atoms are replaced by halogen atoms, wherein the halogen atoms are identical to each other. In another embodiment, the term “haloalkyl” as used herein refers to an alkyl as defined herein, in which two or more hydrogen atoms, for example 2, 3, 4, or 5, or all hydrogen atoms are replaced by halogen atoms, wherein the halogen atoms are different from each other. When the term “haloalkyl” is prefixed with “C_{a-b}”, it means the number of carbon atoms in the haloalkyl, wherein a is the minimum number of carbon atoms in the haloalkyl and b is the maximum number of carbon atoms in the haloalkyl. For example, “C₁₋₆ haloalkyl” means a haloalkyl as defined herein containing 1-6 carbon atoms. “C₁₋₄ haloalkyl” means a haloalkyl as defined herein containing 1-4 carbon atoms. Examples of C₁₋₆ haloalkyl include, but are not limited to, —CF₃, —CHF₂, —CH₂F, —CH₂CH₂F, —CH₂CHF₂, —CH₂CF₃, —CH(CF₃)₂, and the like.

As used herein, the term “imino” or written as “═N—R” refers to an amino group as a divalent substituent, in which two valences of the same nitrogen atom are attached to an atom optionally selected from the rest of the compound to form a double bond, and the third valence of the nitrogen atom is attached to the R group as defined by the context. The nitrogen atom, when attached to a carbon atom, forms an imine, amidine, guanidine, etc., or when attached to a heteroatom, forms a sulfoxide imine, etc.

The term “cycloalkyl” as used herein refers to a saturated or partially unsaturated cyclic hydrocarbyl containing 3-12 ring carbon atoms (C₃₋₁₂) (e.g., 3-10 ring carbon atoms (C₃₋₁₀), 3-9 ring carbon atoms (C₃₋₉), 3-8 ring carbon atoms (C₃₋₈), 5-7 ring carbon atoms (C₇), 4-7 ring carbon atoms (C₄₋₇), or 3-6 ring carbon atoms (C₃₋₆)); it may have one or more rings, for example 1, 2, or 3 rings, preferably 1 or 2 rings. When the term “cycloalkyl” is prefixed with “C_{a-b}”, it means the number of carbon atoms in the cycloalkyl, wherein a is the minimum number of carbon atoms in the cycloalkyl and b is the maximum number of carbon atoms in the cycloalkyl. For example, “C₃₋₈ cycloalkyl” or “3- to 8-membered cycloalkyl” means cycloalkyl having 3-8 ring carbon atoms; “C₃₋₆ cycloalkyl” or “3- to 6-membered cycloalkyl” means cycloalkyl having 3-6 ring carbon atoms. The cycloalkyl can include a fused or bridged ring, or a spirocyclic ring. The rings of the cycloalkyl can be saturated, or can have one or more, for example, one or two double bonds (i.e., partially unsaturated), but not fully conjugated, and not an “aryl” as defined in the invention. Examples of cycloalkyl include, but are not limited to, cyclopropyl, cyclobutyl, cyclopentyl, cyclohexyl, spiro[2.2]pentanyl, cyclopropenyl, cyclobutenyl, cyclopentenyl, cyclopentadienyl, cyclohexenyl, and the like.

The term “heterocyclyl” or “heterocycle” as used herein can be used interchangeably and refers to a saturated or partially unsaturated ring having 3-14 ring atoms (e.g., 4-14 ring atoms (4- to 14-membered heterocyclyl), 4-12 ring atoms (4- to 12-membered heterocyclyl), 3-12 ring atoms (3- to 12-membered heterocyclyl), 4-10 ring atoms (4- to 10-membered heterocyclyl), 3-8 ring atoms (3- to 8-membered heterocyclyl), 4-8 ring atoms (4- to 8-membered heterocyclyl), 3-6 ring atoms (3- to 6-membered heterocyclyl), or 4-5 ring atoms (4- to 5-membered heterocyclyl)), wherein the ring atoms include one or more (e.g., 1, 2, or 3, preferably 1 or 2) heteroatoms independently selected from N, O, and S, and the remaining ring atoms are carbon atoms; it may have one or more rings, for example 1, 2, or 3 rings, preferably 1 or 2 rings. N and S can be optionally oxidized to various oxidation states. The point of attachment of heterocyclyl can be on the N heteroatom or carbon atoms. For example, “4- to 10-membered heterocyclyl” means a heterocyclyl having 4-10 (e.g., 4, 5, 6, 7, 8, 9, or 10) ring atoms comprising at least one, such as 1, 2, 3, or 4, preferably 1, 2, or 3 heteroatoms independently selected from N, O, and S; “3- to 8-membered heterocyclyl” means a heterocyclyl having 3-8 (e.g., 3, 4, 5, 6, 7, or 8) ring atoms comprising at least one, such as 1, 2, or 3, preferably 1 or 2 heteroatoms independently selected from N, O, and S; “3- to 6-membered heterocyclyl” means a heterocyclyl having 3-6 (e.g., 3, 4, 5 or 6) ring atoms comprising at least one, preferably 1 or 2 heteroatoms independently selected from N, O, and S (preferably N and O), which is preferably a monocyclic ring. The heterocyclyl can include a fused or bridged ring, or a spirocyclic ring. The rings of the heterocyclyl may be saturated or can have one or more, for example, one or two double bonds (i.e., partially unsaturated), but not fully conjugated and not a “heteroaryl” as defined in the invention. Examples of heterocyclyl include, but are not limited to: 3- to 12-membered heterocyclyl, 4- to 10-membered heterocyclyl, 3- to 8-membered heterocyclyl, 3- to 6-membered heterocyclyl, and 4- to 5-membered heterocyclyl, for example, oxetanyl, azetidinyl, pyrrolidinyl, tetrahydrofuryl, dioxolanyl, tetrahydropyranyl, morpholinyl, thiomorpholinyl, piperidinyl, piperazinyl, 1-methylpiperazinyl, 4-(2-hydroxyethyl)-piperazin-1-yl, tetrahydropyridinyl, dihydropyrimidinyl, dihydropyrazinyl, pyrazolidinyl, hexahydro-1H-pyrrolizinyl, hexahydrospiro[cyclopropane-pyrrolizinyl](e.g., tetrahydro-1′H,3′H-spiro[cyclopropane-1,2′-pyrrolizinyl], hexahydrospiro[cyclopropane-1,3′-pyrrolizinyl] and hexahydrospiro[cyclopropane-_{1,1}′-pyrrolizinyl]), octahydrocyclopropeno[a]pyrrolidinyl and oxaspiro[3.3]heptanyl, 2,7-diazaspiro[3.5]nonanyl, octahydropyrrolo[3,4-c]pyrrolyl, 3,7-diazabicyclo[4.2.0]octanyl, 4,7-diazaspiro[2.5]octanyl, 2,6-diazaspiro[3.5]nonanyl, 2,7-diazaspiro[4.4]nonanyl, and 2,6-diazaspiro[3.4]octanyl, preferably oxetanyl, azetidinyl, pyrrolidinyl, tetrahydropyranyl, morpholinyl, piperidinyl, 2-(piperazin-1-yl)glycolyl, piperazinyl, hexahydro-1H-pyrrolizinyl, tetrahydro-1′H,3′H-spiro[cyclopropane-1,2′-pyrrolizinyl], octahydrocyclopropeno[a]pyrrolizinyl, 2,7-diazaspiro[3.5]nonanyl, octahydropyrrolo[3,4-c]pyrrolyl, 3,7-diazabicyclo[4.2.0]octanyl, 4,7-diazaspiro[2.5]octanyl, 2,6-diazaspiro[3.5]nonanyl, 2,7-diazaspiro[4.4]nonanyl, and 2,6-diazaspiro[3.4]octanyl.

The terms “aryl” or “aromatic hydrocarbon” as used herein are used interchangeably to refer to a carbocyclic hydrocarbyl consisting of one ring or a plurality of, such as two, fused rings having 6-14 carbon atoms (e.g., 6-14 carbon atoms (6- to 14-membered aryl or C₆-C₁₄ aryl), 6-12 carbon atoms (6- to 12-membered aryl or C₆-C₁₂ aryl), 6-10 carbon atoms (6- to 10-membered aryl or C₆-C₁₀ aryl)), wherein at least one ring is an aromatic ring and the point of attachment to the rest of the molecule is on the aromatic ring.

Examples of aryl include, but are not limited to, phenyl, naphthalenyl, 1,2,3,4-tetrahydronaphthalenyl, phenanthryl, indenyl, indanyl, azulenyl, and benzocyclobutenyl, preferably phenyl and naphthalenyl.

The terms “heteroaryl” and “heteroaryl ring” as used herein are used interchangeably to refer to a monocyclic, dicyclic or tricyclic ring system having 5-15 ring atoms (e.g., 5-14 ring atoms (5- to 14-membered heteroaryl), 5-13 ring atoms (5- to 13-membered heteroaryl), 5-12 ring atoms (5- to 12-membered heteroaryl), 5-6 ring atoms (5- to 6-membered heteroaryl), 8-13 ring atoms (8- to 13-membered heteroaryl), 9-12 ring atoms (9- to 12-membered heteroaryl)), wherein the ring atoms include one or more, such as 1, 2, 3, or 4, preferably 1, 2, or 3 heteroatoms independently selected from N, O, and S, and the remaining ring atoms are carbon atoms, wherein at least one ring is an aromatic ring and the point of attachment to the rest of the molecule is on the aromatic ring, and wherein the S and N heteroatoms can be optionally oxidized to various oxidation states. When the total number of S and O atoms in the heteroaryl exceeds 1, the S and O heteroatoms are not adjacent to one another. Preferably, the heteroaryl is 5- to 13-membered heteroaryl. For example, the heteroaryl includes:

5- to 6-membered monocyclic heteroaryl, i.e., a monocyclic aromatic heterocyclic group having 5 or 6 ring atoms, wherein the ring atoms include one or more, such as 1, 2, or 3 heteroatoms independently selected from N, O, and S (preferably N), and the remaining ring atoms are carbon atoms; and

8- to 13-membered bicyclic or tricyclic heteroaryl, i.e., a bicyclic or tricyclic aromatic heterocyclic group having 8, 9, 10, 11, 12, or 13 ring atoms, wherein the ring atoms include one or more, such as 1, 2, 3, or 4, preferably 1, 2, or 3 heteroatoms independently selected from N, O, and S (preferably N), and the remaining ring atoms are carbon atoms, wherein at least one ring is an aromatic ring.

Examples of heteroaryl include, but are not limited to: 5- to 6-membered monocyclic heteroaryl, for example, pyridinyl, N-oxide pyridinyl, pyrazinyl, pyrimidinyl, triazinyl (e.g., 1,2,4-triazinyl, 1,3,5-triazinyl), pyrazolyl, imidazolyl, oxazolyl, isoxazolyl, oxadiazolyl (e.g., 1,2,4-oxadiazolyl, 1,2,5-oxadiazolyl, and 1,3,4-oxadiazolyl), thiazolyl, isothiazolyl, thiadiazolyl, tetrazolyl, triazolyl, thienyl, furyl, pyranyl, pyrrolyl, and pyridazinyl, preferably triazolyl, pyridinyl, N-oxide pyridinyl, pyrazinyl, pyridazinyl, pyrimidinyl, pyrazolyl, imidazolyl, isoxazolyl, triazinyl (e.g., 1,2,4-triazinyl), oxazolyl, thiadiazolyl, and more preferably pyridinyl (e.g., pyridin-4-yl, pyridin-3-yl), pyrazinyl, pyridazinyl, and pyrimidinyl; and 8- to 13-membered bicyclic or tricyclic heteroaryl, for example benzoxazolyl, benzisoxazolyl, benzothienyl, benzothiazolyl, benzisothiazolyl, imidazopyrimidinyl (e.g., imidazo[1,2-c]pyrimidinyl), imidazopyrazinyl (e.g., imidazo[1,2-a]pyrazinyl and imidazo[1,5-a]pyrazinyl), imidazopyridinyl (e.g., imidazo[1,2-a]pyridinyl), imidazopyridazinyl (e.g., imidazo[1,2-b]pyridazinyl), pyrrolopyrazinyl (e.g., pyrrolo[1,2-a]pyrazinyl), pyrrolopyridinyl (e.g., 1H-pyrrolo[2,3-b]pyridinyl), pyrrolopyrimidinyl (e.g., pyrrolo[3,4-d]pyrimidinyl), pyrazolopyrazinyl (e.g., pyrazolo[1,5-a]pyrazinyl), pyrazolopyridinyl (e.g., 1H-pyrazolo[3,4-b]pyridinyl), pyrazolopyrimidinyl (e.g., pyrazolo[1,5-a]pyrimidinyl), triazolopyrimidinyl (e.g., [1,2,4]triazolo[4,3-c]pyrimidinyl and [1,2,4]triazolo[1,5-c]pyrimidinyl), triazolopyrazinyl (e.g., [1,2,4]triazolo[1,5-a]pyrazinyl), triazolopyridinyl (e.g., [1,2,4]triazolo[4,3-a]pyridinyl and [1,2,4]triazolo[1,5-a]pyridinyl), tetrazolopyridinyl (e.g., tetrazolo[1,5-a]pyridinyl), benzofuryl, benzimidazolyl, indolyl, indazolyl, purinyl, quinolinyl, tetrahydroquinolinyl (e.g., 1,2,3,4-tetrahydroquinolinyl), isoquinolinyl, tetrahydroisoquinolinyl (e.g., 5,6,7,8-tetrahydroisoquinolinyl), tetrahydronaphthyridinyl (e.g., 1,2,3,4-tetrahydro-2,7-naphthyridinyl), dihydro-cyclopentadienopyridinyl (e.g., 6,7-dihydro-5H-cyclopentadieno[c]pyridinyl), dihydro-pyrrolopyridinyl (e.g., 2,3-dihydro-1H-pyrrolo[3,4-c]pyridinyl), 6,7-dihydro-4H-pyrazolo[5,1-c][1,4]oxazinyl, 1,2,3,4-tetrahydro-1,5-naphthyridinyl, benzoindazolyl (e.g., 1H-benzo[f]indazolyl), tetrahydrocyclopentadienoindazolyl (e.g., 1,5,6,7-tetrahydrocyclopentadieno[f]indazolyl), dihydroindenothiazolyl (e.g., 6,7-dihydro-5H-indeno[5,6-d]thiazolyl), hexahydroindenoxazinyl (e.g., 2,3,4,6,7,8-hexahydroindeno[5,6-b][1,4]oxazinyl), tetrahydrocyclopentadienoindolyl (e.g., 1,5,6,7-tetrahydrocyclopentadieno[f]indolyl), tetrahydroindenoimidazolyl (e.g., 1,5,6,7-tetrahydroindeno[5,6-d]imidazolyl), preferably indazolyl, indolyl, benzimidazolyl, benzothiazolyl, quinolinyl, 1,2,3,4-tetrahydroquinolinyl, isoquinolinyl, 5,6,7,8-tetrahydroisoquinolinyl, 1,2,3,4-tetrahydro-2,7-naphthyridinyl, 6,7-dihydro-5H-cyclopentadieno[c]pyridinyl, 2,3-dihydro-1H-pyrrolo[3,4-c]pyridinyl, imidazolo[1,2-c]pyrimidinyl, imidazolo[1,2-a]pyridinyl, 1H-pyrrolo[2,3-b]pyridinyl, [1,2,4]triazolo[1,5-a]pyridinyl, 1,2,3,4-tetrahydro-1,5-naphthyridinyl, 1H-benzo[f]indazolyl, 1,5,6,7-tetrahydrocyclopentadieno[f]indazolyl, 6,7-dihydro-5H-indeno[5,6-d]thiazolyl, 2,3,4,6,7,8-hexahydroindeno[5,6-b][1,4]oxazinyl, 1,5,6,7-tetrahydrocyclopentadieno[f]indolyl), and 1,5,6,7-tetrahydroindeno[5,6-d]imidazolyl.

The term “—OH” as described herein refers to a hydroxyl radical.

The term “—CN” as used herein refers to a cyano radical.

The term “oxo” as used herein refers to ═O.

The term “optional” or “optionally” as used herein means that the subsequently described event or circumstance may or may not occur, and the description includes instances in which the event or circumstance occurs and instances in which it does not occur. For example, “optionally substituted with one or more” includes unsubstituted and substituted with 1 or more substituents as described. It should be appreciated by those skilled in the art that, with respect to any radical containing one or more substituents, the radical is not intended to introduce any substitution pattern or patterns that are sterically impractical, chemically incorrect, synthetically non-feasible and/or inherently unstable.

The term “substituted” or “substituted with . . . ”, as used herein, means that one or more (such as, 1, 2, 3, or 4) hydrogens on the designated atom or group are replaced by one or more (such as 1, 2, 3, or 4) substituents, preferably the substituents selected from the indicated group of substituents or radicals, provided that the designated atom's normal valence is not exceeded. The substituents may be the same or different from each other. The term “substituted with one or more radicals selected from” or “substituted with one or more” as used herein means that one or more hydrogens on the designated atom or radical are independently replaced by one or more radicals selected from the indicated group of substituents or radicals, wherein the radicals may be the same or different from each other. Preferably, “substituted with one or more radicals selected from” or “substituted with one or more” means that the designated atom or radical is substituted with 1, 2, 3, or 4 radicals independently selected from the indicated group of substituents or radicals, wherein the radicals may be the same or different from each other. In some embodiments, when a substituent is oxo (i.e., ═O) or ═NR₃, then two hydrogens on a single atom are replaced. An optional substituent can be any radicals, provided that combinations of substituents and/or variables result in a chemically correct and stable compound. A chemically correct and stable compound is meant to imply a compound that is sufficiently stable so as to be able to survive the isolation from a reaction mixture. Preferably, substituents are those exemplified in the compounds of the examples of the present application.

Unless otherwise specified, substituents are named into the core structure. For example, it should be understood that when (cycloalkyl)alkyl is listed as a possible substituent, the point of attachment of this substituent to the core structure is in the alkyl portion.

A “pharmaceutically acceptable salt” refers to a free acid or base salt of a drug conjugate represented by formula (I) that is non-toxic, biologically tolerable or otherwise biologically suitable for administration to a subject for treatment or prophylactic. For example, acid addition salts include, for example, addition salts derived from inorganic acids and organic acids. For a general description of pharmaceutically acceptable salts, see, for example: S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002.

In addition, if a compound, or a drug conjugate described herein is obtained in the form of an acid addition salt, its free base form can be obtained by basifying a solution of the acid addition salt. Conversely, if the product is in the form of a free base, an acid addition salt, particularly a pharmaceutically acceptable acid addition salt, can be produced by dissolving the free base in a suitable solvent and treating the solution with an acid, in accordance with conventional procedures for preparing acid addition salts from base compounds. A person skilled in the art will recognize various synthetic methodologies that can be used without under experimentation to prepare non-toxic pharmaceutically acceptable acid addition salts or base addition salts.

The term “solvates” means solvent addition forms that contain either stoichiometric or non-stoichiometric amounts of solvent. Some compounds or drug conjugates have a tendency to trap a fixed molar ratio of solvent molecules in the solid state, thereby forming solvates. If the solvent is water, the solvate formed is a hydrate. When the solvent is alcohol, the solvate formed is an alcoholate. Hydrates are formed by the combination of one or more molecules of water, or less than one molecule of water, with one molecule of the substances in which the water retains its molecular state as H₂O, such combination being able to form one or more hydrates, for example, hemihydrate, monohydrate, and dihydrate.

The racemic mixture can be used as such or can be resolved into their individual isomers. The resolution can afford stereo-chemically pure compounds or mixtures enriched in one or more isomers. Methods for separation of isomers are well known (see, Allinger N. L. and Eliel E. L. in “Topics in Stereochemistry”, Vol. 6, Wiley Interscience, 1971) and include physical methods such as chromatography using chiral adsorbents. Individual isomers can be prepared in chiral forms from chiral precursors. Alternatively, individual isomers can be separated chemically from a mixture by: forming diastereomeric salts with a chiral acid (e.g., the individual enantiomers of 10-camphorsulfonic acid, camphoric acid, alpha-bromocamphoric acid, tartaric acid, diacetyltartaric acid, malic acid, pyrrolidone-5-carboxylic acid, and the like), fractionally crystallizing the salts, and then freeing one or both of the resolved bases, optionally repeating this process, so as to obtain either isomer or both isomers substantially free of the other; i.e., an isomer having an optical purity of >95%. Alternatively, the racemates can be covalently attached to a chiral compound (auxiliary) to produce diastereomers which can be separated by chromatography or by fractional crystallization after which time the chiral auxiliary is chemically removed to afford the pure enantiomers.

The term “stereoisomers” as used herein refers to compounds that have the same chemical constitution but differ in the arrangement of atoms or groups in space. Stereoisomers include enantiomers, diastereomers, and the like.

The terms “enantiomers” and “enantiomeric forms” as used herein can be used interchangeably and refer to two stereoisomers of a compound that are non-superimposable mirror images of each other.

The terms “diastereomers” and “diastereomeric forms” as used herein can be used interchangeably and refer to stereoisomers that have two or more chiral centers and are not mirror images of each other. Diastereomers have different physical properties, such as melting points, boiling points, spectral properties, or biological activities. A mixture of diastereomers can be separated by high-resolution analytical methods such as electrophoresis and chromatography such as HPLC.

The term “tautomers” as used herein refer to constitutional isomers of compounds generated by rapid movement of a certain atom in two positions in a molecule. Tautomers readily interconvert into each other, e.g., the enol form and the ketone form are typical tautomers.

The compounds of the present invention also encompass isotopically labeled compounds in which one or more atoms are replaced by atoms having an atomic mass or mass number different from those commonly found in nature. All isotopes of any particular atom or element specified, and uses thereof, are contemplated herein. Exemplary isotopes that may be incorporated into the compounds of the present invention include isotopes of hydrogen, carbon, nitrogen, oxygen, phosphorus, sulfur, fluorine, chlorine, and iodine, for example, ²H, ³H, ¹¹C, ¹³C, ¹⁴C, ¹³N, ¹⁵N, ¹⁵ O, ¹⁷O, ¹⁸O, ³²P, ³³P, ³⁵S, ¹⁸F, ³⁶Cl, ¹²³I, and ¹²⁵I. Certain isotopically labeled compounds of the invention (e.g., those labeled with ³H and ¹⁴C) can be used for the studies on the compound and/or tissue distribution of an organism. Tritium (i.e., ³H) and carbon-14 (i.e., ¹⁴C) isotopes are particularly useful for this purpose because they are easy to prepare and detectable. Furthermore, substitution with heavier isotopes such as deuterium (i.e., ²H) may provide some therapeutic advantages due to higher metabolic stability (e.g., increased half-life in-vivo or reduced dosage required) and is therefore preferred in some cases. It should be appreciated that the hydrogen (¹H) atom present in the compounds described herein can be replaced by deuterium (²H) atom to afford the deuterates. In any given compound, any number of hydrogen atoms can be replaced by the same number of deuterium atoms. Isotopically labeled compounds can typically be prepared by conventional techniques known to those skilled in the art or by procedures analogous to those described in the examples described below using appropriate isotopically labeled reagents instead of non-labeled reagents.

As used herein, the terms “group(s)” and “radical(s)” are synonymous and are intended to indicate the functional groups or molecular fragments attachable to other molecular fragments.

The term “active ingredient” is intended to indicate a chemical substance which has a biological activity. In some embodiments, an “active ingredient” is a chemical substance having a pharmaceutical utility.

The term “pharmaceutical combination” as used herein means a product obtained by mixing or combining two or more active ingredients, including fixed and non-fixed combinations of active ingredients, such as a kit and a pharmaceutical composition. The term “fixed combination” means that two or more active ingredients (such as the drug conjugate of the present invention and additional therapeutic agents) are administered simultaneously to a patient in the form of a single entity or dose. The term “non-fixed combination” means that two or more active ingredients (such as the drug conjugate of the present invention and additional therapeutic agents) are administered simultaneously, in parallel or successively to a patient in separate entities, wherein the administration provides the patient with a therapeutically effective level of the compound.

The terms “treating” or “treatment” or “prevention” of a disease or disorder refer to administering one or more pharmaceutical substances, particularly the drug conjugate of the present invention to a subject that has the disease or disorder, or has a symptom of the disease or disorder, or has a predisposition toward the disease or disorder, with the purpose to cure, heal, alleviate, relieve, alter, remedy, ameliorate, improve, or affect the disease or disorder, the symptom of the disease or disorder, or the predisposition toward the disease or disorder. In some embodiments, the disease or disorder is a cancer, such as a solid tumor or hematologic malignancy.

The terms “treating”, “contacting” and “reacting,” in the context of a chemical reaction, mean adding or mixing two or more reagents under appropriate conditions to produce the indicated and/or desired product. It should be appreciated that the reaction which produces the indicated and/or desired product may not necessarily result directly from the combination of two reagents which were initially added, i.e., there may be one or more intermediates which are produced in the mixture and ultimately lead to the formation of the indicated and/or desired product.

The term “effective amount” refers to an amount or dose of a PI3K and/or PIKK inhibitor sufficient to generally bring about therapeutic benefits in patients in need of treatment for a disease or disorder involving PI3Ks and/or PIKKs. An effective amount or dose of an active ingredient in the present invention can be determined by conventional methods, such as modeling, dose escalation studies, or clinical trials, in conjunction with conventional influencing factors, such as the mode or route of dosing or administration, the pharmacokinetics of the pharmaceutical ingredients, the severity and course of the disease or disorder, the prior or ongoing therapies for the subject, the subject's health status and response to the drug, and the judgment of the attending physician. In the case of cancer, the effective amount can cause any one of the visible or detectable changes of the subject as described above with regard to the definition of “treating” or “treatment” or “preventing”. For example, an effective amount is capable of reducing the number of cancer or tumor cells; reducing the size of tumors; inhibiting or preventing the invasion of tumor cells into peripheral organs, for example, the spread of tumors into soft tissues or bone; inhibiting or preventing the metastasis of tumors; inhibiting or preventing the growth of tumors; alleviating one or more symptoms associated with the cancer to some extent; reducing morbidity and mortality; improving quality of life; or a combination of the above effects. An effective amount can be a dosage that reduces the symptoms of a disease by inhibiting the PI3K and/or PIKK activity. For the treatment of cancers, the effects of in-vivo experiments can be measured by assessments such as survival, time to progression (TTP), response rate (RR), response duration, and/or quality of life. Those skilled in the art have realized that the effective amount can be varied depending on the route of administration, the dosage of excipients, and the combination with other drugs.

The term “inhibit”, “inhibiting” or “inhibition” refers to a decrease in the baseline activity of a biological activity or process. The term “inhibiting the activity of PI3K and/or PIKK” is an actual pharmaceutical activity for the purposes of the present invention, which means that direct or indirect responses to the presence of the drug conjugate of the present invention result in a decrease in the PI3K and/or PIKK activity relative to the PI3K and/or PIKK activity in the absence of the drug conjugate of the present invention. The decrease in activity may be caused by the direct interaction of the drug conjugate of the present invention with PI3K and/or PIKK, or by the interaction of the drug conjugate of the present invention with one or more other factors, which ultimately affects the PI3K and/or PIKK activity. For example, the drug conjugate of the present invention may reduce the PI3K and/or PIKK activity by directly binding to PI3K and/or PIKK, by directly or indirectly affecting another factor, or by directly or indirectly reducing the amount of PI3K and/or PIKK present in a cell or an organism.

In general, the term “about” is used herein to modify a numerical value above or below the stated value by a variance of 20%.

Technical and scientific terms as used and not specifically defined herein have the meaning commonly understood by those skilled in the art to which the present invention belongs.

All numerical ranges herein shall be interpreted as disclosing each numerical value and subset of numerical values within the range, regardless of whether they are specifically otherwise disclosed. For example, when referring to any numerical range, it should be regarded as referring to every numerical value within the numerical range, for example, every integer within the numerical range. For example, C₁₋₆ as used herein represents the inclusion of 1, 2, 3, 4, 5, or 6 C. The present invention relates to all values falling within the ranges, all smaller ranges and the upper or lower limits of the numerical range.

In the present invention, unless otherwise specified, the laboratory operation steps for cell culture, molecular genetics, nucleic acid chemistry, and immunology used herein are all conventional steps widely used in the corresponding fields.

The term “amino acid residue” refers to an amino acid unit in a polypeptide, that is, the part remained after dehydration of an amino acid linked by a peptide bond. The abbreviation for an amino acid residue is a standard three-letter and/or one-letter code used in the art to refer to one of the commonly used amino acids. For example, the amino acids forming amino acid residues can be selected from phenylalanine (Phe), tyrosine (Tyr), leucine (Leu), glycine (Gly), alanine (Ala), valine (Val), lysine (Lys), citrulline (Cit), serine (Ser), glutamic acid (Glu), aspartic acid (Asp), asparagine (Asn), isoleucine (Ile), arginine (Arg), proline (Pro), glutamine (Gln), etc. The amino acids can be L- or D-isomers, preferably L-isomers.

Unless explicitly stated otherwise, singular forms used in the present application (including the claims) include the corresponding plural forms thereof.

When used to connect two or more optional items, the term “and/or” should be understood to mean any one of the optional items or any two or more of the optional items.

The term “comprising” or “including” as used in the present invention means to include the mentioned elements, integers, or steps, but does not exclude any other elements, integers, or steps. As used herein, when the term “comprising” or “including” is used, unless otherwise stated, examples consisting of the mentioned elements, integers, or steps are also encompassed. For example, when referring to an antibody variable region “comprising” a specific sequence, it is also intended to encompass an antibody variable region composed of the specific sequence.

The term “subject” or “patient” as used herein means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and so on. The term “subject” or “patient” does not denote a particular age or sex. In some embodiments, the subject or patient is a human.

In the present invention, the term “conjugate” refers to a substance obtained by linking a payload or active molecule to a targeting moiety (e.g., an antibody or an antigen-binding fragment thereof that specifically binds to EGFR). In some embodiments of the present invention, the payload or active molecule is attached to the targeting moiety via a linker. Preferably, the linker can be cleaved in a specific environment (e.g., a low pH environment in a cell) or under a specific action (e.g., the action of a lysosomal protease), thereby releasing the active molecule from the targeting moiety. In some embodiments of the present invention, the linker comprises a cleavable unit, such as a peptide or a disulfide bond.

The term “stretcher group” is a part of the linker, which is used to link the target moiety (e.g., an antibody or an antigen-binding fragment thereof that specifically binds to EGFR) with the remaining part of the linker.

“Bridged spacer group” described herein refers to a group that helps to separate a part of the linker from other moieties (such as separate a stretcher group of the present invention from the Lp group). In some embodiments, the bridged spacer group can lead to a change in the properties of the linker-payload compound of the present invention. In some embodiments, the bridged spacer group can modulate the hydrophilicity of the linker-payload compound and provide improved overall antibody-drug conjugate properties.

In some embodiments, the payload or active molecule is an active drug molecule. In the present invention, the term “active drug molecule” refers to a substance that can regulate biological processes, especially change or prevent pathological processes, particularly refers to a substance that inhibits or prevents cell functions and/or causes cell death or destruction. In some embodiments of the present invention, the bioactive substance or active drug molecule in a conjugate is a molecule with anti-tumor biological activity, particularly a PI3K and/or PIKK inhibitor, more preferably a PI3K and/or PIKK inhibitor as defined in the present invention.

In the present invention, the term “linker” refers to a fragment that links an active drug molecule to a targeting moiety (e.g., an antibody or an antigen-binding fragment thereof that specifically binds to EGFR). It should be understood that a linker, prior to linking to a targeting moiety (e.g., an antibody or an antigen-binding fragment thereof that specifically binds to EGFR), has a functional group that can form a bond with a functional group of the targeting moiety. Non-limiting examples of linkers include those involved in the embodiments and examples of the present invention.

In the present invention, when an active molecule in an immunoconjugate is an active drug molecule, the immunoconjugate can be referred to as an “antibody-drug conjugate (ADC)”. In the present invention, the “drug conjugate” and “antibody-drug conjugate” can be used interchangeably.

The term “linker-payload” refers to a compound formed by attaching a payload, such as an active drug molecule (e.g., a small molecule drug), to a linker.

The term “EGFR” refers to epidermal growth factor receptor, which refers to any natural form of EGFR derived from any vertebrate source, including mammals such as primates (e.g., humans) and rodents (e.g., mice and rats). The term encompasses “full-length” unprocessed EGFR as well as any form of EGFR or any fragment thereof resulting from intracellular processing (e.g., human natural full-length EGFR; NCBI: reference sequence: NP_005219.2). The term further includes functional variants or fragments of naturally occurring EGFR, including, but not limited to, splice variants, allelic variants, and isoforms that retain one or more biological functions of naturally occurring EGFR (i.e., variants and fragments are also included, unless the context indicates that the term is used only for wild-type proteins).

The term “DAR” refers to the ratio of a drug moiety (D) conjugated to the targeting moiety described herein (an antibody or an antigen-binding fragment thereof that specifically binds to EGFR) to the targeting moiety (an antibody or an antigen-binding fragment thereof that specifically binds to EGFR). In some embodiments described herein, the DAR can be determined by p in formula (I), for example, the DAR can be 1 to 16, for example, 2-16, 4-16, 5-12, 6-10, 2-8, 3-8, 2-6, 4-6, or 6-10, such as about 2, about 3, about 4, about 5, about 6, about 7, about 8, about 9, about 10, about 11, about 12, about 13, about 14, or about 15. The DAR can also be calculated as an average DAR of a molecule population in a product, i.e., the overall ratio of a drug moiety (D) to an Ab moiety described herein in a product as measured by a detection method (e.g., by conventional methods such as mass spectrometry and/or HIC-HPLC), wherein the drug moiety is conjugated to the Ab moiety; and the DAR is referred to herein as an average DAR. In this case, a mixture of the conjugates is described in formula (I), wherein more than 60%, 65%, 70%, 75%, 80%, 85%, 90%, 95%, 96%, 97% or 98% of the conjugates have a p value ranging from integers or decimals that may be selected from 1 to 16. In some embodiments, the average DAR value of the conjugate of the present invention is an integer or decimal of 1 to 16, e.g., 2-16, 4-16, 5-12, 6-10, 2-8, 3-8, 2-6, 4-6 or 6-10, e.g., 1.0-8.0 or 2.0-6.0, e.g., about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9 or about 10.0, and any range with any two of these values as endpoints. It should be understood that when referring to an average DAR value, the ADC of the present invention refers to an ADC molecule population or an ADC molecule mixture, containing ADC molecules with the same and/or different DARs.

As used herein, the “pharmaceutically acceptable” and “pharmaceutical” can be used interchangeably unless there is any contradiction in the context.

As used herein, the term “cancer” refers to a cellular disorder characterized by uncontrolled or dysregulated cell proliferation, decreased cellular differentiation, inappropriate invasion of surrounding tissues, and/or ability to establish new growth foci at other sites. The term “cancer” includes, but is not limited to, solid tumors and hematologic malignancies, such as leukemia, lymphoma, or myeloma. The term “cancer” includes cancers of skin, tissues, organs, bones, cartilage, blood, and vessels. The term “cancer” further includes primary cancer, and metastatic cancer, recurrent cancer, and refractory cancer.

The term “pharmaceutical composition” refers to a composition that exists in a form that allows the biological activity of the active ingredient(s) contained therein to be effective and does not contain additional components that have unacceptable toxicity to the subject to whom the composition is administered. In addition to the active ingredient, e.g., ADCs, it generally contains components such as pharmaceutically acceptable carriers, stabilizers, diluents, dispersants, suspending agents, thickeners, and/or excipients. The pharmaceutical compositions provided herein are in a form that permits administration and subsequently provides the desired biological activity of active ingredient(s) and/or achieves therapeutic effects.

The terms “pharmaceutically acceptable carrier” and “physiologically acceptable carrier” refer to carriers or diluents that do not cause significant irritation to a subject and do not abrogate the biological activity and properties of the administered active ingredients such as ADC compounds or compositions and/or any additional therapeutic agents in the compositions. The pharmaceutically acceptable carrier can enhance or stabilize the composition, or can be used to facilitate the preparation of the composition.

The term “antibody” refers to an antibody in any form having a desired bioactivity. Therefore, it is used in the broadest sense, specifically including but not limited to a monoclonal antibody (including a full-length monoclonal antibody), a polyclonal antibody, a multispecific antibody (such as a bispecific antibody), a humanized antibody, a human antibody, a chimeric antibody, a CrossMab antibody, or a camelized single-domain antibody.

The term “monoclonal antibody” refers to an antibody obtained from a population of substantially homogeneous antibodies, i.e., the various antibodies constituting the population are identical except for possible naturally occurring mutations that may be present in minor amounts. The modifier “monoclonal” indicates the feature of the antibody as being obtained from a substantially homogeneous population of antibodies, and is not to be constructed as requiring any particular method to produce the antibody.

The term “isolated antibody or antigen-binding fragment” refers to the purified state of the antibody or antigen-binding fragment. For example, “isolated” may mean that the molecule is substantially free of other biomolecules, such as nucleic acids, proteins, lipids, sugars or other substances, such as cell debris and growth medium. However, as is known to a person skilled in the art, the term “isolated” does not mean the complete absence of such substances unless they are present in an amount that significantly interferes with the experimental or therapeutic application of the antibodies described herein. In some embodiments, the isolated antibody or antigen-binding fragment can have a purity of greater than 95%, greater than 96%, greater than 97%, greater than 98%, or greater than 99%, which purity is determined by, for example, electrophoresis (e.g., SDS-PAGE, isoelectric focusing (IEF), or capillary electrophoresis) or chromatography (e.g., ion exchange or reverse phase HPLC). For a review of methods of evaluating antibody purity, see, for example, Flatman, S. et al., J. Chrom. B 848 (2007) 79-87.

The terms “full-length antibody” and “intact antibody” can be used interchangeably herein and refer to an antibody that has a structure substantially similar to a native antibody structure. “Native antibodies” refer to naturally occurring immunoglobulin molecules with varied structures. For example, native IgG class antibodies are heterotetrameric glycoproteins of approximately 150,000 daltons, composed of two light chains and two heavy chains interconnected by disulfide bonds. Each of the heavy chains is comprised of a heavy chain variable region (abbreviated herein as VH) and a heavy chain constant region. The heavy chain constant region consists of 3 domains (CH1, CH2, and CH3). Each of the light chains is comprised of a light chain variable region (abbreviated herein as VL) and a light chain constant region. The light chain constant region consists of the domain CL. The VH and VL regions can be further divided into hypervariable regions (complementarity determining regions or CDRs) interspersed with regions that are more conserved (framework regions or FRs). Each VH or VL consists of three CDRs and four FRs, arranged in the following order from N-terminus to C-terminus: FR1, CDR1, FR2, CDR2, FR3, CDR3, and FR4. The constant region is not directly involved in the binding of an antibody to an antigen, but shows multiple effector functions. The heavy chain of an antibody can be classified into one of five types, i.e., a (IgA), δ (IgD), ε (IgE), γ (IgG), or μ(IgM), some of which can be further divided into subtypes, such as γ1 (IgG1), γ2 (IgG2), γ3 (IgG3), γ4 (IgG4), α1 (IgA1), and α2 (IgA2). The light chains of the antibody can be classified into one of two types, called κ (kappa) and λ (lambda), based on the amino acid sequences of their constant regions.

A “complementary determining region” or “CDR region” or “CDR” is a region in an antibody variable region, which is hypervariable in sequence and forms a structurally established loop (“hypervariable loop”) and/or contains an antigen contact residue (“antigen contact point”). CDR is mainly responsible for binding to epitopes. CDRs of heavy chain and light chain are generally called CDR1, CDR2, and CDR3, which are numbered sequentially from the N-terminus. The CDRs located in the antibody heavy chain variable region are called HCDR1, HCDR2, and HCDR3, respectively, while the CDRs located in the antibody light chain variable region are called LCDR1, LCDR2, and LCDR3, respectively. Each VH or VL consists of three CDRs and 4 FRs, which are arranged in the following order from the amino terminus to the carboxyl terminus: FR1, CDR1, FR2, CDR2, FR3, CDR3, and FR4. In a designated VH or VL amino acid sequence, the exact amino acid sequence boundary of each CDR can be determined by using any one of or a combination of various well-known schemes, including, for example: Chothia scheme (Chothia et al., Canonical structures for the hypervariable regions of immunoglobulins, Journal of Molecular Biology, 196, 901-917 (1987)); Kabat scheme (Kabat et al., Sequences of Proteins of Immunological Interest, 4th edition, U.S. Department of Health and Human Services, National Institutes of Health (1987)), AbM (University of Bath) and Contact (University College London); North scheme (North et al., A New Clustering of Antibody CDR Loop Conformations, Journal of Molecular Biology, 406, 228-256 (2011)). The boundary of the CDRs of the antibody in the present invention can be determined according to any schemes or a combination thereof in the art and personal evaluation. In some embodiments, the CDRs of the antibody of the present invention can be determined according to the Kabat scheme.

The term “antibody fragment” refers to a molecule other than a full antibody, which contains a portion of the full antibody that binds to the antigen that antigen bound by the full antibody. Examples of an antibody fragment include, but are not limited to, Fv, such as a disulfide-stabilized Fv fragment (dsFv), (dsFv)₂, bispecific dsFv (dsFv-dsFv′), Fab, Fab′, Fab′-SH, and F(ab′)₂; a diabody such as a disulfide-stabilized diabody (ds diabody), a triabody, a tetrabody, and a crossFab fragment; a linear antibody; a single-chain antibody molecule (e.g., scFv) and an scFv dimer (bivalent diabody); and a single-domain antibody. With regard to a review of certain antibody fragments, reference is made to Hudson et al., Nat Med 9, 129-134 (2003). Antibody fragments can be prepared by a variety of techniques including, but not limited to, proteolytic digestion of intact antibodies and production by recombinant host cells (e.g., Escherichia coli or phage).

The term “antigen-binding fragment” includes a fragment of an antibody that binds to the antigen. Generally, the antigen-binding fragment includes at least one fragment (such as one or more CDRs) of the antigen-binding region or variable region of the antibody, and maintains at least some of the binding properties of the antibody. Examples of an antigen-binding fragment include, but are not limited to, Fv, such as a disulfide-stabilized Fv fragment (dsFv), (dsFv)₂, bispecific dsFv (dsFv-dsFv′), Fab, Fab′, Fab′-SH, and F(ab′)₂; a diabody such as a disulfide-stabilized diabody (ds diabody), a triabody, a tetrabody, and a crossFab fragment; a linear antibody; a single-chain antibody molecule (e.g., scFv) and an scFv dimer (bivalent diabody); a single-domain antibody; a nanobody and a multispecific antibody. When the antigen binding activity is expressed on a molar concentration basis, the antigen binding fragments generally maintain at least 10% of the antigen binding activity of the antibody from which they are derived. Preferably, the antigen-binding fragments maintain at least 20%, 50%, 70%, 80%, 90%, 95%, or 100% or more of the antigen binding activity of the antibody from which they are derived.

The term “biosimilar” refers to a biological product that is highly similar to an approved reference biological product (Reference Biologic) in terms of quality, safety and efficacy, and has no clinically meaningful differences. For example, a “biosimilar of cetuximab” is a monoclonal antibody that is highly similar to cetuximab in terms of quality, safety and efficacy, and has no clinically meaningful differences. In some embodiments, the biosimilar has the same heavy and light chain amino acid sequences as the monoclonal antibody, but differs in post-translational modifications such as glycosylation.

The term “conservative substitution” or “conservative amino acid substitution” refers to the substitution of one or more amino acids with one or more chemically or functionally similar amino acids. Conservative substitutions tables providing similar amino acids are well known in the art. Polypeptide sequences having such substitutions are referred to as “conservatively modified variants” or “variants”. Such conservatively modified variants are in addition to, and do not exclude polymorphic variants, interspecific homologs and alleles. Table A illustrates conservative substitutions of one another.

TABLE A

| Original | | Preferred conservative |
|---|---|---|
| residue | Exemplary substitution | amino acid substitution |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | norleucine | |
| Leu (L) | norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | norleucine | |

### BRIEF DESCRIPTION OF THE DRAWINGS

FIG. 1 Effects of the antibody-drug conjugates of the present invention on growth of NCI-H1650 subcutaneous xenograft model.

### SPECIFIC EMBODIMENTS

### Embodiment I

Embodiment 1. An antibody-drug conjugate having a structure represented by formula (I):

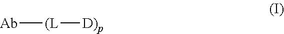

or a pharmaceutically acceptable salt thereof, or a solvate, a racemic mixture, an enantiomer, a diastereomer or a tautomer thereof, wherein
Ab is an antibody that specifically binds to EGFR or an antigen-binding fragment thereof; D is a PI3K and/or PIKK inhibitor;
L is a linker, which links Ab to D; and
p is an integer from 1 to 20.

Embodiment 2. The antibody-drug conjugate or the pharmaceutically acceptable salt thereof, or the solvate, the racemic mixture, the enantiomer, the diastereomer or the tautomer thereof according to embodiment 1, wherein the structure of the D is as represented by formula (II):

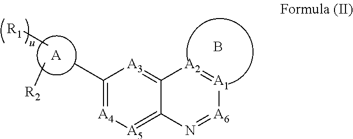

wherein
A₃, A₄, A₅, and A₆ are independently N or CR₅; R₅ is independently selected from: H, C₁₋₆ alkyl, C₃₋₉ cycloalkyl, —CN, —NH₂, —OH, —SH, —O(C₁₋₆ alkyl), halogen, or C₁₋₆ haloalkyl;

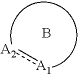

represents a 5- to 6-membered heteroaryl ring or a 3- to 8-membered heterocyclyl ring, and the 5- to 6-membered heteroaryl or 3- to 8-membered heterocyclyl is optionally substituted with one or more groups selected from oxo, ═NCN, or R₃;
ring A is 5- to 14-membered heteroaryl;
R₁, R₂, and R₃ are independently selected from: H, C₁₋₆ alkyl, C₂₋₈ alkenyl, C₂₋₈ alkynyl, C₆-C₁₀ aryl, C₃₋₉ cycloalkyl, —C(O)R^{a}, —C(O)OR^{b}, —CN, —C(O)NR^{c}R^{d}, —NR^{c}R^{d}, —NR^{c}C(O)R^{a}, —NR^{c}S(O)ₙR^{e}, —NR^{c}S(O)ₙNR^{f}R^{g}, —NR^{c}C(O)OR^{b}, —NR^{c}C(O)NR^{d}R^{e}, —NO₂, —OR^{b}, —SR^{b}, —S(O)ₙR^{e}, —S(O)ₙR^{c}R^{d}, halogen, C₁₋₆ haloalkyl, 5- to 6-membered heteroaryl, and 3- to 12-membered heterocyclyl;
R^{a}, R^{b}, R^{e}, R^{d}, R^{e}, R^{f}, and R^{g} are each independently selected from hydrogen, C₁₋₆ alkyl, C₂₋₈ alkenyl, C₂₋₈ alkynyl, C₆-C₁₀ aryl, C₃₋₉ cycloalkyl, C₁₋₆ haloalkyl, 5- to 6-membered heteroaryl, and 3- to 12-membered heterocyclyl;
or R^{a} and R^{e}, and/or R^{c} and R^{d}, and/or R^{c} and R^{e}, and/or R^{c} and R^{f}, and/or R^{d} and R^{e}, and/or R^{g} and R^{f} together with the atoms to which they are attached form 3- to 12-membered heterocyclyl;
wherein the C₁₋₆ alkyl, C₂₋₈ alkenyl, C₂₋₈ alkynyl, C₁₋₆ haloalkyl, C₆-C₁₀ aryl, C₃₋₉ cycloalkyl, 5- to 6-membered heteroaryl, and 3- to 12-membered heterocyclyl, at each occurrence, can each independently and optionally be substituted with one or more groups selected from: deuterium, C₁₋₆ alkyl, C₂₋₈ alkenyl, C₂₋₈ alkynyl, C₆-C₁₀ aryl, C₃₋₉ cycloalkyl, —C(O)R^{a}, —C(O)OR^{b}, —CN, —C(O)NR^{c}R^{d}, —NR^{c}R^{d}, —NR^{c}C(O)R^{a}, —NR^{c}S(O)ₙR^{e}, —NR^{c}S(O) NR^{f}R^{g}, —NR^{c}C(O)OR^{b}, —NR^{c}C(O)NR^{d}R^{e}, —NO₂, —C₁₋₆ alkylene-OR^{b}, —OR^{b}, —S(O)ₙR^{e}, —S(O)ₙR^{c}R^{d}, halogen, C₁₋₆ haloalkyl, 5- to 6-membered heteroaryl, and 3- to 8-membered heterocyclyl;
n, at each occurrence, is independently 0, 1, or 2;
u, at each occurrence, is independently 0, 1, 2, or 3; and
[symbol] represents a single bond or a double bond; and
wherein one of the R₁, R₂ or R₃, when present, is covalently linked to the L, and the valence of the atom at the linking site does not exceed the normal valence of the atom due to the one or more substituents to which it is linked.

Embodiment 3. The antibody-drug conjugate or the pharmaceutically acceptable salt thereof, or the solvate, the racemic mixture, the enantiomer, the diastereomer or the tautomer thereof according to embodiment 2, wherein

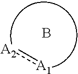

is selected from the following structures:

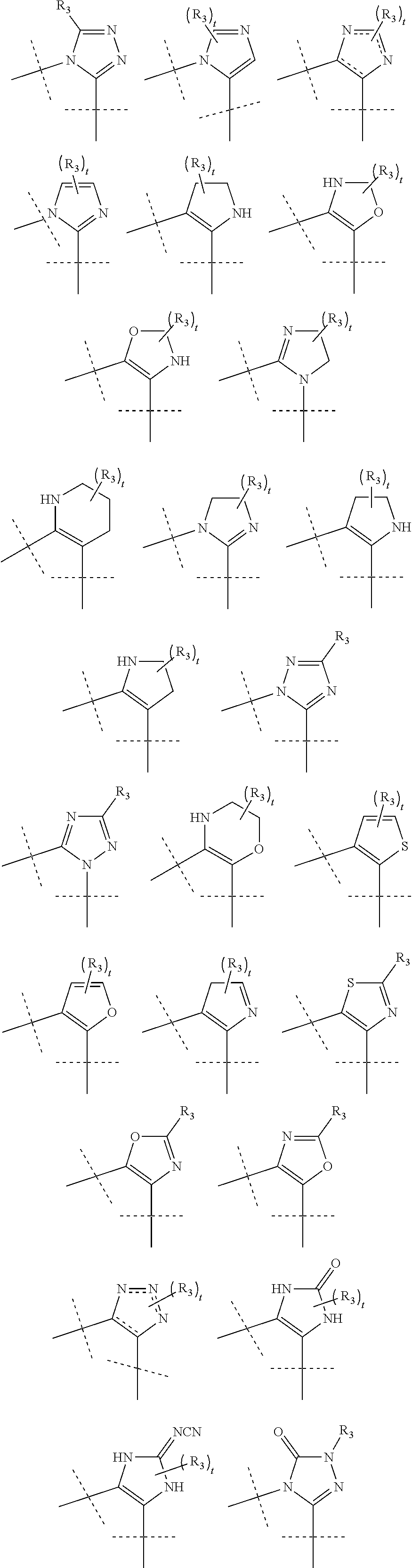

wherein t, at each occurrence, is independently 0, 1, 2, or 3; R³ is as defined in embodiment 2;
[symbol] represents a single bond or a double bond; and
the dashed lines represent the positions to which the rings are fused.

Embodiment 4. The antibody-drug conjugate or the pharmaceutically acceptable salt thereof, or the solvate, the racemic mixture, the enantiomer, the diastereomer or the tautomer thereof according to embodiment 3, wherein

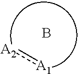

is selected from the following structures:

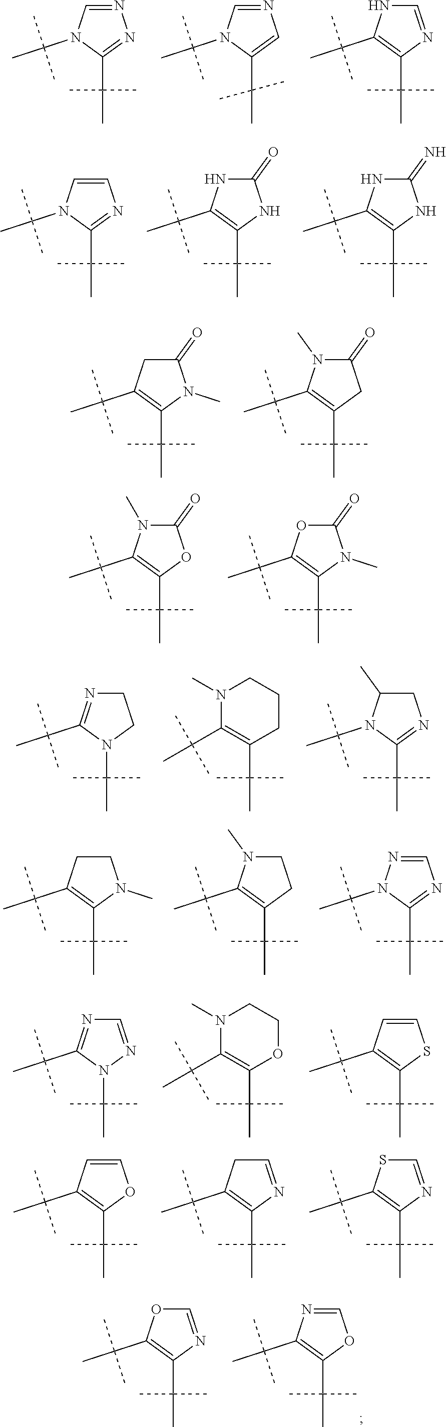

the above structures are optionally substituted with one or more R₃, for example, substituted with 1, 2 or 3 R³, and R³ is as defined in embodiment 2.

Embodiment 5. The antibody-drug conjugate or the pharmaceutically acceptable salt thereof, or the solvate, the racemic mixture, the enantiomer, the diastereomer or the tautomer thereof according to embodiment 3 or 4, wherein

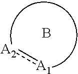

is selected from the following structures:

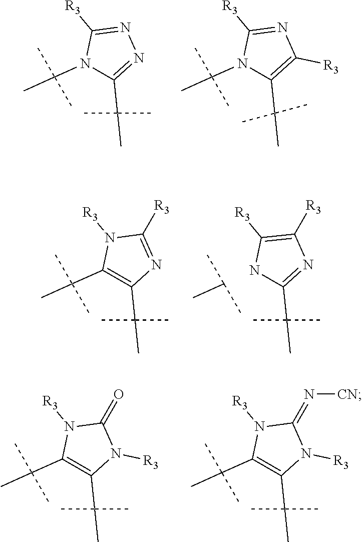

wherein R₃ is as defined in embodiment 2, and may be the same or different from each other.

Embodiment 6. The antibody-drug conjugate or the pharmaceutically acceptable salt thereof, or the solvate, the racemic mixture, the enantiomer, the diastereomer or the tautomer thereof according to any one of embodiments 2 to 5, wherein the ring A is derived from the following structures:

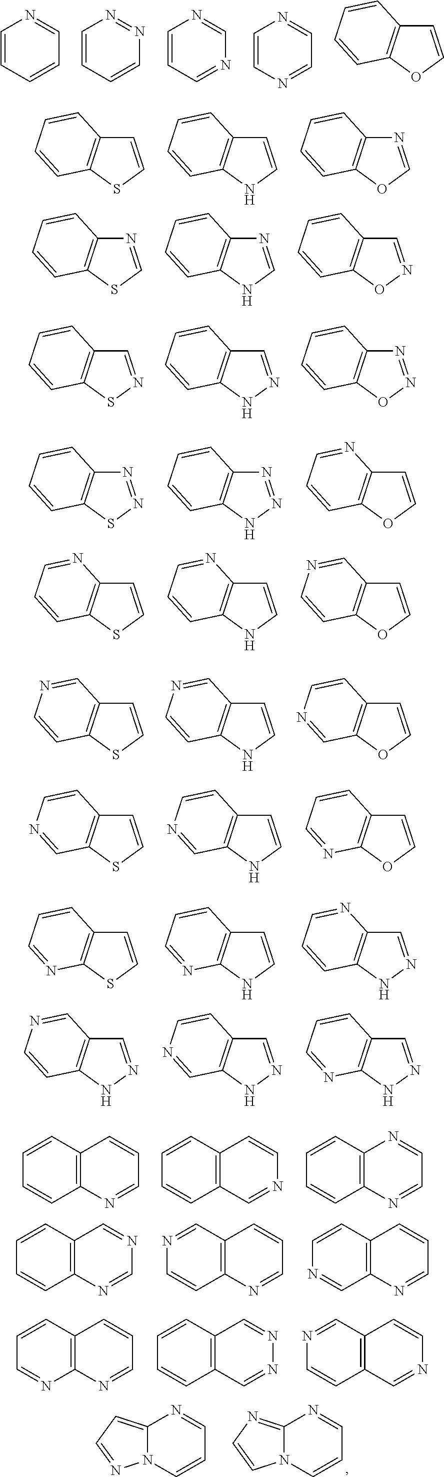

each of which is optionally substituted with u R₁ groups and R₂ group;
wherein u, R₁ and R₂ are as defined in embodiment 2.

Embodiment 7. The antibody-drug conjugate or the pharmaceutically acceptable salt thereof, or the solvate, the racemic mixture, the enantiomer, the diastereomer or the tautomer thereof according to any one of embodiments 2 to 6, wherein the ring A is derived from the following structures:

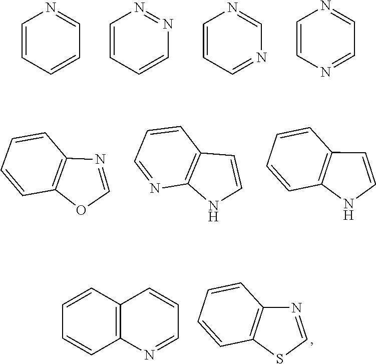

each of which is optionally substituted with u R₁ groups and R₂ group;
wherein u, R₁ and R₂ are as defined in embodiment 2.

Embodiment 8. The antibody-drug conjugate or the pharmaceutically acceptable salt thereof, or the solvate, the racemic mixture, the enantiomer, the diastereomer or the tautomer thereof according to any one of embodiments 2 to 7, wherein the structure of the ring A is:

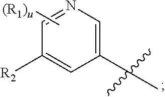

wherein R₁, R₂ and u are as defined in embodiment 2; and
the wavy line represents the site where the ring A is linked to the rest of the molecule.

In some embodiments, the structure of the ring A is:

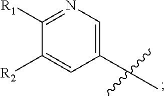

wherein R₁ and R₂ are as defined in embodiment 2; and
the wavy line represents the site where the ring A is linked to the rest of the molecule.

Embodiment 9. The antibody-drug conjugate or the pharmaceutically acceptable salt thereof, or the solvate, the racemic mixture, the enantiomer, the diastereomer or the tautomer thereof according to any one of embodiments 2 to 8, wherein A₃ is N or CH.

Embodiment 10. The antibody-drug conjugate or the pharmaceutically acceptable salt thereof, or the solvate, the racemic mixture, the enantiomer, the diastereomer or the tautomer thereof according to any one of embodiments 2 to 9, wherein A₄ is N or CH.

Embodiment 11. The antibody-drug conjugate or the pharmaceutically acceptable salt thereof, or the solvate, the racemic mixture, the enantiomer, the diastereomer or the tautomer thereof according to any one of embodiments 2 to 10, wherein A₅ is N or CH.

Embodiment 12. The antibody-drug conjugate or the pharmaceutically acceptable salt thereof, or the solvate, the racemic mixture, the enantiomer, the diastereomer or the tautomer thereof according to any one of embodiments 2 to 11, wherein A₆ is N or CH.

Embodiment 13. The antibody-drug conjugate or the pharmaceutically acceptable salt thereof, or the solvate, the racemic mixture, the enantiomer, the diastereomer or the tautomer thereof according to any one of embodiments 2 to 12, wherein A₃ is N, and A₄, A₅ and A₆ are all CH.

Embodiment 14. The antibody-drug conjugate or the pharmaceutically acceptable salt thereof, or the solvate, the racemic mixture, the enantiomer, the diastereomer or the tautomer thereof according to any one of embodiments 2 to 13, wherein A₃, A₄, A₅ and A₆ are all CH.

Embodiment 15. The antibody-drug conjugate or the pharmaceutically acceptable salt thereof, or the solvate, the racemic mixture, the enantiomer, the diastereomer or the tautomer thereof according to any one of embodiments 2 to 14, wherein R₁ is selected from: —C₁₋₆ alkyl, —O(C₁₋₆ alkyl), —OC₁₋₆ haloalkyl, or C₃₋₉ cycloalkyl, wherein the C₁₋₆ alkyl may optionally be substituted with one or more deuterium; in some specific embodiments, R₁ is selected from —C₁₋₆ alkyl, —OC₁₋₆ haloalkyl, or —O(C₁₋₆ alkyl), particularly —O(C₁₋₆ alkyl) and —OC₁₋₆ haloalkyl, e.g., —OCH₃ or —OCH₂CF₃.

Embodiment 16. The antibody-drug conjugate or the pharmaceutically acceptable salt thereof, or the solvate, the racemic mixture, the enantiomer, the diastereomer or the tautomer thereof according to any one of embodiments 2 to 15, wherein the linker L is linked to an active drug molecule via R₂, and R₂ is

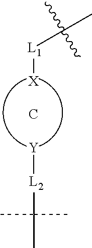

wherein [symbol] represents the site to which the ring A is linked;
- - - represents the site to which the linker L is linked;
L₁ is absent or is selected from: —C₁₋₆ alkylene-, —C(O)—, —C(O)O—, —NH—, —NHC(O)—, —NHS(O)ₙ₂—, —NHC(O)O—, —C(O)NH—, —C₁₋₆ alkylene-O—, —O—, —S—, or —S(O)ₙ₂—;
X and Y are each independently C, CH, or N;
L₂ is absent or is selected from: —C₁₋₆ alkylene-NH—, —NH—, —C₁₋₆ alkylene-O—, or —O—; ring C is selected from C₆-C₁₀ aryl, 5- to 6-membered heteroaryl, or a 3- to 12-membered heterocyclic ring, each of which is independently and optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH; and
n₂, at each occurrence, is independently 1 or 2.

Embodiment 17. The antibody-drug conjugate or the pharmaceutically acceptable salt thereof, or the solvate, the racemic mixture, the enantiomer, the diastereomer or the tautomer thereof according to embodiment 16, wherein L₁ is —NHS(O)₂—, X and Y are both N, and L₂ is absent.

Embodiment 18. The antibody-drug conjugate or the pharmaceutically acceptable salt thereof, or the solvate, the racemic mixture, the enantiomer, the diastereomer or the tautomer thereof according to embodiment 16, wherein L₁ is —NHS(O)₂—, X is —CH— or N, Y is N, and L₂ is —C₁₋₆ alkylene-NH—, —NH—, —C₁₋₆ alkylene-O—, or —O—.

Embodiment 19. The antibody-drug conjugate or the pharmaceutically acceptable salt thereof, or the solvate, the racemic mixture, the enantiomer, the diastereomer or the tautomer thereof according to any one of embodiments 16 to 18, wherein the ring C is a 3- to 12-membered heterocyclic ring which is optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH.

Embodiment 20. The antibody-drug conjugate or the pharmaceutically acceptable salt thereof, or the solvate, the racemic mixture, the enantiomer, the diastereomer or the tautomer thereof according to embodiment 19, wherein
the structure of

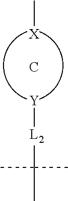

is selected from:

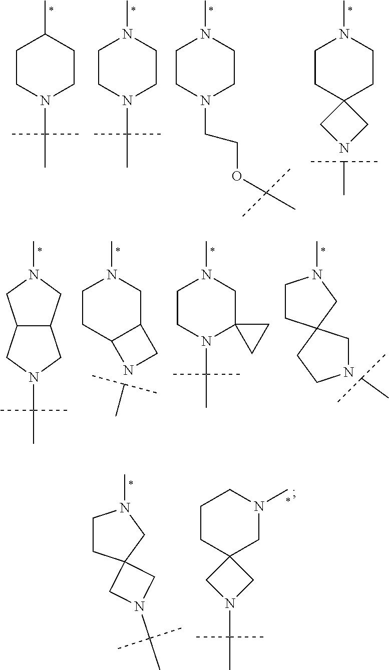

in some specific embodiments, the structure of

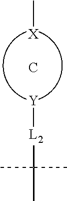

is selected from:

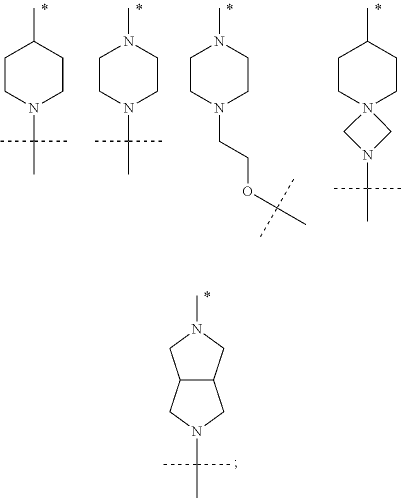

wherein * represents the site to which L₁ is linked; and
- - - represents the site to which the linker L is linked.

Embodiment 21. The antibody-drug conjugate or the pharmaceutically acceptable salt thereof, or the solvate, the racemic mixture, the enantiomer, the diastereomer or the tautomer thereof according to any one of embodiments 2 to 15, wherein the linker L is linked to an active drug molecule via R₁ or R₃, and R₂ is selected from:

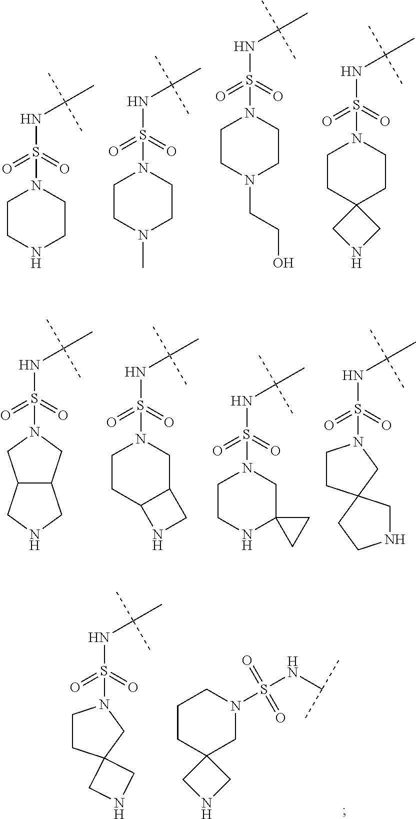

preferably, the structure of R₂ is:

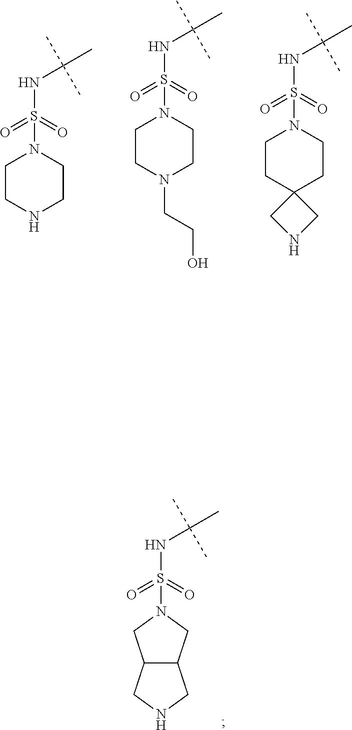

and
the dashed lines represent the position where R₂ is linked to the ring A.

Embodiment 22. The antibody-drug conjugate or the pharmaceutically acceptable salt thereof, or the solvate, the racemic mixture, the enantiomer, the diastereomer or the tautomer thereof according to any one of embodiments 2 to 21, wherein each R₃ is independently selected from: H, CN, NO₂, halogen, C₁₋₆ alkyl, C₆-C₁₀ aryl, C₃₋₉ cycloalkyl, 5- to 6-membered heteroaryl, and 3- to 12-membered heterocyclyl, wherein the C₁₋₆ alkyl, C₆-C₁₀ aryl, C₃₋₉ cycloalkyl, 5- to 6-membered heteroaryl, and 3- to 12-membered heterocyclyl can each optionally be substituted with one or more groups independently selected from: deuterium, C₁₋₆ alkyl, C₂₋₈ alkenyl, C₂₋₈ alkynyl, C₃₋₉ cycloalkyl, —CN, —C(O)NH₂, —NH₂, —NHC(O)C₁₋₆ alkyl, —NO₂, —OH, —C₁₋₆ alkylene-OH, halogen, C₁₋₆ haloalkyl, or —C₁₋₆ alkylene-OC₁₋₆ alkyl.

Embodiment 23. The antibody-drug conjugate or the pharmaceutically acceptable salt thereof, or the solvate, the racemic mixture, the enantiomer, the diastereomer or the tautomer thereof according to embodiment 22, wherein each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, C₁₋₆ alkyl, —CN, —NH₂, —NO₂, —OH, —C₁₋₆ alkylene-OH, halogen, C₁₋₆ haloalkyl, or —C₁₋₆ alkylene-OC₁₋₆alkyl;

In some embodiments, each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, —OH, F, or —C₁₋₆ alkylene-OH; and

In some specific embodiments, each R³ is independently selected from: hydrogen, —CH₃, —CD₃, —CH(CH₃)₂, —CH₂CF₃, or the following groups:

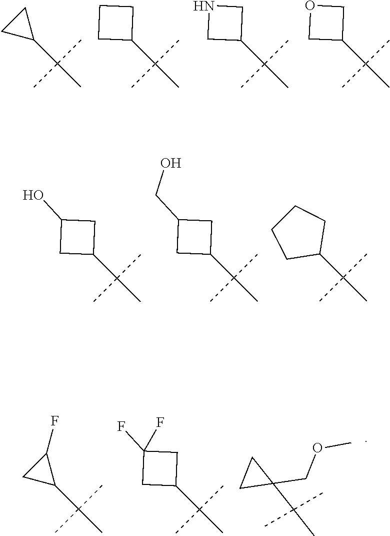

Embodiment 24. The antibody-drug conjugate or the pharmaceutically acceptable salt thereof, or the solvate, the racemic mixture, the enantiomer, the diastereomer or the tautomer thereof according to any one of embodiments 2 to 15 or 21, wherein the linker L is linked to an active drug molecule via R₃, and the structure of R₃ is:

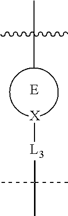

wherein [symbol] represents the site to which ring B is linked;
- - - represents the site to which the linker L is linked;
X is —CH— or N;
L₃ is absent or is selected from —C₁₋₆ alkylene-NH—, —NH—, —C₁₋₆ alkylene-O—, or —O—;
ring E is selected from C₃₋₉ cycloalkyl and a 3- to 12-membered heterocyclic ring, which are optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH;
in some embodiments, X is N, and L₃ is absent;
in some embodiments, X is —CH—, and L₃ is —C₁₋₆ alkylene-O— or —O—.

Embodiment 25. The antibody-drug conjugate or the pharmaceutically acceptable salt thereof, or the solvate, the racemic mixture, the enantiomer, the diastereomer or the tautomer thereof according to embodiment 24, wherein the ring E is selected from:

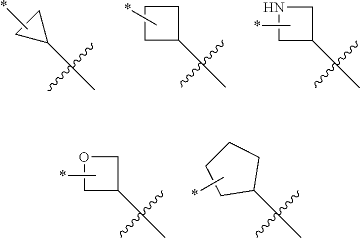

wherein [symbol] represents the site to which ring B is linked; and
* represents the site to which L₃ is linked.

Embodiment 26. The antibody-drug conjugate or the pharmaceutically acceptable salt thereof, or the solvate, the racemic mixture, the enantiomer, the diastereomer or the tautomer thereof according to any one of embodiments 2 to 15, wherein the structure of the D is:

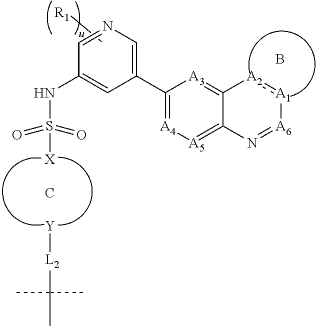

- - - represents the site to which the linker L is linked;
X and Y are each independently C, —CH—, or N;
L₂ is absent or is selected from —C₁₋₆ alkylene-NH—, —NH—, —C₁₋₆ alkylene-O—, or —O—;
ring C is selected from C₆-C₁₀ aryl, 5- to 6-membered heteroaryl, or a 3- to 12-membered heterocyclic ring, each of which is independently and optionally substituted with one or more groups selected from: —CN, —NO₂, halogen, —C₁₋₆ alkyl, —C₁₋₆ haloalkyl, C₃₋₉ cycloal, —C₁₋₆ alkylene-NH₂, —NH₂, —C₁₋₆ alkylene-OH, or —OH;
u, at each occurrence, is independently 0, 1, 2, or 3; and
R₁, A₃, A₄, A₅, A₆ and

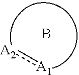

are as defined in embodiment 2.

Embodiment 27. The antibody-drug conjugate or the pharmaceutically acceptable salt thereof, or the solvate, the racemic mixture, the enantiomer, the diastereomer or the tautomer thereof according to embodiment 26, wherein A₃, A₄, A₅ and A₆ are all CH, or A₃ is N, and A₄, A₅ and A₆ are all CH.

Embodiment 28. The antibody-drug conjugate or the pharmaceutically acceptable salt thereof, or the solvate, the racemic mixture, the enantiomer, the diastereomer or the tautomer thereof according to embodiment 26 or 27, wherein

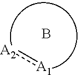

is selected from the following structures:

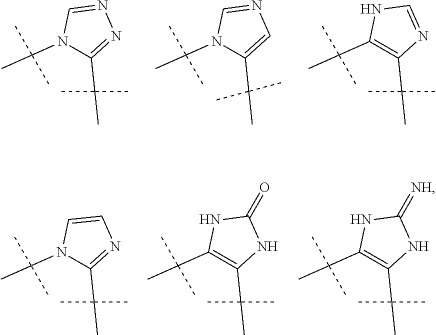

each of which is optionally substituted with one or more R₃, wherein R₃ is as defined in embodiment 2.

Embodiment 29. The antibody-drug conjugate or the pharmaceutically acceptable salt thereof, or the solvate, the racemic mixture, the enantiomer, the diastereomer or the tautomer thereof according to embodiment 28, wherein each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, C₁₋₆ alkyl, —CN, —NH₂, —NO₂, —OH, —C₁₋₆ alkylene-OH, halogen, C₁₋₆ haloalkyl, or —C₁₋₆ alkylene-OC₁₋₆ alkyl.

Embodiment 30. The antibody-drug conjugate or the pharmaceutically acceptable salt thereof, or the solvate, the racemic mixture, the enantiomer, the diastereomer or the tautomer thereof according to embodiment 29, wherein each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, —OH, F, or —C₁₋₆ alkylene-OH; and
in some specific embodiments, each R₃ is independently selected from: hydrogen, —CH₃, —CD₃, —CH(CH₃)₂, —CH₂CF₃, or the following groups:

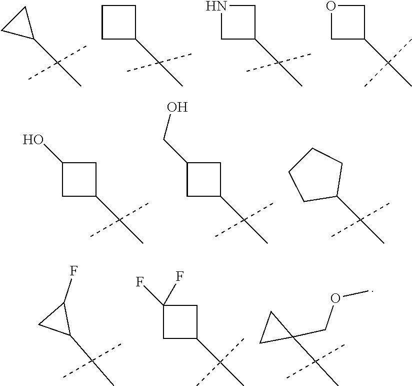

Embodiment 31. The antibody-drug conjugate or the pharmaceutically acceptable salt thereof, or the solvate, the racemic mixture, the enantiomer, the diastereomer or the tautomer thereof according to any one of embodiments 26 to 30, wherein

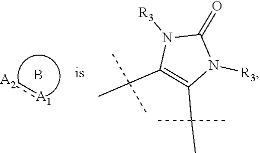

and each R³ is independently as defined in embodiments 26 to 30.

Embodiment 32. The antibody-drug conjugate or the pharmaceutically acceptable salt thereof, or the solvate, the racemic mixture, the enantiomer, the diastereomer or the tautomer thereof according to any one of embodiments 26 to 30, wherein

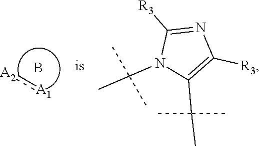

and each R₃ is independently as defined in embodiments 26 to 30; and
in some specific embodiments, each R₃ is independently H, —CH₃, or C₃₋₉ cycloalkyl.

Embodiment 33. The antibody-drug conjugate or the pharmaceutically acceptable salt thereof, or the solvate, the racemic mixture, the enantiomer, the diastereomer or the tautomer thereof according to any one of embodiments 26 to 32, wherein X and Y are both N, and L₂ is absent.

Embodiment 34. The antibody-drug conjugate or the pharmaceutically acceptable salt thereof, or the solvate, the racemic mixture, the enantiomer, the diastereomer or the tautomer thereof according to any one of embodiments 26 to 32, wherein X is —CH— or N, Y is N, and L₂ is —C₁₋₆ alkylene-NH—, —NH—, —C₁₋₆ alkylene-O— or —O—.

Embodiment 35. The antibody-drug conjugate or the pharmaceutically acceptable salt thereof, or the solvate, the racemic mixture, the enantiomer, the diastereomer or the tautomer thereof according to any one of embodiments 26 to 34, wherein the ring C is a 3- to 12-membered heterocyclic ring which is optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH.

Embodiment 36. The antibody-drug conjugate or the pharmaceutically acceptable salt thereof, or the solvate, the racemic mixture, the enantiomer, the diastereomer or the tautomer thereof according to embodiment 35, wherein the structure of

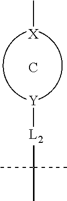

is selected from:

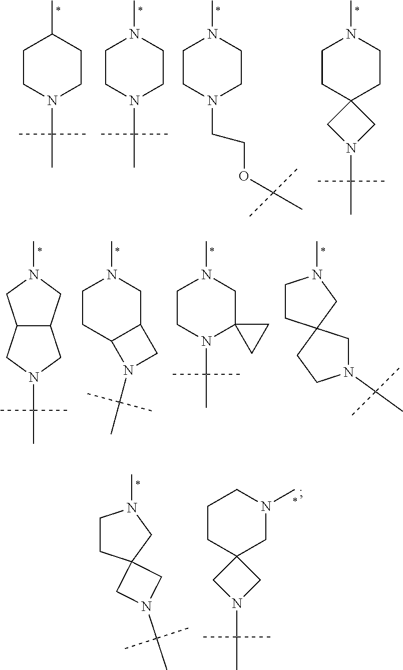

in some specific embodiments, the structure of

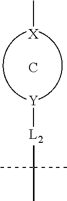

is selected from:

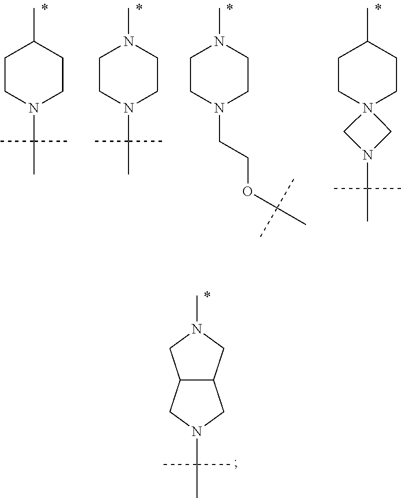

wherein * represents the site to which the parent core structure is linked; and
- - - represents the site to which the linker L is linked.

Embodiment 37. The antibody-drug conjugate or the pharmaceutically acceptable salt thereof, or the solvate, the racemic mixture, the enantiomer, the diastereomer or the tautomer thereof according to any one of embodiments 26 to 36, wherein R₁ is selected from: —O(C₁₋₆ alkyl), —OC₁₋₆ haloalkyl such as —OCH₂CF₃, or C₃₋₉ cycloalkyl, wherein the C₁₋₆ alkyl may optionally be substituted with one or more deuterium; in some specific embodiments, R₁ is —O(C₁₋₆ alkyl); and in some specific embodiments, R₁ is —O—CH₂—CF₃.

Embodiment 38. The antibody-drug conjugate or the pharmaceutically acceptable salt thereof, or the solvate, the racemic mixture, the enantiomer, the diastereomer or the tautomer thereof according to any one of embodiments 1 to 37, wherein D is selected from:

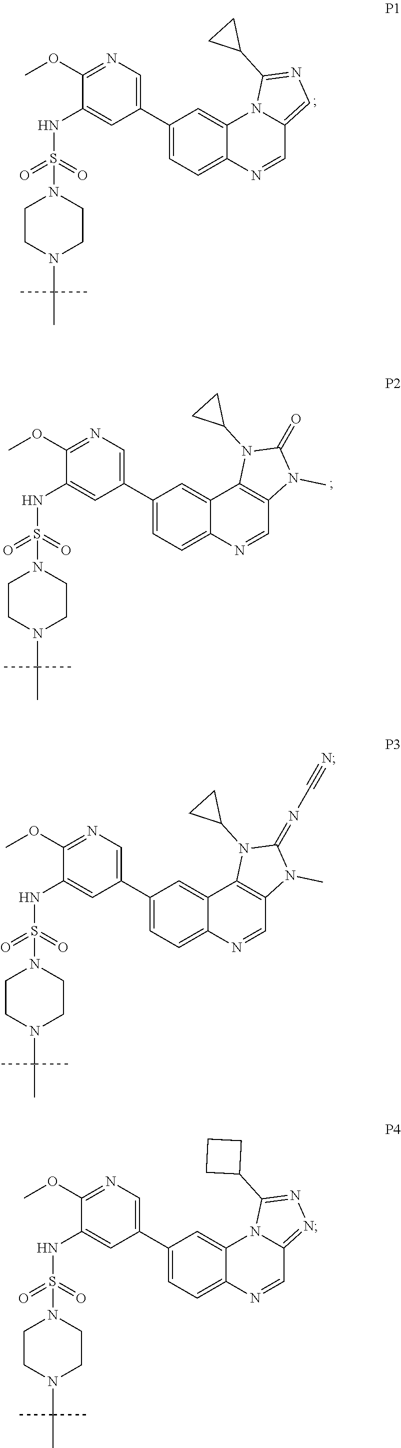

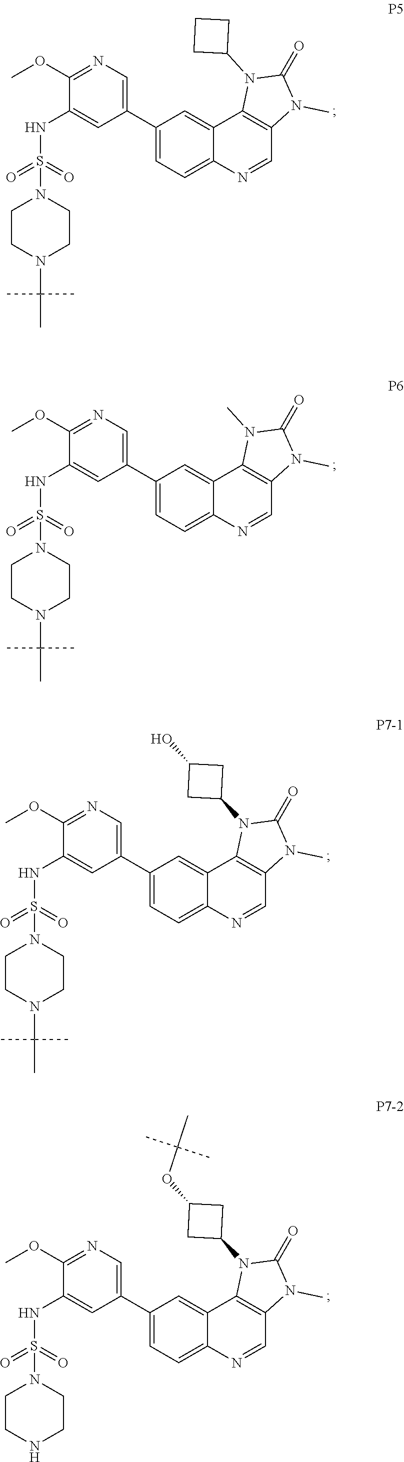

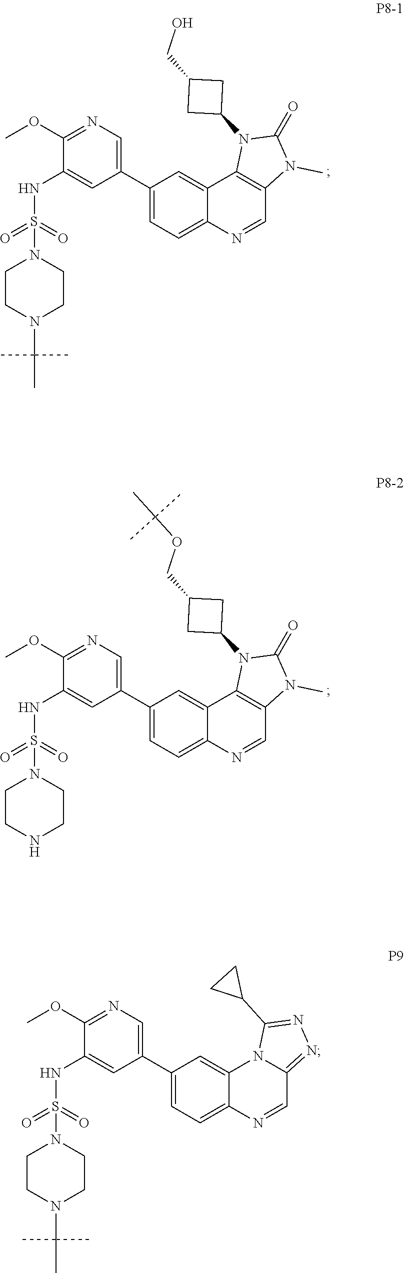

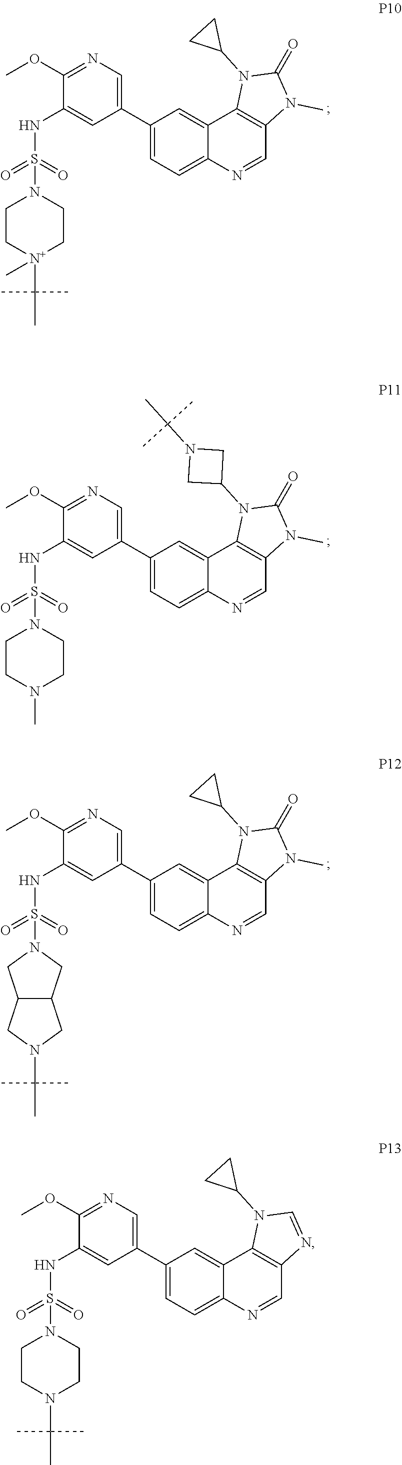

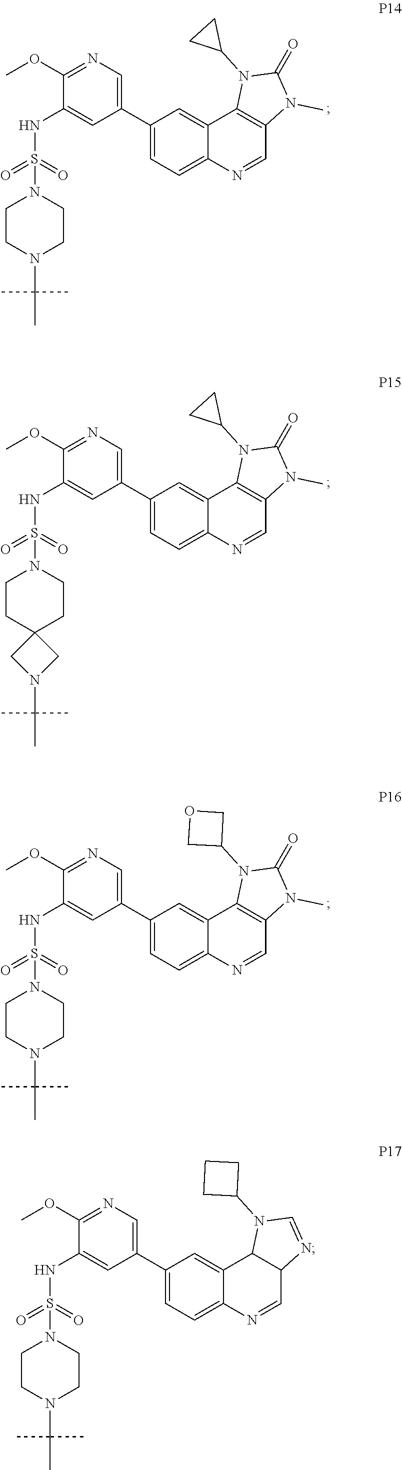

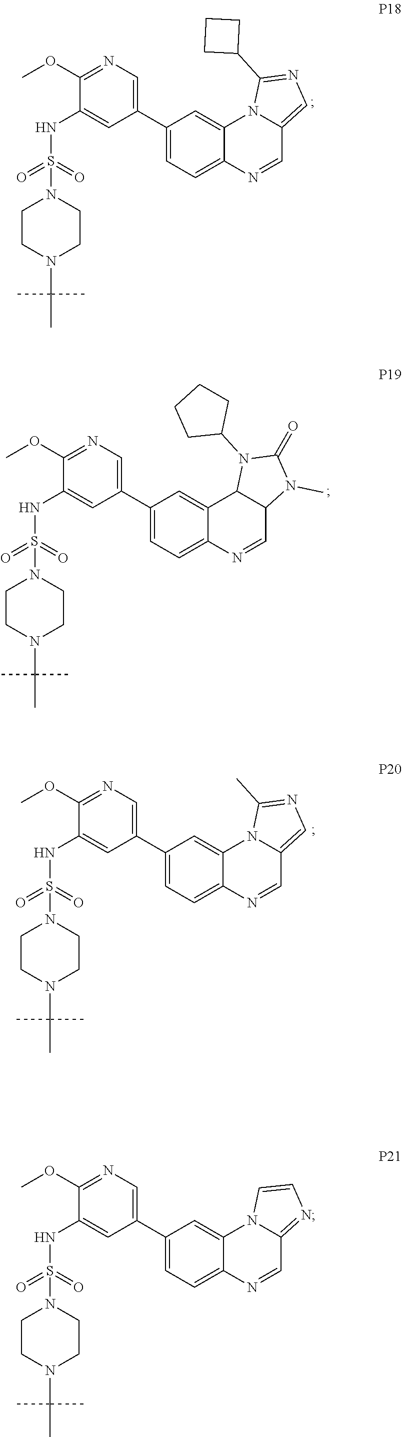

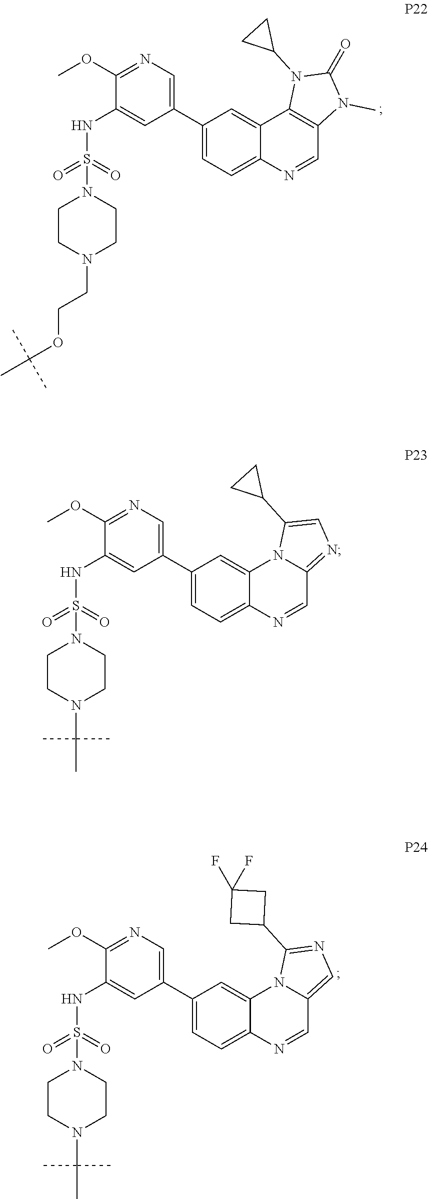

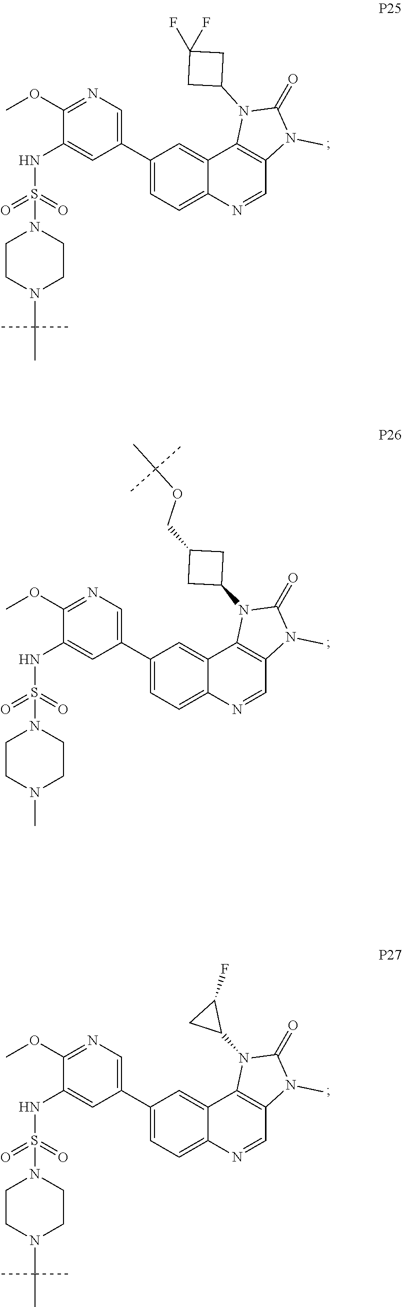

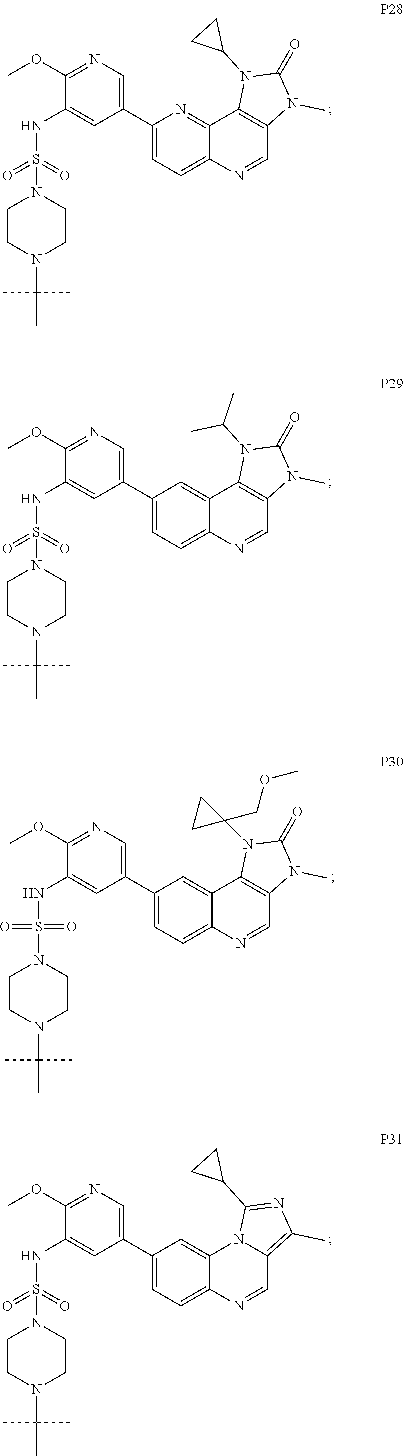

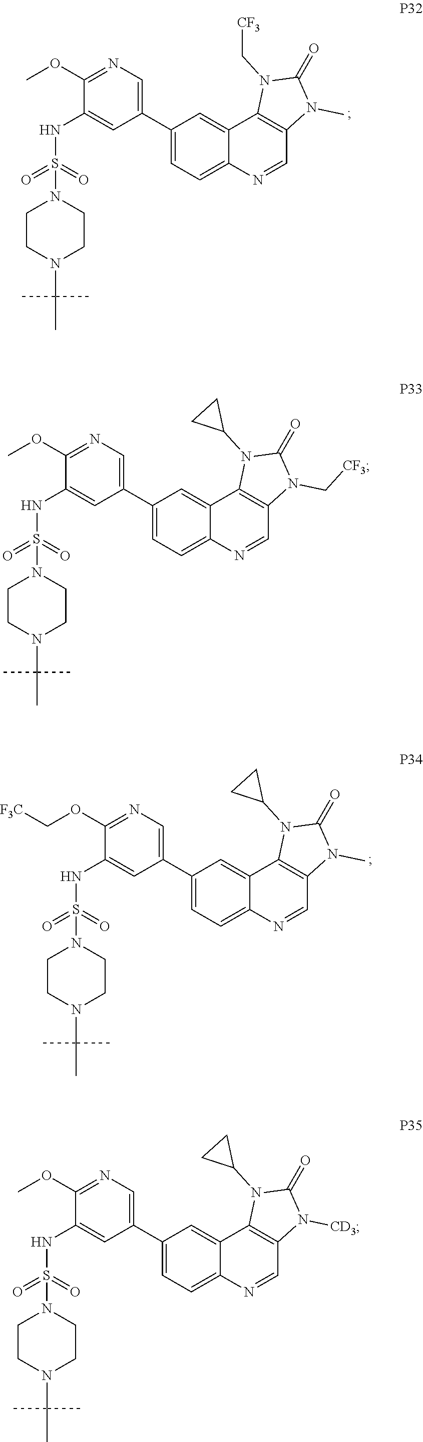

wherein the dashed lines represent the site where D is linked to the linker L.

Embodiment 39. The antibody-drug conjugate or the pharmaceutically acceptable salt thereof, or the solvate, the racemic mixture, the enantiomer, the diastereomer or the tautomer thereof according to any one of embodiments 1 to 38, wherein -L- has a structure represented by formula (III):

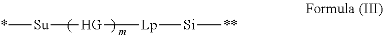

wherein
Su is a stretcher group;
HG is a bridged spacer group;
Lp is a short chain polypeptide consisting of 2-10 amino acids, wherein the amino acids are natural amino acids or non-natural amino acids;
Si is a self-immolative group;
m is 0 or 1;
the symbol * represents the site to which Ab is linked; and the symbol ** represents the site to which D is linked.

Embodiment 40. The antibody-drug conjugate or the pharmaceutically acceptable salt thereof, or the solvate, the racemic mixture, the enantiomer, the diastereomer or the tautomer thereof according to embodiment 39, wherein the Su is selected from the following structures:

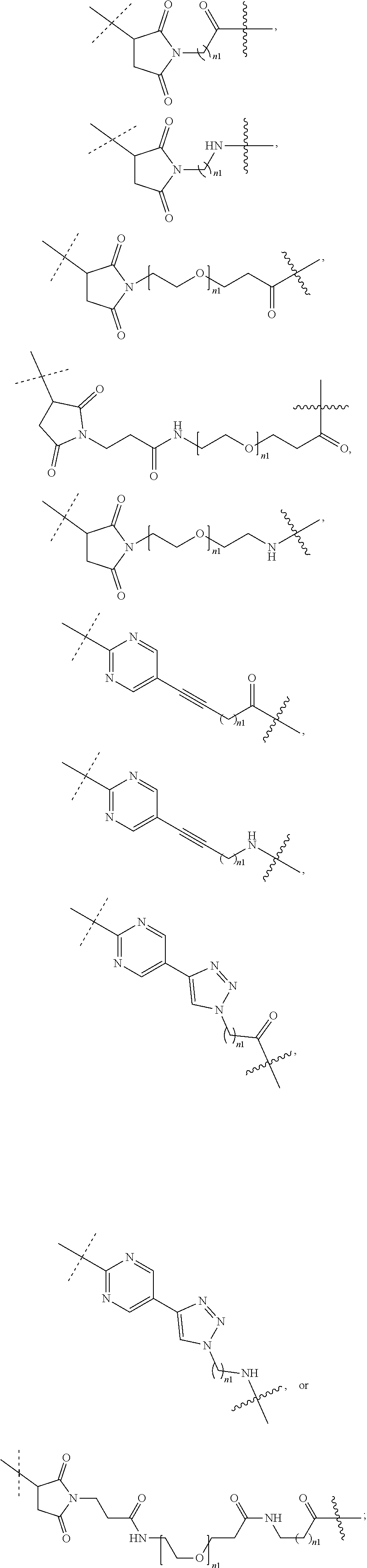

each n₁ is independently selected from an integer from 1 to 10, e.g., 1, 2, 3, 4, 5, 6, 7, 8, 9, or 10;
wherein - - - represents the site to which the Ab is linked; and
[symbol] represents the site to which the structure (e.g., HG or Lp) on the right side of Su is linked;
in some specific embodiments, Su is selected from:

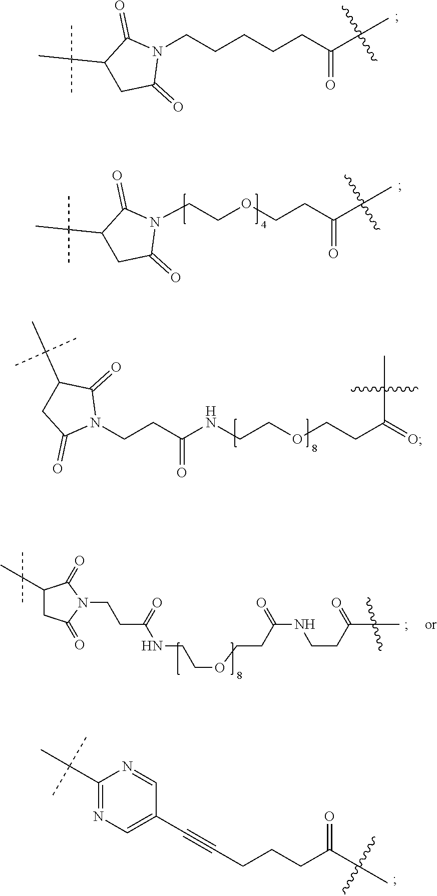

and the remaining symbols are as defined in the embodiments hereinbefore.

Embodiment 41. The antibody-drug conjugate or the pharmaceutically acceptable salt thereof, or the solvate, the racemic mixture, the enantiomer, the diastereomer or the tautomer thereof according to embodiment 39 or 40, wherein amino acid residues contained in the Lp are independently selected from L- or D-isomers of the following amino acids: alanine (including β-alanine), arginine, aspartic acid, asparagine, cysteine, histidine, glycine, glutamic acid, valine, glutamine, phenylalanine, lysine, leucine, isoleucine, methionine, serine, tyrosine, threonine, tryptophan, proline, ornithine, penicillamine, aminoalkynoic acid, aminoalkanedioic acid, heterocyclyl-carboxylic acid, citrulline, selenocysteine, pyrrolysine, diaminoalkanoic acid, and a derivative thereof; preferably, Lp is selected from: -Phe-Lys-, -Val-Lys-, -Phe-Phe-Lys-, -D-Phe-Phe-Lys-, -Gly-Phe-Lys-, -Ala-Lys-, -Val-Cit-, -Phe-Cit-, -Leu-Cit-, -Iie-Cit-, -Trp-Cit-, -Phe-Ala-, -Gly-Phe-Leu-Gly-, -Ala-Leu-Ala-Leu-, -Val-Ala-, -Gly-Gly-Phe-Gly, -Gly-Cit-, -Cit-Val-, -Cit-Ala-, -Lys-Val-, -Val-Lys(Ac)-, -Phe-Lys(Ac)-, -Ala-Ala-, -Ala-Ala-Ala-, -Ala-Ala-Asn-, -Ala-Ala-Asp-, -Lys-Ala-Ala-Asn-, -Lys-Ala-Ala-Asp-, -D-Val-Leu-Lys-, -Gly-Gly-Arg-, -Gly-Gly-Phe-, -Val-Lys-Gly-, -Val-Lys-Gly-Gly-, and -Lys-Ala-Asn-, or a derivative thereof; and more preferably, Lp is -Val-Cit-, -Val-Ala-, -Gly-Gly-Phe-Gly, or a derivative thereof.

Embodiment 42. The antibody-drug conjugate or the pharmaceutically acceptable salt thereof, or the solvate, the racemic mixture, the enantiomer, the diastereomer or the tautomer thereof according to embodiment 39 or 40, wherein the structure of Si is:

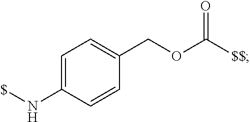

the symbol $ represents the site to which Lp is linked, and the symbol $$ represents the site to which D is linked.

Embodiment 43. The antibody-drug conjugate or the pharmaceutically acceptable salt thereof, or the solvate, the racemic mixture, the enantiomer, the diastereomer or the tautomer thereof according to any one of embodiments 39 to 42, wherein the structure of HG is:

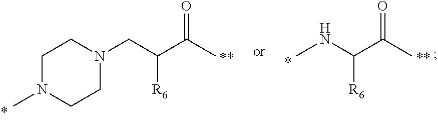

each R₆ is independently selected from: —OR^{x1}, —NR^{x1}R^{x2}, —C₁₋₆ alkylene-OR^{x1}, —C₁₋₆ alkylene-NR^{x1}R^{x2}, —C₁₋₆ alkyl, or —C₁₋₆ alkylene-COOH, wherein R^{x1} and R^{x2} are each independently selected from H or —C₁₋₆ alkyl;
the symbol * represents the site to which the Su group is linked; the symbol ** represents the site to which the Lp group is linked; and
in some specific embodiments, each R₆ is independently —OH or —C₁₋₆ alkylene-COOH.

Embodiment 44. The antibody-drug conjugate or the pharmaceutically acceptable salt thereof, or the solvate, the racemic mixture, the enantiomer, the diastereomer or the tautomer thereof according to embodiment 43, wherein HG is selected from:

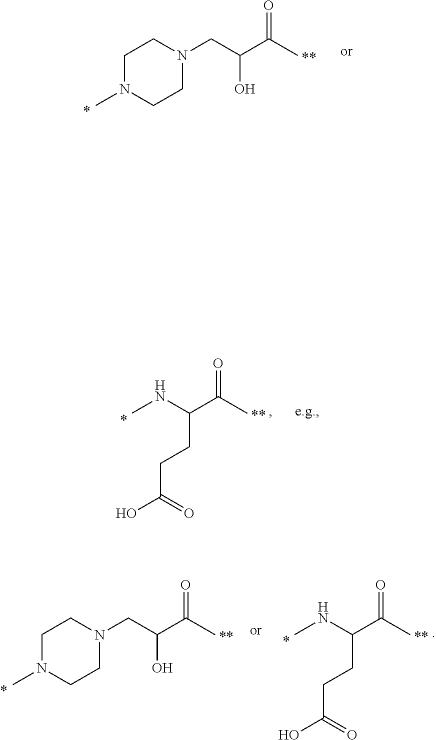

Embodiment 45. The antibody-drug conjugate or the pharmaceutically acceptable salt thereof, or the solvate, the racemic mixture, the enantiomer, the diastereomer or the tautomer thereof according to any one of embodiments 1 to 44, wherein the structure of -L-D is selected from:

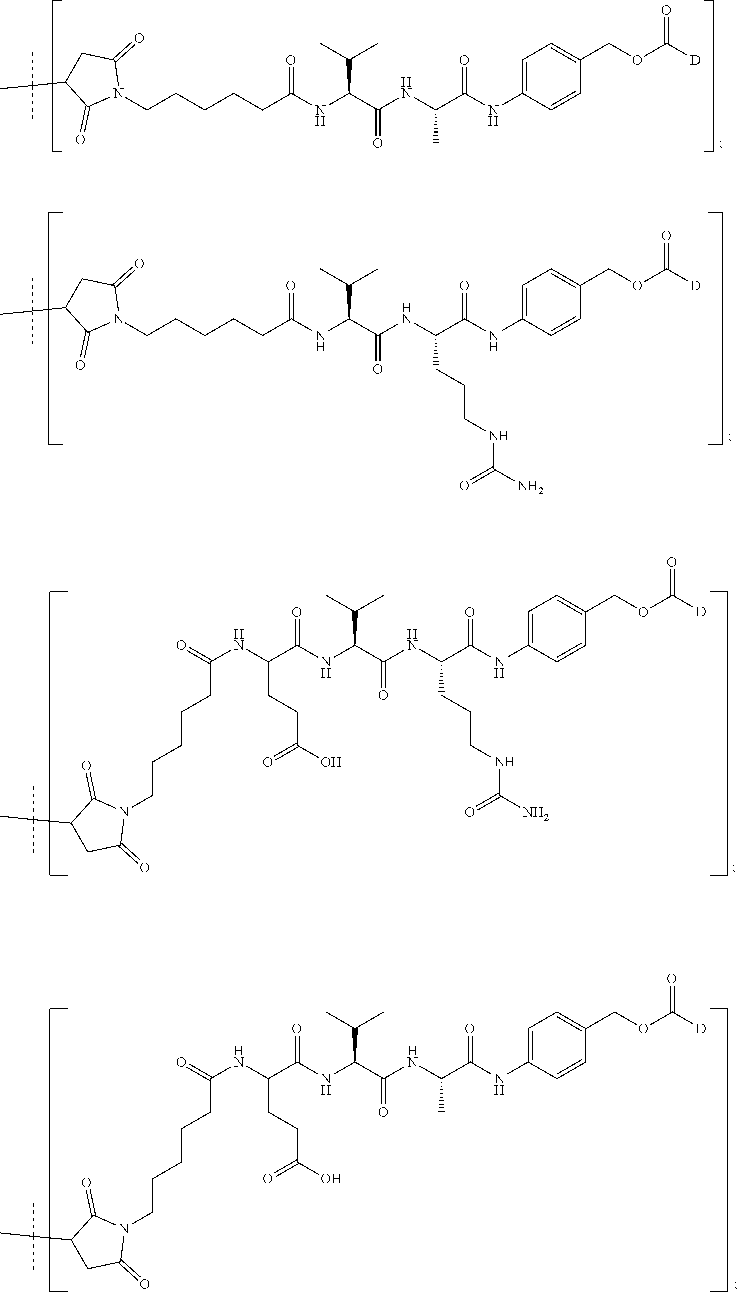

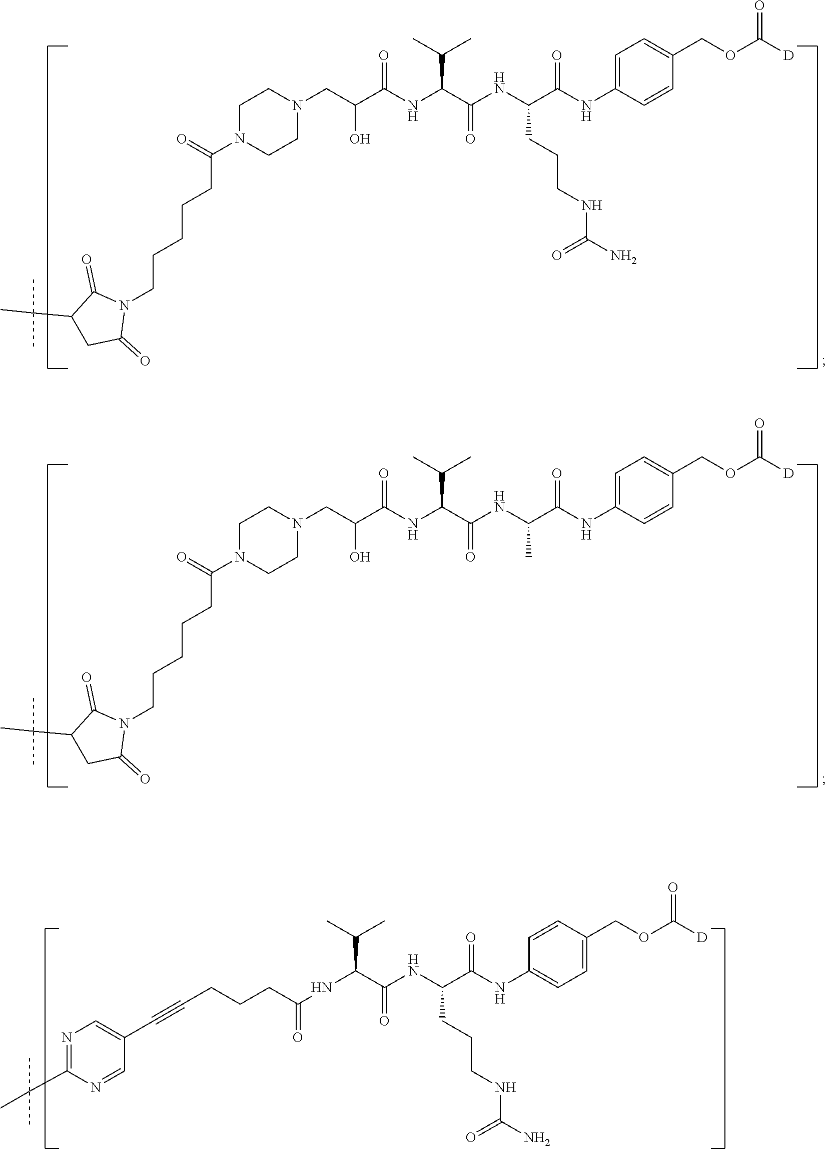

wherein the dashed lines represent the site to which Ab is linked; and
D is as defined in embodiments 1 to 38.

Embodiment 46. The antibody-drug conjugate or the pharmaceutically acceptable salt thereof, or the solvate, the racemic mixture, the enantiomer, the diastereomer or the tautomer thereof according to any one of embodiments 1 to 45, being selected from:

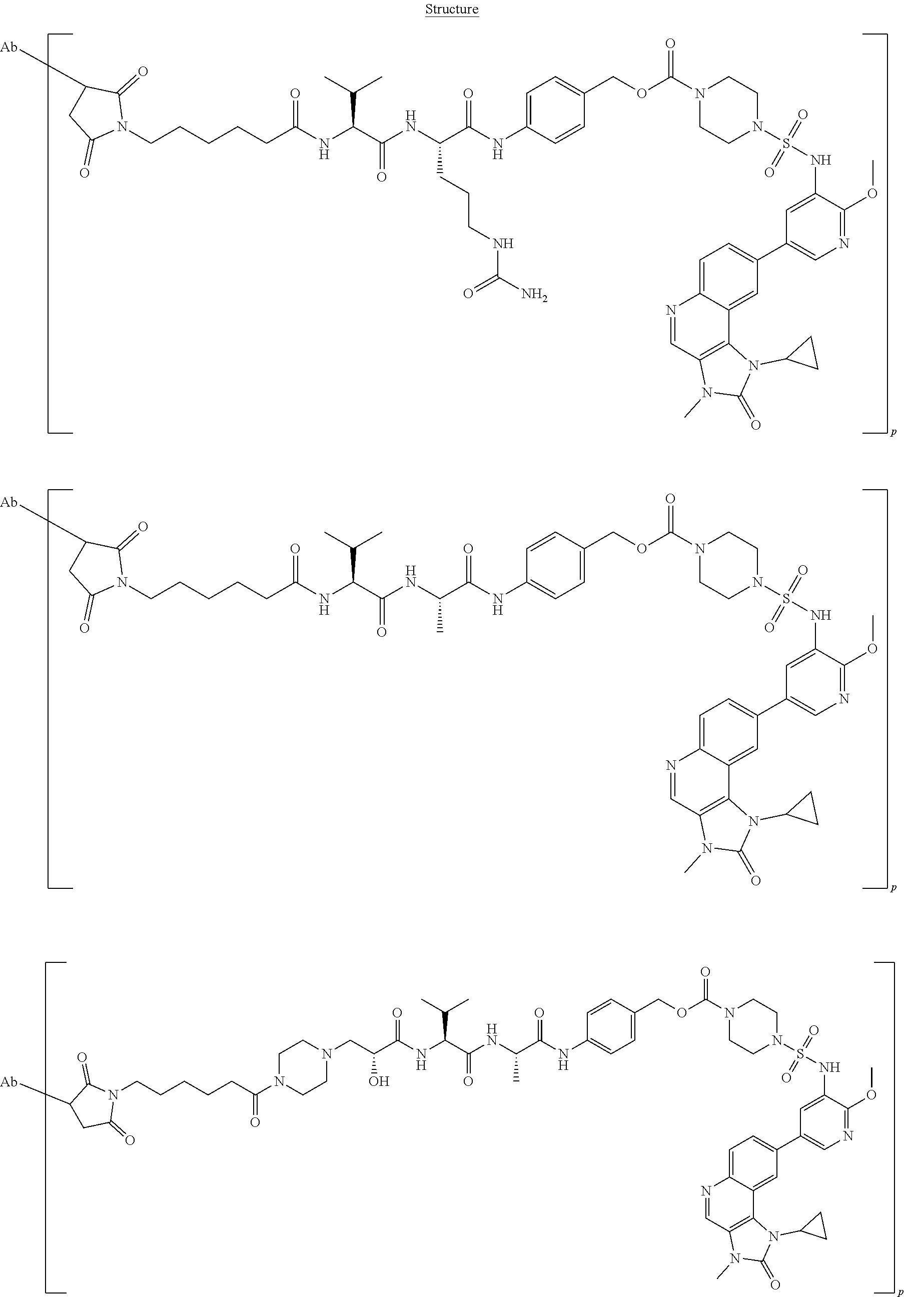

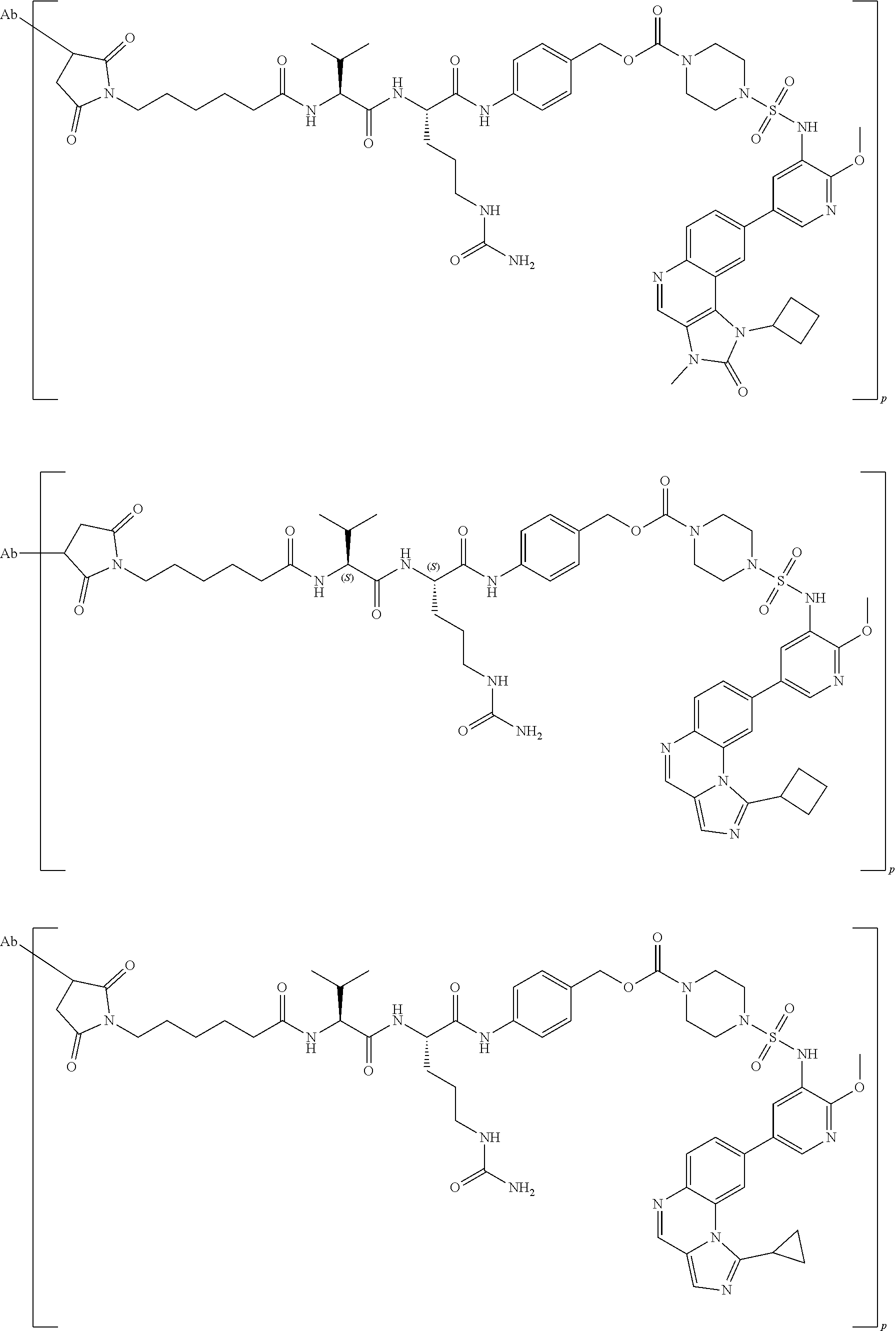

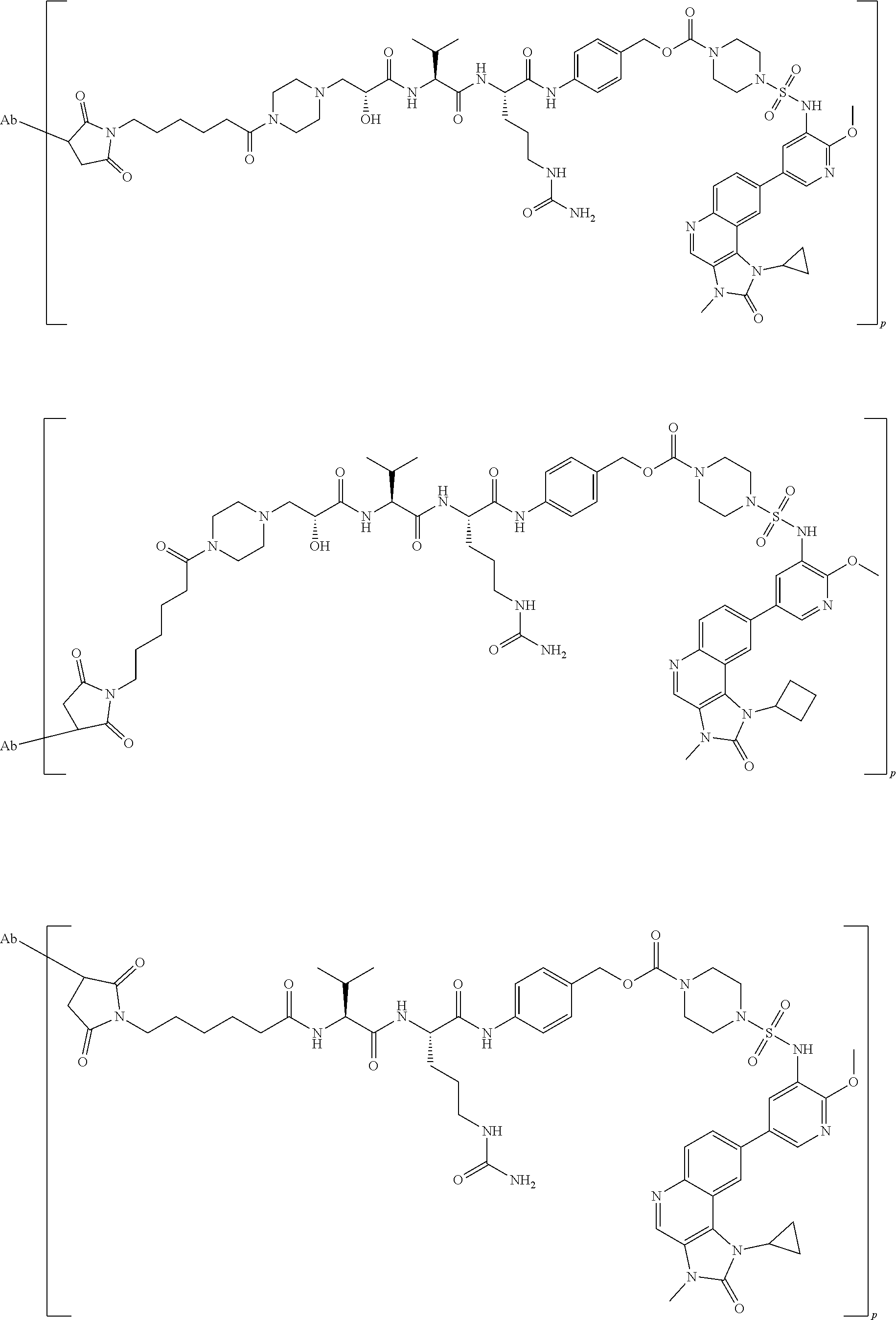

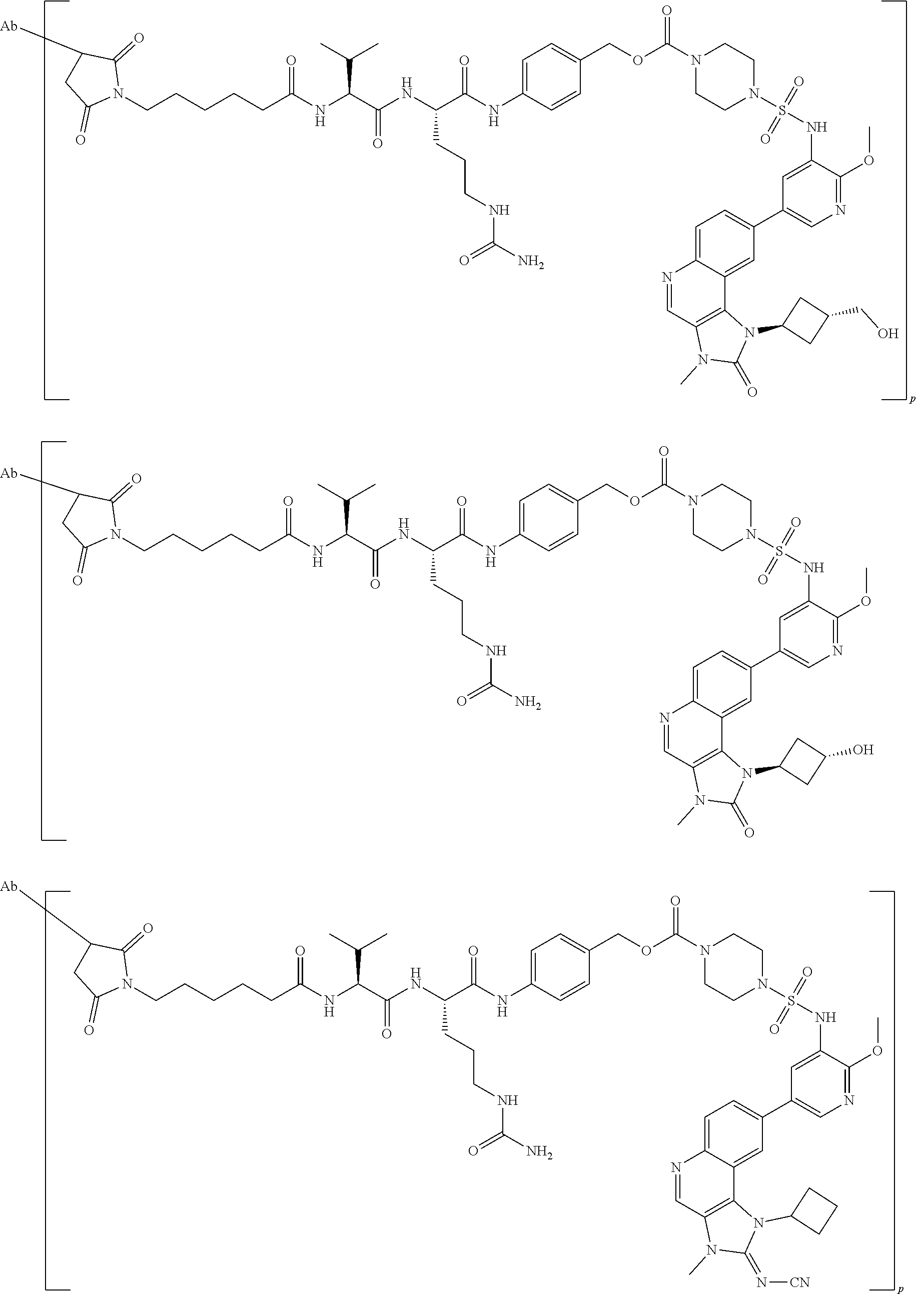

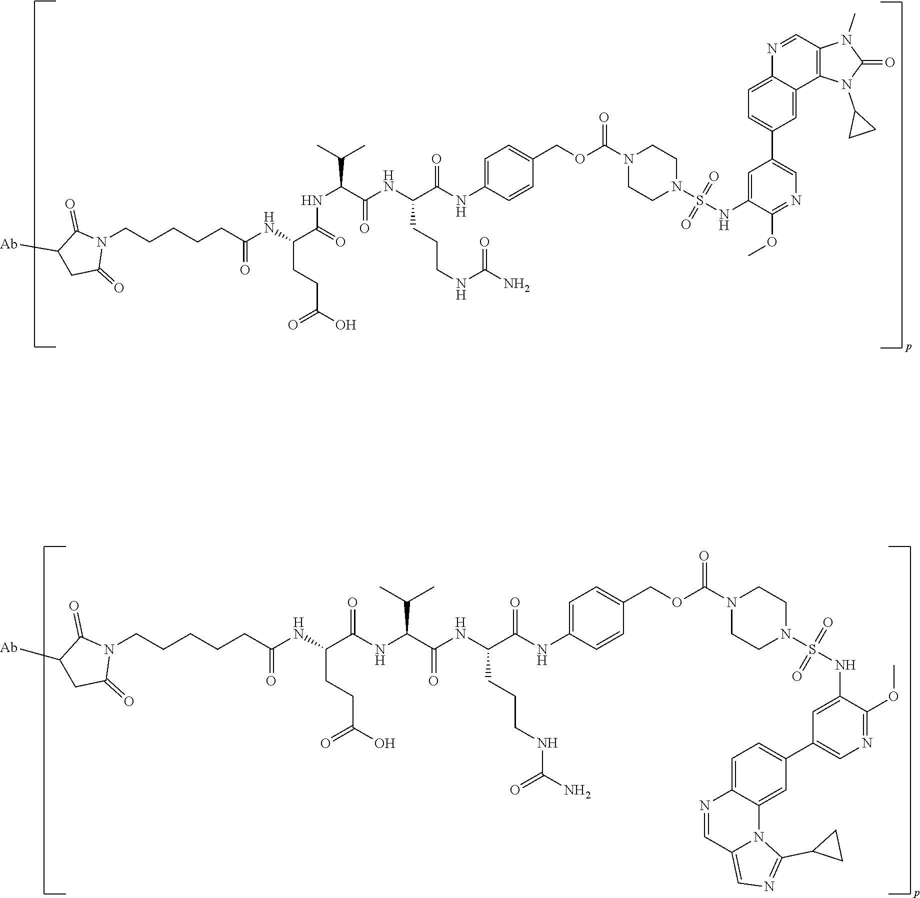

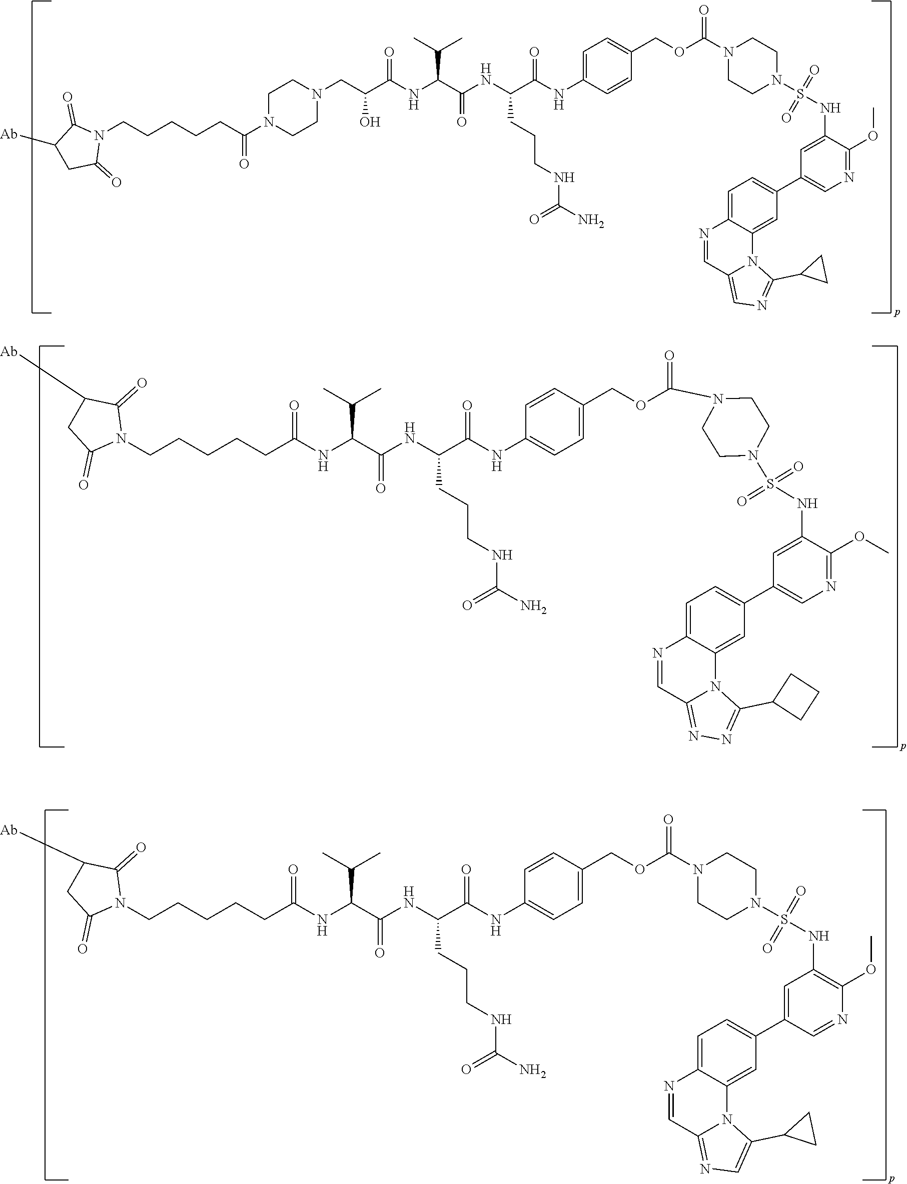

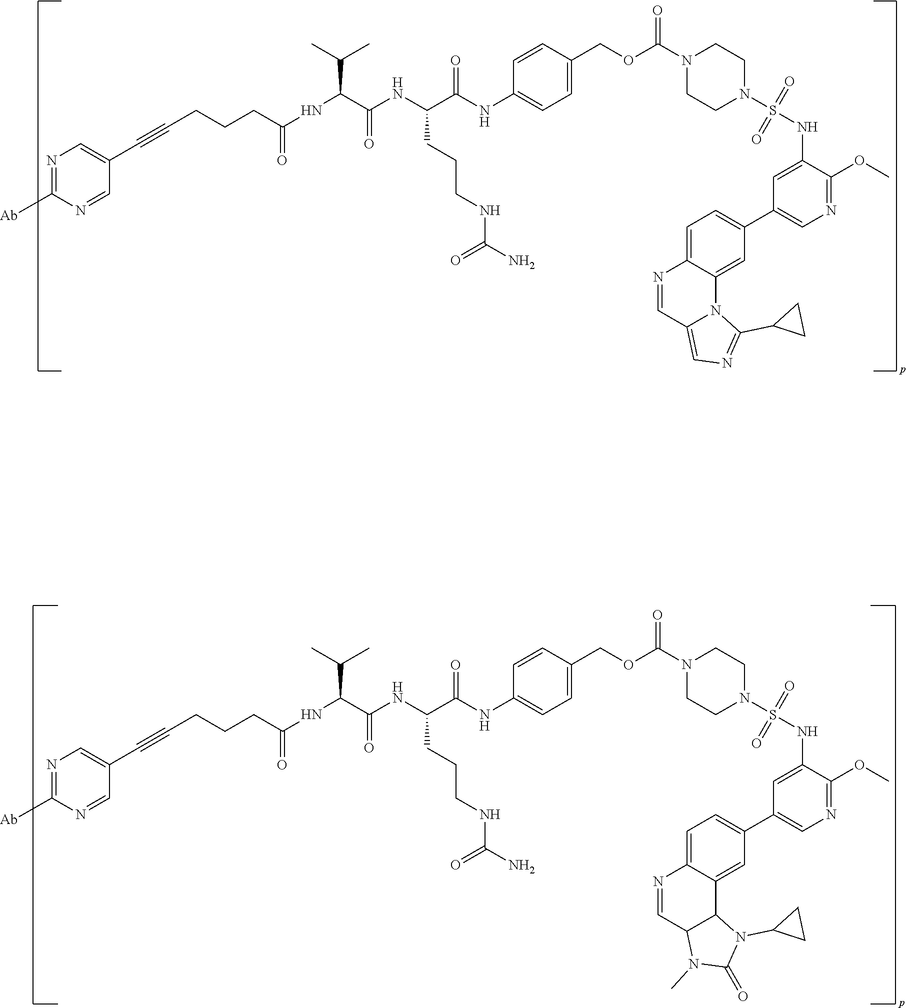

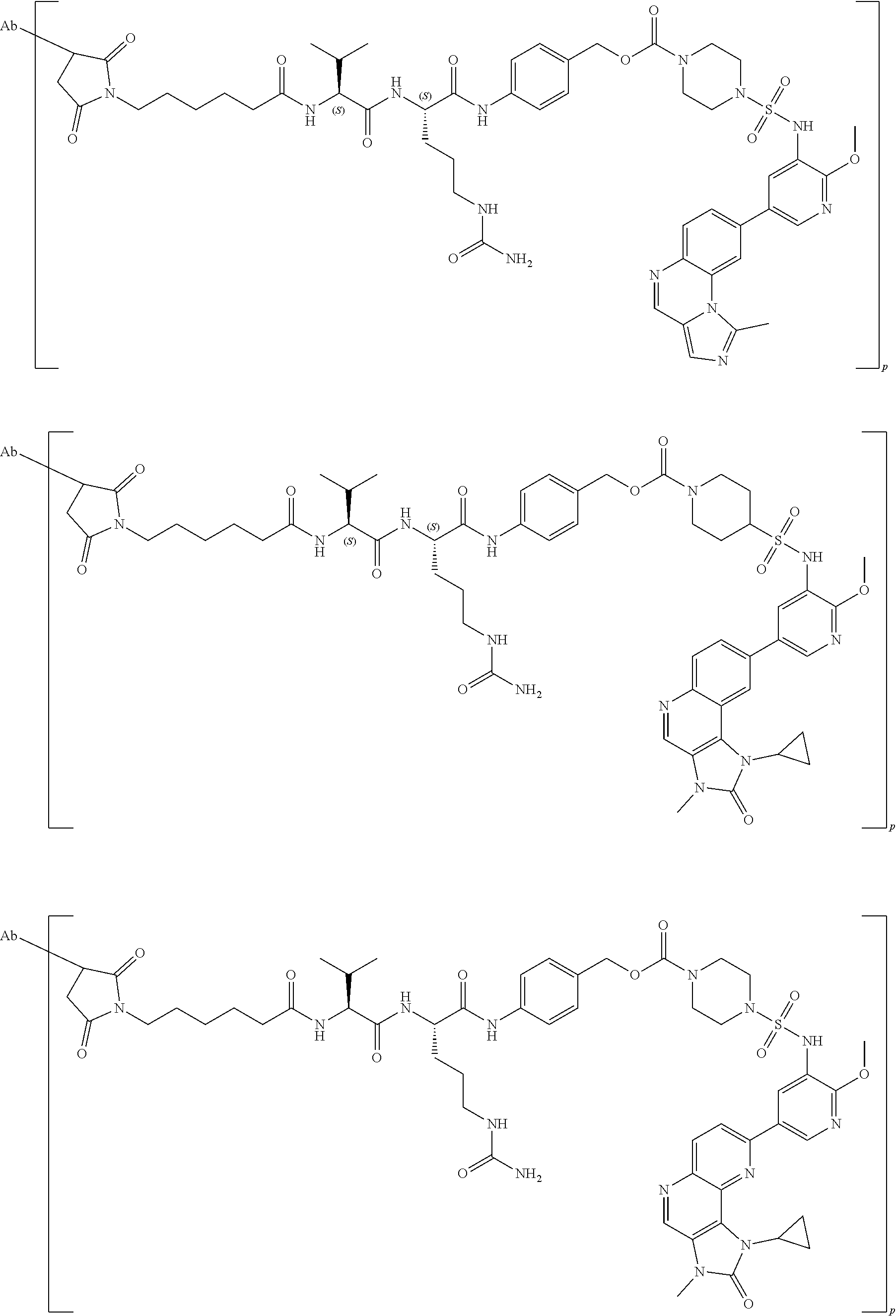

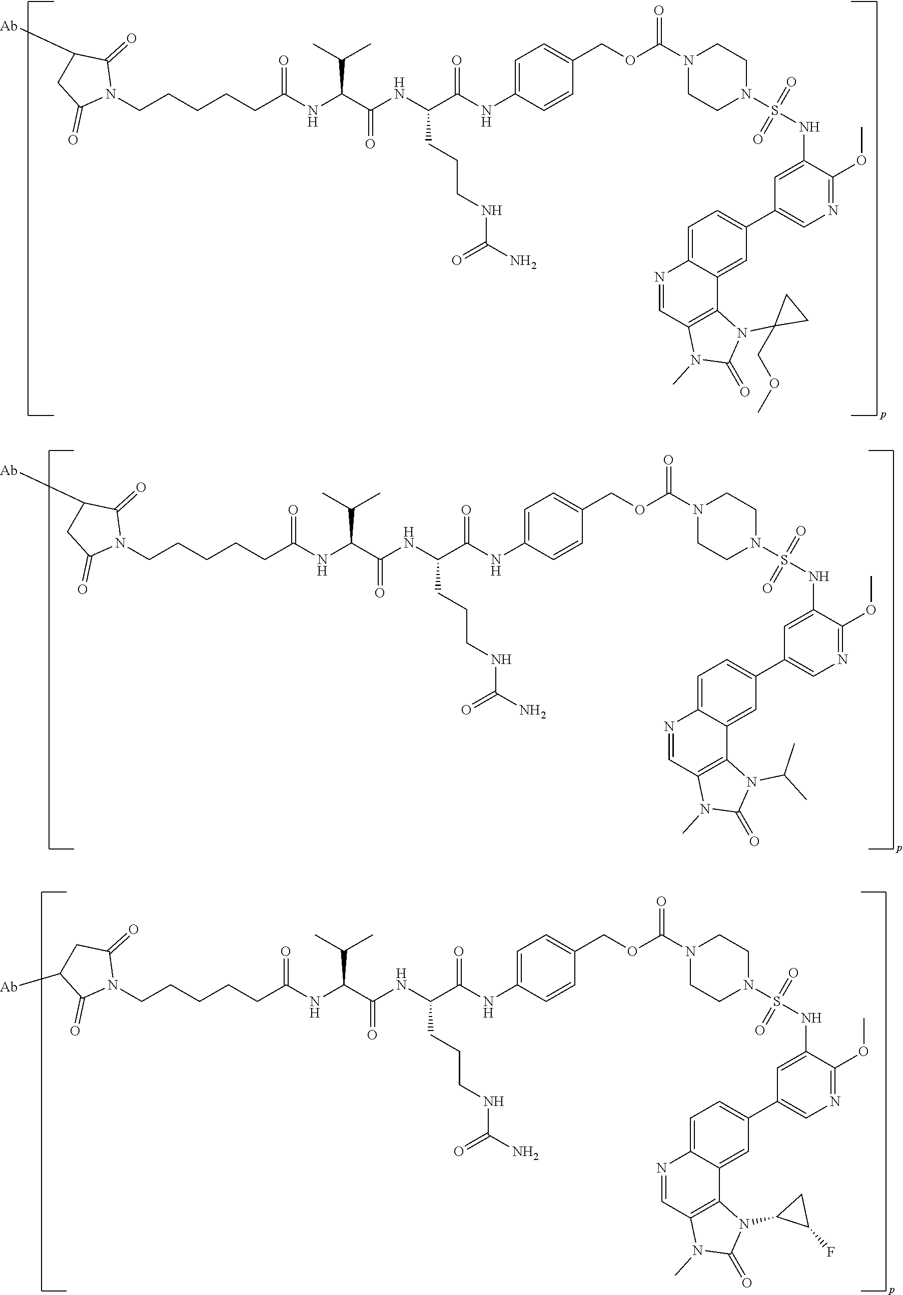

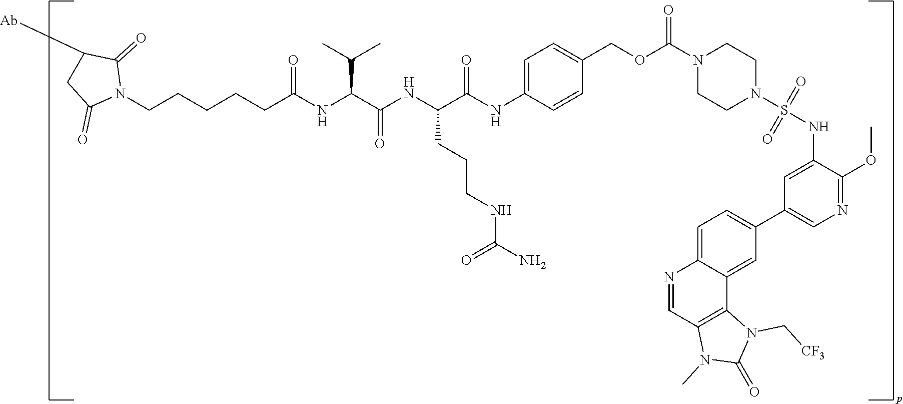

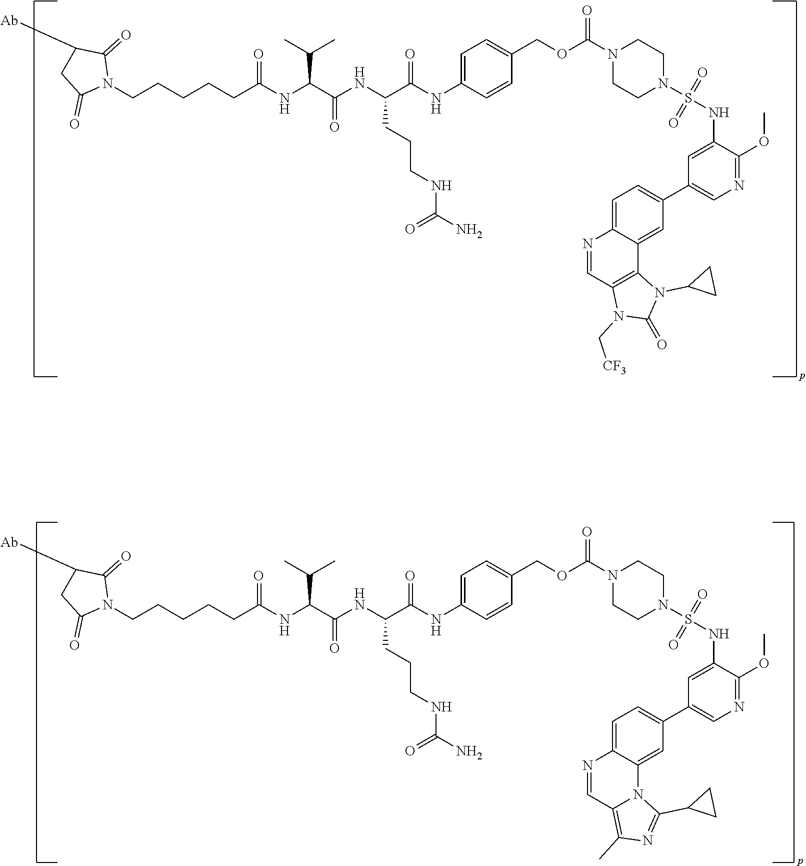

wherein Ab and p are as defined in embodiment 1.

Embodiment 47. The antibody-drug conjugate or the pharmaceutically acceptable salt thereof, or the solvate, the racemic mixture, the enantiomer, the diastereomer or the tautomer thereof according to embodiment 46, wherein p is selected from 2, 3, 4, 5, 6, 7 and 8.

Embodiment 48. The antibody-drug conjugate or the pharmaceutically acceptable salt thereof, or the solvate, the racemic mixture, the enantiomer, the diastereomer or the tautomer thereof according to any one of embodiments 1 to 47, wherein Ab is an antibody that specifically binds to EGFR or an antigen-binding fragment thereof, e.g., cetuximab or necitumumab.

Embodiment 49. The antibody-drug conjugate or the pharmaceutically acceptable salt thereof, or the solvate, the racemic mixture, the enantiomer, the diastereomer or the tautomer thereof according to any one of embodiments 1 to 48, wherein
in some embodiments, the antibody that specifically binds to EGFR or the antigen-binding fragment thereof is any antibody known in the art that specifically binds to EGFR or an antigen-binding fragment thereof; for example, the antibody that specifically binds to EGFR or the antigen-binding fragment thereof comprises 1, 2, 3, 4, 5 or 6 CDRs of a known antibody that specifically binds to EGFR (e.g., cetuximab or necitumumab). In some embodiments, the antibody that specifically binds to EGFR or the antigen-binding fragment thereof comprises 1, 2 and 3 CDRs of the heavy chain variable region, i.e., HCDR1, HCDR2 and HCDR3, that is known to specifically bind to EGFR (e.g., cetuximab or necitumumab). In some embodiments, the antibody that specifically binds to EGFR or the antigen-binding fragment thereof comprises 1, 2 and 3 CDRs of the light chain variable region, i.e., LCDR1, LCDR2 and LCDR3, that is known to specifically bind to EGFR (e.g., cetuximab or necitumumab). In some embodiments, the antibody that specifically binds to EGFR or the antigen-binding fragment thereof comprises three CDRs of the heavy chain variable region and three CDRs of the light chain variable region of the antibody that is known to specifically bind to EGFR (e.g., cetuximab or necitumumab). In some embodiments, the antibody that specifically binds to EGFR or the antigen-binding fragment thereof comprises a heavy chain variable region of the antibody that is known to specifically bind to EGFR (e.g., cetuximab or necitumumab). In some embodiments, the antibody that specifically binds to EGFR or the antigen-binding fragment thereof comprises a light chain variable region of the antibody that is known to specifically bind to EGFR (e.g., cetuximab or necitumumab). In some embodiments, the antibody that specifically binds to EGFR or the antigen-binding fragment thereof comprises a heavy chain variable region and a light chain variable region of the antibody that is known to specifically bind to EGFR (e.g., cetuximab or necitumumab). In some embodiments, the antibody that specifically binds to EGFR or the antigen-binding fragment thereof comprises a heavy chain of the antibody that is known to specifically bind to EGFR (e.g., cetuximab or necitumumab). In some embodiments, the antibody that specifically binds to EGFR or the antigen-binding fragment thereof comprises a light chain of the antibody that is known to specifically bind to EGFR (e.g., cetuximab or necitumumab). In some embodiments, the antibody that specifically binds to EGFR or the antigen-binding fragment thereof comprises a heavy chain and a light chain of the antibody that is known to specifically bind to EGFR (e.g., cetuximab or necitumumab). In some embodiments, the antibody that specifically binds to EGFR or the antigen-binding fragment thereof comprises or consists of two heavy chains and two light chains of the antibody that is known to specifically bind to EGFR (e.g., cetuximab or necitumumab). In some embodiments, the antibody that specifically binds to EGFR or the antigen-binding fragment thereof comprises complementarity determining regions HCDR1, HCDR2, HCDR3, LCDR1, LCDR2 and LCDR3; wherein the amino acid sequences of the HCDR1, HCDR2, HCDR3, LCDR1, LCDR2 and LCDR3 are the same as those of HCDR1, HCDR2, HCDR3, LCDR1, LCDR2 and LCDR3 of cetuximab or necitumumab, respectively; or the HCDR1, HCDR2, HCDR3, LCDR1, LCDR2 and LCDR3 are HCDR1, HCDR2, HCDR3, LCDR1, LCDR2 and LCDR3 of cetuximab or necitumumab, respectively. In some embodiments, the antibody that specifically binds to EGFR or the antigen-binding fragment thereof comprises a heavy chain variable region and a light chain variable region, and the amino acid sequence of the heavy chain variable region and the amino acid sequence of light chain variable region are the same as the amino acid sequence of the heavy chain variable region and the amino acid sequence of the light chain variable region of cetuximab or necitumumab, respectively; or the heavy chain variable region and light chain variable region are the heavy chain variable region and light chain variable region of cetuximab or necitumumab, respectively. In some embodiments, the antibody that specifically binds to EGFR or the antigen-binding fragment thereof comprises a heavy chain and a light chain, and the amino acid sequence of the heavy chain and the amino acid sequence of light chain are the same as the amino acid sequence of the heavy chain and the amino acid sequence of the light chain of cetuximab or necitumumab, respectively; or the heavy chain and light chain are the heavy chain and the light chain of cetuximab or necitumumab, respectively. In some embodiments, the antibody that specifically binds to EGFR has the same heavy chain and the same light chain as cetuximab or necitumumab.

In some embodiments, the antibody that specifically binds to EGFR or the antigen-binding fragment thereof comprises LCDR1, LCDR2, LCDR3, HCDR1, HCDR2 and HCDR3, or comprises a light chain variable region comprising LCDR1, LCDR2 and LCDR3 and a heavy chain variable region comprising HCDR1, HCDR2 and HCDR3; wherein the HCDR1 comprises an amino acid sequence as set forth in SEQ ID NO: 1 or SEQ ID NO: 11, the HCDR2 comprises an amino acid sequence as set forth in SEQ ID NO: 2 or SEQ ID NO: 12, and the HCDR3 comprises an amino acid sequence as set forth in SEQ ID NO: 3 or SEQ ID NO: 13; and the LCDR1 comprises an amino acid sequence as set forth in SEQ ID NO: 4 or SEQ ID NO: 14, the LCDR2 comprises an amino acid sequence as set forth in SEQ ID NO: 5 or SEQ ID NO: 15, and the LCDR3 comprises an amino acid sequence as set forth in SEQ ID NO: 6 or SEQ ID NO: 16; or
the HCDR1 consists of an amino acid sequence as set forth in SEQ ID NO: 1 or SEQ ID NO: 11, the HCDR2 consists of an amino acid sequence as set forth in SEQ ID NO: 2 or SEQ ID NO: 12, and the HCDR3 consists of an amino acid sequence as set forth in SEQ ID NO: 3 or SEQ ID NO: 13; and the LCDR1 consists of an amino acid sequence as set forth in SEQ ID NO: 4 or SEQ ID NO: 14, the LCDR2 consists of an amino acid sequence as set forth in SEQ ID NO: 5 or SEQ ID NO: 15, and the LCDR3 consists of an amino acid sequence as set forth in SEQ ID NO: 6 or SEQ ID NO: 16; or
the HCDR1 comprises an amino acid sequence as set forth in SEQ ID NO: 1, the HCDR2 comprises an amino acid sequence as set forth in SEQ ID NO: 2, and the HCDR3 comprises an amino acid sequence as set forth in SEQ ID NO: 3; and the LCDR1 comprises an amino acid sequence as set forth in SEQ ID NO: 4, the LCDR2 comprises an amino acid sequence as set forth in SEQ ID NO: 5, and the LCDR3 comprises an amino acid sequence as set forth in SEQ ID NO: 6; or
the HCDR1 consists of an amino acid sequence as set forth in SEQ ID NO: 1, the HCDR2 consists of an amino acid sequence as set forth in SEQ ID NO: 2, and the HCDR3 consists of an amino acid sequence as set forth in SEQ ID NO: 3; and the LCDR1 consists of an amino acid sequence as set forth in SEQ ID NO: 4, the LCDR2 consists of an amino acid sequence as set forth in SEQ ID NO: 5, and the LCDR3 consists of an amino acid sequence as set forth in SEQ ID NO: 6; or
the HCDR1 comprises an amino acid sequence as set forth in SEQ ID NO: 11, the HCDR2 comprises an amino acid sequence as set forth in SEQ ID NO: 12, and the HCDR3 comprises an amino acid sequence as set forth in SEQ ID NO: 13; and the LCDR1 comprises an amino acid sequence as set forth in SEQ ID NO: 14, the LCDR2 comprises an amino acid sequence as set forth in SEQ ID NO: 15, and the LCDR3 comprises an amino acid sequence as set forth in SEQ ID NO: 16; or
the HCDR1 consists of an amino acid sequence as set forth in SEQ ID NO: 11, the HCDR2 consists of an amino acid sequence as set forth in SEQ ID NO: 12, and the HCDR3 consists of an amino acid sequence as set forth in SEQ ID NO: 13; and the LCDR1 consists of an amino acid sequence as set forth in SEQ ID NO: 14, the LCDR2 consists of an amino acid sequence as set forth in SEQ ID NO: 15, and the LCDR3 consists of an amino acid sequence as set forth in SEQ ID NO: 16.

In some embodiments, the antibody that specifically binds to EGFR or the antigen-binding fragment thereof comprises a heavy chain variable region and a light chain variable region, wherein the heavy chain variable region comprises an amino acid sequence as set forth in SEQ ID NO: 7 or SEQ ID NO: 17, and the light chain variable region comprises an amino acid sequence as set forth in SEQ ID NO: 8 or SEQ ID NO: 18; or the heavy chain variable region consists of an amino acid sequence as set forth in SEQ ID NO: 7 or SEQ ID NO: 17, and the light chain variable region consists of an amino acid sequence as set forth in SEQ ID NO: 8 or SEQ ID NO: 18.

In some embodiments, the antibody that specifically binds to EGFR or the antigen-binding fragment thereof comprises a heavy chain variable region and a light chain variable region, wherein the heavy chain variable region comprises an amino acid sequence as set forth in SEQ ID NO: 7, and the light chain variable region comprises an amino acid sequence as set forth in SEQ ID NO: 8; or the heavy chain variable region consists of an amino acid sequence as set forth in SEQ ID NO: 7, and the light chain variable region consists of an amino acid sequence as set forth in SEQ ID NO: 8.

In some embodiments, the antibody that specifically binds to EGFR or the antigen-binding fragment thereof comprises a heavy chain variable region and a light chain variable region, wherein the heavy chain variable region comprises an amino acid sequence as set forth in SEQ ID NO: 17, and the light chain variable region comprises an amino acid sequence as set forth in SEQ ID NO: 18; or the heavy chain variable region consists of an amino acid sequence as set forth in SEQ ID NO: 17, and the light chain variable region consists of an amino acid sequence as set forth in SEQ ID NO: 18.

In some embodiments, the antibody that specifically binds to EGFR or the antigen-binding fragment thereof comprises a heavy chain and a light chain, wherein the heavy chain comprises an amino acid sequence as set forth in SEQ ID NO: 9 or SEQ ID NO: 19, and the light chain comprises an amino acid sequence as set forth in SEQ ID NO: 10 or SEQ ID NO: 20; or the heavy chain consists of an amino acid sequence as set forth in SEQ ID NO: 9 or SEQ ID NO: 19, and the light chain consists of an amino acid sequence as set forth in SEQ ID NO: 10 or SEQ ID NO: 20. In some embodiments, the antibody that specifically binds to EGFR or the antigen-binding fragment thereof comprises two of the heavy chains and two of the light chains.

In some embodiments, the antibody that specifically binds to EGFR or the antigen-binding fragment thereof comprises a heavy chain and a light chain, wherein the heavy chain comprises an amino acid sequence as set forth in SEQ ID NO: 9, and the light chain comprises an amino acid sequence as set forth in SEQ ID NO: 10; or the heavy chain consists of an amino acid sequence as set forth in SEQ ID NO: 9, and the light chain consists of an amino acid sequence as set forth in SEQ ID NO: 10. In some embodiments, the antibody that specifically binds to EGFR or the antigen-binding fragment thereof comprises two of the heavy chains and two of the light chains.

In some embodiments, the antibody or the antigen-binding fragment thereof that specifically binds to EGFR comprises a heavy chain and a light chain, wherein the heavy chain comprises an amino acid sequence as set forth in SEQ ID NO: 19, and the light chain comprises an amino acid sequence as set forth in SEQ ID NO: 20; or the heavy chain consists of an amino acid sequence as set forth in SEQ ID NO: 19, and the light chain consists of an amino acid sequence as set forth in SEQ ID NO: 20. In some embodiments, the antibody that specifically binds to EGFR or the antigen-binding fragment thereof comprises two of the heavy chains and two of the light chains.

In some embodiments, the antigen-binding fragment is selected from Fv, e.g., a disulfide-stabilized Fv fragment (dsFv), (dsFv)₂, bispecific dsFv (dsFv-dsFv′), Fab, Fab′, Fab′-SH or F(ab′)₂; a diabody, such as a disulfide-stabilized diabody (ds diabody), a triabody, a tetrabody, and a crossFab fragment; a linear antibody; a single-chain antibody molecule (e.g., scFv) and an scFv dimer (bivalent diabody); and a single-domain antibody.

In some embodiments, the antibody that specifically binds to EGFR is a full-length antibody. In some embodiments, the antibody that specifically binds to EGFR also encompasses multispecific antibodies, e.g., bispecific antibodies. In some embodiments, the antibody that specifically binds to EGFR is a CrossMab antibody or a camelized single-domain antibody.

In some embodiments, the antibody that specifically binds to EGFR is a chimeric antibody, a humanized antibody or a human antibody.

In some embodiments, the antibody that specifically binds to EGFR is selected from cetuximab, a biosimilar of cetuximab, necitumumab, a biosimilar of necitumumab, or the anti-EGFR antibody or an antigen-binding fragment thereof as defined herein.

Embodiment 50. The antibody-drug conjugate or the pharmaceutically acceptable salt thereof, or the solvate, the racemic mixture, the enantiomer, the diastereomer or the tautomer thereof according to any one of embodiments 1 to 49, having an average DAR of 2-8, e.g., an average DAR of 3-4, 3.8-4.2, 3-5, 4-5.5, 5-7, 6.5-8, or 6-8, preferably, the antibody-drug conjugate is selected from ADC-1 to ADC-13 disclosed in the examples herein.

Embodiment 51. A pharmaceutical composition, comprising the antibody-drug conjugate or the pharmaceutically acceptable salt thereof, or the solvate, the racemic mixture, the enantiomer, the diastereomer or the tautomer thereof according to any one of embodiments 1 to 50, and optionally comprising a pharmaceutically acceptable carrier.

Embodiment 52. Use of the antibody-drug conjugate or the pharmaceutically acceptable salt thereof, or the solvate, the racemic mixture, the enantiomer, the diastereomer or the tautomer thereof according to any one of embodiments 1 to 50 in the manufacture of a drug for the treatment of a disease or condition mediated at least in part by PI3K and/or PIKK.

Embodiment 53. The use according to embodiment 52, wherein the disease or condition mediated at least in part by PI3K and/or PIKK is selected from: a cancer or an autoimmune disease.

Embodiment 54. The use according to embodiment 53, wherein the autoimmune disease is selected from: rheumatoid arthritis, chronic obstructive pulmonary disease, allergic rhinitis, asthma, acquired hemophilia A (AHA), idiopathic thrombocytopenia (ITP), and activated phosphoinositide 3-kinase-delta syndrome (APDS).

Embodiment 55. The use according to embodiment 53, wherein the cancer is a solid tumor or a hematological malignancy, preferably non-small cell lung cancer or breast cancer.

The compound obtained in the present invention can be further modified at their peripheral positions to provide other target compounds of the present invention. Synthetic chemistry transformations are techniques well known in the art, as published in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette (ed.), Encyclopedia of Reagents for Organic Synthesis, John Wiley and Sons (1995), and subsequent versions thereof.

Before use, the at least one compound and/or at least one pharmaceutically acceptable salt described herein can be purified by column chromatography, high performance liquid chromatography, crystallization, or other suitable methods.

The antibody-drug conjugate (ADC) of the present invention can be prepared by methods well known in the art, for example, a commonly used ADC conjugation method which comprises the following steps:
(a) Ab (a targeting moiety, preferably an antibody or an antigen-binding fragment thereof) was added to a buffer solution, and a reducing agent was added, and then incubation was performed;
(b) A linker-payload compound or a pharmaceutically acceptable salt thereof, or a solvate thereof described in the present invention was added to the reaction solution in step (a) for conjugation, thus to obtain a crude product;
(c) The crude product was optionally purified to afford the antibody-drug conjugate represented by formula (I).

In some embodiments, the antibody-drug conjugate represented by formula (I) can be obtained by conjugating the linker moiety of the linker-payload compound of the present invention to the active group of the antibody moiety (e.g., cetuximab) in water and/or an organic solvent. Suitable organic solvents can be selected from N,N-dimethylformamide, dimethyl sulfoxide, N-methylpyrrolidone, nitriles (such as acetonitrile), alcohols (such as methanol or ethanol), and any combination thereof. The conjugation method may involve opening a disulfide bond of the antibody by using a reducing agent (e.g., TCEP) to yield sulfydryl, then reacting with the linker moiety of the linker-payload compound to form a C—S bond and then performing conjugation. The resulting conjugation product can be purified by a chromatography method (such as one or more of ion exchange chromatography, hydrophobic chromatography, reverse phase chromatography, or affinity chromatography).

In some embodiments, the method comprises the following two steps:
Step I: A buffer, a TCEP solution, and a DTPA solution were successively added to a monoclonal antibody solution of an appropriate concentration formulated with a reaction buffer, and the resulting solution was reacted at 15-37° C. for 60-180 minutes. The reaction buffer was one of PBS-D buffer (10 mmol/L, pH 7.0), histidine-acetic acid buffer (20 mmol/L, pH 5.5), histidine-hydrochloric acid buffer (10 mmol/L, pH 6.5), histidine buffer (10 mmol/L, pH 7.0), or histidine buffer (10 mmol/L, pH 9.0). TCEP was dissolved in water or a corresponding reaction buffer;
Step II: A solution of the linker-payload compound in DMSO:H₂O was added, and the reaction was further performed at 15-37° C. for 1-18 h. The reaction solution was transferred to an ultrafiltration centrifuge tube (Millipore Amicon® Ultra), and the corresponding buffer solution was added for solution exchange, thus a target product was obtained. The reaction buffer was one of PBS-D buffer (10 mmol/L, pH 7.0), histidine-acetic acid buffer (20 mmol/L, pH 5.5), histidine-hydrochloric acid buffer (10 mmol/L, pH 6.5), histidine buffer (10 mmol/L, pH 7.0), or histidine buffer (10 mmol/L, pH 9.0).

Suitable in-vitro assays can be used to preliminarily evaluate the effect of the at least one antibody-drug conjugate of formula (I) and/or at least one pharmaceutically acceptable salt thereof described herein, in inhibiting the activity of PI3K and/or PIKK kinases, and their efficacy in treating cancer or inflammatory diseases can be further examined by in-vivo experiments. For example, the antibody-drug conjugate of formula (I) and/or the pharmaceutically acceptable salt thereof can be administered to an animal (e.g., a mouse model) suffering from cancer or inflammatory diseases, and then their therapeutic effects can be accessed. Based on the above results, an appropriate dosage range and administration route for animals (such as humans) can also be determined.

The present invention further provides a method of inhibiting the activity of PI3K and/or PIKK kinases, the method comprises contacting an effective amount of at least one antibody-drug conjugate of formula (I) and/or at least one pharmaceutically acceptable salt thereof described herein with at least one kinase. The antibody-drug conjugate of formula (I) and/or the at least one pharmaceutically acceptable salt thereof described herein can be used to achieve a beneficial therapeutic or prophylactic effect.

The present invention further provides a method of treating a disease state or a condition associated with the PI3K and/or PIKK activity or mediated in part by PI3K and/or PIKK (mediated at least in part by PI3K and/or PIKK), comprising administering to a subject in need thereof a therapeutically effective amount of the antibody-drug conjugate of formula (I) described herein. Any disease or condition that is ameliorated by inhibition of phosphoinositide 3-kinase (referred to herein as a “disease mediated at least in part by PI3K and/or PIKK”) can be treated using the antibody-drug conjugate of formula (I) or the composition thereof described herein.

The term “diseases mediated at least in part by PI3Ks and/or PIKKs” refers to diseases with a pathogenesis comprising at least a portion of factors associated with the PI3Ks and/or PIKKs, these diseases including but not limited to: cancers, neurodegenerative diseases, viral infections, bacterial infections, ocular diseases, kidney diseases, autoimmune diseases, cardiovascular and cerebrovascular diseases, and psychological diseases.

The term “cancer (or tumor)” as used refers to a cellular disorder characterized by uncontrolled or dysregulated cell proliferation, decreased cellular differentiation, inappropriate ability to invade surrounding tissues, and/or ability to establish new growth at ectopic sites. Cancers include, but are not limited to, solid tumors and hematological tumors.

Non-limiting examples of solid tumors include cholangiocarcinoma, pancreatic cancer, bladder cancer, colorectal cancer, breast cancer (including metastatic breast cancer), prostate cancer (including androgen-dependent and non-androgen-dependent prostate cancer), kidney cancer (including metastatic renal cell carcinoma), hepatocellular carcinoma, lung cancer (including non-small cell lung cancer (NSCLC), bronchioloalveolar carcinoma (BAC)), esophageal cancer, urothelial carcinoma, ovarian cancer (including progressive ovarian carcinoma or progressive primary peritoneal carcinoma), cervical cancer, endometrial cancer, gastric cancer, esophageal cancer, head and neck tumors (including head and neck squamous cell carcinoma), skin cancer (including malignant melanoma), neuroendocrine cancer (including metastatic neuroendocrine neoplasma), brain tumor (including for example glioma, anaplastic oligodendroglioma, and adult glioblastoma multiforme), bone cancer, soft-tissue sarcoma, and thyroid cancer.

Non-limiting examples of hematological tumors include acute myeloid leukemia (AML), chronic myelogenous leukemia (CML), including accelerated phase of chronic myelogenous leukemia and blast phase of chronic myelogenous leukemia (CML-BP), lymphoma, acute lymphoblastic leukemia (ALL), chronic lymphocytic leukemia (CLL), Hodgkin's disease (HD), non-Hodgkin's lymphoma (NHL) (including follicular lymphoma and mantle cell lymphoma), B-cell lymphoma, T-cell lymphoma, multiple myeloma (MM), diffuse large B-cell lymphoma (DLBL or DLBCL), Waldenstrom's macroglobulinemia, Burkitt's lymphoma, myelodysplastic syndromes (MDS) (including refractory anemia (RA), refractory anemia with ringed siderblasts (RARS), refractory anemia with excess blasts (RAEB), and RAEB in transformation (RAEB-T), and myeloproliferative syndrome.

In some embodiments, the present invention provides a method of delivering a PI3K and/or PIKK inhibitor to a cell expressing EGFR, comprising conjugating the PI3K and/or PIKK inhibitor to an antibody that immunospecifically binds to an EGFR epitope to obtain an antibody-drug conjugate (“ADC”) and exposing the cell to the ADC.

In some embodiments, the ADC provided by the present invention can be administered in any cell or tissue that expresses EGFR, such as cancer cells or tissues that express EGFR. Exemplary embodiments include a method for killing cancer cells or tissues that express EGFR. The method can be used in any cell or tissue that expresses EGFR, such as cancer cells or metastatic lesions.

In some embodiments, non-limiting examples of EGFR-expressing cancers that can be treated include: esophageal cancer, lung cancer, head and neck tumor, pancreatic cancer, colorectal cancer, cholangiocarcinoma cancer, breast cancer, ovarian cancer, uterine cancer, cervical cancer, prostate cancer, bladder cancer, urothelial carcinoma, gastric cancer, kidney cancer, liver cancer, brain cancer, bone cancer, lymphoma and leukemia.

The term “autoimmune disease” refers to a disease or condition caused by the immune response of body to its own antigens leading to damage to its own tissues or organs.

Non-limiting examples of autoimmune diseases include, but are not limited to, chronic obstructive pulmonary disease (COPD), allergic rhinitis, myasthenia gravis, systemic lupus erythematosus, autoimmune hematological diseases (e.g., hemolytic anemia, acquired hemophilia A (AHA), aplastic anemia, pure erythrocytic anemia and idiopathic thrombocytopenia (ITP), autoimmune inflammatory bowel disease (e.g., ulcerative colitis and Crohn's disease)), asthma and idiopathic thrombocytopenic purpura, Grave's disease, multiple sclerosis (MS), psoriasis, psoriatic arthritis, rheumatoid arthritis (RA), activated phosphoinositide 3-kinase-delta syndrome (APDS), and myeloproliferative disease (e.g., myelofibrosis, and post-polycythemia vera/essential thrombocytosis myelofibrosis (post-PV/ET myelofibrosis));
the neurodegenerative diseases include, but are not limited to, Alzheimer's disease, Huntington's disease, cerebellar atrophy, and Pick's disease; the viral infections include, but are not limited to, acquired immunodeficiency syndrome (AIDS); the bacterial infections include, but are not limited to, streptococcal infections; the ocular diseases include, but are not limited to, uveitis (anterior and posterior), retinopathy (e.g., diabetic retinopathy or hyperbaric oxygen-induced retinopathy), and conditions characterized by aqueous humor secretion or raised intraocular pressure (e.g., glaucoma); the kidney diseases include, but are not limited to, glomerulonephritis; the cardiovascular and cerebrovascular diseases include, but are not limited to, atherosclerosis, hypertension, deep vein thrombosis, stroke, myocardial infarction, unstable angina pectoris, thromboembolism, pulmonary embolism, thrombolytic disease, acute arterial ischemia, peripheral thrombotic occlusion, coronary artery disease, and reperfusion injury; the psychological diseases include, but are not limited to, depression, anxiety, and psychological disorders.

### DETAILED DESCRIPTION OF EMBODIMENTS

The examples below are intended to be purely exemplary and should not be considered to be limiting the invention in any way. Efforts have been made to ensure the accuracy with respect to numbers used (such as amounts, temperature, etc.), but a person skilled in the art should understand that some experimental errors and deviations should be accounted for. Unless indicated otherwise, parts are parts by weight, temperature is in degrees Centigrade, and pressure is at or near atmospheric. All MS data were determined by Agilent 6120 and Agilent 1100. All NMR data were generated using a Varian 400 MR machine. All reagents and starting materials, except synthesized intermediates, used in the present invention were commercially available. All compound names except the reagents were generated by Chemdraw 20.1.

If there is any atom with empty valence(s) in any one of the structures disclosed herein, the empty balance(s) is (are) the hydrogen atom(s) which is (are) omitted for convenience purpose.

In the present application, in the case of inconsistency of the name and structure of a compound, when the two of which are both given for the compound, it is subject to the structures of the compound, unless the context shows that the structure of the compound is incorrect, and the name is correct.

List of abbreviations used in the following examples:

| | |
|---|---|
| ADC | Antibody-drug conjugate |
| B₂pin₂ | Bis(pinacolato)diboron |
| CDI | N,N′-carbonyl diimidazole |
| CH₃CN | Acetonitrile |
| DBU | 1,8-diazabicyclo-undec-7-ene |
| DCM | Dichloromethane |
| DIEA | N,N-diisopropylethylamine |
| DMA | N,N-dimethylacetylamide |
| DMAP | 4-dimethylaminopyridine |
| DMF | N,N-dimethylformamide |
| DMSO | Dimethyl sulfoxide |
| Dowtherm A | Diphenyl ether-biphenyl eutectic |
| DTPA | Diethylenetriaminepentaacetic acid |
| EA | Ethyl acetate |
| EDCI | 1-ethyl-(3-dimethylaminopropyl)carbodiimide |
| Et₂N | Diethylamine |
| Et₃N | Triethylamine |
| Fmoc-OSU | 9-fluorenylmethyl-N-succinimidyl carbonate |
| g | Gram |
| h | Hour |
| HATU | 2-(7-azabenzotriazol-1-yl)-N,N,N′,N′-tetramethyluronium |
| | hexafluorophosphate |
| His | Histidine |
| HIC-HPLC | Hydrophobic interaction chromatography |
| H₂O | Water |
| HOBt | 1-hydroxybenzotriazole |
| ISCO | Fast preparative liquid chromatography |
| L | Liter |
| M | Mole/liter |
| MeOH | Methanol |
| mg | Milligram |
| min | Minute |
| mL | Milliliter |
| mmol | Millimole |
| mol | Mole |
| MS | Mass spectrometry |
| PBS-D | Phosphate buffered solution- |
| | Diethylenetriaminepentaacetic acid |
| Pd(dppf)Cl₂ | [1,1′-bis(diphenylphosphino)ferrocene]palladium |
| | dichloride |
| PdCl₂(PPh₃)₂ | Bis(triphenylphosphine)palladium (II) dichloride |
| PE | Petroleum ether |
| pH | Power of hydrogen |
| POCl₃ | Phosphorus oxychloride |
| SO₂Cl₂ | Sulfonyl chloride |
| TCEP | Tris(2-carboxyethyl)phosphine |
| TFA | Trifluoroacetic acid |
| THF | Tetrahydrofuran |

### Example 1: Preparation of Intermediates

### Preparation of Intermediate-1:

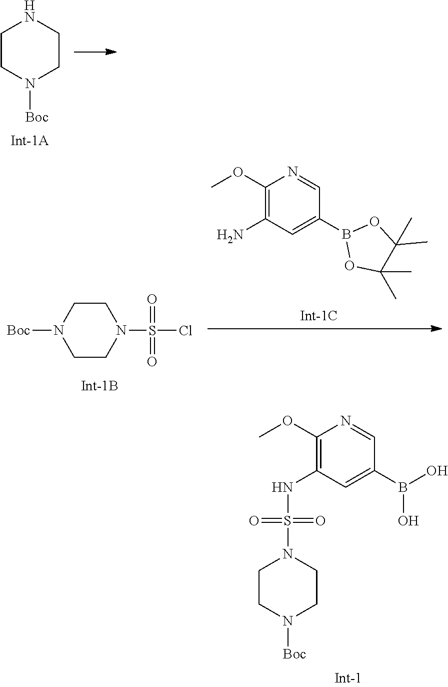

### 1) Synthesis of Compound Int-1B

In an ice-water bath, SO₂Cl₂ (19.93 g, 147.65 mmol) was added to a solution of compound Int-1A (commercially available, CAS: 57260-71-6) (25 g, 134.22 mmol) and pyridine (15.91 g, 201.34 mmol) in dichloromethane. After the completion of dropwise addition, the reaction solution was stirred continuously in the ice-water bath for 2 hours. After the reaction was completed, the reaction solution was poured into water, and extracted with dichloromethane (200 mL×3). The extract was washed once with 1 N hydrochloric acid (100 mL). The organic phases were combined, dried over anhydrous sodium sulfate, and concentrated under reduced pressure to afford compound Int-1B as a yellow solid (18 g, 47% yield).

### 2) Synthesis of Intermediate-1

At room temperature, to a solution of compound Int-1B (17.08 g, 59.97 mmol) and compound Int-1C (commercially available, CAS: 1000339-10-5) (10 g, 39.98 mmol) in 1,2-dichloroethane were added DMAP (9.77 g, 79.96 mmol) and Et₃N (6.06 g, 59.97 mmol). After the addition was completed, the reaction solution was stirred at 75° C. for 24 hours. After the reaction was completed, the mixture was filtered, and the resulting filtrate was concentrated. The resulting residue was dissolved in ethyl acetate (200 mL), and washed with 1 N hydrochloric acid (60 mL). The organic phase was collected. At the same time, the aqueous phase was extracted with ethyl acetate (100 mL×3). The organic phases were combined and dried over anhydrous sodium sulfate. The solvent was removed by concentration under reduced pressure. The resulting residue was purified by ISCO (mobile phase: water (0.1% formic acid):methanol=100:0-0:100) to afford intermediate-1 (abbreviated as Int-1) as a yellow solid (7 g, 42% yield). MS(m/z)=439.0 [M+Na]+

### Preparation of Intermediate-2:

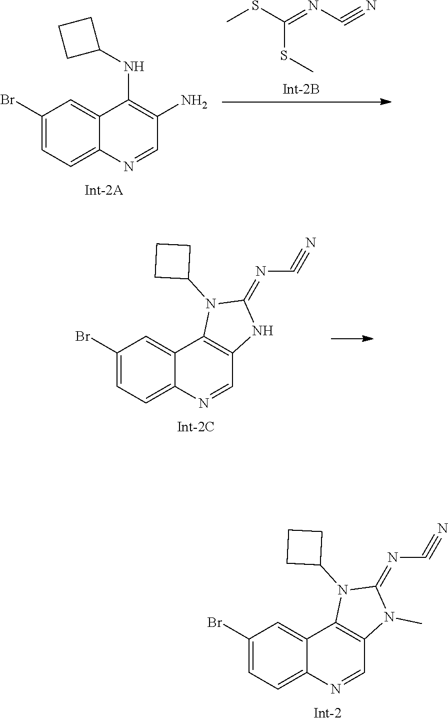

### 1) Synthesis of Compound Int-2C

Under nitrogen protection, a solution of compound Int-2A (according to the preparation procedure of intermediate-5) (190 mg, 0.65 mmol), compound Int-2B (commercially available, CAS: 10191-60-3) (190 mg, 1.30 mmol) and DBU (297 mg, 1.95 mmol) in dioxane was stirred at 100° C. overnight. After the reaction was completed, the mixture was concentrated and the residue was purified by ISCO (mobile phase: MeOH:H₂O=0:100-100:0) to afford compound Int-2C as a white solid (102 mg, 46% yield). MS(m/z)=318, 320 [M−24]+

### 2) Synthesis of Intermediate-2

In an ice-water bath and under nitrogen protection, to a solution of compound Int-2C (102 mg, 0.30 mmol) in DMF was added NaH (14 mg, 0.60 mmol). After the addition was completed, the reaction solution was stirred under nitrogen protection in an ice-water bath for 10 minutes. Then, CH₃I (85 mg, 0.60 mmol) was added to the reaction solution in one portion. The reaction was stirred at room temperature for 1 h. After the reaction was completed, the reaction solution was poured into water, and extracted with ethyl acetate (20 mL×3). The organic phases were combined, dried over anhydrous sodium sulfate, and concentrated under reduced pressure. The residue was purified by ISCO (mobile phase: MeOH:H₂O=0:100-100:0) to afford intermediate-2 (abbreviated as Int-2) as a white solid (23 mg, 22% yield). MS(m/z)=332.0, 334.0 [M−24]+

### Preparation of Intermediate-3

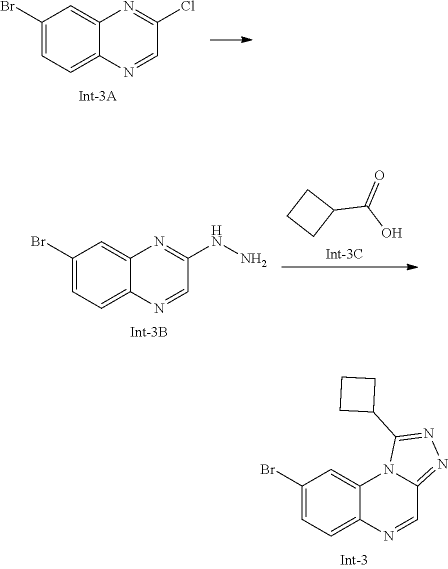 [SMILES: Clc1cnc2ccc(Br)cc2n1 ; O=C(O)C1CCC1 ; NNc1cnc2ccc(Br)cc2n1 ; Brc1ccc2ncc3nnc(C4CCC4)n3c2c1]

### 1) Synthesis of Compound Int-3B

Compound Int-3A (5 g, 20.53 mmol) and hydrazine hydrate (10 mL) were dissolved in ethanol (100 mL). The reaction was stirred at reflux overnight. After the reaction was completed, the mixture was concentrated under reduced pressure to make the volume of the reaction solution reduce to half of the original volume. The solid was collected by filtration, and dried to afford Int-3B as a light-yellow solid (3.9 g, 79.0% yield). MS(m/z)=239.0, 241.0 [M+H]⁺

### 2) Synthesis of Intermediate-3

Compound Int-3B (500 mg, 2.09 mmol) and compound Int-3C (2 mL) were mixed, heated to 110° C. and stirred for 24 h. After the reaction was completed, purification was conducted by using ISCO (mobile phase: dichloromethane:methanol=100:0-75:25) to afford compound intermediate-3 (abbreviated as Int-3) as a light-yellow solid (460 mg, 73% yield). MS(m/z)=303.0, 305.0 [M+H]⁺

The following compounds were prepared according to the preparation procedure of intermediate-3 using the corresponding intermediates and reagents under appropriate conditions recognized by a person skilled in the art.

| Intermediate No. | Structural formula | MS (M + H)⁺ |
|---|---|---|
| Intermediate-4 | 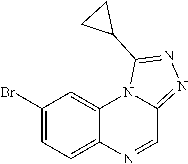 [SMILES: Brc1ccc2ncc3nnc(C4CC4)n3c2c1] | 289.0 [M + 1]⁺ |

### Preparation of Intermediate-5

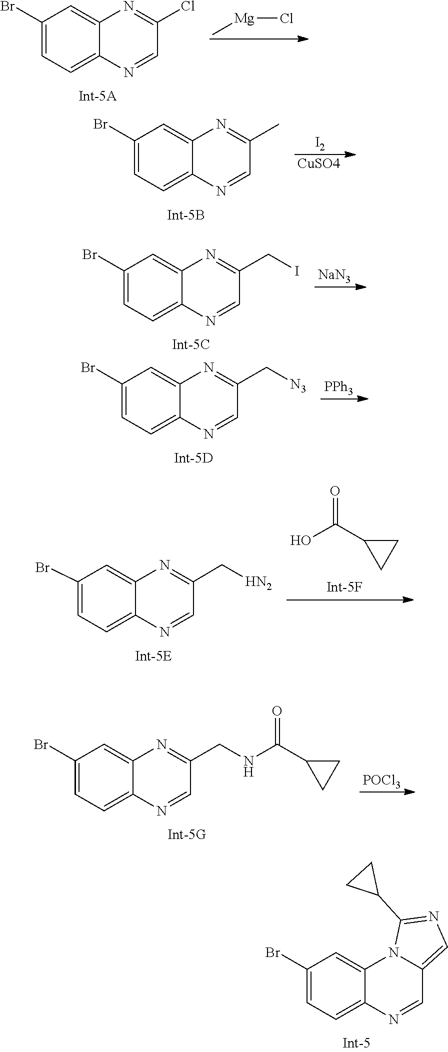

### 1) Synthesis of Compound Int-5B

In an ice-water bath and under nitrogen protection, methyl magnesium chloride (9 mL, 27.1 mmol) was slowly added to a solution of compound Int-5A (CAS: 89891-65-6) (6 g, 24.6 mmol) and iron triacetylacetonate (0.9 g, 2.5 mmol) in THF. The reaction was stirred at 0° C. for 2 h. After the reaction was completed, the reaction solution was extracted with ethyl acetate (200 mL×3). The organic phases were combined, dried over anhydrous sodium sulfate, and concentrated under reduced pressure. The residue was purified by ISCO (water (0.1% TFA):methanol=100:0-0:100 gradient elution) to afford compound Int-5B as a yellow solid (5.0 g, 90.9% yield). MS(m/z)=223.2 [M+H]⁺

### 2) Synthesis of Compound Int-5C

Under nitrogen protection, iodine (8.53 g, 33.6 mmol), compound Int-5B (5 g, 22.4 mmol) and copper sulfate (3.56 g, 22.4 mmol) was dissolved in acetonitrile, and the reaction was stirred at 70° C. for 3 h. After the reaction was completed, the reaction solution was concentrated and the residue was purified by ISCO (water (0.1% TFA):methanol=100:0-0:100 gradient elution) to afford compound Int-5C as a yellow solid (1.0 g, 12.8% yield). MS(m/z)=349.2.2 [M+H]⁺

### 3) Synthesis of Compound Int-5D

Under nitrogen protection, sodium azide (224 mg, 3.4 mmol) was added to a solution of compound Int-5C (1 g, 2.8 mmol) in DMF, and the reaction was stirred at room temperature for 2 h. After the reaction was completed, the reaction solution was extracted with ethyl acetate (100 mL×3). The organic phases were combined, dried over anhydrous sodium sulfate, and concentrated under reduced pressure. The residue was purified by ISCO (water (0.1% TFA):methanol=100:0-0:100 gradient elution) to afford compound Int-5D as a white solid (480 mg, 63.4% yield). MS(m/z)=263.8 [M+H]⁺

### 4) Synthesis of Compound Int-5E

Under nitrogen protection, triphenylphosphine (572 mg, 2.2 mmol) was added to a solution of compound Int-5D (480 mg, 1.8 mmol) in THF:H₂O (2:1), and the reaction was stirred at room temperature for 12 h. After the reaction was completed, the reaction solution was concentrated, and the residue was purified by ISCO (water (0.1% TFA):methanol=100:0-0:100 gradient elution) to afford compound Int-5E as a black solid (270 mg, 62.4% yield). MS(m/z)=237.8 [M+H]⁺

### 5) Synthesis of Compound Int-5G

Under nitrogen protection, HATU (210 mg, 0.55 mmol), DIEA (195 mg, 1.5 mmol), and compound Int-5F (43 mg, 0.5 mmol) were added to a solution of compound Int-5E (120 mg, 0.5 mmol) in DMF, and the reaction was stirred at room temperature for 1 h. After the reaction was completed, the reaction solution was concentrated and the residue was purified by ISCO (water (0.1% TFA):methanol=100:0-0:100 gradient elution) to afford compound Int-5G as a white solid (100 mg, 64.8% yield). MS(m/z)=305.8 [M+H]⁺

### 6) Synthesis of Intermediate-5

Under nitrogen protection, compound Int-5G (100 mg, 0.33 mmol) was added to POCl₃, and the reaction was stirred at 75° C. for 3 h. After the reaction was completed, the reaction solution was concentrated and the residue was purified by ISCO (water (0.1% TFA):methanol=100:0-0:100 gradient elution) to afford intermediate-5 (abbreviated as Int-5) as a white solid (85 mg, 90.3% yield). MS(m/z)=287.8 [M+H]⁺

The following compounds were prepared according to the preparation procedure of intermediate-5 using the corresponding intermediates and reagents under appropriate conditions recognized by a person skilled in the art.

| Intermediate No. | Structural formula | MS (M + H)⁺ |
|---|---|---|
| Intermediate-6 | 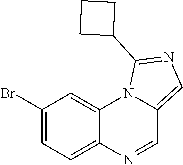 [SMILES: Brc1ccc2ncc3cnc(C4CCC4)n3c2c1] | 301.8 [M + H]⁺ |
| Intermediate-7 | 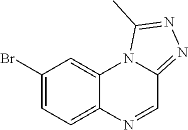 | 261.8 [M + H]⁺ |
| Intermediate-24 | 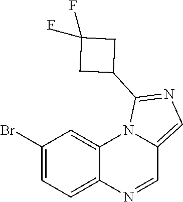 | 338.0 [M + H]⁺ |

### Preparation of Intermediate-8:

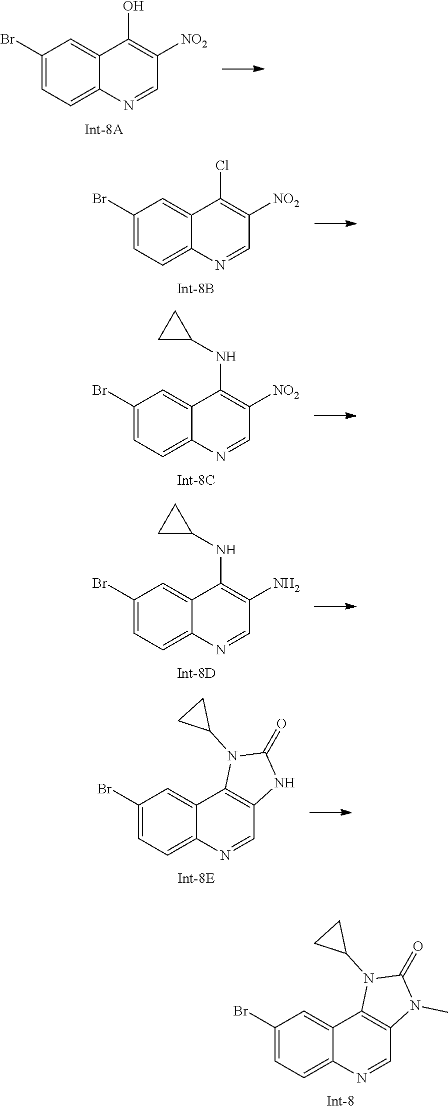 [SMILES: O=[N+]([O-])c1cnc2ccc(Br)cc2c1O ; O=[N+]([O-])c1cnc2ccc(Br)cc2c1Cl ; O=[N+]([O-])c1cnc2ccc(Br)cc2c1NC1CC1 ; Nc1cnc2ccc(Br)cc2c1NC1CC1 ; O=c1[nH]c2cnc3ccc(Br)cc3c2n1C1CC1 ; Cn1c(=O)n(C2CC2)c2c3cc(Br)ccc3ncc21]

### 1) Synthesis of Compound Int-8B

Under nitrogen protection, compound Int-8A (CAS: 853908-50-6) (2.69 g, 10 mmol) was dissolved in phosphorus oxychloride (15 mL). The reaction solution was stirred for 5 hours while being heated to reflux. After the reaction was completed, the reaction solution was slowly poured into ice water, and extracted with dichloromethane (30 mL×3). The organic phases were combined and concentrated to afford compound Int-8B as a yellow solid (2.58 g, 90% yield). MS(m/z)=287.0 [M+H]⁺

### 2) Synthesis of Compound Int-8C

Compound Int-8B (2.58 g, 9 mmol) and cyclopropylamine (514 mg, 9 mmol) were dissolved in isopropanol (15 mL). The reaction was stirred at 80° C. for 4 h. After the reaction was completed, the reaction solution was naturally cooled to room temperature, and a solid was precipitated out obviously. The solid was collected by filtration, washed with isopropanol, and dried in vacuo to afford a yellow solid (1.94 g, 70% yield). MS(m/z)=308.0 [M+H]⁺

### 3) Synthesis of Compound Int-8D

Tin dichloride dihydrate (5.68 g, 25.2 mmol) was added to a solution of compound Int-8C (1.94 g, 6.3 mmol) in ethyl acetate (40 mL) and the reaction was stirred at reflux for 4 h. After the reaction was completed, the reaction was cooled to room temperature and the pH was adjusted to pH 9.0 with 2 N aqueous sodium hydroxide solution. The reaction solution was then filtered. The filtrate was collected, and extracted with ethyl acetate (30 mL×3). The organic phases were combined, dried over anhydrous sodium sulfate, and concentrated under reduced pressure to afford compound Int-8D as a yellow solid (1.41 g, 80% yield). MS(m/z)=278.0 [M+H]⁺

### 4) Synthesis of Compound Int-8E

In an ice-water bath, to a solution of compound Int-8D (556 mg, 2 mmol) and triethylamine (607 mg, 6 mmol) in dichloromethane (10 mL) was added triphosgene (415 mg, 1.4 mmol) in batches. After the addition was completed, the mixture was stirred at room temperature for 3 h. After the reaction was completed, the reaction solution was slowly poured into water, and extracted with dichloromethane (30 mL×3). The organic phases were combined, dried over anhydrous sodium sulfate, and concentrated under reduced pressure to afford compound Int-8E as a brown solid (610 mg, 99% yield). MS(m/z)=304.0 [M+H]⁺

### 5) Synthesis of Intermediate-8

To a solution of compound Int-8E (610 mg, 2 mmol) in DMF (10 mL) was added potassium carbonate (829 mg, 6 mmol) followed by methyl iodide (710 mg, 5 mmol). After the addition was completed, the reaction solution was stirred at room temperature for 18 hours. After the reaction was completed, the reaction solution was concentrated, and the residue was purified by ISCO (mobile phase: water (0.1% formic acid):acetonitrile=100:0-0:100) to afford compound intermediate-8 (abbreviated as Int-8) as a yellow solid (382 mg, 60% yield). MS(m/z)=318.0 [M+H]⁺.

The following compounds were prepared according to the preparation procedure of intermediate-8 using the corresponding intermediates and reagents under appropriate conditions recognized by a person skilled in the art.

| Intermediate | | MS |
|---|---|---|
| No. | Structural formula | (M + H)⁺ |
| Inter- mediate-9 | 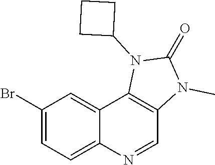 [SMILES: Cn1c(=O)n(C2CCC2)c2c3cc(Br)ccc3ncc21] | 332.2 [M + H]⁺ |
| Inter- mediate-10 | 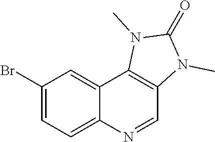 [SMILES: Cn1c(=O)n(C)c2c3cc(Br)ccc3ncc21] | 294.0 [M + H]⁺ |
| Inter- mediate-11 | 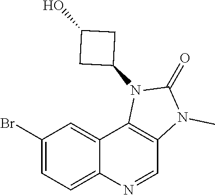 [SMILES: Cn1c(=O)n([C@H]2C[C@H](O)C2)c2c3cc(Br)ccc3ncc21] | 348.0 [M + H]⁺ |
| Inter- mediate-12 | 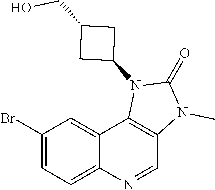 [SMILES: Cn1c(=O)n([C@H]2C[C@H](CO)C2)c2c3cc(Br)ccc3ncc21] | 362.0 [M + H]⁺ |
| Inter- mediate-13 | 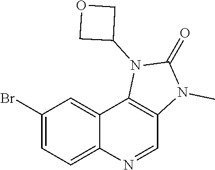 [SMILES: Cn1c(=O)n(C2COC2)c2c3cc(Br)ccc3ncc21] | 334.0 [M + H]⁺ |
| Inter- mediate-14 | 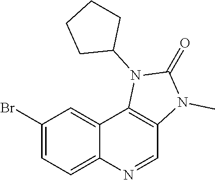 | 336.2 [M + H]⁺ |
| Inter- mediate-25 | 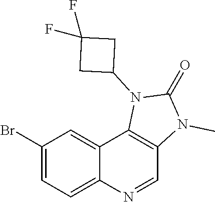 [SMILES: Cn1c(=O)n(C2CC(F)(F)C2)c2c3cc(Br)ccc3ncc21] | 368.0 [M + H]⁺ |
| Inter- mediate-27 | 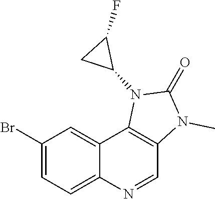 [SMILES: Cn1c(=O)n([C@@H]2C[C@@H]2F)c2c3cc(Br)ccc3ncc21] | 336.0 [M + H]⁺ |
| Inter- mediate-29 | 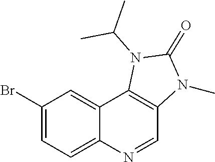 [SMILES: CC(C)n1c(=O)n(C)c2cnc3ccc(Br)cc3c21] | 320.2 [M + H]⁺ |
| Inter- mediate-30 | 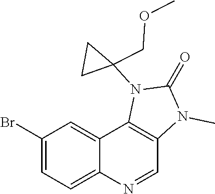 [SMILES: COCC1(n2c(=O)n(C)c3cnc4ccc(Br)cc4c32)CC1] | 364.1 [M + H]⁺ |
| Inter- mediate-32 | 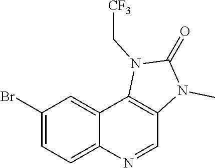 [SMILES: Cn1c(=O)n(CC(F)(F)F)c2c3cc(Br)ccc3ncc21] | 360.0 [M + H]⁺ |
| Inter- mediate-33 | 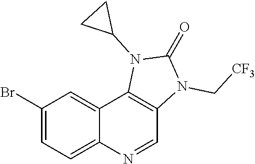 [SMILES: O=c1n(CC(F)(F)F)c2cnc3ccc(Br)cc3c2n1C1CC1] | 385.9 [M + H]⁺ |
| Inter- mediate-35 | 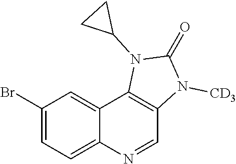 [SMILES: [2H]C([2H])([2H])n1c(=O)n(C2CC2)c2c3cc(Br)ccc3ncc21] | 321.0 [M + H]⁺ |

### Preparation of Intermediate-15:

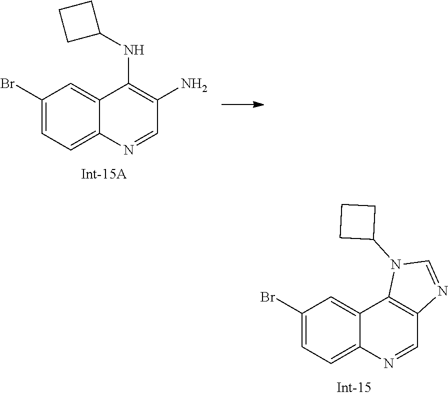 [SMILES: Nc1cnc2ccc(Br)cc2c1NC1CCC1 ; Brc1ccc2ncc3ncn(C4CCC4)c3c2c1]

Compound Int-15A (157 mg, 0.54 mmol) (prepared according to the preparation procedure of intermediate-5), triethyl orthoformate (119 mg, 0.81 mmol), and pyridine hydrochloride (1 mg, 0.005 mmol) were dissolved in toluene (20 mL). The reaction was stirred at reflux for 3 h. After the reaction was completed, the reaction solution was concentrated under reduced pressure and the residue was purified by ISCO (mobile phase: (ACN:H₂O (TFA)=0:100-100:0) to afford intermediate-15 (abbreviated as Int-15) as a white solid (100 mg, 62% yield). MS(m/z)=302.0 [M+1]⁺

The following compounds were prepared according to the preparation procedure of intermediate-15 above using the corresponding intermediates and reagents under appropriate conditions recognized by a person skilled in the art.

| Intermediate No. | Structural formula | MS (M + H)⁺ |
|---|---|---|
| Intermediate-16 | 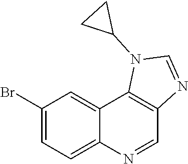 [SMILES: Brc1ccc2ncc3ncn(C4CC4)c3c2c1] | 288.2 [M + 1]⁺ |

### Preparation of Intermediate-17:

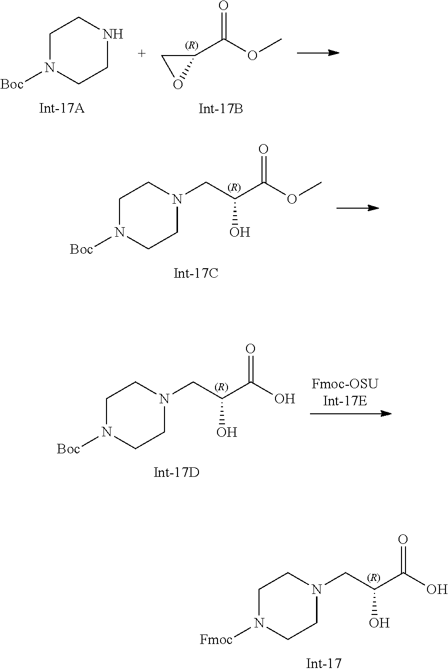

### 1) Synthesis of Compound Int-17C

Compound Int-17A (CAS: 57260-71-6) (4 g, 21.5 mmol), methyl (R)-2,3-epoxypropionate (2.63 g, 25.8 mmol), and DIEA (11.2 mL, 64.5 mmol) were dissolved in DMF (30 mL), and the reaction solution was stirred at 80° C. for 18 hours. After the reaction was cooled to room temperature, water (40 mL) was added, the mixture was extracted with ethyl acetate (25 mL×3). The organic phases were combined, washed once with water (40 mL), dried over anhydrous sodium sulfate and filtered. The resulting filtrate was concentrated to afford Int-17C as a light-yellow solid (5.26 g, 85% yield). MS(m/z): 289.2 [M+H]⁺

### 2) Synthesis of Compound Int-17D

Compound Int-17C (2.88 g, 10 mmol) and lithium hydroxide monohydrate (2 g, 50 mmol) were dissolved in a mixed solution of methanol/water (15 mL/6 mL), and the reaction solution was heated to 40° C. and stirred for 18 hours. The reaction solution was cooled to room temperature, further cooled in an ice-water bath and then adjusted to pH 7.0 with 5 N hydrochloric acid in the ice-water bath. The mixture was concentrated under reduced pressure, and separated by ISCO (mobile phase: H₂O (0.1% HCOOH):CH₃CN=100:0-0:100) to afford compound Int-17D as a light-yellow solid (2.41 g, 88.0% yield). MS(m/z): 275.2 [M+H]⁺

### 3) Synthesis of Intermediate-17

Under nitrogen protection, compound Int-17D (500 mg, 1.8 mmol) was added to a mixed solution of TFA:DCM (1:1, 5 mL) and the mixture was stirred for 20 minutes. After the reaction was completed, the reaction solution was concentrated to afford a residue. The residue was dissolved in a mixed solvent of dioxane and water (1:1, 10 mL). Sodium bicarbonate (460 mg, 5.5 mmol) and Fmoc-OSU (Int-17E, CAS: 82911-69-1, 677 mg, 2.0 mmol) were added to the above solution in an ice bath. The reaction was carried out at room temperature for 3 hours. After the reaction was completed, the reaction solution was extracted with ethyl acetate (20 mL×3). The organic phases were combined, dried over anhydrous sodium sulfate, and concentrated under reduced pressure, and then the residue was purified by ISCO (water (0.1% TFA):acetonitrile=100:0-0:100 gradient elution) to afford intermediate-17 (abbreviated as Int-17) as a white solid (650 mg, 90.0% yield). MS(m/z)=397.6 [M+H]⁺

### Preparation of Intermediate-18:

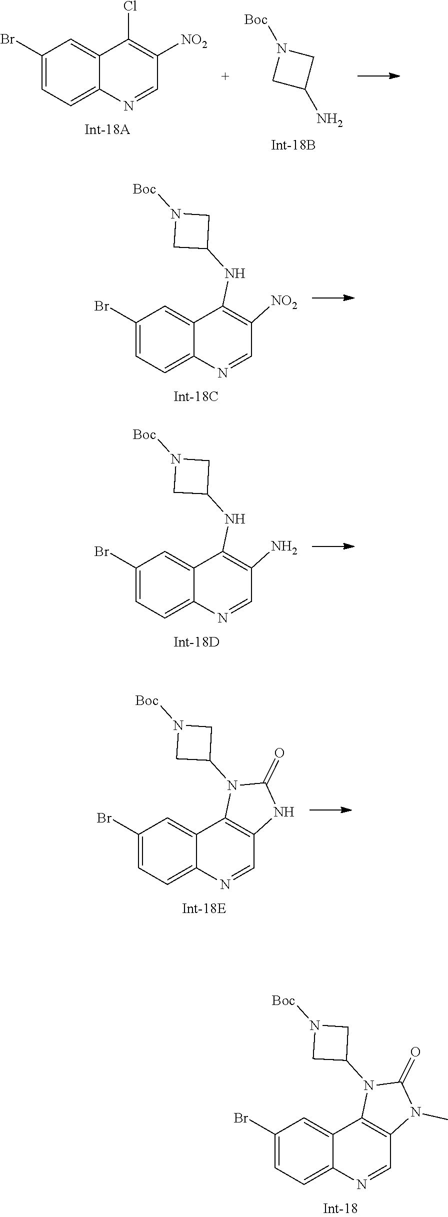 [SMILES: O=[N+]([O-])c1cnc2ccc(Br)cc2c1Cl ; CC(C)(C)OC(=O)N1CC(N)C1 ; CC(C)(C)OC(=O)N1CC(Nc2c([N+](=O)[O-])cnc3ccc(Br)cc23)C1 ; CC(C)(C)OC(=O)N1CC(Nc2c(N)cnc3ccc(Br)cc23)C1 ; CC(C)(C)OC(=O)N1CC(n2c(=O)[nH]c3cnc4ccc(Br)cc4c32)C1 ; Cn1c(=O)n(C2CN(C(=O)OC(C)(C)C)C2)c2c3cc(Br)ccc3ncc21]

### 1) Synthesis of Compound Int-18C

Compound Int-18A (prepared in step 1 of the preparation for Int-8) (1.7 g, 6 mmol) and compound Int-18B (1.03 g, 6 mmol) were dissolved in isopropanol (15 mL). The reaction was stirred at 80° C. for 4 h. After the reaction was completed, the reaction solution was naturally cooled to room temperature, and a solid was precipitated out obviously. A filter cake was obtained by filtration, which was washed with isopropanol to afford a yellow solid (2.2 g, 86% yield). MS(m/z)=423.0 [M+H]⁺

### 2) Synthesis of Compound Int-18D

Compound Int-18C (2 g, 4.73 mmol), iron powder (1.59 g, 28.4 mmol), and ammonium chloride (1.53 g, 28.4 mmol) were added to a mixed solution of ethanol and water (30 mL/10 mL), and the reaction was stirred at 90° C. for 5 h. After the reaction was completed, the reaction solution was cooled to room temperature, and then was filtered. The filtrate was collected and concentrated under reduced pressure to afford compound Int-18D as a yellow solid (1.5 g, 80% yield). MS(m/z)=393.2 [M+H]⁺

### 3) Synthesis of Compound Int-18E

In an ice-water bath, to a solution of compound Int-18D (1.5 g, 3.81 mmol) and triethylamine (1.16 g, 3 mmol) in dichloromethane (10 mL) was added triphosgene (407 mg, 1.37 mmol) in batches. After the addition was completed, the mixture was stirred at room temperature for 3 h. After the reaction was completed, the reaction solution was slowly poured into water, and extracted with dichloromethane. The organic phase was collected, dried and concentrated to afford a brown solid (1.98 g, 99% yield). MS(m/z)=419.0 [M+H]⁺

### 4) Synthesis of Intermediate-18

To a solution of compound Int-18E (1.98 mg, 4.72 mmol) in DMF (15 mL) was added potassium carbonate (2.09 g, 15.1 mmol) followed by methyl iodide (0.44 mL, 7.09 mmol). After the addition was completed, the reaction solution was stirred at room temperature for 18 hours. After the reaction was completed, the reaction solution was concentrated, and the residue was purified by ISCO (mobile phase: water (0.1% trifluoroacetic acid):methanol=100:0-0:100) to afford intermediate-18 (abbreviated as Int-18) as a yellow solid (1.43 g, 70% yield). MS(m/z)=433.0 [M+H]⁺

### Preparation of Intermediate-19:

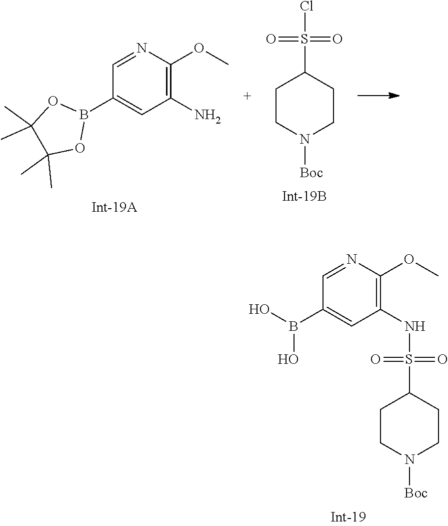

At room temperature and under nitrogen protection, a solution of DIEA (312 mg, 2.40 mmol), DMAP (20 mg, 0.16 mmol), and compound Int-19A (CAS: 893440-50-1) (400 mg, 1.60 mmol) in DCM (10 mL) was added dropwise to a solution of compound Int-19B (CAS: 782501-25-1) (544 mg, 1.92 mmol) in DCM (20 mL). After the addition was completed, the reaction solution was stirred at room temperature for 1 h. After the reaction was completed, the reaction solution was poured into water, and extracted with DCM (15 mL×3). The organic phases were combined, dried over anhydrous sodium sulfate, and concentrated under reduced pressure. The residue was purified by ISCO (mobile phase: CH₃CN:H₂O=0:100-100:0) to afford intermediate-19 (abbreviated as Int-19) as a white solid (158 mg, 24% yield). MS(m/z)=360.1 [M−56]⁺

### Preparation of Intermediate-20:

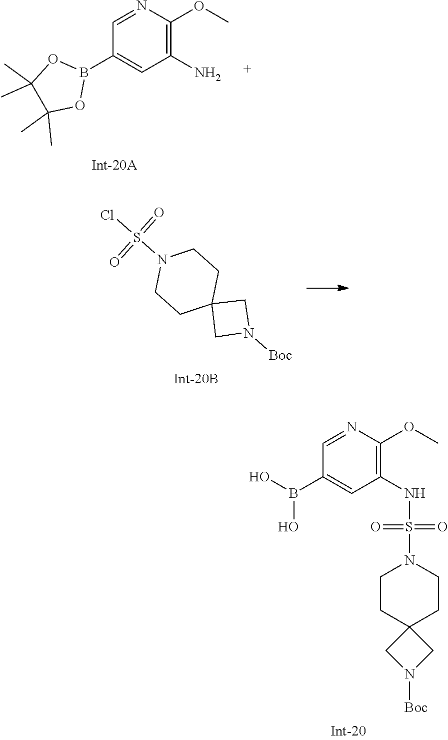

At room temperature and under nitrogen protection, pyridine (0.5 mL), DMAP (15 mg, 0.12 mmol) and compound Int-20A (CAS: 893440-50-1) (400 mg, 1.17 mmol) were added to a solution of compound Int-20B (380 mg, 1.12 mmol) in 1,2 dichloroethane. After the addition was completed, the reaction solution was stirred under nitrogen protection at 70° C. for 24 h. After the reaction was completed, the reaction solution was poured into water, and extracted with DCM (15 mL×3). The organic phases were combined, dried over anhydrous sodium sulfate, and concentrated under reduced pressure. The residue was purified by ISCO (mobile phase: CH₃CN:H₂O=0:100-100:0) to afford intermediate-20 (abbreviated as Int-20) as a white solid (190 mg, 35% yield). MS(m/z)=401.2 [M−56]⁺

### Preparation of Intermediate-21:

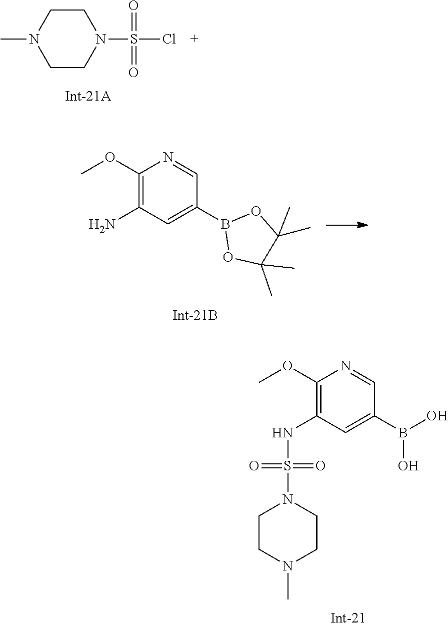

Compound Int-21B (CAS: 893440-50-1) (50 mg, 0.2 mmol) and compound Int-21A (CAS: 893440-50-1) (120 mg, 0.6 mmol) were dissolved in pyridine (4 mL) at room temperature, and placed in a sealed tube. The reaction solution was stirred at 60° C. for 18 hours. After the reaction was completed, the reaction solution was concentrated under reduced pressure, and the residue was purified by ISCO (mobile phase: water (0.1% formic acid):acetonitrile=100:0-0:100) to afford intermediate-21 (abbreviated as Int-21) as a yellow solid (66 mg, 99% yield). MS(m/z)=331.2 [M+1]⁺.

### Preparation of Intermediate-22:

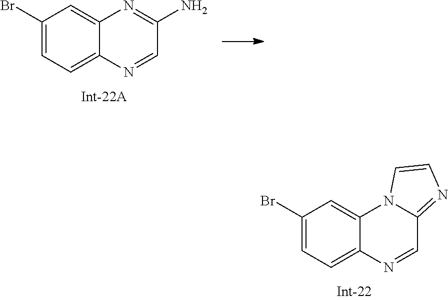 [SMILES: Nc1cnc2ccc(Br)cc2n1 ; Brc1ccc2ncc3nccn3c2c1]

Compound Int-22A (CAS: 212327-11-2) (500 mg, 2.2 mmol) and chloroacetaldehyde (1.7 g, 22 mmol) were added to an ethanol solution, and the reaction was stirred at 105° C. for 12 h. After the reaction was completed, the reaction solution was concentrated, and the residue was purified by ISCO (mobile phase: CH₃CN:H₂O (0.1% TFA)=0:100-100:0) to afford intermediate-22 (abbreviated as Int-22) as a yellow solid (400 mg, 72% yield). MS(m/z)=248.0 [M+H]⁺

### Preparation of Intermediate-23

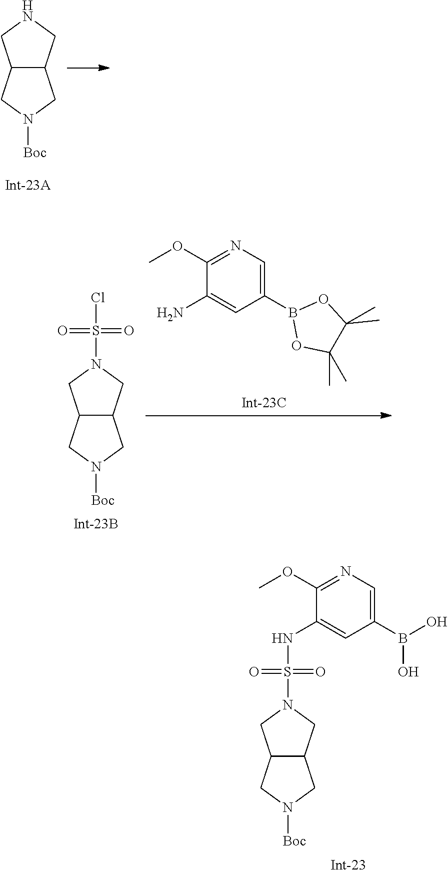

### 1) Synthesis of Compound Int-23B

In an ice-water bath, SO₂Cl₂ (3.5 g, 7.09 mmol) was added to a solution of compound Int-23A (CAS: 141449-85-6) (5 g, 23.55 mmol) and triethylamine (6.55 mL, 15.1 mmol) in dichloromethane. After the completion of dropwise addition, the reaction solution was stirred continuously at room temperature for 2 hours. After the reaction was completed, the reaction solution was poured into water, and extracted with dichloromethane (200 mL×3). The extract was washed once with 1 N hydrochloric acid (100 mL). The organic phases were combined, dried over anhydrous sodium sulfate, and concentrated under reduced pressure to afford compound Int-23B as a yellow solid (5.28 g, 72% yield). MS(m/z)=255.0 [M−55]⁺

### 2) Synthesis of Intermediate-23

Compound Int-23B (2.98 g, 9.6 mmol) and compound Int-23C (CAS: 893440-50-1) (400 mg, 1.6 mmol) were dissolved in pyridine (15 mL) at room temperature, and placed in a sealed tube. The reaction solution was stirred at 60° C. for 18 hours. After the reaction was completed, the reaction solution was concentrated under reduced pressure, and the residue was purified by ISCO (mobile phase: water (0.1% TFA):methanol=100:0-0:100) to afford intermediate-23 (abbreviated as Int-23) as a yellow solid (353 mg, 50% yield). MS(m/z)=441.2 [M−1]⁻

### Preparation of Intermediate-26

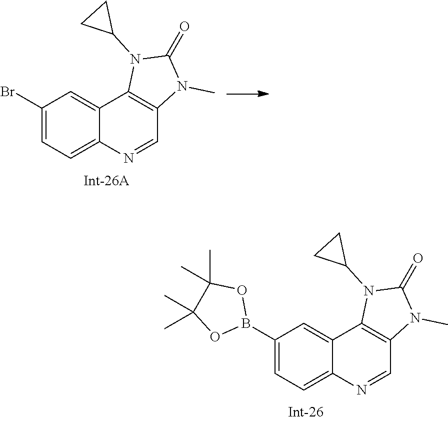

Int-26A (6.0 g, 18.8 mmol) (prepared according to the preparation method for Int-8) was charged into a reaction flask, and 1,4-dioxane (900 mL), B₂Pin₂ (7.2 g, 28.3 mmol), potassium acetate (4.6 g, 46.9 mmol), and Pd(dppf)Cl₂ (0.68 g, 0.93 mmol) were added, purged with N₂, heated to 90° C. and reacted for 16 hours. The reaction solution was cooled to room temperature, and filtered with celite. The filtrate was concentrated to dryness under reduced pressure. A mixed solvent of 20 mL of DCM and 90 mL of hexane was added, and the mixture was slurried at room temperature overnight and then filtered. The filter cake was dried to afford compound intermediate-26 (abbreviated as Int-26) as a gray solid (5.6 g, 81.3% yield). MS(m/z)=366.2 [M+H]⁺

### Preparation of Intermediate-28

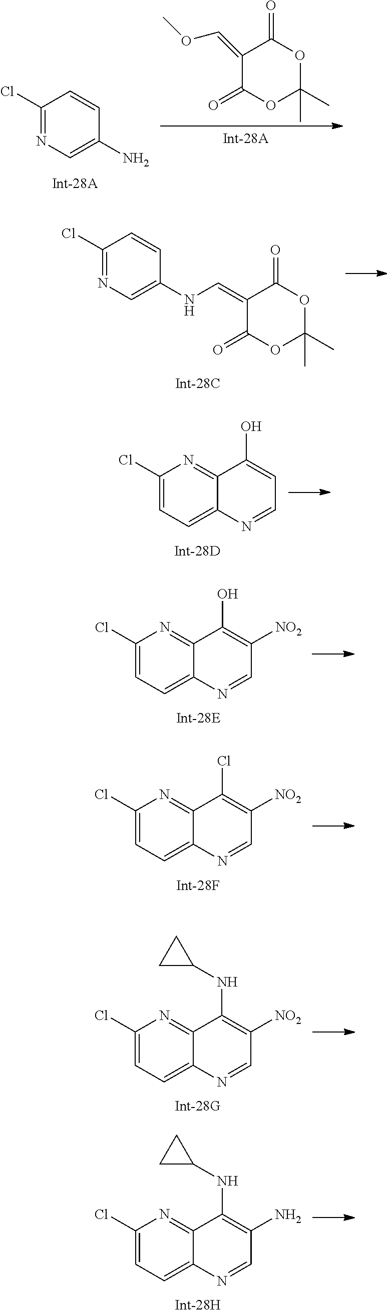

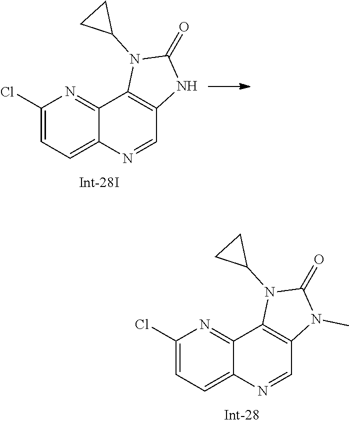 [SMILES: O=c1[nH]c2cnc3ccc(Cl)nc3c2n1C1CC1 ; Cn1c(=O)n(C2CC2)c2c3nc(Cl)ccc3ncc21]

### 1) Synthesis of Compound Int-28C

Compound Int-28A (CAS: 5350-93-6) (6.00 g, 46.67 mmol) and compound Int-28B (CAS: 15568-85-1) (17.38 g, 93.34 mmol) were dissolved in isopropanol (40 mL) at room temperature, and the mixture was refluxed at 90° C. for two hours. After the reaction was completed, the reaction solution was concentrated. The residue was slurried with PE (100 mL) and then filtered. The filter cake was collected to afford compound Int-28C as a brown solid (13.00 g, 98% yield). MS(m/z)=283.0 [M+H]⁺

### 2) Synthesis of Compound Int-28D

At 220° C. and under nitrogen protection, to a solution of Dowtherm A (850 mL) was added compound Int-28C (13.00 g, 45.98 mmol) in batches. After the addition was completed, the reaction solution was stirred at 220° C. and under nitrogen protection for 5 min. Then the reaction was cooled to room temperature. The reaction solution was poured into PE (1000 mL), stirred for 30 min, and filtered. The filter cake was collected to afford compound Int-28D as a brown solid (2.90 g, 34% yield). MS(m/z)=181.2 [M+H]⁺

### 3) Synthesis of Compound Int-28E

KNO₃ (6.49 g, 64.23 mmol) was added to a solution of compound Int-28D (2.90 g, 16.02 mmol) in concentrated H₂SO₄ (9 mL). After the addition was completed, the reaction was placed at 100° C. and stirred for 4 h. After the reaction was completed, the reaction solution was poured into ice water and filtered. The filter cake was collected and dried to afford compound Int-28E as a yellow solid (2.3 g, 61% yield). MS(m/z)=208.2 [M−17]⁺

### 4) Synthesis of Compound Int-28F

In an ice-water bath, to a solution of compound Int-28E (500 mg, 2.21 mmol) in DMF was slowly added POCl₃ (0.8 mL). After the addition was completed, the reaction solution was stirred at room temperature for 48 hours. After the reaction was completed, the reaction solution was concentrated to afford compound Int-28F as a yellow solid (540 mg, 99% yield). MS(m/z)=244.0, 246.0 [M+H]⁺

### 5) Synthesis of Compound Int-28G

In an ice-water bath, to a solution of compound Int-28F (540 mg, 2.21 mmol) in dichloromethane (20 mL) were added DIEA (863 mg, 6.63 mmol) and cyclopropylamine (252 mg, 4.42 mmol). After the addition was completed, the reaction solution was stirred at room temperature overnight. After the reaction was completed, the reaction solution was concentrated under reduced pressure. The residue was washed with water (10 mL×2) and filtered. The solid was dried and collected to afford compound Int-28G as a yellow solid (580 mg, 99% yield). MS(m/z)=265.0 [M+H]⁺

### 6) Synthesis of Compound Int-28H

To a solution of compound Int-28G (580 mg, 2.19 mmol) in ethyl acetate (30 mL) was added stannous chloride (415 mg, 2.19 mmol). After the addition was completed, the reaction solution was stirred at room temperature for 3 h. After the reaction was completed, the reaction system was adjusted to pH 10-11 with 2 mol/L NaOH, and extracted with EA (30 mL×3). The organic phases were combined, dried over anhydrous sodium sulfate, and concentrated. The residue was purified by ISCO (mobile phase: MeOH:H₂O (TFA)=0:100-100:0) to afford compound Int-28H as a yellow solid (320 mg, 62% yield). MS(m/z)=235.2 [M+H]⁺

### 7) Synthesis of Compound Int-28I

To a solution of compound Int-28H (200 mg, 0.85 mmol) in toluene (20 mL) was added CDI (276 mg, 1.70 mmol). After the addition was completed, the reaction solution was stirred at 100° C. for 1 h. After the reaction was completed, the reaction system was concentrated. The residue was slurried with water (10 mL) and filtered. The solid was collected and dried to afford compound Int-28I as a yellow solid (190 mg, 85% yield). MS(m/z)=261.2 [M+H]⁺

### 8) Synthesis of Intermediate-28

In an ice-water bath and under nitrogen protection, to a solution of compound Int-28I (80 mg, 0.30 mmol) in anhydrous DMF (3 mL) was added NaH (25 mg, 0.61 mmol). After the addition was completed, the reaction solution was stirred at 0° C. for 15 min. CH₃I (65 mg, 0.46 mmol) was added to the reaction solution in one portion. After the addition was completed, the reaction solution was stirred at room temperature for 1 h. The reaction solution was poured into water, and extracted with EA (30 mL×3). The organic phases were combined, dried over anhydrous sodium sulfate, and concentrated. The residue was purified by ISCO (mobile phase: MeOH:H₂O (TFA)=0:100-100:0) to afford intermediate-28 (abbreviated as Int-28) as a yellow solid (70 mg, 83% yield). MS(m/z)=275.2 [M+H]⁺

### Preparation of Intermediate-31

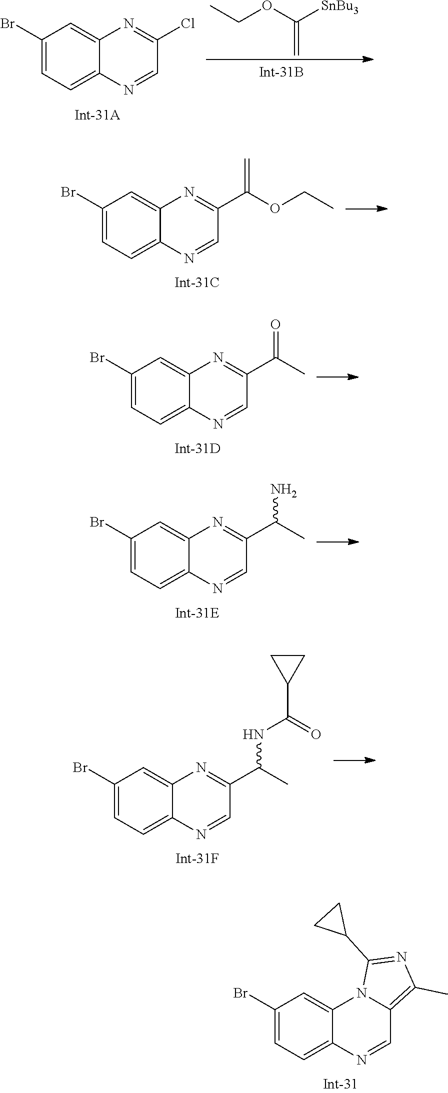

### 1) Synthesis of Compound Int-31C

Compound Int-31A (CAS: 89891-65-6) (5.84 g, 24 mmol), tributyl(1-ethoxyethylene)tin (Int-31B) (10.83 g, 30 mmol), and PdCl₂(PPh₃)₂ (1.68 g, 2.4 mmol) were dissolved in toluene (60 mL). The reaction was stirred at 80° C. for 3 h. After the reaction was completed, the reaction solution was concentrated under reduced pressure and the residue was purified by ISCO (mobile phase: (MeOH:H₂O (TFA)=0:100-100:0) to afford compound Int-31C as a yellow solid (5.9 g, 88% yield). MS(m/z)=279.2 [M+H]⁺

### 2) Synthesis of Compound Int-31D

Compound Int-31C (5.9 g, 21.1 mmol) was dissolved in dioxane (60 mL) and 2 M (60 mL) hydrochloric acid. The reaction was stirred at room temperature for 1 h. After the reaction was completed, the reaction solution was concentrated under reduced pressure and the residue was purified by ISCO (mobile phase: (PE:EA=100:0-0:100) to afford compound Int-31D as a yellow solid (3.86 g, 72% yield). MS(m/z)=251.0 [M+H]⁺

### 3) Synthesis of Compound Int-31E

Compound Int-31D (3.86 g, 15.4 mmol), ammonium acetate (11.85 g, 153.7 mmol), and NaBH₃CN (0.97 g, 15.37 mmol) were dissolved in methanol (60 mL). The reaction was stirred at room temperature overnight. After the reaction was completed, the reaction solution was concentrated under reduced pressure and the residue was purified by ISCO (mobile phase: (MeOH:H₂O (TFA)=0:100-100:0) to afford compound Int-31E as a yellow solid (2.06 g, 53% yield). MS(m/z)=252.2 [M+H]⁺

### 4) Synthesis of Compound Int-31F

Compound Int-31E (2.06 g, 8.17 mmol), cyclopropylcarboxylic acid (0.91 g, 10.62 mmol), DIEA (3.17 g, 24.5 mmol), and HATU (3.57 g, 9.39 mmol) were dissolved in DMF (25 mL). The reaction was stirred at room temperature for 1 hour. After the reaction was completed, the reaction solution was concentrated under reduced pressure and the residue was purified by ISCO (mobile phase: (MeOH:H₂O (TFA)=0:100-100:0) to afford intermediate Int-31F as a yellow solid (2.09 g, 80% yield). MS(m/z)=320.2 [M+H]⁺

### 5) Synthesis of Intermediate-31

Compound Int-31F (2.09 g, 6.52 mmol) was dissolved in phosphorus oxychloride (12 mL). The reaction was stirred at 80° C. for 3 h. After the reaction was completed, the reaction solution was concentrated under reduced pressure and the residue was purified by ISCO (mobile phase: (MeOH:H₂O (TFA)=0:100-100:0) to afford intermediate-31 (abbreviated as Int-31) as a yellow solid (1.6 g, 81% yield). MS(m/z)=302.0 [M+H]⁺

### Preparation of Intermediate-34

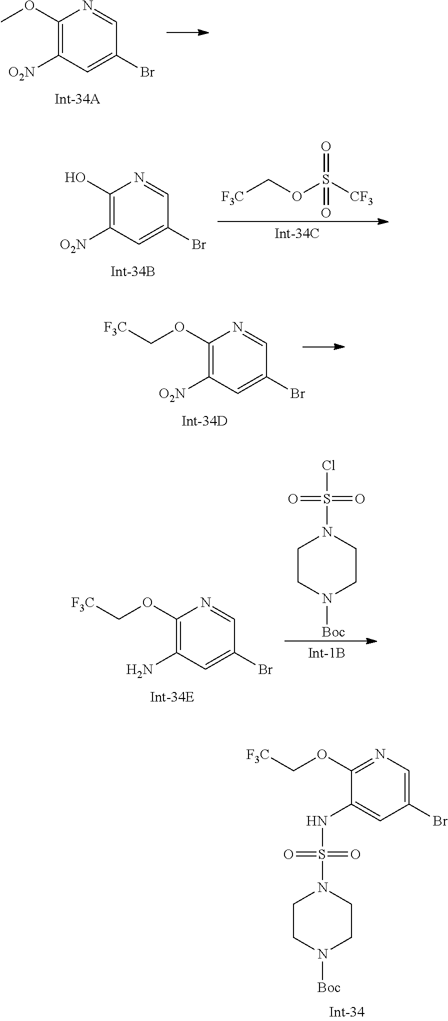

### 1) Synthesis of Compound Int-34B

Compound Int-34A (CAS: 152684-30-5) (5 g, 21.5 mmol) was dissolved in 1,4-dioxane (6 mL), and concentrated hydrochloric acid (3 mL) was slowly added thereto. The reaction was stirred at 100° C. overnight. After the reaction was completed, the reaction solution was cooled to room temperature, and filtered. The filter cake was washed with water (10 mL×3), and dried in vacuo to afford compound Int-34B as a yellow solid (4.5 g, 95.8% yield). MS(m/z)=219.0 [M+H]⁺

### 2) Synthesis of Compound Int-34D

Under nitrogen protection, compound Int-34B (2 g, 9.1 mmol) and potassium carbonate (2.52 g, 18.3 mmol) were dissolved in DMF (10 mL). Compound Int-34C (CAS: 6226-25-1) (2.54 g, 11.0 mmol) was slowly added in an ice bath, and the reaction was stirred at room temperature overnight. After the reaction was completed, the reaction solution was poured into water (20 mL), and extracted with ethyl acetate (50 mL×3). The organic phases were combined, dried over anhydrous sodium sulfate, and concentrated under reduced pressure. The residue was purified by ISCO (petroleum ether:ethyl acetate=100:0-0:100 gradient elution) to afford compound Int-34D as a yellow solid (680 mg, 24.7% yield). MS(m/z)=303.0 [M+H]⁺

### 3) Synthesis of Compound Int-34E

Under nitrogen protection, stannous chloride dihydrate (1.53 g, 6.8 mmol) was added to a solution of compound Int-34D (680 mg, 2.3 mmol) in ethyl acetate (50 mL), and the reaction was stirred at 40° C. overnight. After the reaction was completed, the reaction solution was adjusted to pH 9 by adding 2 N NaOH solution and extracted with ethyl acetate (15 mL×3). The organic phases were combined, dried over anhydrous sodium sulfate, and concentrated under reduced pressure. The residue was purified by ISCO (petroleum ether:ethyl acetate=100:0-0:50 gradient elution) to afford compound Int-34E as a brown solid (220 mg, 35.9% yield). MS(m/z)=272.9 [M+H]⁺

### 4) Synthesis of Intermediate-34

Under nitrogen protection, compound Int-1B (367.7 mg, 1.3 mmol) was added to a solution of compound Int-34E (175 mg, 0.6 mmol) in pyridine (3 mL), and the reaction was stirred at 60° C. overnight. After the reaction was completed, the reaction solution was concentrated, and the residue was purified by ISCO (water (0.1% TFA):acetonitrile=100:0-0:100 gradient elution) to afford compound intermediate-34 (abbreviated as Int-34) as a gray solid (100 mg, 30.0% yield). MS(m/z)=419.0 [M−100]⁺

### Example 2: Preparation of Payloads

### Preparation of Compound-1:

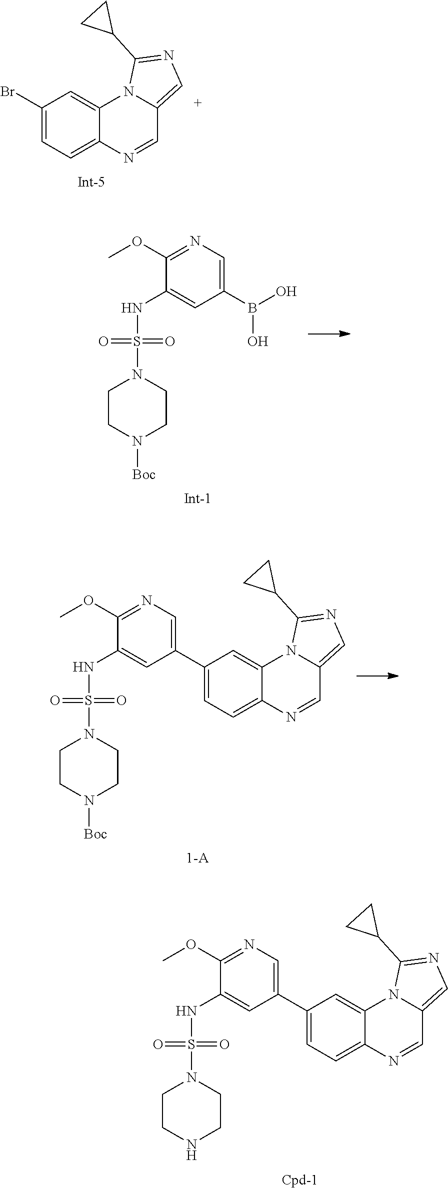 [SMILES: Brc1ccc2ncc3cnc(C4CC4)n3c2c1 ; COc1ncc(B(O)O)cc1NS(=O)(=O)N1CCN(C(=O)OC(C)(C)C)CC1 ; COc1ncc(-c2ccc3ncc4cnc(C5CC5)n4c3c2)cc1NS(=O)(=O)N1CCN(C(=O)OC(C)(C)C)CC1 ; COc1ncc(-c2ccc3ncc4cnc(C5CC5)n4c3c2)cc1NS(=O)(=O)N1CCNCC1]

### 1) Synthesis of Compound1-A

Under nitrogen protection, to a solution of Int-5 (85 mg, 0.3 mmol) in DMF:H₂O (3:1, 4 mL) were added Int-1 (147 mg, 0.35 mmol), Pd(dppf)Cl₂ (10.8 mg, 0.015 mmol), and potassium carbonate (163 mg, 1.2 mmol), and the reaction was stirred at 100° C. for 2 h. After the reaction was completed, the reaction solution was concentrated, and the residue was purified by ISCO (water (0.1% TFA):methanol=100:0-0:100 gradient elution) to afford compound 1-A as a white solid (100 mg, 58.5% yield). MS(m/z)=580.2 [M+H]⁺

### 2) Synthesis of Compound-1

Under nitrogen protection, compound 1-A (100 mg, 0.17 mmol) was added to a mixed solution of TFA/DCM (1:1, 5 mL), and the mixture was stirred for 20 minutes. After the reaction was completed, the reaction solution was concentrated, and the residue was purified by ISCO (water (0.1% TFA):acetonitrile=100:0-0:100 gradient elution) to afford compound-1 (abbreviated as Cpd-1) as a white solid (50 mg, 60.4% yield). MS(m/z)=479.8 [M+H]⁺

The following compounds were prepared according to the preparation procedure of compound-1 using the corresponding intermediates and reagents under appropriate conditions recognized by a person skilled in the art.

| | Intermediates | | |
|---|---|---|---|
| Compound | used | Structural formula | MS (M + H)⁺ |
| Compound 2 | Intermediate-8; Intermediate-1; | 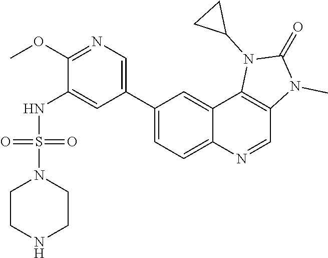 [SMILES: COc1ncc(-c2ccc3ncc4c(c3c2)n(C2CC2)c(=O)n4C)cc1NS(=O)(=O)N1CCNCC1] | 510.2 [M + H]⁺ |
| Compound 3 | Intermediate-2; Intermediate-1; | 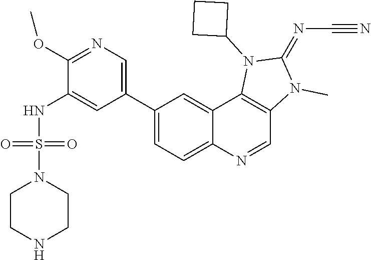 [SMILES: COc1ncc(-c2ccc3ncc4c(c3c2)n(C2CCC2)/c(=N/C#N)n4C)cc1NS(=O)(=O)N1CCNCC1] | 523.8 [M − 24]⁺ |
| Compound 4 | Intermediate-3; Intermediate-1; | 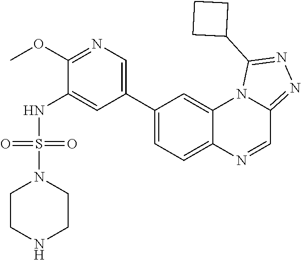 [SMILES: COc1ncc(-c2ccc3ncc4nnc(C5CCC5)n4c3c2)cc1NS(=O)(=O)N1CCNCC1] | 495.2 [M + H]⁺ |
| Compound 5 | Intermediate-9; Intermediate-1; | 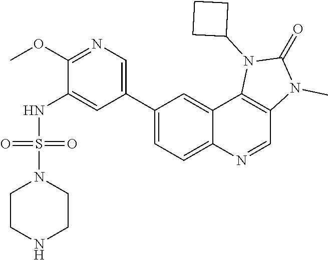 [SMILES: COc1ncc(-c2ccc3ncc4c(c3c2)n(C2CCC2)c(=O)n4C)cc1NS(=O)(=O)N1CCNCC1] | 524.2 [M + H]⁺ |
| Compound 6 | Intermediate-10; Intermediate-1; | 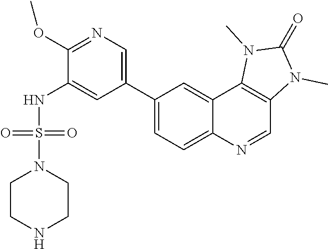 [SMILES: COc1ncc(-c2ccc3ncc4c(c3c2)n(C)c(=O)n4C)cc1NS(=O)(=O)N1CCNCC1] | 484.4 [M + H]⁺ |
| Compound 7 | Intermediate-11; Intermediate-1; | 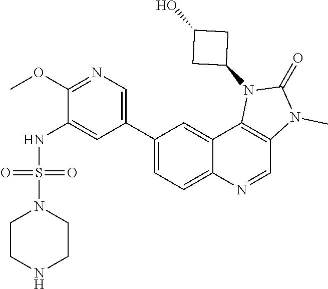 [SMILES: COc1ncc(-c2ccc3ncc4c(c3c2)n([C@H]2C[C@H](O)C2)c(=O)n4C)cc1NS(=O)(=O)N1CCNCC1] | 540.0 [M + H]⁺ |
| Compound 8 | Intermediate-12; Intermediate-1; | 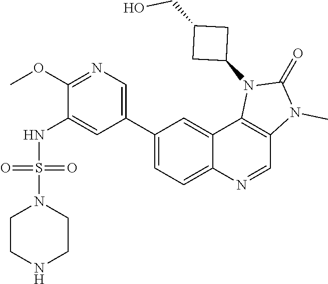 [SMILES: COc1ncc(-c2ccc3ncc4c(c3c2)n([C@H]2C[C@H](CO)C2)c(=O)n4C)cc1NS(=O)(=O)N1CCNCC1] | 554.8 [M + H]⁺ |
| Compound 9 | Intermediate-4; Intermediate-1; | 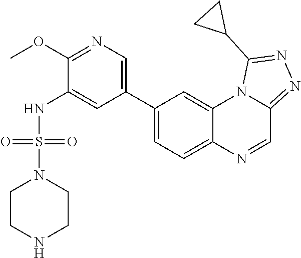 | 418.2 [M + H]⁺ |
| Compound 10 | Intermediate-8; Intermediate-21; | 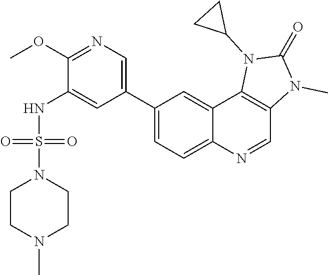 [SMILES: COc1ncc(-c2ccc3ncc4c(c3c2)n(C2CC2)c(=O)n4C)cc1NS(=O)(=O)N1CCN(C)CC1] | 524.4 [M + H]⁺ |
| Compound 11 | Intermediate-18; Intermediate-21; | 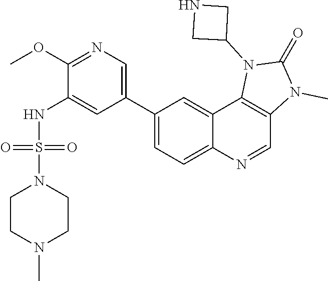 [SMILES: COc1ncc(-c2ccc3ncc4c(c3c2)n(C2CNC2)c(=O)n4C)cc1NS(=O)(=O)N1CCN(C)CC1] | 538.8 [M + H]⁺ |
| Compound 12 | Intermediate-8; Intermediate-23; | 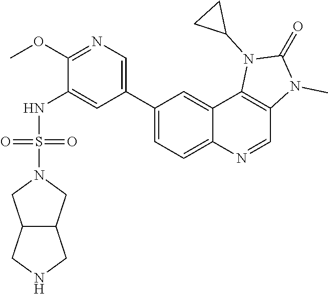 [SMILES: COc1ncc(-c2ccc3ncc4c(c3c2)n(C2CC2)c(=O)n4C)cc1NS(=O)(=O)N1CC2CNCC2C1] | 535.8 [M + H]⁺ |
| Compound 13 | Intermediate-16; Intermediate-1; | 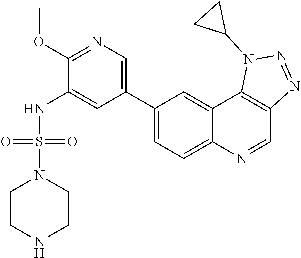 [SMILES: COc1ncc(-c2ccc3ncc4nnn(C5CC5)c4c3c2)cc1NS(=O)(=O)N1CCNCC1] | 480.2 [M + H]⁺ |
| Compound 14 | Intermediate-8; Intermediate-19; | 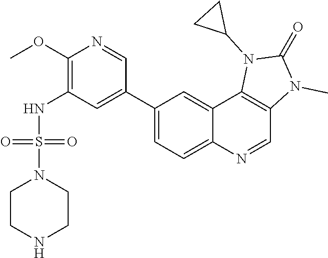 [SMILES: COc1ncc(-c2ccc3ncc4c(c3c2)n(C2CC2)c(=O)n4C)cc1NS(=O)(=O)N1CCNCC1] | 509.0 [M + H]⁺ |
| Compound 15 | Intermediate-8; Intermediate-20; | 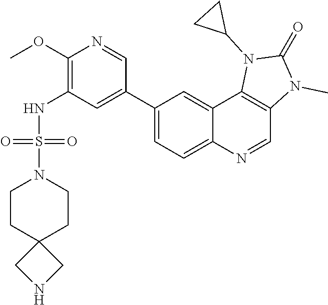 [SMILES: COc1ncc(-c2ccc3ncc4c(c3c2)n(C2CC2)c(=O)n4C)cc1NS(=O)(=O)N1CCC2(CC1)CNC2] | 549.8 [M + H]⁺ |
| Compound 16 | Intermediate-13; Intermediate-1; | 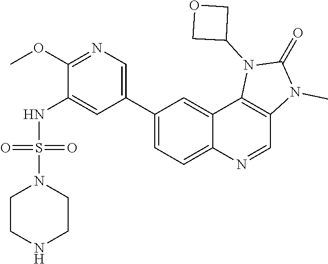 [SMILES: COc1ncc(-c2ccc3ncc4c(c3c2)n(C2COC2)c(=O)n4C)cc1NS(=O)(=O)N1CCNCC1] | 526.4 [M + H]⁺ |
| Compound 17 | Intermediate-15; Intermediate-1; | 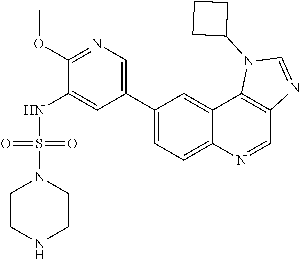 [SMILES: COc1ncc(-c2ccc3ncc4ncn(C5CCC5)c4c3c2)cc1NS(=O)(=O)N1CCNCC1] | 494.2 [M + H]⁺ |
| Compound 18 | Intermediate-6; Intermediate-1; | 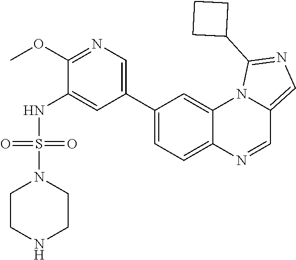 [SMILES: COc1ncc(-c2ccc3ncc4cnc(C5CCC5)n4c3c2)cc1NS(=O)(=O)N1CCNCC1] | 493.8 [M + H]⁺ |
| Compound 19 | Intermediate-14; Intermediate-1; | 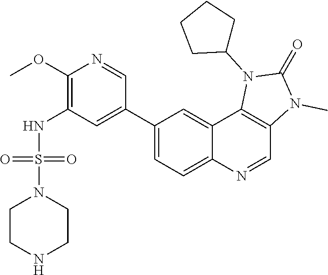 [SMILES: COc1ncc(-c2ccc3ncc4c(c3c2)n(C2CCCC2)c(=O)n4C)cc1NS(=O)(=O)N1CCNCC1] | 538.4 [M + H]⁺ |
| Compound 20 | Intermediate-7; Intermediate-1; | 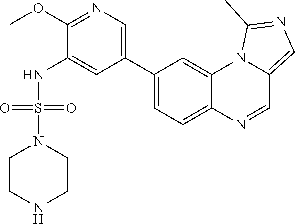 [SMILES: COc1ncc(-c2ccc3ncc4cnc(C)n4c3c2)cc1NS(=O)(=O)N1CCNCC1] | 453.8 [M + H]⁺ |
| Compound 21 | Intermediate-22; Intermediate-1; | 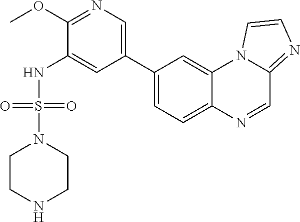 [SMILES: COc1ncc(-c2ccc3ncc4nccn4c3c2)cc1NS(=O)(=O)N1CCNCC1] | 440.2 [M + H]⁺ |
| Compound 24 | Intermediate-24; Intermediate-1; | 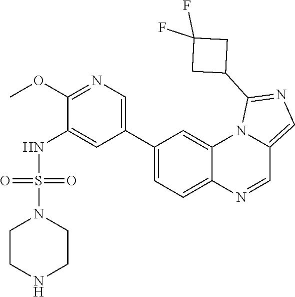 [SMILES: COc1ncc(-c2ccc3ncc4cnc(C5CC(F)(F)C5)n4c3c2)cc1NS(=O)(=O)N1CCNCC1] | 529.8 [M + H]⁺ |
| Compound 25 | Intermediate-25; Intermediate-1; | 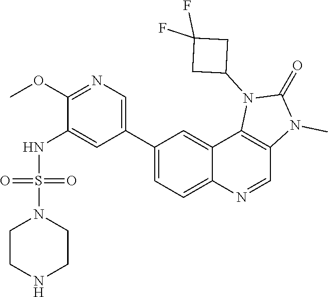 [SMILES: COc1ncc(-c2ccc3ncc4c(c3c2)n(C2CC(F)(F)C2)c(=O)n4C)cc1NS(=O)(=O)N1CCNCC1] | 560.2 [M + H]⁺ |
| Compound 27 | Intermediate-27; Intermediate-1; | 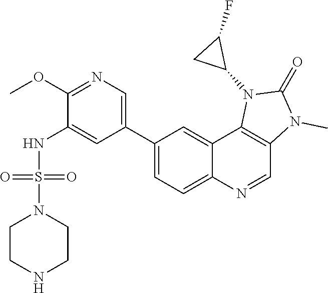 [SMILES: COc1ncc(-c2ccc3ncc4c(c3c2)n([C@@H]2C[C@@H]2F)c(=O)n4C)cc1NS(=O)(=O)N1CCNCC1] | 528.2 [M + H]⁺ |
| Compound 28 | Intermediate-28; Intermediate-1; | 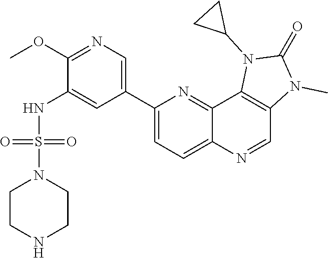 [SMILES: COc1ncc(-c2ccc3ncc4c(c3n2)n(C2CC2)c(=O)n4C)cc1NS(=O)(=O)N1CCNCC1] | 511.4 [M + H]⁺ |
| Compound 29 | Intermediate-29; Intermediate-1; | 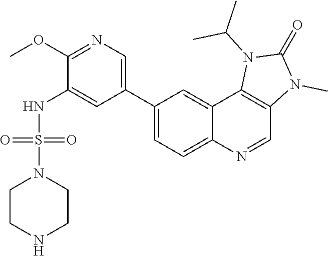 [SMILES: COc1ncc(-c2ccc3ncc4c(c3c2)n(C(C)C)c(=O)n4C)cc1NS(=O)(=O)N1CCNCC1] | 512.2 [M + H]⁺ |
| Compound 30 | Intermediate-30; Intermediate-1; | 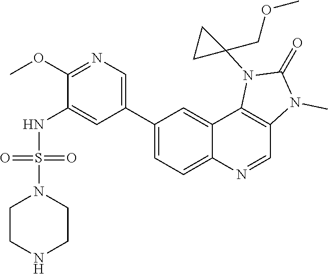 [SMILES: COCC1(n2c(=O)n(C)c3cnc4ccc(-c5cnc(OC)c(NS(=O)(=O)N6CCNCC6)c5)cc4c32)CC1] | 554.2 [M + H]⁺ |
| Compound 31 | Intermediate-31; Intermediate-1; | 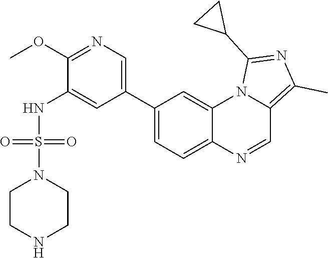 [SMILES: COc1ncc(-c2ccc3ncc4c(C)nc(C5CC5)n4c3c2)cc1NS(=O)(=O)N1CCNCC1] | 494.2 [M + H]⁺ |
| Compound 32 | Intermediate-32; Intermediate-1; | 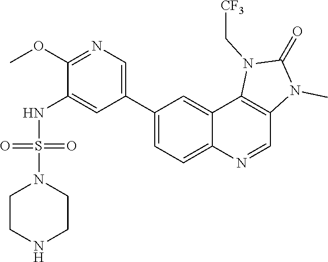 [SMILES: COc1ncc(-c2ccc3ncc4c(c3c2)n(CC(F)(F)F)c(=O)n4C)cc1NS(=O)(=O)N1CCNCC1] | 552.2 [M + H]⁺ |
| Compound 33 | Intermediate-33; Intermediate-1; | 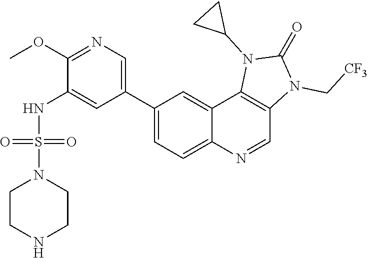 [SMILES: COc1ncc(-c2ccc3ncc4c(c3c2)n(C2CC2)c(=O)n4CC(F)(F)F)cc1NS(=O)(=O)N1CCNCC1] | 578.1 [M + H]⁺ |
| Compound 34 | Intermediate-34; Intermediate-26; | 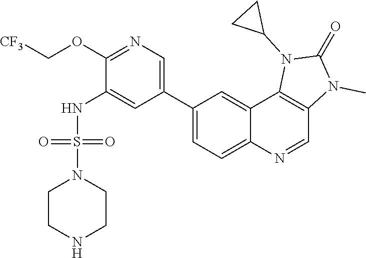 [SMILES: Cn1c(=O)n(C2CC2)c2c3cc(-c4cnc(OCC(F)(F)F)c(NS(=O)(=O)N5CCNCC5)c4)ccc3ncc21] | 578.2 [M + H]⁺ |
| Compound 35 | Intermediate-35; Intermediate-1; | 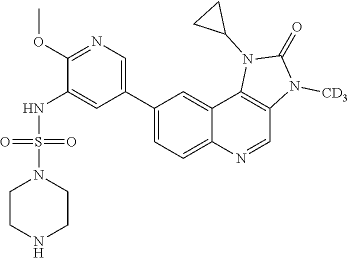 [SMILES: [2H]C([2H])([2H])n1c(=O)n(C2CC2)c2c3cc(-c4cnc(OC)c(NS(=O)(=O)N5CCNCC5)c4)ccc3ncc21] | 513.2 [M + H]⁺ |

### Preparation of Compound 22:

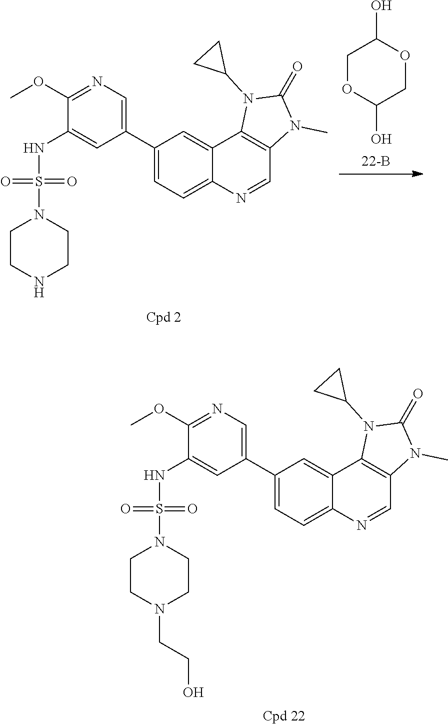 [SMILES: COc1ncc(-c2ccc3ncc4c(c3c2)n(C2CC2)c(=O)n4C)cc1NS(=O)(=O)N1CCNCC1 ; OC1COC(O)CO1 ; COc1ncc(-c2ccc3ncc4c(c3c2)n(C2CC2)c(=O)n4C)cc1NS(=O)(=O)N1CCN(CCO)CC1]

Under nitrogen protection, compound 2 (Cpd 2, 100 mg, 0.196 mmol), compound 22-B (CAS: 23147-58-2) (58 mg, 0.392 mmol), and sodium cyanoborohydride (62 mg, 0.98 mmol) were dissolved in methanol (8 mL). The reaction solution was stirred at room temperature for 18 hours. After the reaction was completed, the reaction solution was concentrated under reduced pressure, and the residue was purified by ISCO (mobile phase: water (0.1% trifluoroacetic acid):acetonitrile=100:0-0:100) to afford compound 22 (abbreviated as Cpd 22) as a yellow solid (32 mg, 29% yield). MS(m/z)=554.0 [M+1]⁺.

### Preparation of Compound 23:

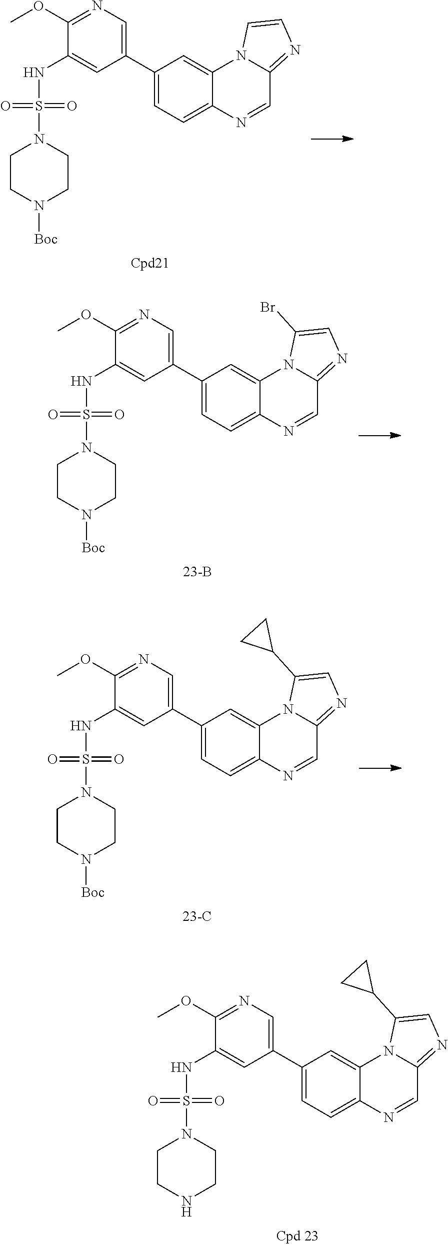 [SMILES: COc1ncc(-c2ccc3ncc4nccn4c3c2)cc1NS(=O)(=O)N1CCN(C(=O)OC(C)(C)C)CC1 ; COc1ncc(-c2ccc3ncc4ncc(Br)n4c3c2)cc1NS(=O)(=O)N1CCN(C(=O)OC(C)(C)C)CC1 ; COc1ncc(-c2ccc3ncc4ncc(C5CC5)n4c3c2)cc1NS(=O)(=O)N1CCN(C(=O)OC(C)(C)C)CC1 ; COc1ncc(-c2ccc3ncc4ncc(C5CC5)n4c3c2)cc1NS(=O)(=O)N1CCNCC1]

### 1) Synthesis of Compound 23-B

Under nitrogen protection, compound 21 (Cpd 21, 100 mg, 0.185 mmol) and N-bromosuccinimide (36 mg, 0.2 mmol) were added to chloroform. A drop of TFA was added, and the reaction was stirred at room temperature for 12 h. After the reaction was completed, the reaction solution was concentrated, and the residue was purified by ISCO (mobile phase: CH₃CN:H₂O (TFA)=0:100-100:0) to afford compound 23-B as a yellow solid (60 mg, 52% yield). MS(m/z)=617.6 [M+H]⁺

### 2) Synthesis of Compound 23-C

At room temperature and under nitrogen protection, compound 23-B (50 mg, 0.08 mmol), cyclopropylboronic acid (14 mg, 0.16 mmol), palladium acetate (4 mg, 0.016 mmol), potassium phosphate (51 mg, 0.24 mmol), and tricyclohexylphosphine (5 mg, 0.016 mmol) were sequentially added to a mixed solution of toluene and water (5:1), and the reaction was stirred at 90° C. for 3 hours. After the reaction was completed, the reaction solution was concentrated, and the residue was purified by ISCO (mobile phase: CH₃CN:H₂O (TFA)=0:100-100:0) to afford compound 23-C as a yellow solid (20 mg, 43% yield). MS(m/z)=579.8 [M+H]⁺

### 3) Synthesis of Compound 23

Under nitrogen protection, compound 23-C(20 mg, 0.035 mmol) was dissolved in a mixed solution of TFA:DCM (1:1, 5 mL), and the mixture was stirred for 20 minutes. After the reaction was completed, the reaction solution was concentrated, and the residue was purified by ISCO (water (0.1% TFA):acetonitrile=100:0-0:100 gradient elution) to afford compound 23 (abbreviated as Cpd 23) as a white solid (10 mg, 60.4% yield). MS(m/z)=479.8 [M+H]⁺.

### Preparation of Compound 26:

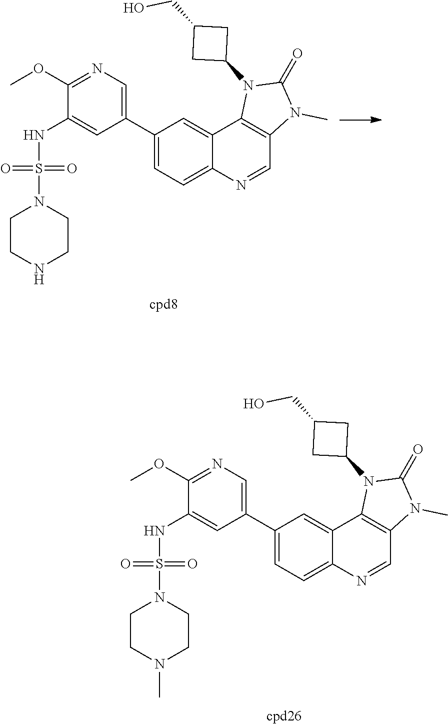 [SMILES: COc1ncc(-c2ccc3ncc4c(c3c2)n([C@H]2C[C@H](CO)C2)c(=O)n4C)cc1NS(=O)(=O)N1CCNCC1 ; COc1ncc(-c2ccc3ncc4c(c3c2)n([C@H]2C[C@H](CO)C2)c(=O)n4C)cc1NS(=O)(=O)N1CCN(C)CC1]

Compound 8 (Cpd 8, 30 mg, 0.054 mmol), NaBH₃CN (20 mg, 0.325 mmol), and 37% aqueous formaldehyde solution (0.5 ml) were dissolved in methanol (4 mL). The mixture was reacted at room temperature for 3 hours. After the reaction was completed, the reaction solution was concentrated under reduced pressure and the residue was purified by ISCO (mobile phase: (MeOH:H₂O (TFA)=0:100-100:0) to afford compound 26 as a light-yellow solid (12 mg, 39% yield). MS(m/z)=568.2 [M+H]⁺

The ¹H NMR data of the compounds prepared in Example 2 are shown in the following table:

| Compound No. | ¹H NMR |
|---|---|
| Compound 1 | ¹H NMR (400 MHz, DMSO) δ 8.93 (d, J = 1.9 Hz, 1H), 8.90 (s, |
| | 1H), 8.09 (s, 1H), 8.00-7.93 (m, 2H), 7.81 (dd, J = 8.5, 1.9 Hz, |
| | 1H), 7.74 (s, 1H), 3.88 (s, 3H), 2.94-2.89 (m, 4H), 2.70-2.60 (m, |
| | 5H), 1.35-1.30 (m, 2H), 1.16-1.12 (m, 2H). |
| Compound 2 | ¹H NMR (400 MHz, DMSO) δ 8.89 (s, 1H), 8.83 (s, 1H), 8.39-8.32 |
| | (m, 1H), 8.11-8.05 (m, 2H), 7.93-7.86 (m, 1H), 3.95 (s, 3H), 3.50- |
| | 3.49 (m, 1H), 3.45 (s, 3H), 3.02-2.98 (s, 4H), 2.67-2.63 (s, 4H), |
| | 1.36-1.31 (m, 2H), 1.22-1.18 (m, 2H). |
| Compound 3 | ¹H NMR (400 MHz, DMSO) δ 8.84 (s, 1H), 8.35 (d, J = 2.2 Hz, |
| | 1H), 8.34 (d, J = 1.7 Hz, 1H), 8.08 (d, J = 8.9 Hz, 1H), 8.05 (d, J = |
| | 2.3 Hz, 1H), 7.85 (dd, J = 8.9, 1.8 Hz, 1H), 5.44-5.33 (m, 1H), |
| | 3.95 (s, 3H), 3.46 (s, 3H), 3.10-3.04 (m, 2H), 3.03-2.99 (m, 4H), |
| | 2.73-2.61 (m, 4H), 2.45-2.38 (m, 2H), 1.87 (dt, J = 19.3, 8.6 Hz, 2H). |
| Compound 4 | ¹H NMR (400 MHz, DMSO) δ 9.29 (s, 1H), 8.39 (d, J = 2.3 Hz, |
| | 1H), 8.19-8.10 (m, 2H), 8.07 (d, J = 2.2 Hz, 1H), 7.96 (d, J = 8.4 |
| | Hz, 1H), 4.51 (t, J = 7.8 Hz, 1H), 3.98 (s, 3H), 3.06 (s, 4H), 2.74 (s, |
| | 4H), 2.68-2.61 (m, 4H), 2.22-2.14 (m, 1H), 2.05-1.96 (m, 1H). |
| Compound 5 | ¹H NMR (400 MHz, DMSO) δ 8.84 (s, 1H), 8.40-8.32 (m, 2H), |
| | 8.12-8.04 (m, 2H), 7.88-7.82 (m, 1H), 5.46-5.35 (m, 1H), 3.97 (s, |
| | 3H), 3.47 (s, 3H), 3.12-3.03 (m, 4H), 3.02-2.97 (m, 4H), 2.68-2.61 |
| | (m, 4H), 1.93-1.82 (m, 2H). |
| Compound 6 | ¹H NMR (400 MHz, DMSO) δ 8.85 (s, 1H), 8.41 (d, J = 1.8 Hz, |
| | 1H), 8.35 (d, J = 2.2 Hz, 1H), 8.09 (d, J = 8.9 Hz, 1H), 8.01 (d, J = |
| | 2.3 Hz, 1H), 7.83 (dd, J = 8.9, 1.9 Hz, 1H), 3.94 (s, 3H), 3.89 (s, |
| | 3H), 3.51 (s, 3H), 3.03-2.98 (m, 4H), 2.68-2.64 (m, 4H). |
| Compound 7 | ¹H NMR (400 MHz, DMSO) δ 8.85 (s, 1H), 8.37 (d, J = 2.2 Hz, |
| | 1H), 8.24 (d, J = 1.6 Hz, 1H), 8.09 (d, J = 8.8 Hz, 1H), 8.00 (d, J = |
| | 2.2 Hz, 1H), 7.81 (dd, J = 8.8, 1.6 Hz, 1H), 5.54-5.46 (m, 1H), |
| | 4.51-4.44 (m, 1H), 3.97 (s, 3H), 3.47 (s, 3H), 3.23-3.19 (m, 2H), |
| | 3.09-3.04 (m, 4H), 2.75-2.71 (m, 4H), 2.44-2.38 (m, 2H). |
| Compound 8 | ¹H NMR (400 MHz, DMSO) δ 8.85 (s, 1H), 8.35-8.29 (m, 2H), |
| | 8.10 (d, J = 8.8 Hz, 1H), 7.99 (d, J = 1.8 Hz, 1H), 7.81 (dd, J = 8.8, |
| | 1.8 Hz, 1H), 5.60-5.56 (m, 1H), 3.95 (s, 3H), 3.58 (m, 2H), 3.48 (s, |
| | 3H), 3.14-3.07 (m, 3H), 3.02-2.98 (m, 4H), 2.67-2.64 (m, 4H), |
| | 2.35-2.27 (m, 2H). |
| Compound 9 | ¹H NMR (400 MHz, DMSO) δ 9.28 (s, 1H), 8.90 (s, 1H), 8.39 (s, |
| | 1H), 8.18 (d, J = 8.4 Hz, 1H), 8.08 (d, J = 1.8 Hz, 1H), 8.01 (d, J = |
| | 8.4 Hz, 1H), 3.96 (s, 3H), 3.02 (s, 4H), 2.81 (s, 1H), 2.71 (s, 4H), |
| | 1.35 (d, J = 7.9 Hz, 1H), 1.22 (d, J = 6.0 Hz, 4H). |
| Compound 10 | ¹H NMR (400 MHz, CD3OD) δ 8.89-8.83 (m, 1H), 8.70 (s, 1H), |
| | 8.31-8.29 (m, 1H), 8.18-8.13 (m, 1H), 8.07-8.01 (m, 1H), 7.89-7.83 |
| | (m, 1H), 4.08 (s, 3H), 3.53 (s, 3H), 3.41-3.34 (m, 1H), 3.32-3.30 |
| | (m, 4H), 2.69-2.61 (m, 4H), 2.40 (s, 3H), 1.48-1.42 (m, 2H), 1.19- |
| | 1.14 (m, 2H). |
| Compound 11 | ¹H NMR (400 MHz, DMSO) δ 8.89 (s, 1H), 8.63 (s, 1H), 8.45-8.39 |
| | (m, 1H), 8.14-8.09 (m, 1H), 8.07-8.04 (m, 1H), 7.87-7.83 (m, 1H), |
| | 5.90-5.79 (m, 1H), 4.38-4.31 (m, 2H), 4.10-4.04 (m, 2H), 3.94 (s, |
| | 3H), 3.50 (s, 3H), 3.08-3.04 (m, 4H), 2.28-2.23 (m, 4H), 2.10 (s, 3H). |
| Compound 12 | ¹H NMR (400 MHz, DMSO) δ 8.90-8.84 (m, 1H), 8.80 (s, 1H), |
| | 8.08-8.02 (m, 2H), 7.96-7.92 (m, 1H), 7.88-7.83 (m, 1H), 3.89 (s, |
| | 3H), 3.45 (s, 3H), 3.43-3.39 (m, 1H), 3.34-3.26 (m, 4H), 3.05- |
| | 3.00 (m, 2H), 2.97-2.91 (m, 2H), 2.87-2.80 (m, 2H), 1.43-1.35 (m, |
| | 2H), 1.15-1.07 (m, 2H). |
| Compound 13 | ¹H NMR (400 MHz, DMSO) δ 9.15 (s, 1H), 8.96 (d, J = 2.1 Hz, |
| | 1H), 8.43 (s, 1H), 8.23-8.16 (m, 2H), 8.07 (d, J = 2.3 Hz, 1H), |
| | 8.00-7.95 (m, 1H), 4.10-4.04 (m, 1H), 3.89 (s, 3H), 2.93 (t, J = |
| | 4.8 Hz, 4H), 2.62 (t, J = 4.8 Hz, 4H), 1.47-1.41 (m, 2H), 1.33- |
| | 1.28 (m, 2H). |
| Compound 14 | ¹H NMR (400 MHz, DMSO) δ 8.89 (d, J = 1.9 Hz, 1H), 8.82 (s, |
| | 1H), 8.27 (d, J = 2.0 Hz, 1H), 8.07 (d, J = 8.9 Hz, 1H), 8.05 (d, J = |
| | 2.3 Hz, 1H), 7.91 (dd, J = 8.9, 2.0 Hz, 1H), 3.91 (s, 3H), 3.52- |
| | 3.49 (m, 1H), 3.45 (s, 3H), 3.07 (dd, J = 14.9, 11.9 Hz, 3H), 2.51 |
| | (d, J = 12.4 Hz, 2H), 1.97 (d, J = 11.0 Hz, 2H), 1.56 (dd, J = 12.4, |
| | 3.8 Hz, 2H), 1.35 (d, J = 5.4 Hz, 2H), 1.13 (dd, J = 6.4, 4.1 Hz, 2H). |
| Compound 15 | ¹H NMR (400 MHz, DMSO) δ 8.87 (d, J = 1.8 Hz, 1H), 8.81 (s, |
| | 1H), 8.38 (s, 1H), 8.05 (d, J = 8.9 Hz, 2H), 7.98 (d, J = 2.2 Hz, |
| | 1H), 7.86 (dd, J = 8.9, 1.8 Hz, 1H), 3.86 (s, 3H), 3.54 (s, 4H), 3.45 |
| | (s, 3H), 2.97-2.92 (m, 4H), 1.74-1.63 (m, 4H), 1.38 (q, J = 6.9 |
| | Hz, 2H), 1.13 (m, 2H). |
| Compound 16 | ¹H NMR (400 MHz, DMSO) δ 8.78 (s, 1H), 8.39 (d, J = 2.3 Hz, |
| | 1H), 8.05 (d, J = 2.0 Hz, 1H), 8.01-7.96 (m, 2H), 7.82-7.78 (m, |
| | 1H), 4.81-4.74 (m, 1H), 4.42-4.38 (m, 1H), 4.00 (s, 4H), 3.63- |
| | 3.60 (m, 1H), 3.39-3.27 (m, 5H), 3.20 (s, 3H), 3.17-3.11 (m, 4H). |
| Compound 17 | ¹H NMR (400 MHz, DMSO) δ 9.45 (s, 1H), 8.82 (s, 1H), 8.51 (dd, |
| | J = 8.3, 2.1 Hz, 2H), 8.31 (d, J = 8.8 Hz, 1H), 8.19-8.09 (m, 2H), |
| | 5.62 (s, 1H), 4.00 (s, 3H), 3.33-3.28 (m, 4H), 3.14-3.04 (m, 4H), |
| | 2.74-2.59 (m, 4H), 2.07-1.90 (m, 2H). |
| Compound 18 | ¹H NMR (400 MHz, DMSO) δ 8.95 (s, 1H), 8.42 (d, J = 2.2 Hz, |
| | 1H), 8.12 (d, J = 1.9 Hz, 1H), 8.06 (d, J = 2.3 Hz, 1H), 7.96 (d, J = |
| | 8.3 Hz, 1H), 7.89-7.81 (m, 2H), 4.47-4.37 (m, 1H), 3.99 (s, 3H), |
| | 3.19-3.15 (m, 4H), 2.95-2.90 (m, 4H), 2.63-2.53 (m, 4H), 2.19- |
| | 2.08 (m, 1H), 2.01-1.90 (m, 1H). |
| Compound 19 | ¹H NMR (400 MHz, DMSO) δ 8.84 (s, 1H), 8.32 (s, 1H), 8.23 (s, |
| | 1H), 8.09 (d, J = 8.8 Hz, 1H), 7.98 (d, J = 2.1 Hz, 1H), 7.83 (d, J = |
| | 8.7 Hz, 1H), 5.44-5.31 (m, 1H), 3.92 (s, 3H), 3.47 (s, 3H), 2.97 |
| | (m, 4H), 2.64 (m, 4H), 2.30 (m, 2H), 2.12-2.02 (m, 2H), 1.93 (m, |
| | 2H), 1.77-1.66 (m, 2H). |
| Compound 20 | ¹H NMR (400 MHz, DMSO) δ 8.92 (s, 1H), 8.39 (d, J = 2.3 Hz, |
| | 1H), 8.35 (d, J = 1.9 Hz, 1H), 8.01 (d, J = 2.3 Hz, 1H), 7.95 (d, J = |
| | 8.3 Hz, 1H), 7.80-7.75 (m, 2H), 3.95 (s, 3H), 3.10 (s, 3H), 3.06 (t, |
| | J = 4.9 Hz, 4H), 2.75 (t, J = 4.9 Hz, 4H). |
| Compound 21 | ¹H NMR (400 MHz, DMSO) δ 9.09 (s, 1H), 8.96 (s, 1H), 8.68 (d, |
| | J = 1.9 Hz, 1H), 8.46 (d, J = 2.3 Hz, 1H), 8.14-8.07 (m, 2H), 7.88- |
| | 7.83 (m, 2H), 3.97 (s, 3H), 2.99 (t, J = 4.9 Hz, 4H), 2.66 (t, J = 4.9 |
| | Hz, 4H). |
| Compound 22 | ¹H NMR (400 MHz, DMSO) δ 9.94 (s, 1H), 9.17 (s, 1H), 9.11-9.01 |
| | (m, 1H), 8.61-8.51 (m, 1H), 8.31-8.19 (m, 2H), 8.17-8.09 (m, 1H), |
| | 4.01 (s, 3H), 3.80-3.60 (m, 5H), 3.58-3.41 (m, 5H), 3.28-2.91 (m, |
| | 6H), 1.42-1.32 (m, 2H), 1.21-1.17 (m, 2H). |
| Compound 23 | ¹H NMR (400 MHz, DMSO) δ 9.09 (d, J = 1.9 Hz, 1H), 8.99 (d, |
| | J = 0.8 Hz, 1H), 8.36 (d, J = 2.3 Hz, 1H), 8.12 (d, J = 8.5 Hz, 1H), |
| | 8.05 (d, J = 2.3 Hz, 1H), 7.93 (dd, J = 8.6, 1.9 Hz, 1H), 7.61 (s, |
| | 1H), 3.94 (s, 3H), 3.03-2.98 (m, 4H), 2.69-2.57 (m, 5H), 1.30- |
| | 1.24 (m, 2H), 1.01-0.94 (m, 2H). |
| Compound 24 | 1H NMR (400 MHz, CD3OD) δ 8.94 (d, J = 1.2 Hz, 1H), 8.40 (dd, |
| | J = 2.4, 1.1 Hz, 1H), 8.22-8.15 (m, 2H), 8.06-8.00 (m, 1H), 7.92 |
| | (d, J = 1.0 Hz, 1H), 7.85 (dd, J = 8.3, 1.7 Hz, 1H), 4.42-4.31 (m, |
| | 1H), 4.12 (s, 3H), 3.53-3.47 (m, 4H), 3.37-3.31 (m, 4H), 3.27- |
| | 3.23 (m, 4H). |
| Compound 25 | 1H NMR (400 MHz, CD3OD) δ 8.81 (s, 1H), 8.43-8.37 (m, 1H), |
| | 8.35-8.30 (m, 1H), 8.21-8.18 (m, 1H), 8.17-8.13 (m, 1H), 7.90 (m, |
| | 1H), 5.51-5.39 (m, 1H), 4.12 (s, 3H), 3.98-3.86 (m, 2H), 3.58 (s, |
| | 3H), 3.52-3.48 (m, 4H), 3.26-3.23 (m, 4H), 3.21-3.12 (m, 2H). |
| Compound 26 | 1H NMR (400 MHz, CD3OD) δ 8.62 (s, 1H), 8.22-8.18 (m, 1H), |
| | 8.13-8.10 (m, 1H), 8.04-8.01 (m, 1H), 7.97-7.91 (m, 1H), 7.71-7.66 |
| | (m, 1H), 5.47-5.36 (m, 1H), 4.09 (s, 3H), 3.79-3.73 (m, 2H), 3.51 |
| | (s, 3H), 3.28-3.17 (m, 6H), 2.66-2.56 (m, 1H), 2.46-2.37 (m, |
| | 6H), 2.23 (s, 3H). |
| Compound 27 | 1H NMR (400 MHz, DMSO) δ 8.87 (s, 1H), 8.78 (s, 1H), 8.37 (d, |
| | J = 2.2 Hz, 1H), 8.11 (d, J = 8.9 Hz, 1H), 8.07 (d, J = 2.3 Hz, 1H), |
| | 7.90 (dd, J = 8.9, 2.0 Hz, 1H), 5.42 (s, 1H), 5.24 (s, 1H), 3.96 (s, |
| | 3H), 3.67-3.62 (m, 1H), 3.51 (s, 3H), 3.04-2.98 (m, 4H), 2.69- |
| | 2.63 (m, 4H), 1.93-1.73 (m, 2H). |
| Compound 28 | 1H NMR (400 MHz, DMSO) δ 8.89 (s, 1H), 8.80 (d, J = 2.1 Hz, |
| | 1H), 8.64 (d, J = 2.2 Hz, 1H), 8.42 (d, J = 9.0 Hz, 1H), 8.24 (d, J = |
| | 9.0 Hz, 1H), 3.98 (s, 3H), 3.66-3.62 (m, 1H), 3.47 (s, 3H), 3.00- |
| | 2.95 (m, 4H), 2.66-2.60 (m, 4H), 1.30-1.25 (m, 2H), 1.14-1.09 |
| | (m, 2H). |
| Compound 29 | 1H NMR (400 MHz, DMSO) δ 8.86 (s, 1H), 8.36 (dd, J = 5.4, 2.0 |
| | Hz, 2H), 8.12 (d, J = 8.8 Hz, 1H), 8.04 (d, J = 2.3 Hz, 1H), 7.86 |
| | (dd, J = 9.0, 1.8 Hz, 1H), 5.28 (dt, J = 13.8, 6.8 Hz, 1H), 3.96 (s, |
| | 3H), 3.48 (s, 3H), 3.03-3.00 (m, 4H), 2.67-2.64 (m, 4H), 1.65 |
| | (d, J = 6.8 Hz, 6H). |
| Compound 30 | 1H NMR (400 MHz, DMSO) δ 10.18 (s, 1H), 8.99 (d, J = 2.0 Hz, |
| | 1H), 8.85 (s, 1H), 8.41 (d, J = 2.2 Hz, 1H), 8.16 (d, J = 2.4 Hz, 1H), |
| | 8.08 (d, J = 8.8 Hz, 1H), 7.93 (dd, J = 8.9, 2.0 Hz, 1H), 3.99 (s, |
| | 3H), 3.87 (d, J = 10.7 Hz, 1H), 3.53 (s, 1H), 3.49 (s, 3H), 3.18 (s, |
| | 3H), 3.04 (t, J = 5.1 Hz, 4H), 2.71 (d, J = 5.6 Hz, 4H), 1.49 (s, 1H), |
| | 1.35 (q, J = 7.1 Hz, 2H), 1.22 (s, 1H). |
| Compound 31 | 1H NMR (400 MHz, DMSO) δ 8.89 (s, 1H), 8.86-8.82 (m, 1H), |
| | 8.36-8.32 (m, 1H), 8.06-8.01 (m, 1H), 7.93-7.90 (m, 1H), 7.81-7.76 |
| | (m, 1H), 3.96 (s, 3H), 3.02-2.98 (m, 4H), 2.69-2.65 (m, 5H), |
| | 2.47 (s, 3H), 1.28-1.23 (m, 2H), 1.15-1.10 (m, 2H). |
| Compound 32 | 1H NMR (400 MHz, DMSO) δ 8.96 (s, 1H), 8.39-8.35 (m, 2H), |
| | 8.15 (d, J = 8.9 Hz, 1H), 8.05 (d, J = 2.3 Hz, 1H), 7.87 (dd, J = 8.9, |
| | 1.9 Hz, 1H), 5.39 (q, J = 9.0 Hz, 2H), 3.98 (s, 3H), 3.57 (s, 3H), |
| | 3.04-2.98 (m, 4H), 2.70-2.63 (m, 4H). |
| Compound 33 | 1H NMR (400 MHz, DMSO) δ 8.97-8.92 (m, 2H), 8.39 (d, J = |
| | 2.3 Hz, 1H), 8.14-8.08 (m, 2H), 7.99-7.93 (m, 1H), 5.04-4.93 |
| | (m, 2H), 3.97 (s, 3H), 3.63-3.56 (m, 1H), 3.06-3.00 (m, 4H), |
| | 2.71-2.67 (m, 4H), 1.41-1.31 (m, 2H), 1.19-1.15 (m, 2H). |
| Compound 34 | 1H NMR (400 MHz, DMSO) δ 8.91 (d, J = 2.0 Hz, 1H), 8.84 (d, |
| | J = 0.8 Hz, 1H), 8.22 (s, 1H), 8.14 (d, J = 2.3 Hz, 1H), 8.11 (d, J = |
| | 2.3 Hz, 1H), 8.09 (d, J = 8.9 Hz, 1H), 7.90 (dd, J = 8.8, 2.0 Hz, |
| | 1H), 5.04-4.94 (m, 2H), 3.46 (s, 3H), 3.0-3.01 (m, 4H), 2.80-2.72 |
| | (m, 4H), 2.01-1.90 (m, 1H), 1.41-1.33 (m, 2H), 1.15-1.10 (m, 2H). |
| Compound 35 | 1H NMR (400 MHz, dmso) δ 8.89 (s, 1H), 8.83 (s, 1H), 8.36 (s, |
| | 1H), 8.11-8.05 (m, 2H), 7.93-7.87 (m, 1H), 3.96 (s, 3H), 3.51-3.47 |
| | (m, 1H), 3.04-2.98 (m, 4H), 2.69-2.63 (m, 4H), 1.37-1.30 (m, 2H), |
| | 1.16-1.09 (m, 2H). |

The following reference compounds were also synthesized in the present invention according to the method in WO 2012034526 A1:

| No. | Structure |
|---|---|
| Reference compound-1 | 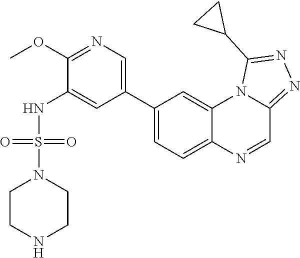 [SMILES: COc1ncc(-c2ccc3ncc4nnc(C5CC5)n4c3c2)cc1NS(=O)(=O)N1CCNCC1] |

### Example 3: Preparation of Linker-Payloads (LPs)

### Preparation of LP-1:

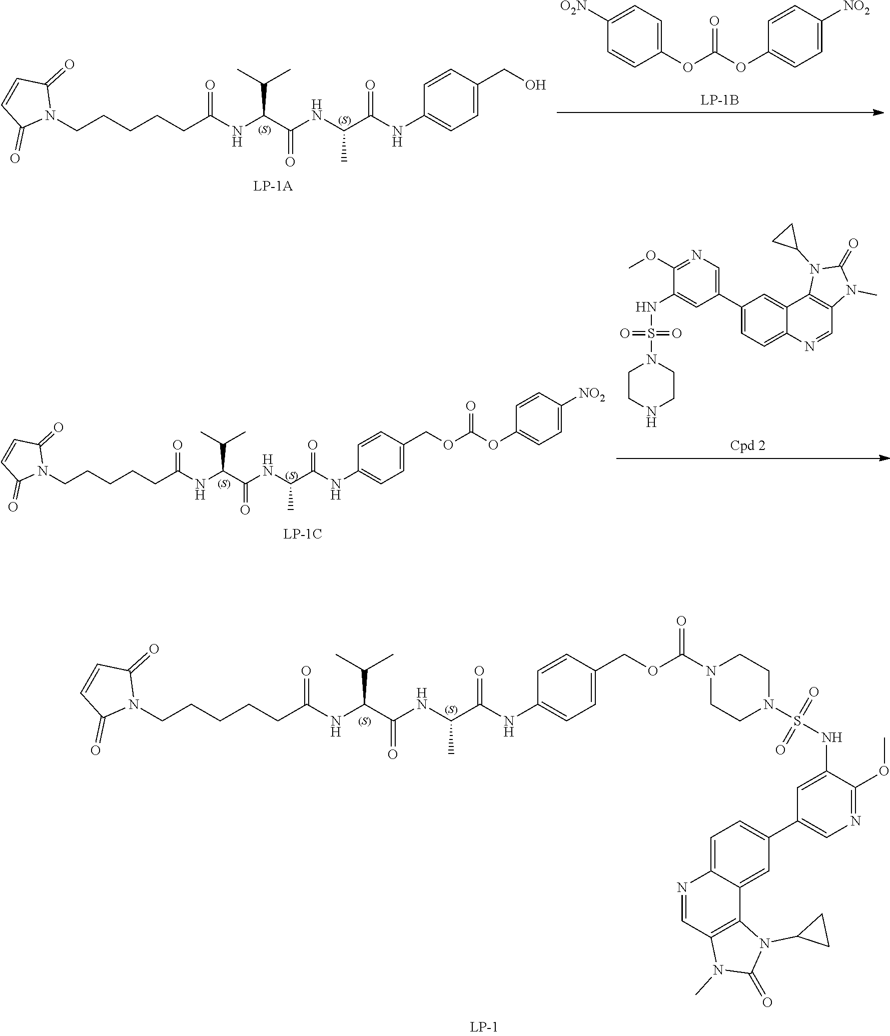

### 1) Synthesis of Compound LP-1C

In an ice-water bath and under nitrogen protection, DIEA (108 μL, 0.62 mmol) was added to a solution of compound LP-1A (CAS: 1870916-87-2) (150 mg, 0.31 mmol) and compound LP-1B (CAS: 5070-13-3) (188 mg, 0.62 mmol) in DMA (3 mL). After the addition was completed, the reaction was stirred at room temperature overnight. After the reaction was completed, the reaction solution was concentrated to remove the solvent, and the residue was purified by ISCO (mobile phase: MeOH:DCM=0:100-50:50) to afford compound LP-1C as a yellow solid (100 mg, 50% yield). MS(m/z)=673.8 [M+23]⁺

### 2) Synthesis of LP-1

In an ice-water bath and under nitrogen protection, to a solution of compound LP-1C (70 mg, 0.11 mmol) and compound 2 (Cpd 2, 60 mg, 0.12 mmol) in DMF (2 mL) was added pyridine (0.5 mL), followed by HOBt (7.4 mg, 0.055 mmol) and DIEA (38 μL, 0.22 mmol). After the addition was completed, the reaction solution was stirred at room temperature overnight. After the reaction was completed, the reaction solution was directly purified by ISCO (mobile phase: MeOH:H₂O (0.1% TFA)=0:100-100:0) to afford compound LP-1 as a white solid (12 mg, 11% yield). MS(m/z)=1021.8 [M+H]⁺

### Preparation of LP-2:

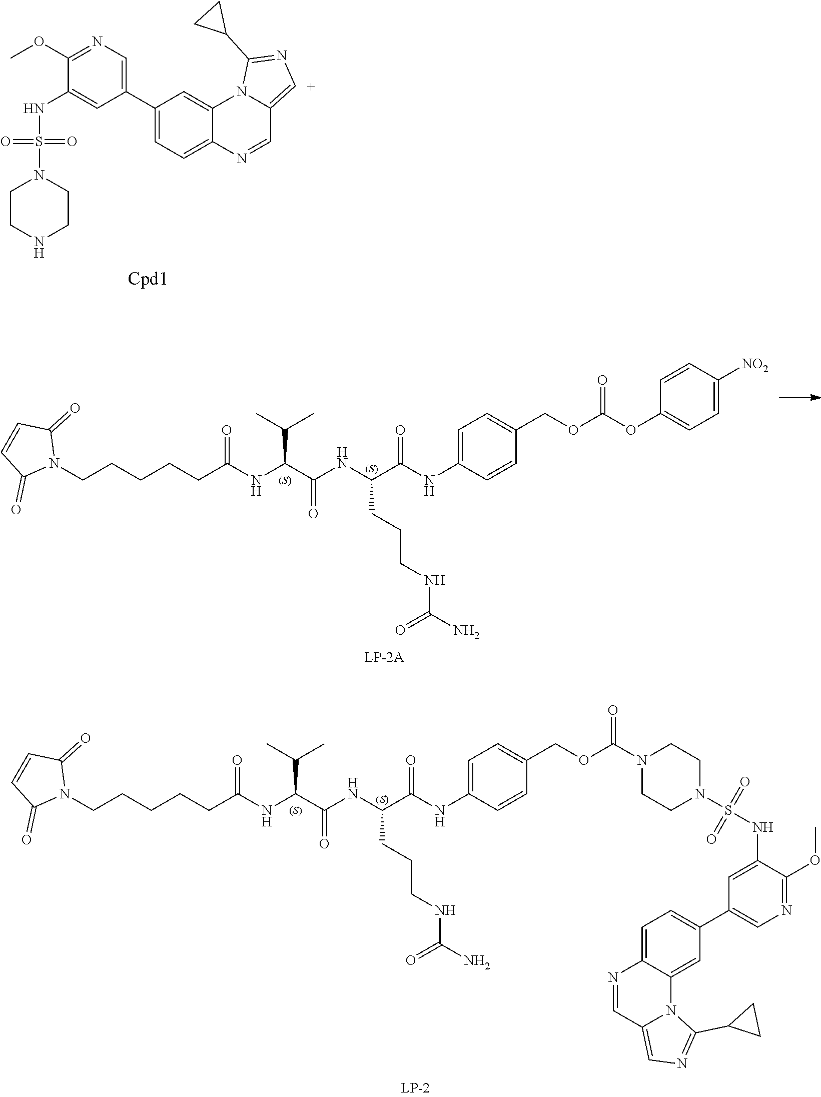

Under nitrogen protection, compound 1 (Cpd 1, 41 mg, 0.085 mmol), compound LP-2A (52 mg, 0.07 mmol) (CAS: 159857-81-5), HOBT (5.7 mg, 0.04 mmol), pyridine (6 mg, 0.07 mmol), and DIEA (27 mg, 0.21 mmol) were added to a DMF solution, and the mixture was stirred at room temperature overnight. After the reaction was completed, the reaction solution was concentrated, and the residue was purified by ISCO (water (0.1% TFA):acetonitrile 100:0-0:100 gradient elution) to afford compound LP-2 as a white solid (66 mg, 86.9% yield). MS(m/z)=539.4 [1/2M+H]⁺

The following compounds were prepared according to the preparation procedure of compound LP-2 using the corresponding intermediates and reagents under appropriate conditions recognized by a person skilled in the art.

| LP No. | Payloads used | Structural formula | MS |
|---|---|---|---|
| LP-3 | Compound 2 | 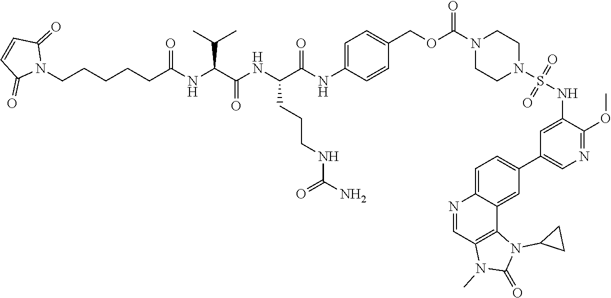 [SMILES: COc1ncc(-c2ccc3ncc4c(c3c2)n(C2CC2)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | 1108.0 [M + H]⁺ |
| LP-4 | Compound 3 | 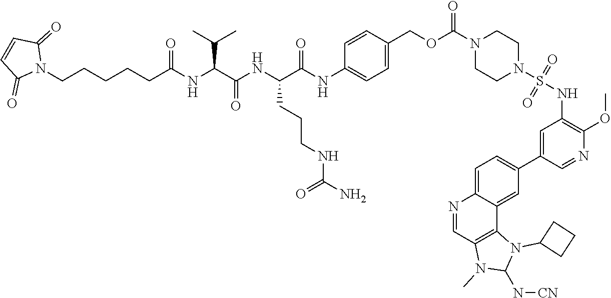 | 1122.6 [M + H]⁺ |
| LP-5 | Compound 4 | 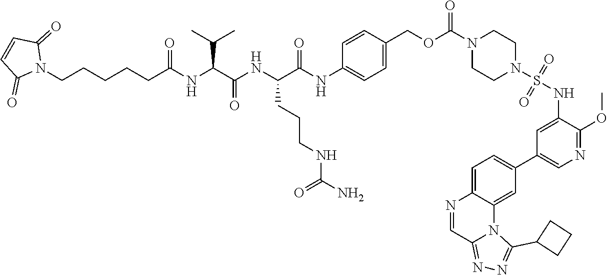 [SMILES: COc1ncc(-c2ccc3ncc4nnc(C5CCC5)n4c3c2)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | 1092.8 [M + H]⁺ |
| LP-6 | Compound 5 | 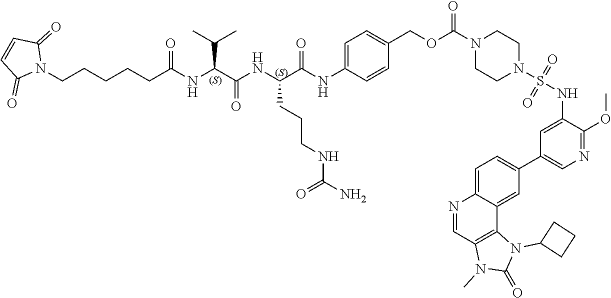 | 561.4 [1/2M + H]⁺ |
| LP-7 | Compound 6 | 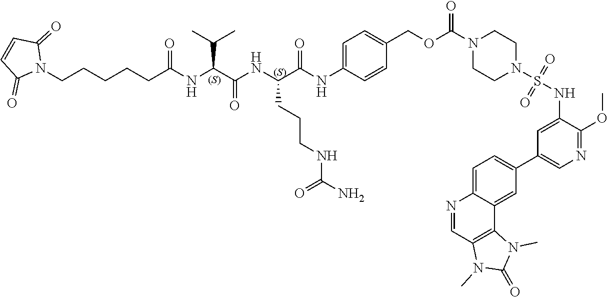 | 541.4 [1/2M + H]⁺ |
| LP-8 | Compound 7 | 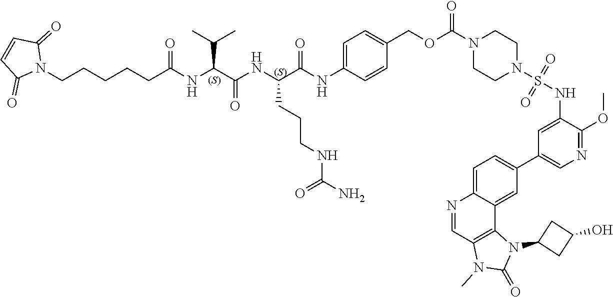 | 569.4 [1/2M + H]⁺ |
| LP-9 | Compound 8 | 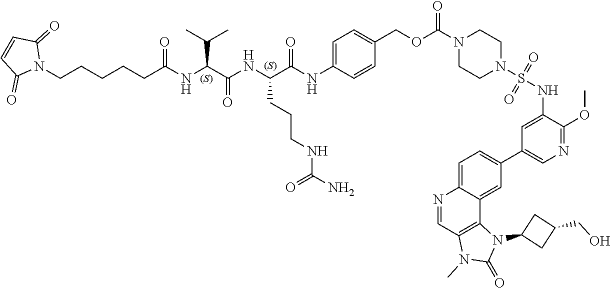 | 576.4 [1/2M + H]⁺ |
| LP-11 | Compound 23 | 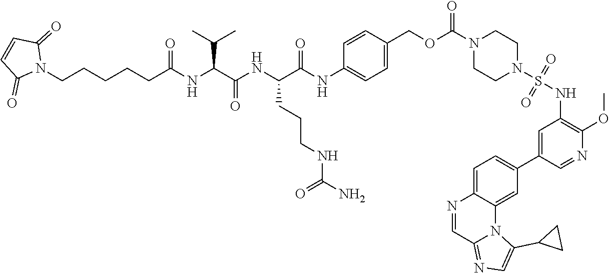 [SMILES: COc1ncc(-c2ccc3ncc4ncc(C5CC5)n4c3c2)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | 1078.6 [M + H]⁺ |
| LP-17 | Compound 18 | 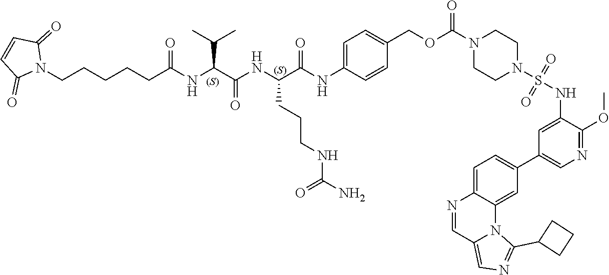 | 546.4 [1/2M + H]⁺ |
| LP-18 | Compound 20 | 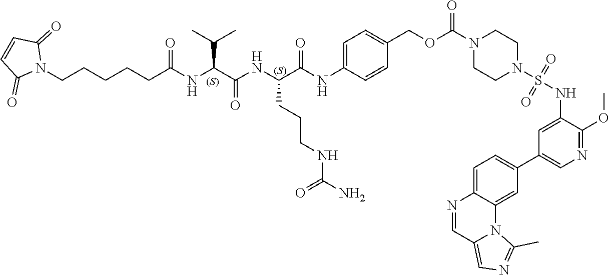 | 526.4 [1/2M + H]⁺ |
| LP-19 | Compound 14 | 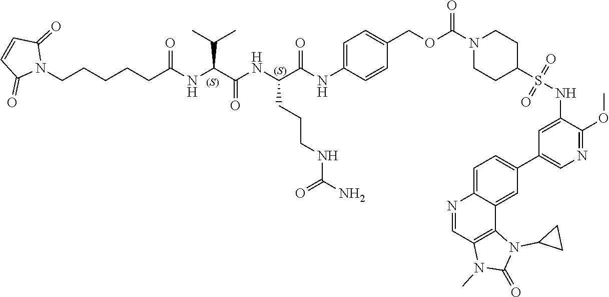 | 553.8 [1/2M + H]⁺ |
| LP-22 | Compound 28 | 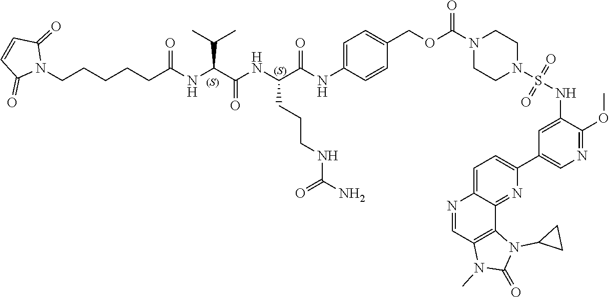 | 1109.8 [M + H]⁺ |
| LP-23 | Compound 30 | 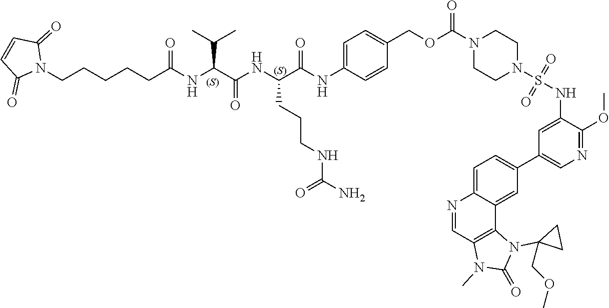 | 576.9 [1/2M + H]⁺ |
| LP-24 | Compound 29 | 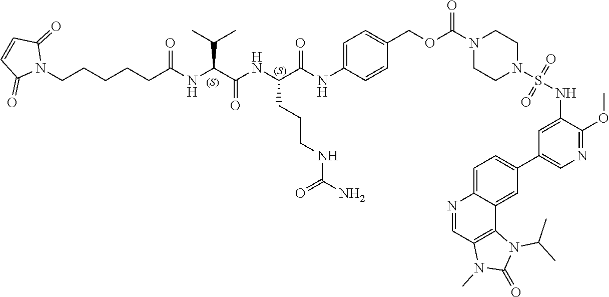 | 1110.4 [M + H]⁺ |
| LP-25 | Compound 27 | 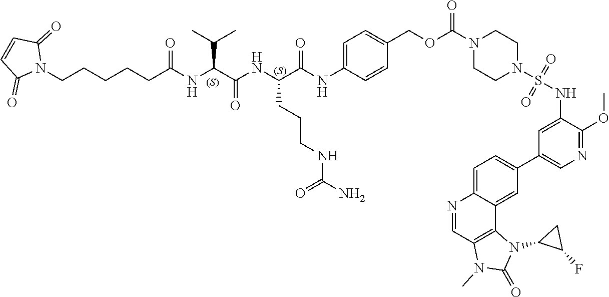 | 1126.4 [M + H]⁺ |
| LP-26 | Compound 32 | 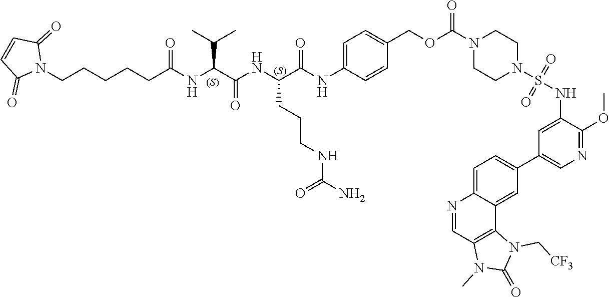 | 1150.4 [M + H]⁺ |
| LP-27 | Compound 33 | 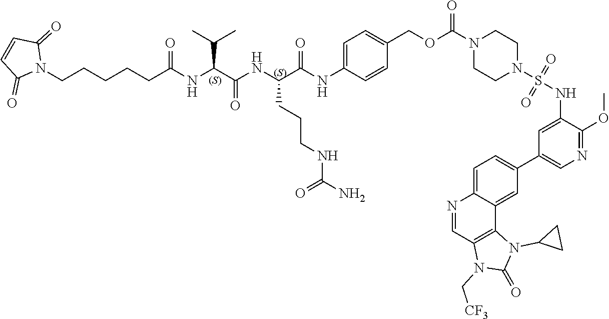 | 1176.6 [M + H]⁺ |
| LP-28 | Compound 31 | 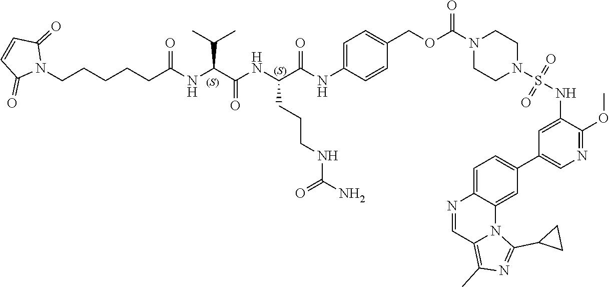 | 1092.6 [M + H]⁺ |

### Preparation of LP-10:

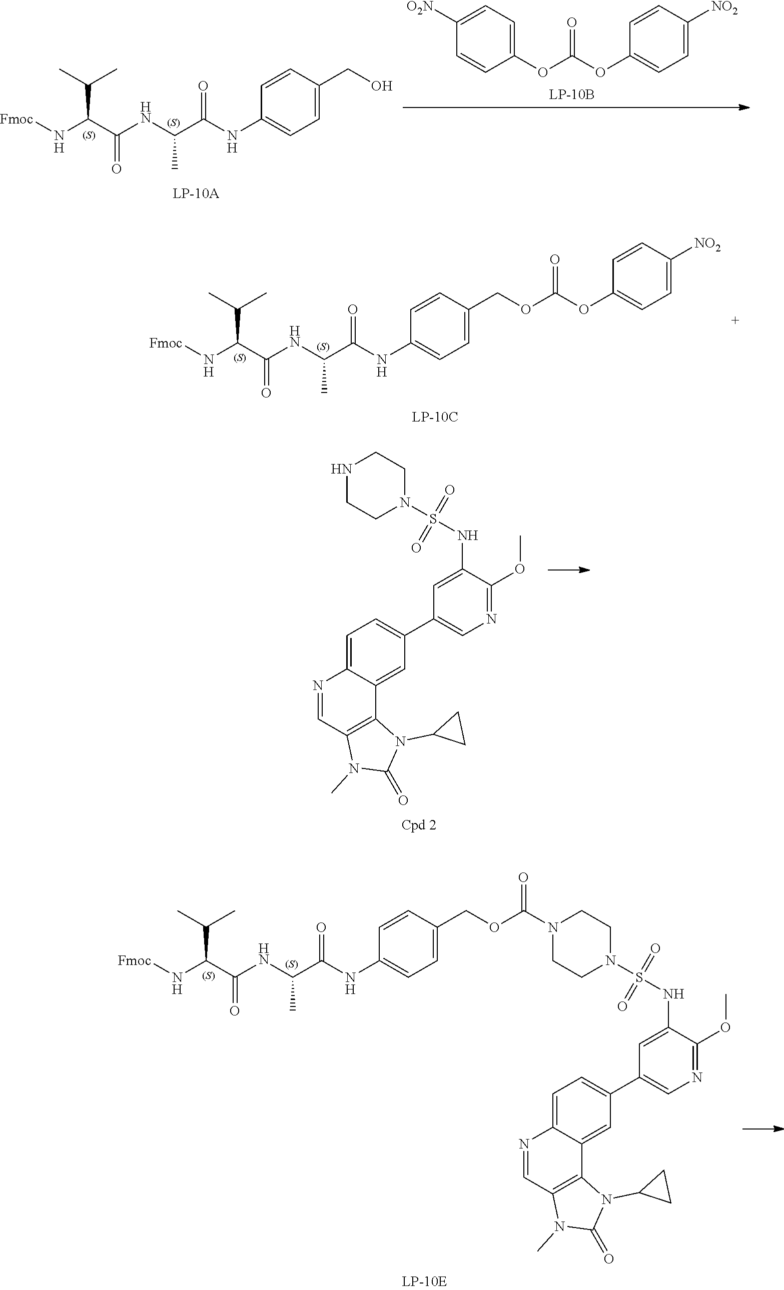

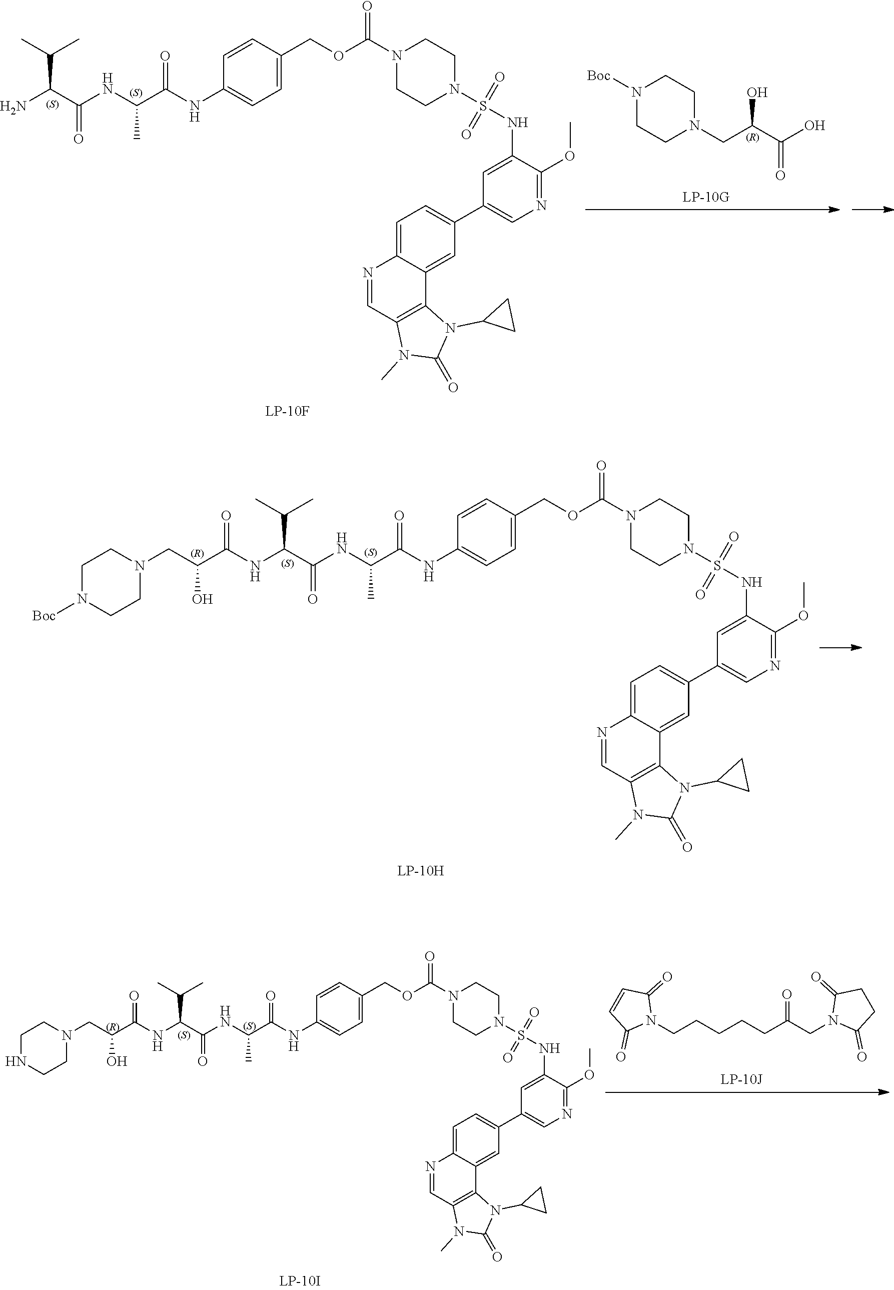

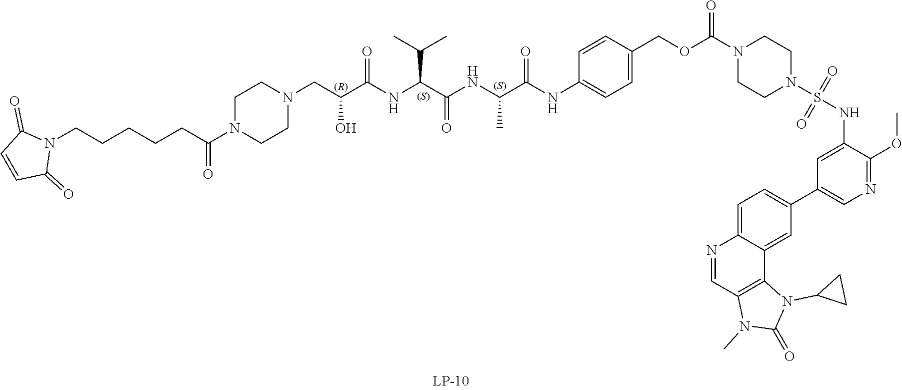

### 1) Synthesis of Compound LP-10C

In an ice-water bath and under nitrogen protection, DIEA (151 μL, 0.87 mmol) was added to a solution of compound LP-10A (CAS: 1394238-91-5) (300 mg, 0.58 mmol) and compound LP-10B (CAS: 5070-13-3) (353 mg, 1.16 mmol) in DMF (3 mL). After the addition was completed, the reaction was stirred at room temperature overnight. After the reaction was completed, the reaction solution was concentrated to remove the solvent, and the residue was purified by ISCO (mobile phase: MeOH:DCM=0:100-50:50) to afford compound LP-10C as a yellow solid (203 mg, 51% yield). MS(m/z)=703.3 [M+23]⁺

### 2) Synthesis of Compound LP-10E

In an ice-water bath and under nitrogen protection, to a solution of compound LP-10C (150 mg, 0.22 mmol) and compound 2 (Cpd2, 135 mg, 0.26 mmol) in DMF (3 mL) was added pyridine (0.5 mL) followed by HOBt (15 mg, 0.11 mmol) and DIEA (77 μL, 0.44 mmol). After the addition was completed, the reaction solution was stirred at room temperature overnight. After the reaction was completed, the reaction solution was directly purified by ISCO (mobile phase: MeOH:H₂O (0.1% TFA)=0:100-100:0) to afford compound LP-10E as a white solid (110 mg, 48% yield). MS(m/z)=1050.8 [M+H]⁺

### 3) Synthesis of Compound LP-10F

In an ice-water bath and under nitrogen protection, to a solution of compound LP-10E (110 mg, 0.11 mmol) in DMA (3 mL) was added DEA (0.5 mL). After the addition was completed, the reaction solution was stirred at room temperature for 1 h. After the reaction was completed, the reaction solution was directly purified by ISCO (mobile phase: CH₃CN:H₂O (0.1% TFA)=0:100-100:0) to afford compound LP-10F as a yellow solid (98 mg). MS(m/z)=829.0 [M+H]⁺

### 4) Synthesis of Compound LP-10H

In an ice-water bath and under nitrogen protection, to a solution of compound LP-10F (98 mg, 0.12 mmol) and compound LP-10G (synthesized according to the preparation of intermediate-17) (42 mg, 0.16 mmol) in DMA (3 mL) was added HOBt (24 mg, 0.18 mmol) and EDCI (68 mg, 0.36 mmol), the mixture was stirred for 2 minutes, and then DIEA (82 μL, 0.47 mmol) was added. After the addition was completed, the mixture was stirred at room temperature for 5 h. After the reaction was completed, the reaction solution was directly purified by ISCO (mobile phase: CH₃CN:H₂O (0.1% TFA)=0:100-100:0) to afford compound LP-10H as a white solid (105 mg, 82% yield). MS(m/z)=1085.8 [M+H]⁺

### 5) Synthesis of Compound LP-10I

In an ice-water bath and under nitrogen protection, to a solution of compound LP-10H (105 mg, 0.10 mmol) in DCM (2 mL) was added TFA (1 mL), and the mixture was stirred for 15 minutes. After the reaction was completed, the reaction solution was concentrated, and the residue was purified by ISCO (mobile phase: CH₃CN:H₂O (0.1% TFA)=0:100-100:0) to afford compound LP-10I as a yellow solid (87 mg, 91% yield). MS(m/z)=984.8 [M+H]⁺

### 6) Synthesis of LP-10

Compound LP-10I (87 mg, 0.088 mmol), compound LP-10J (CAS: 55750-63-5) (35 mg, 0.115 mmol), and DIEA (31 μl, 0.176 mmol) were dissolved in DMA (2 mL), and the mixture was stirred at room temperature for 4 hours. After the reaction was completed, the reaction solution was directly separated by ISCO (mobile phase: CH₃CN:H₂O (0.1% TFA)=0:100-100:0) to afford LP-10 as a yellow solid (14 mg, 13% yield). MS(m/z)=1177.8 [M+H]⁺

The following compounds were prepared according to the preparation procedure of compound LP-10 using the corresponding intermediates and reagents under appropriate conditions recognized by a person skilled in the art.

| | Payloads | | |
|---|---|---|---|
| LP No. | used | Structural formula | MS |
| LP-12 | Compound 1 | 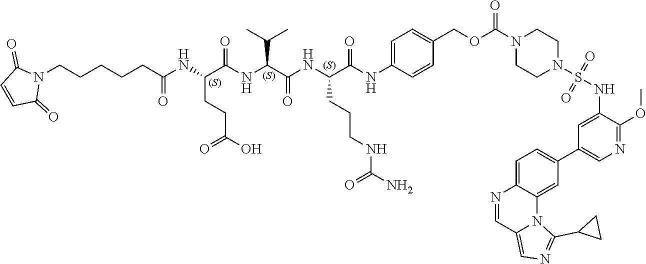 | 604.4 [1/2M + H]⁺ |
| LP-13 | Compound 2 | 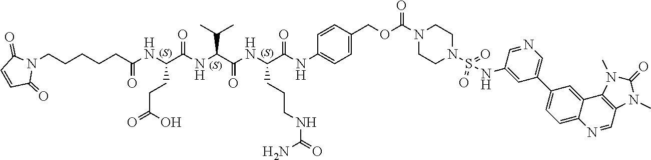 | 1237.8 [M + H]⁺ |

### Preparation of LP-14:

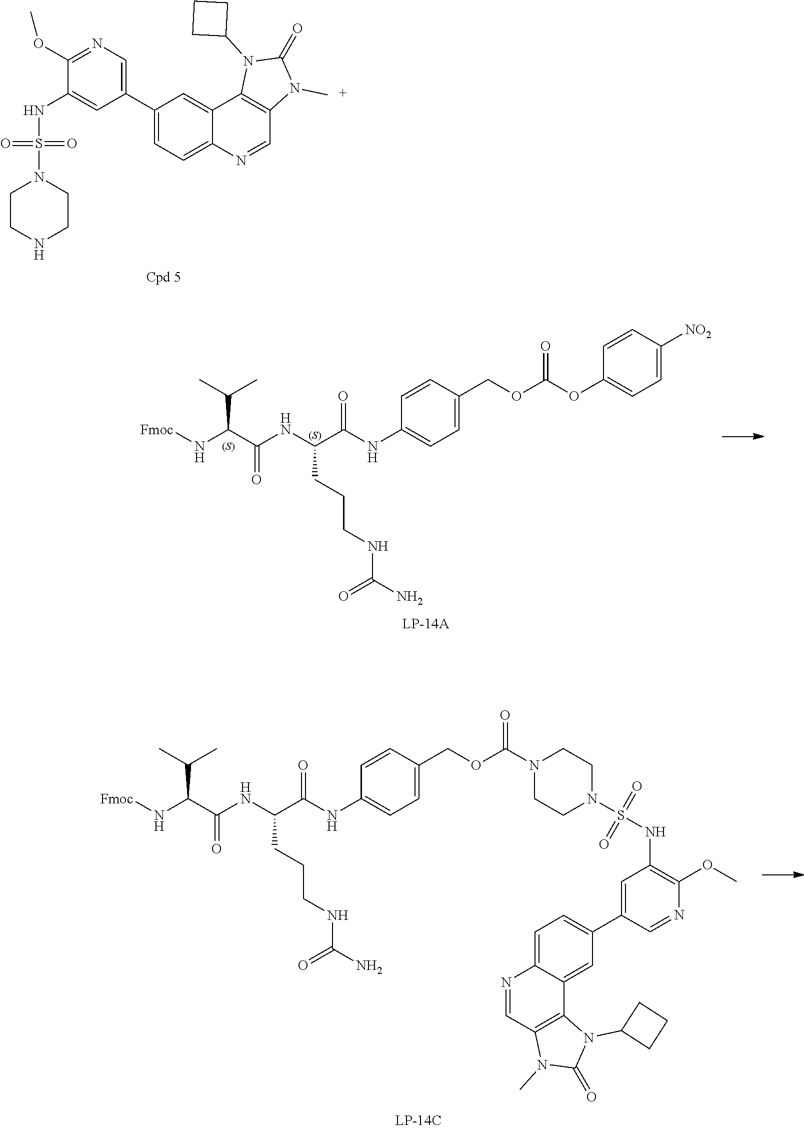

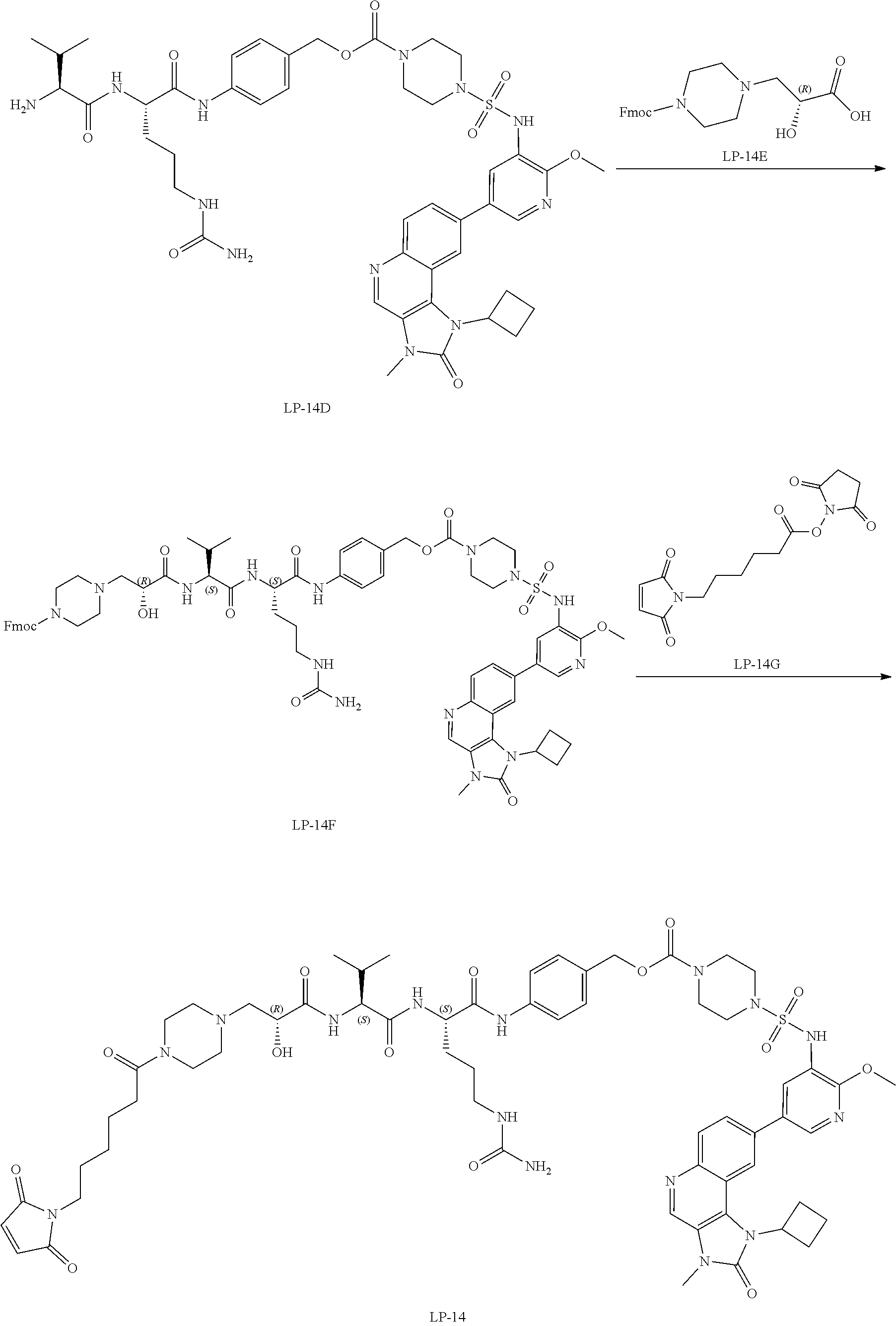

### 1) Synthesis of Compound LP-14C

At room temperature and under nitrogen protection, pyridine (25 mg, 0.32 mmol) and HOBt (15 mg, 0.11 mmol) were added to a solution of compound 5 (Cpd5, 55 mg, 0.11 mmol) and compound LP-14A (CAS: 863971-53-5) (81 mg, 0.11 mmol) in anhydrous DMF, respectively. After the addition was completed, the reaction solution was stirred at room temperature overnight. After the reaction was completed, the reaction solution was concentrated under reduced pressure, and the residue was purified by ISCO (mobile phase: MeOH:H₂O=0:100-100:0) to afford compound LP-14C as a white solid (58 mg, 48% yield). MS(m/z)=1151.8 [M+H]⁺

### 2) Synthesis of Compound LP-14D

Diethylamine (1 mL) was added to a solution of compound LP-14C (58 mg, 0.050 mmol) in DCM/MeOH (2 mL, 3:1) at room temperature. The reaction was stirred at room temperature for 3 h. After the reaction was completed, the reaction solution was concentrated under reduced pressure. The residue was stirred with THF/PE (20 mL, 1:5) for 30 minutes, and filtered. The filter cake was collected and dried to afford compound LP-14D as a white solid (45 mg, 96% yield). MS(m/z)=929.4 [M+H]⁺

### 3) Synthesis of Compound LP-14F

In an ice-water bath and under nitrogen protection, DIEA (19 mg, 0.15 mmol), and HATU (22 mg, 0.058 mmol) were added to a solution of compound LP-14D (45 mg, 0.048 mmol) and compound LP-14E (intermediate-17) (20 mg, 0.048 mmol) in DCM (10 mL), respectively. The reaction solution was stirred at room temperature overnight. After the reaction was completed, the reaction solution was poured into water, and extracted with DCM (20 mL×3). The organic phases were combined and dried over anhydrous sodium sulfate, concentrated under reduced pressure, and purified by ISCO (mobile phase: MeOH:H₂O=0:100-100:0) to afford compound LP-14F as a white solid (32 mg, 51% yield). MS(m/z)=1307.8 [M+H]⁺

### 4) Synthesis of LP-14

Compound LP-14F (32 mg, 0.024 mmol), compound LP-14G (CAS: 55750-63-5) (15 mg, 0.049 mmol), and triethylamine (75 mg, 0.734 mmol) were dissolved in DMF (2 mL), and the mixture was stirred at room temperature overnight. After the reaction was completed, the reaction solution was concentrated under reduced pressure and separated by ISCO (mobile phase: H₂O (0.1% HCOOH):CH₃CN=100:0-0:100) to afford compound LP-14 as a white solid (3 mg, 9% yield). MS(m/z)=1278.6 [M+H]⁺

The following compounds were prepared according to the preparation procedure of compound LP-14 using the corresponding intermediates and reagents under appropriate conditions recognized by a person skilled in the art.

| LP | Payloads | | |
|---|---|---|---|
| No. | used | Structural formula | MS |
| LP- 15 | Compound 1 | 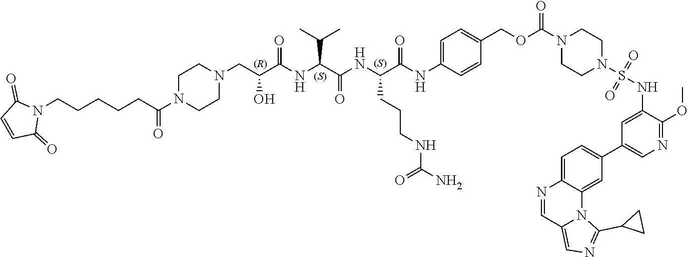 | 617.8 [1/2M + H]⁺ |
| LP- 16 | Compound 2 | 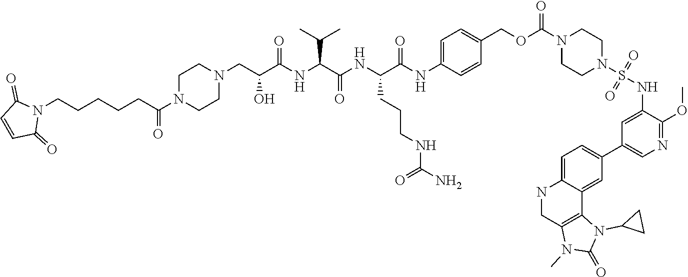 [SMILES: COc1ncc(-c2ccc3c(c2)-c2c(n(C)c(=O)n2C2CC2)CN3)cc1NS(=O)(=O)N1CCN(C(=O)OCc2ccc(NC(=O)[C@H](CCCNC(N)=O)NC(=O)[C@@H](NC(=O)[C@H](O)CN3CCN(C(=O)CCCCCN4C(=O)C=CC4=O)CC3)C(C)C)cc2)CC1] | 1264.0 [M + H]⁺ |

### Preparation of LP-21:

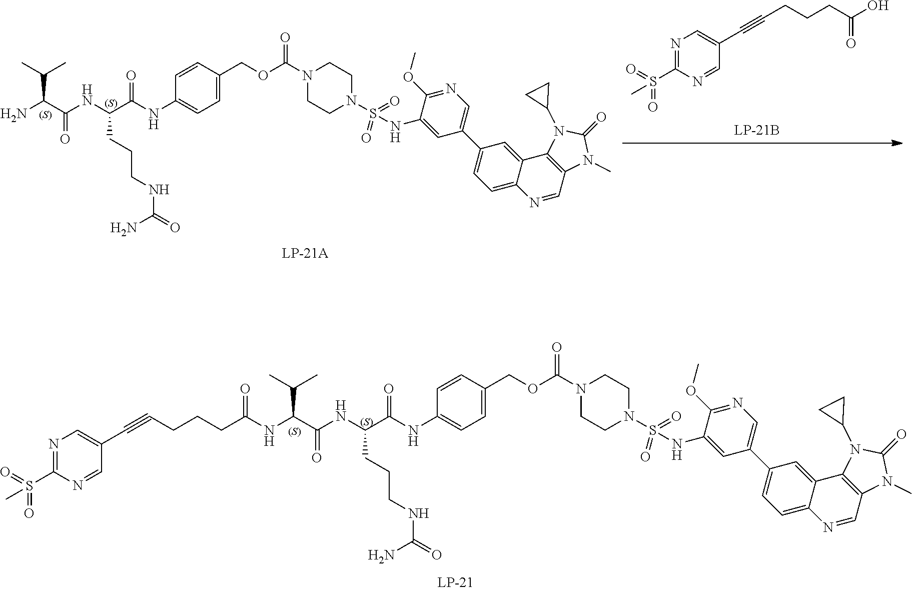

DIEA (8 mg, 0.054 mmol) and HATU (16 mg, 0.041 mmol) were added to a solution of compound LP-21A (25 mg, 0.027 mmol) (synthesized according to the synthesis of compound LP-10F in the synthesis of LP-10) and compound LP-21B (CAS: 2356229-58-6) (10 mg, 0.037 mmol) in DCM, and the mixture was stirred overnight at room temperature under nitrogen protection. After the reaction was completed, the reaction solution was poured into water, and extracted with DCM (20 mL×3). The organic phases were combined, dried over anhydrous sodium sulfate, concentrated under reduced pressure, and separated by ISCO (mobile phase: water (0.1% formic acid): acetonitrile=100:0-0:100) to afford compound LP-21 as a white solid (8 mg, 24% yield). MS(m/z)=1165.6 [M+H]⁺

The following compounds were prepared according to the preparation procedure of compound LP-21 using the corresponding intermediates and reagents under appropriate conditions recognized by a person skilled in the art.

| LP | Payloads | | |
|---|---|---|---|
| No. | used | Structural formula | MS |
| LP-20 | Compound 1 | 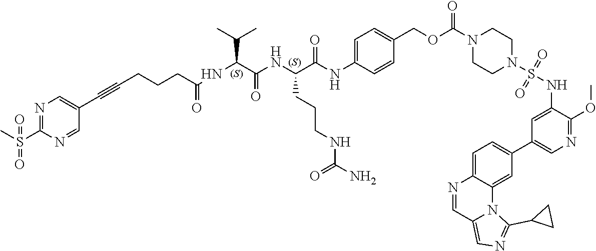 | 1136.9 [M + H]⁺ |

### Example 4: Preparation and Characterization of ADC

### Preparation of ADC-1:

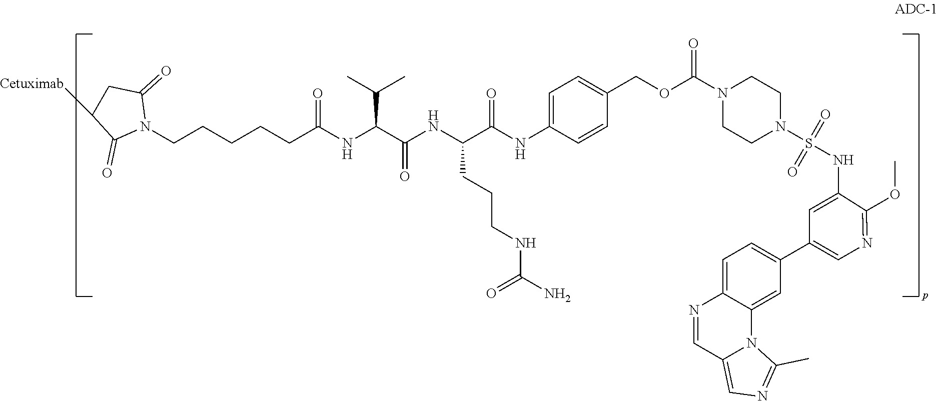

His buffer (605.1 μL), TCEP solution (7.34 μL, 10 mmol/L in His buffer, 2.2 eq), and DTPA solution (12.5 μL, 10 mmol/L in His buffer) were sequentially added to 8 mg/mL cetuximab (with sequence information shown in the sequence listing) solution (625 μL, 5 mg) formulated with His buffer (10 mmol/L, pH 7.0). The resulting solution was reacted at 22° C. for 60 minutes, and then a solution of compound LP-2 in DMSO:H₂O (volume ratio: 3:1) (66.67 μL, 5 mmol/L, 10 eq) was added. The mixture was further reacted at 22° C. for 60 minutes. The reaction solution was transferred to an ultrafiltration centrifuge tube (Millipore Amicon® Ultra), and a His/His·HCl buffer (10 mmol/L, pH 6.2) was added for solution exchange, so that a target product was obtained. As detected by HIC-HPLC, the drug/antibody ratio (DAR value) was calculated as 3.9.

The following antibody-drug conjugates were prepared according to the preparation procedure of ADC-1 using the corresponding antibodies and linker-payloads under appropriate conditions recognized by a person skilled in the art (e.g., the commonly used conjugation methods described in the specification).

| ADC No. | Structure | Measured DAR |
|---|---|---|
| ADC-2 | 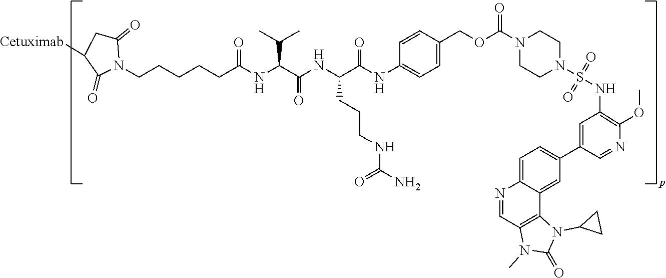 | 3.9 |
| ADC-3 | 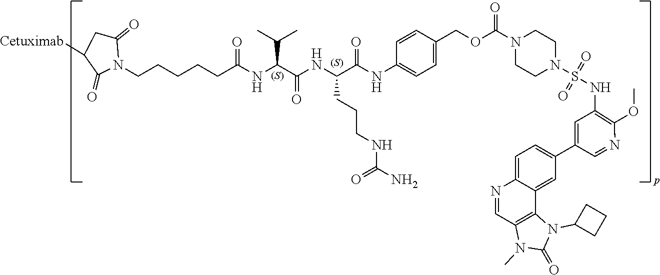 | 3.8 |
| ADC-4 | 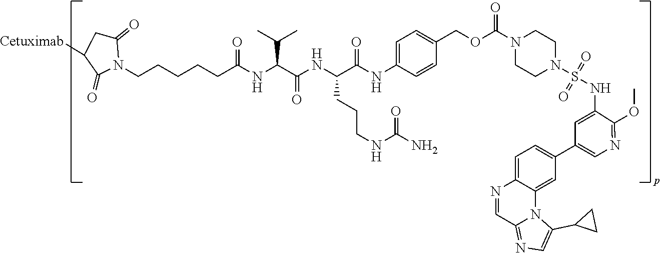 | 3.8 |
| ADC-5 | 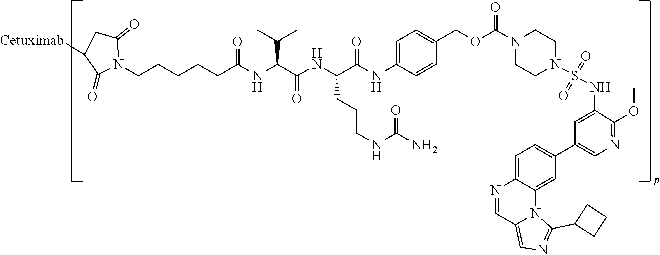 | 3.8 |
| ADC-6 | 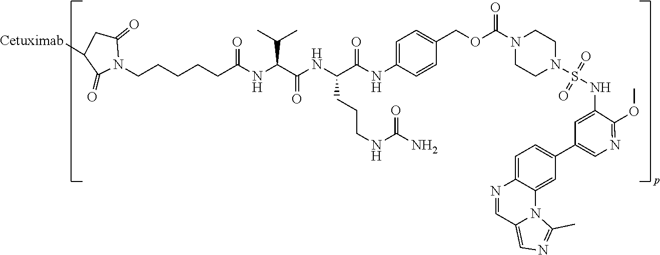 | 3.9 |
| ADC-7 | 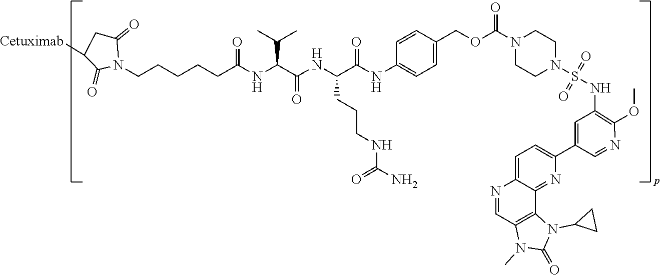 | 3.9 |
| ADC-8 | 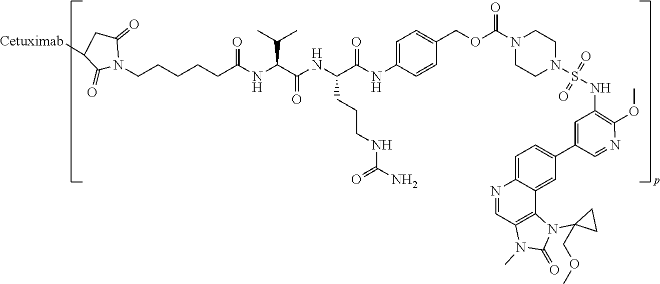 | 3.9 |
| ADC-9 | 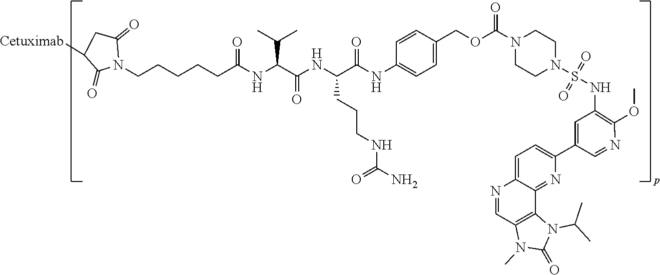 | 4.0 |
| ADC-10 | 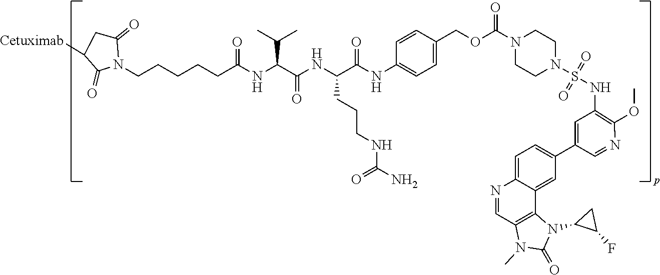 | 4.0 |
| ADC-11 | 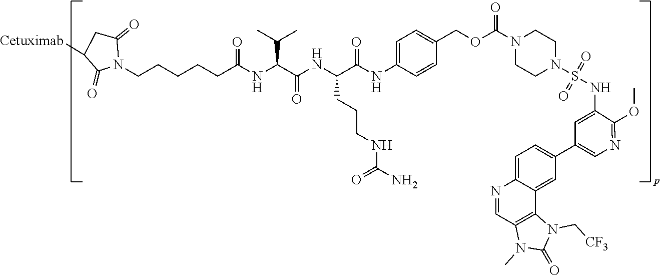 | 4.0 |
| ADC-12 | 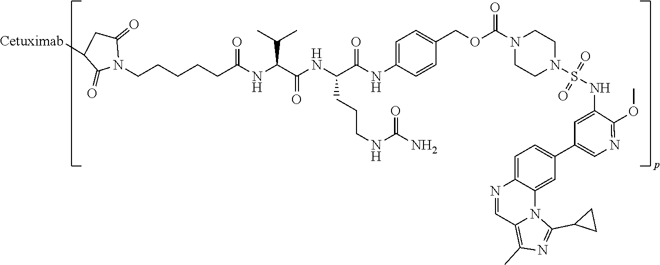 | 4.0 |
| ADC-13 | 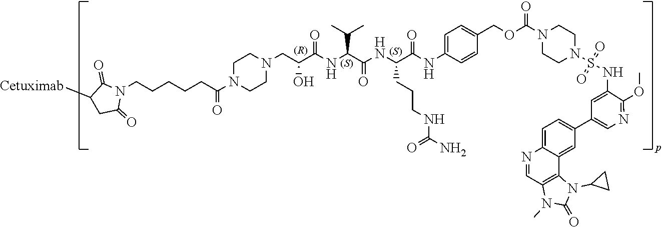 | 4.0 |

### Example 5: Testing the Binding Activity of the ADCs of the Present Invention to Corresponding Positive Cells

### Experimental Methods

First of all, HCC827 cells (EGFR positive, Cat. No. CRL-2868) (purchased from the cell bank of American Type Culture Collection, ATCC) were normally cultured in an RPMI-1640 medium (Gibco, Cat. No. A1049101) with 10% fetal bovine serum (FBS) (Gibco, Cat. No. A5669701) in a cell culture incubator at 5% CO₂ and 37° C. The adhered cells were digested with 0.25% Trypsin-EDTA (Gibco, Cat. No. 25200-072). The dissociated cells were collected, and centrifuged. The cells were resuspended in a staining buffer (PBS (Gibco, Cat. No. C20012500BT)+2% FBS) at 4° C., and were added to a V-bottom 96-well plate (BD Falcon) at 1.0×10⁵ cells/well and 50 μL/well. The antibody or the antibody-drug conjugate was diluted with a staining buffer in a 4-fold gradient to the corresponding concentrations, and then 50 μL/well of the diluted antibody or antibody-drug conjugate at different concentrations (final concentrations: 150, 37.5, 9.38, 2.34, 0.586, 0.146, 0.037, and 0.0092 nM) or 50 μL/well of the human IgG1 isotype reference antibody (Biolegend, 403502) were added to the corresponding cell wells, respectively. The plate was incubated at 4° C. for 1.0 hour. After the centrifugation at 300 g for 5 minutes, the supernatant was aspirated, and the pellet was resuspended in 200 μL/well of staining buffer and washed twice. The PE-labeled anti-human IgG Fc antibody (Biolegend, 410708) diluted in a staining buffer was added at 100 μL/well and incubated in the dark at 4° C. for 45 minutes. After the centrifugation at 300 g for 5 minutes, the supernatant was aspirated, and the pellet was resuspended in 200 μL/well of staining buffer and washed twice. Then, the cells were resuspended in 200 μL/well of staining buffer.

### Detection and Data Analysis

Fluorescence signals were detected on a BD LSRFortessa flow cytometer (BD Biosciences) to obtain the mean fluorescence intensity (MFI) of the binding of the antibody or antibody-drug conjugate to cells, and the EC₅₀ values were then obtained using the Log (agonist) vs. response equation in the GraphPad Prism software (GraphPad Software).

The resulting data are shown in the following table:

TABLE 1

| Antibody or ADC | EC₅₀, nM HCC827 cell line |
|---|---|
| Cetuximab | 0.22 |
| ADC-1 | 0.25 |
| ADC-2 | 0.24 |
| ADC-3 | 0.22 |
| ADC-4 | 0.27 |
| ADC-5 | 0.28 |
| ADC-6 | 0.24 |
| ADC-7 | 0.24 |
| ADC-8 | 0.22 |
| ADC-9 | 0.22 |
| ADC-10 | 0.23 |
| ADC-11 | 0.24 |
| ADC-12 | 0.24 |
| ADC-13 | 0.24 |

Conclusions: As can be seen from the data in Table 1, the ADC products of the present invention exhibit a cell binding activity similar to the naked antibody (cetuximab) in the HCC827 EGFR-positive cell line.

### Example 6: Inhibition of Cell Proliferation by Payloads Prepared According to the Present Invention In Vitro

### Experimental Methods

MDA-MB-453 (Cat. No. HTB-131, purchased from the cell bank of ATCC) and MDA-MB-468 (purchased from Cell Resource Center, Shanghai Academy of Biological Sciences, Chinese Academy of Sciences) were normally cultured in a DMEM medium (Gibco, Cat. No. 11965-092) with 10% fetal bovine serum (FBS) in a cell culture incubator at 5% CO₂ and 37° C. The cells were added to a 96-well plate at 1.0×10³ cells/well and 100 μL/well and incubated in a cell culture incubator at 5% CO₂ and 37° C. The test compounds were diluted with a DMEM medium containing 2% DMSO (Sigma-Aldrich, Cat. No. 34869) in a 4-fold gradient to the corresponding concentrations. Then 10 μL/well of the diluted test compounds at different concentrations (final concentrations: 250, 62.5, 15.6, 3.91, 0.98, 0.24, 0.061, and 0.015 nM) or 10 μL/well of the control solution (DMEM containing 2% DMSO) were added to a 100 μL/well cell culture system. The final concentration of DMSO was 0.2%. The cells were incubated in a cell culture incubator at 5% CO₂ and 37° C. for 6 days.

After 6 days of culture, 10 μL/well of Cell Counting Kit-8 reagent (CCK-8, Dojindo, Cat. No. CK04) was added to the wells and mixed well with shaking. The cells were incubated in a cell culture incubator at 5% CO₂ and 37° C. for 0.50-4.0 hours.

### Detection and Data Analysis

The OD450 optical density value was read on an EnVision™ multi-function microplate reader (PerkinElmer), and the inhibition rates (%) of the compounds were calculated. Then, the half maximal inhibitory concentration (IC₅₀) values were obtained using the XLfit5 software (ID Business Solutions Limited). All samples were tested in duplicate wells.

$$Inhibition rate \% = ( 1 - ( O D {450}_{test compound} - O D {450}_{background} ) / ( O D {450}_{cell} - O D {450}_{background} ) ) \times 100 \%$$

wherein
OD450_{test compound} represents the optical density value in the wells of the test compounds;
OD450_{cell} represents the optical density value in the wells of cell controls;
OD450_{background} represents the optical density value in the wells of cell-free controls.

The resulting data are shown in the following table:

TABLE 2

IC₅₀, nM

| Compound No. | MDA-MB-453 | MDA-MB-468 |
|---|---|---|
| Reference compound 1 | 7.9 | 6.0 |
| Compound 1 | 0.23 | 0.39 |
| Compound 2 | 0.31 | 0.41 |
| Compound 3 | 0.074 | 0.076 |
| Compound 4 | 0.89 | 0.71 |
| Compound 5 | 0.064 | 0.090 |
| Compound 6 | 0.25 | 0.32 |
| Compound 7 | 0.30 | 0.22 |
| Compound 8 | 0.32 | 0.21 |
| Compound 9 | 0.79 | 1.0 |
| Compound 10 | 2.3 | 1.5 |
| Compound 11 | 25.2 | 28.1 |
| Compound 12 | 3.3 | 7.9 |
| Compound 13 | 1.3 | 1.8 |
| Compound 14 | 0.39 | 0.41 |
| Compound 15 | 14.9 | 18.4 |
| Compound 16 | 61.6 | 91.4 |
| Compound 17 | 1.8 | 3.4 |
| Compound 18 | 0.15 | 0.091 |
| Compound 19 | 0.12 | 0.055 |
| Compound 20 | 0.43 | 1.0 |
| Compound 21 | 6.6 | 4.9 |
| Compound 22 | 6.5 | 9.2 |
| Compound 23 | 0.55 | 1.4 |
| Compound 24 | 0.24 | 0.20 |
| Compound 25 | 0.34 | 0.20 |
| Compound 26 | 1.8 | 1.9 |
| Compound 27 | 0.24 | 0.29 |
| Compound 28 | 0.16 | 0.24 |
| Compound 29 | 0.056 | 0.078 |
| Compound 30 | 0.41 | 0.64 |
| Compound 31 | 0.24 | 0.47 |
| Compound 32 | 0.22 | 0.24 |
| Compound 33 | 2.3 | 3.0 |
| Compound 34 | 2.8 | 3.4 |
| Compound 35 | 0.22 | 0.20 |

### Example 7: Kinase Activity Determination for Payloads of the Present Invention

Determination of FRAP1 (mTOR) Kinase Activity

### 1. Reagents and Materials

Human FRAP1 (mTOR) recombinant protein (GST tag): Invitrogen, Cat. No. PV4753;
GFP-4E-BP1: Thermo Scientific, Cat. No. PV4759;
LanthaScreen™ Tb-p4E-BP1 (pThr46) antibody: Thermo Scientific, Cat. No. PV4755;
TR-FRET dilution buffer: Thermo Scientific, Cat. No. PV3574;
ATP solution (10 mM): Thermo Scientific, Cat. No. PV3227;
HEPES (1 M): Gibco, Cat. No. 15630-106;
EDTA (0.5 M), pH 8.0, RNase-free: Invitrogen, Cat. No. AM9260G;
Manganese chloride solution: Sigma-Aldrich, Cat. No. M1787;
EGTA: Sigma-Aldrich, Cat. No. E4378;
TWEEN®20: Sigma-Aldrich, Cat. No. P2287;
DTT: Sigma-Aldrich, Cat. No. DTT-RO;
384-well plate: Corning, Cat. No. 3676;
384-well plate: Corning, Cat. No. 4514;
Envision multi-mode plate reader: PerkinElmer;

### 2. Methods

### (1) Principle:

4EBP1 is the substrate of mTOR, and when mTOR is activated, it phosphorylates 4EBP1 at residue Thr⁴⁶. LanthaScreen™ kinase assay is used to evaluate the inhibitory effects of compounds on mTOR kinase activity through quantitative detection of phosphorylated GFP-4E-BP1 levels. LanthaScreen™ kinase assay is typically divided into two steps: the kinase first converts ATP into ADP and phosphorylates the substrate GFP-4E-BP1; then a mixture of EDTA (to stop the reaction) and Terbium-labeled (Tb)-p4E-BP1 (pThr46) antibody (to detect phosphorylated product) is added. Addition of the Tb-p4E-BP1 (pThr46) antibody results in FRET from terbium to GFP. The TR-FRET signal ratio reflects the amount of phosphorylated substrate present, and inversely correlates with compound efficacy.

### (2) Reagent Preparation:

1.33× kinase buffer: 5× kinase buffer (250 mM HEPES, 0.05% Tween 20, 5 mM EGTA, and 50 mM manganese chloride) was diluted with water to 1.33× kinase buffer, and 2.67 mM DTT was added;
FRAP1 (mTOR) enzyme solution: the kinase was dissolved in the 1.33× kinase buffer to achieve a working concentration of 0.15 ng/μl;
ATP/GFP-4EBP1 mixture: The ATP stock solution (10 mM aqueous solution) and the kinase substrate GFP-4EBP1 were dissolved in the 1.33× kinase buffer, to achieve a working concentration of 10 μM for ATP and a working concentration of 0.4 μM for the kinase substrate GFP-4EBP1;
EDTA and Tb-p4E-BP1 (pThr46) antibody mixture: EDTA solution and Tb-p4E-BP1 (pThr46) antibody were dissolved in TR-FRET dilution buffer, to achieve a working concentration of 10 mM for EDTA and a working concentration of 2 nM for the Tb-p4E-BP1 (pThr46) antibody;
4× compound formulation: the compound was 3-fold serially diluted in 4% DMSO aqueous solution. The final concentrations of the test compound were 3 μM, 1 μM, 0.33 μM, 0.11 μM, 0.037 μM, 0.012 μM, 0.0041 μM, 0.0014 μM, 0.00046 μM, 0.00015 μM, and 0.000051 μM.

### (3) Experimental Procedure:

In the experiment, there were two control groups (16 technical replicates each), one was 100% inhibition group (no kinase); the other was 0% inhibition group. The serially diluted compound was added to the indicated wells, and 4% aqueous DMSO solution was added to the control wells. 2.5 μl of FRAP1 (mTOR) enzyme solution was added to the test compound wells, an equivalent volume of kinase-free reaction buffer was added to the 100% inhibition wells; then, 5 μl of ATP/GFP-4EBP1 mixture was added to each well. The 384-well plate was centrifuged at 1000 rpm for 60 seconds, followed by a light-protected incubation at room temperature for 1 hour. Once the enzymatic reaction was completed, 10 μl of a mixture of EDTA and Tb-p4E-BP1 (pThr46) antibody was added. The plate was centrifuged at 1000 rpm for 60 seconds, followed by a light-protected incubation at room temperature for 1 hour.

### 3. Detection

Read plate using an Envision multi-mode plate reader (the excitation light wavelength was 320 nm and the emission light wavelengths were 495 nm and 520 nm).

### 4. Calculation

The inhibition rate was calculated using the formula as below:

$$Inhibition rate ( \% ) = 100 \% \times ( mean signal value of 0 \% inhibition group - signal value of testing well ) / ( mean signal value of 0 \% inhibition group - mean signal value of 100 \% inhibition group )$$

The mean signal values of the 100% inhibition group and the 0% inhibition group were used as the reference values, the inhibition rate (%) of each concentration of every compound was calculated according to the signal value of each well, and the IC₅₀ value was obtained by model 205 in XL-Fit 5.5 software (ID Business Solutions Limited).

### Determination of PIK3CA/PIK3R1 (P110a/p85α) Kinase Activity

### 1. Reagents and Materials

Human PIK3CA/PIK3R1 (p110a/p85α) recombinant protein (His tag): Invitrogen, Cat. No. PV4788
Transcreener® ADP2 FP kit: BellBrook Labs, Cat. No. 3010-10K;
CHAPS hydrate: Sigma-Aldrich, Cat. No. C₅₀₇₀;
EGTA: Sigma-Aldrich, Cat. No. E4378;
DTT: Sigma-Aldrich, Cat. No. DTT-RO;
HEPES (1 M): Gibco, Cat. No. 15630-106;
EDTA (0.5 M), pH 8.0, RNase-free: Invitrogen, Cat. No. AM9260G;
ATP solution (10 mM): Thermo Scientific, Cat. No. PV3227;
PIP2: PS lipid kinase substrate: Thermo Scientific, Cat. No. PV5100;
MgCl₂ (1 M): Invitrogen, Cat. No. AM9530G;
NaCl (5 M), no RNase: Invitrogen, Cat. No. AM9759;
96-well plate: Greiner, Cat. No. 675076;
Envision multi-mode plate reader: PerkinElmer;

### 2. Methods

### (1) Principle:

Transcreener® ADP2 FP kit is employed to determine the inhibitory effects of test compounds on PIK3CA/PIK3R1 (p110 α/p85 α) kinase activities through detection of the amount of ADP. Transcreener® ADP2 FP assay is typically divided into two steps: the kinase first converts ATP into ADP and phosphorylates the substrate PIP2: PS; then, the ADP detection mixture (the mixture contains stop and detect buffer, as well as the ADP Alexa633 Tracer and ADP² antibody) is added. The complex formation of the ADP Alexa633 Tracer to the ADP² antibody results in production of fluorescence polarization. The kinase-produced ADP competes with the tracer-labeled ADP to bind the ADP² antibody, resulting in a decrease in the fluorescence polarization signal. The intensity of fluorescence polarization signal inversely correlates with ADP produced in the reaction.

### (2) Reagent Preparation:

1.25× kinase buffer: comprising 62.5 mM HEPES, 125 mM sodium chloride, 1.25 mM EGTA, 0.0375% CHAPS, 3.75 mM magnesium chloride, and 1.25 mM DTT;
PIK3CA/PIK3R1 (p110a/p85α) enzyme solution: the kinase was dissolved in the 1.25× kinase buffer to achieve a working concentration of 0.096 ng/ul;
A mixed solution of ATP and PIP2:PS lipid kinase substrate: the ATP stock solution (10 mM aqueous solution) and PIP2:PS lipid kinase substrate were dissolved in the 1.25× kinase buffer, to achieve a working concentration of 10 μM for ATP and a working concentration of 30 μM for the PIP2:PS lipid kinase substrate;
5× compound formulation: the compound was 3-fold serially diluted in 10% DMSO aqueous solution. The final concentrations of the test compound were 100 nM, 33.3 nM, 11.1 nM, 3.70 nM, 1.23 nM, 0.41 nM, 0.14 nM, and 0.046 nM.

### (3) Experiment Procedure:

In the experiment, there were two control groups (4 technical replicates each), one was 100% inhibition group (with 90 mM EDTA); the other was 0% inhibition group (no inhibitor). 5 μl of serially diluted compound was added to the indicated wells in 96-well plate. 5 μl of 10% aqueous DMSO solution was added to 0% inhibition wells and 5 μl of a mixture of 0.45 M EDTA and 10% DMSO was added to 100% inhibition wells. After that, 10 μl of PIK3CA/PIK3R1 (p110α/p85α) enzyme solution was added; then, 10 μl of a mixed solution of ATP and PIP2:PS lipid kinase substrate was added. The 96-well plate was centrifuged at 1000 rpm for 60 seconds, followed by a light-protected incubation at room temperature for 1 hour. Once the enzymatic reaction was completed, 25 μl of ADP detection mixture was added. The 96-well plate was centrifuged at 1000 rpm for 60 seconds, followed by a light-protected incubation at room temperature for 1.5 hours.

10 μM ATP/ADP standard curve: 5 μl of 10% DMSO solution and 10 μl of 1.25× kinase buffer were added to the wells of the standard curve, respectively. Next, ATP and ADP were proportionally mixed to mimic 0.1-100% conversion efficiency. 10 μl of the mixture (single well) was added to the indicated wells. Once the enzymatic reaction was completed, 25 μl of ADP detection mixture was added. The 96-well plate was centrifuged at 1000 rpm for 60 seconds, followed by a light-protected incubation at room temperature for 1.5 hours. In the standard curve, the ATP conversion rates were 100%, 50%, 20%, 10%, 5%, 2.5%, 1%, 0.8%, 0.6%, 0.4%, 0.2%, and 0.1%, respectively.

Fluorescence polarization signal values were calculated for the control groups, including negative control, positive control, and blank control. 5 μl of 10% DMSO solution and 20 μl of 1.25× kinase buffer were added to the 96-well plate, respectively. Once the enzymatic reaction was completed, 25 μl of ADP detection mixture was added to the positive control wells, 25 μl of ADP detection mixture without ADP² antibody was added to the negative control wells, and 25 μl of ADP detection mixture without tracer-label ADP and ADP² antibody was added to the blank control wells. Then 96-well plate was centrifuged at 1000 rpm for 60 seconds, followed by a light-protected incubation at room temperature for 1.5 hours.

### 3. Detection

Read plates using an Envision multi-mode plate reader to detect the vertical polarization S value and the horizontal polarization P value (the excitation light wavelength was 620 nm and the emission light wavelength was 688 nm).

### 4. Calculation

$$Calculation of G factor : G = ( ( 1000 - tracer mP ) \times mean corrected S of negative control group ) / ( ( 1000 + tracer mP ) \times mean corrected P of negative control group )$$

Corrected S is the S value of the detection well−the mean S of the blank control group

Corrected P is the P value of the test well−the mean P of the blank control group

Fluorescence polarization value mP:mP=[(corrected S−corrected P×G)/(corrected S+corrected P×G)]×1000

The inhibition rate was calculated using the formula as below:

$$\% inhibition rate ( I R ) = [ ( mP test sample - mP 0 \% inhibition group ) / ( mP 100 \% inhibition group - mP 0 \% inhibition group ) ] \times 100 \%$$

The mean signal values of the 100% inhibition group and the 0% inhibition group were used as the reference value, the inhibition rate (%) of each concentration of every compound was calculated according to the signal value of each well, and the IC₅₀ value was obtained by model 205 in XL-Fit 5.5 software (ID Business Solutions Limited).

### Test Results

TABLE 3

| | PIK3CA/PIK3R1 | FRAP1 |
|---|---|---|
| | (p110α/p85α) | (mTOR) |
| Compound No. | (Enzyme, IC₅₀, μM) | (Enzyme, IC₅₀, μM) |
| Reference compound 1 | 0.0011 | 0.0053 |
| Compound 1 | 0.0005 | 0.0036 |
| Compound 2 | 0.0007 | 0.0063 |
| Compound 5 | 0.0016 | 0.0075 |
| Compound 7 | 0.0005 | 0.0027 |
| Compound 14 | 0.0006 | 0.0112 |
| Compound 18 | 0.0021 | 0.0062 |
| Compound 19 | 0.0014 | 0.0086 |
| Compound 23 | 0.0007 | 0.0034 |
| Compound 24 | 0.0018 | 0.0053 |
| Compound 25 | 0.0026 | 0.0108 |
| Compound 27 | 0.0015 | 0.0064 |
| Compound 28 | 0.0006 | 0.0028 |
| Compound 29 | 0.0014 | 0.0014 |
| Compound 30 | 0.0023 | 0.0046 |
| Compound 32 | 0.0028 | 0.0022 |

### Determination of ATM(h)/ATR/ATRIP(h)/DNA-PK(h) Kinase Activity

### 1. Reagents and Materials

The kinase sources are shown in the table below.

| Kinase | | | Kinase | |
|---|---|---|---|---|
| name | Cat. No. | Species | type | Registration No. |
| ATM(h) | 14-933 | Human | Atypical | GenBank NM_000051.3 |
| | | derived | | |
| ATR/ | 14-953 | Human | Atypical | GenBank NM_001184.3 |
| ATRIP(h) | | derived | | (ATR)/GenBank |
| | | | | AF451323.1 (ATRIP) |
| DNA-PK(h) | 14-950 | Human | Atypical | GenBank NM_006904.6 |
| | | derived | | |

### 2. Methods

The compounds synthesized according to the present invention were commissioned to an external institution Eurofins Cerep (https://www.eurofins.com/) to perform corresponding protein kinase experiments. The ATM(h), ATR/ATRIP(h), and DNA-PK (h) were detected by HTRF®.

### 3. Data Analysis

The calculation formula was as follows:
HTRF® method:

$$Remained kinase activity = \frac{\begin{matrix}Average readout of sample wells - \\ Average readout of wells without enzymes\end{matrix}}{\begin{matrix}Average readout of control wells - \\ Average readout of wells without enzymes\end{matrix}}$$

For IC₅₀ determination, the data were analyzed using XLFit version 5.3 (ID Business Solutions).

### 4. Test Results

TABLE 4

| | ATM(h) | ATR/ATRIP(h) | DNA-PK(h) |
|---|---|---|---|
| | (enzyme, | (enzyme, | (enzyme, |
| Compound | IC₅₀, μM) | IC₅₀, μM) | IC₅₀, μM) |
| Compound 1 | 0.0005 | 0.009 | 0.0001 |
| Compound 2 | 0.001 | 0.013 | 0.0004 |
| Compound 27 | 0.0007 | 0.017 | 0.0002 |
| Compound 28 | 0.0006 | 0.013 | 0.0003 |
| Compound 29 | 0.0007 | 0.020 | 0.0003 |
| Compound 30 | 0.001 | 0.036 | 0.0006 |
| Compound 31 | 0.0004 | 0.015 | 0.0002 |
| Compound 32 | 0.0006 | 0.017 | 0.0003 |

### Example 8: Inhibition of Cell Proliferation by the ADC Products of the Present Invention In Vitro

### Experimental Methods

HCC827 cells (EGFR-positive, purchased from the cell bank of American Type Culture Collection, ATCC, Cat. No. CRL-2868), H1650 cells (EGFR-positive, purchased from the cell bank of American Type Culture Collection, ATCC, Cat. No. CRL-5883), and H1770 cells (EGFR-negative, purchased from the cell bank of American Type Culture Collection, ATCC, Cat. No. CRL-5893) were all cultured in an RPMI-1640 medium (Gibco, Cat. No. A1049101) with 10% fetal bovine serum (FBS) (Gibco, Cat. No. A5669701). The cells were normally cultured in a cell culture incubator at 5% CO₂ and 37° C. The cells were added to a 96-well plate at 1.0×10³ cells/well, 2.0×10³ cells/well and 3.0×10³ cells/well, and 100 μL/well, respectively, and incubated in a cell culture incubator at 5% CO₂ and 37° C. The test antibody-drug conjugates were diluted with an RPMI-1640 medium in a 4-fold gradient to the corresponding concentrations, and then 10 μL/well of the diluted test antibody-drug conjugates at different concentrations (final concentrations: 200, 50, 12.5, 3.1, 0.78, 0.20, 0.049, and 0.012 nM) or 10 μL/well of the control solution (RPMI-1640) were added to a 100 μL/well cell culture system. The cells were incubated in a cell culture incubator at 5% CO₂ and 37° C. for 6 days.

After 6 days of culture, the test was performed using the CCK8 or 3D CTG as follows:
1) CCK8: 10 μL/well of Cell Counting Kit-8 reagent (CCK-8, Life-iLab, Cat. No. AC11L057) was added to the wells and mixed well with shaking. The cells were incubated in a cell culture incubator at 5% CO₂ and 37° C. for 1.0-4.0 hours.
2) 3D CTG: 50 μL/well of CellTiter-Glo® reagent (Promega, Cat. No. G9683) was added to the wells, mixed well with shaking, and then incubated for 30 minutes.

### Detection and Data Analysis

1) CCK8: The OD₄₅₀ optical density values were read on an EnVision™ multi-function microplate reader (PerkinElmer), and the inhibition rates (%) of the test antibody-drug conjugates were calculated. Then, the half maximal inhibitory concentration (IC₅₀) values were obtained using the XLfit5 software (ID Business Solutions Limited). All samples were tested in duplicate wells.

$$Inhibition rate \% = ( 1 - ( O {D}_{450 test antibody - drug conjugate} - O {D}_{450 background} ) / ( O {D}_{450 cell} - O {D}_{450 background} ) ) \times 100 \%$$

wherein
OD_{450 test antibody-drug conjugate} represents the optical density value in the wells of test antibody-drug conjugates;
OD_{450 cell} represents the optical density value in the wells of cell controls;
OD_{450 background} represents the optical density value in the wells of cell-free controls.
2) 3D CTG: The luminescent signal was read on an EnVision™ multi-function microplate reader (PerkinElmer), and the inhibition rates (%) of the test antibody-drug conjugates were calculated. Then, the half maximal inhibitory concentration (IC₅₀) values were obtained using the XLfit5 software (ID Business Solutions Limited). The fitting formula was as follows: #205. All samples were tested in duplicate wells.

$$Inhibition rate \% = ( 1 - ( {Lum}_{test antibody - drug conjugate} - {Lum}_{background} ) / ( {Lum}_{cell} - {Lum}_{background} ) ) \times 100 \%$$

wherein
Lum_{test antibody-drug conjugate} represents the luminescent signal in the wells of test antibody-drug conjugates;
Lum_{cell} represents the luminescent signal in the wells of cell controls;
Lum_{background} represents the luminescent signal in the wells of cell-free controls.

TABLE 5

IC₅₀, nM

| | HCC827 cell | H1650 cell | H1770 cell |
|---|---|---|---|
| ADC No. | line (EGFR+) | line (EGFR+) | line (EGFR−) |
| ADC-1 | 0.2 | 0.5 | 108.9 |
| ADC-2 | 0.3 | 0.8 | 96.1 |
| ADC-3 | 0.2 | 0.9 | 79.0 |
| ADC-4 | 0.8 | 3.8 | >200 |
| ADC-5 | 0.2 | 0.6 | 163.2 |
| ADC-6 | 0.8 | 2.6 | >200 |
| ADC-7 | 0.2 | 0.4 | 83.5 |
| ADC-8 | 0.7 | 1.5 | >200 |
| ADC-9 | 0.2 | 0.3 | 121.1 |
| ADC-10 | 0.3 | 1.1 | >200 |
| ADC-11 | 0.6 | 2.4 | >200 |
| ADC-12 | 0.3 | 2.2 | >200 |
| ADC-13 | 0.3 | 1.0 | 175.6 |

Conclusions: According to the data in Table 5, the ADC products of the present invention exhibit proliferation inhibitory activity in-vitro dependent on cellular EGFR expression, with strong proliferation inhibitory activity against EGFR-positive cells and weak proliferation inhibitory activity against EGFR-negative cells.

### Example 9: Plasma Stability Study of the Antibody-Drug Conjugates of the Present Invention

9.1 Objective: The stability of the antibody-drug conjugates of the present invention in plasma was evaluated by measuring the percent of payload release from the antibody-drug conjugates of the present invention in human plasma.

### Experimental Methods:

First, the ADC product solution was added to human plasma to achieve a concentration of 100 nM;
A 50 μL aliquot was added to a 96-well deep-well plate, and was incubated in a 5% CO₂ incubator at 37° C.;
The samples were transferred to ice at preset time points (0, 24, and 96 hours) to terminate the reaction, and then the samples were stored in a refrigerator at −80° C.; in which 0 hour sample was collected within one minute of adding the ADC sample to the plasma
The concentration of the free payloads was determined by LC-MS/MS and the percent of payload release was calculated using the following formula:

$$Percent of payload release = free payload concentration / nominal conjugated payload concentration$$

The resulting data are shown in Table 6-1.

TABLE 6-1

| | Percent of payload release in human | | |
|---|---|---|---|
| | plasma at different incubation times (%) | | |
| ADC No. | 0 hour | 24 hours | 96 hours |
| ADC-2 | ND (percent of | ND (percent of | 0.140% |
| | release <0.0584%) | release <0.0584%) | |

ND: Not detectable

Conclusions: As can be seen from the data in Table 6-1, the percent release of the free payload from ADC-2 prepared in the present invention is low at the preset time points, indicating that the antibody-drug conjugates of the present invention are stable in human plasma.

Example 9.2 Objective: The stability of the antibody-drug conjugates of the present invention in plasma was evaluated by measuring the percent of payload release from the antibody-drug conjugates of the present invention in human plasma.

### Experimental Methods:

First, the ADC product solution was added to human plasma to achieve a concentration of 100 nM;
A 50 μL aliquot was added to a 96-well deep-well plate, and was incubated in a 5% CO₂ incubator at 37° C.;
The samples were transferred to ice at a preset time point (96 hour) to terminate the reaction, and then the samples were stored in a refrigerator at −80° C.;
The concentration of the free payloads was determined by LC-MS/MS and the percent of the payload release was calculated using the following formula:

$$Percent of the payload release = free payload concentration / nominal conjugated payload concentration$$

The resulting data are shown in Table 6-2.

TABLE 6-2

| | Percent of payload release in human |
|---|---|
| | plasma at different incubation times (%) |
| ADC No. | 96 hours |
| ADC-7 | ND (percent of release <0.0641%) |
| ADC-9 | ND (percent of release <0.0625%) |
| ADC-1 | ND (percent of release <0.0128%) |
| ADC-5 | ND (percent of release <0.0656%) |

ND: Not detectable.

Conclusions: As can be seen from the data in Table 6-2, the percent of the free payload release from ADC-7, ADC-9, ADC-1, and ADC-5 prepared in the present invention is low at the preset time points, indicating that the antibody-drug conjugates of the present invention are stable in human plasma.

### Example 10 In-Vivo Anti-Tumor Activity of Antibody-Drug Conjugate of the Present Invention

The anti-tumor activity of the antibody-drug conjugates of the present invention was evaluated in NCI-H1650 subcutaneous xenograft model in BALB/c nude mice.

Methods: Human lung cancer cell line NCI-H1650 (purchased from ATCC) was cultured and passaged in the RPMI1640 medium containing 10% fetal bovine serum. Tumor cells in the logarithmic growth phase were inoculated subcutaneously into the right flank of BALB/c nude mice (Shanghai Lingchang Biotechnology Co., Ltd.) at 5×10⁶ cells per mouse.

Mice were randomized into treatment and control groups according to tumor volume. The day of grouping was defined as Day 0, and the next day after grouping, when the first dose was administered, was defined as Day 1. The antibody-drug conjugates ADC-2, ADC-7, ADC-9, ADC-1 and ADC-5 prepared according to Example 4 of the present invention were diluted with normal saline and administered according to the study design shown in Table 7.

Two orthogonal diameters of subcutaneous tumors and mouse body weights were measured 2-3 times per week. Tumor volume was calculated by the following formula:

$$T V = 1 / 2 a {b}^{2}$$

wherein a and b represent the length and width diameters of the tumor, respectively.

The tumor growth inhibition rate (TGI) was calculated:

$$T G I = [ 1 - {( T {V}_{t} - T {V}_{0} )}_{treatment} / {( T {V}_{t} - T {V}_{0} )}_{vehicle control} ] \times 100 \%$$

wherein TV₀ represents the average tumor volume on the grouping day (Day 0), and TVₜ represents the average tumor volume of the group in a certain day (Day t) after treatment.

Statistical analysis between the drug treatment group and the vehicle control group was performed with Student's t-test, and p<0.05 were considered statically significant.

### Results:

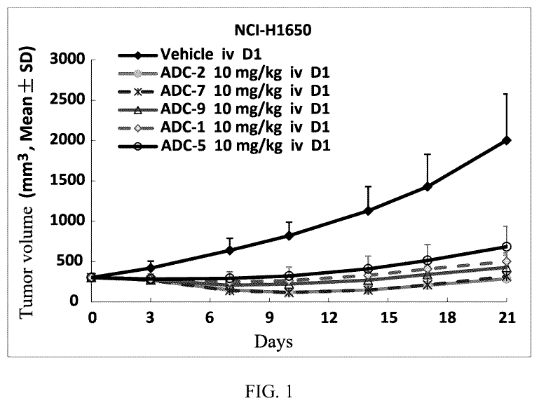

In the NCI-H1650 subcutaneous model, the test articles ADC-2, ADC-7, ADC-9, ADC-1, and ADC-5 were administered once at 10 mg/kg, IV (injection via tail vein) on Day 1. On Day 21, all of ADC-2, ADC-7, ADC-9, ADC-1, and ADC-5 treatment groups exhibited potent anti-tumor activity (FIG. 1), with tumor growth inhibition rates (TGIs) of 100.8%, 99.3%, 92.7%, 88.3%, and 77.5%, respectively. All treatment groups induced tumor regression with TGIs of 146.1%, 148.5%, 128.7%, 116.2%, and 103.0%, respectively, on Day 7. At the end of the experiment, no decrease in the average body weight was observed in each ADC treatment group, indicating well tolerance of animals.

In conclusion, ADC-2, ADC-7, ADC-9, ADC-1, and ADC-5 all exhibited potent anti-tumor effects in the NCI-H1650 subcutaneous xenograft model (p<0.01).

TABLE 7
Dosage regimens of ADCs of the present invention
in NCI-H1650 subcutaneous xenograft models

| | Dosage of | | |
|---|---|---|---|
| | administration | Dosage | Number of |
| Group | (mg/kg) | regimen | animals |
| Vehicle | 0 | IV, Day 1 | 9 |
| ADC-2 | 10 | IV, Day 1 | 9 |
| ADC-7 | 10 | IV, Day 1 | 9 |
| ADC-9 | 10 | IV, Day 1 | 9 |
| ADC-1 | 10 | IV, Day 1 | 9 |
| ADC-5 | 10 | IV, Day 1 | 9 |

Notes:
Vehicle: vehicle control; IV: intravenous injection; Day 1, the first day.

Sequence Listing

| SEQ | | |
|---|---|---|
| ID | | |
| NO | Cetuximab | |
| 1 | HCDR1 | NYGVH |
| 2 | HCDR2 | VIWSGGNTDYNTPFTS |
| 3 | HCDR3 | ALTYYDYEFAY |
| 4 | LCDR1 | RASQSIGTNIH |
| 5 | LCDR2 | YASESIS |
| 6 | LCDR3 | QQNNNWPTT |
| 7 | Heavy | QVQLKQSGPGLVQPSQSLSITCTVSGFSLTNYGVHWVRQSPGK |
| | chain | GLEWLGVIWSGGNTDYNTPFTSRLSINKDNSKSQVFFKMNSLQ |
| | variable | SNDTAIYYCARALTYYDYEFAYWGQGTLVTVSA |
| | region | |
| 8 | Light | DILLTQSPVILSVSPGERVSFSCRASQSIGTNIHWYQQRTNGSPR |
| | chain | LLIKYASESISGIPSRESGSGSGTDFTLSINSVESEDIADYYCQQN |
| | variable | NNWPTTFGAGTKLELK |
| | region | |
| 9 | Heavy | QVQLKQSGPGLVQPSQSLSITCTVSGFSLTNYGVHWVRQSPGK |
| | chain | GLEWLGVIWSGGNTDYNTPFTSRLSINKDNSKSQVFFKMNSLQ |
| | | SNDTAIYYCARALTYYDYEFAYWGQGTLVTVSAASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSCDKTHTCPPCPAPELLGGPSVFLFPPKPKDTLMISRTPEV |
| | | TCVVVDVSHEDPEVKFNWYVDGVEVHNAKTKPREEQYNSTY |
| | | RVVSVLTVLHQDWLNGKEYKCKVSNKALPAPIEKTISKAKGQP |
| | | REPQVYTLPPSREEMTKNQVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPGK |
| 10 | Light | DILLTQSPVILSVSPGERVSFSCRASQSIGTNIHWYQQRTNGSPR |
| | chain | LLIKYASESISGIPSRFSGSGSGTDFTLSINSVESEDIADYYCQQN |
| | | NNWPTTFGAGTKLELKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| 11 | HCDR1 | SGDYYWS |
| 12 | HCDR2 | YIYYSGSTDYNPSLKS |
| 13 | HCDR3 | VSIFGVGTFDY |
| 14 | LCDR1 | RASQSVSSYLA |
| 15 | LCDR2 | DASNRAT |
| 16 | LCDR3 | HQYGSTPLT |
| 17 | Heavy | QVQLQESGPGLVKPSQTLSLTCTVSGGSISSGDYYWSWIRQPP |
| | chain | GKGLEWIGYIYYSGSTDYNPSLKSRVTMSVDTSKNQFSLKVN |
| | variable | SVTAADTAVYYCARVSIFGVGTEDYWGQGTLVTVSS |
| | region | |
| 18 | Light | EIVMTQSPATLSLSPGERATLSCRASQSVSSYLAWYQQKPGQ |
| | chain | APRLLIYDASNRATGIPARFSGSGSGTDFTLTISSLEPEDFA |
| | variable | VYYCHQYGSTPLTFGGGTKAEIK |
| | region | |
| 19 | Heavy | QVQLQESGPGLVKPSQTLSLTCTVSGGSISSGDYYWSWIRQPP |
| | chain | GKGLEWIGYIYYSGSTDYNPSLKSRVTMSVDTSKNQFSLKVN |
| | | SVTAADTAVYYCARVSIFGVGTFDYWGQGTLVTVSSASTKGP |
| | | SVLPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKRVEPKSCDKTHTCPPCPAPELLGGPSVFLFPPKPKDTLM |
| | | ISRTPEVTCVVVDVSHEDPEVKFNWYVDGVEVHNAKTKPREE |
| | | QYNSTYRVVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 20 | Light | EIVMTQSPATLSLSPGERATLSCRASQSVSSYLAWYQQKPGQA |
| | chain | PRLLIYDASNRATGIPARFSGSGSGTDFTLTISSLEPEDFAVY |
| | | YCHQYGSTPLTFGGGTKAEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGE |
| | | C |

The invention claimed is:

1.-55. (canceled)

56. An antibody-drug conjugate having a structure represented by formula (I): 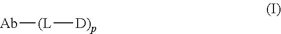
or a pharmaceutically acceptable salt thereof, or a solvate, a racemic mixture, an enantiomer, a diastereomer or a tautomer thereof, wherein
Ab is an antibody that can specifically bind to EGFR or an antigen-binding fragment thereof comprises 1, 2, 3, 4, 5 or 6 CDRs of cetuximab; 1, 2 and 3 CDRs of the heavy chain variable region, i.e., HCDR1, HCDR2 and HCDR3 of cetuximab; 1, 2 and 3 CDRs of the light chain variable region, i.e., LCDR1, LCDR2 and LCDR3 of cetuximab; three CDRs of the heavy chain variable region and three CDRs of the light chain variable region of cetuximab; a heavy chain variable region of cetuximab; a light chain variable region of cetuximab; a heavy chain variable region and a light chain variable region of cetuximab; a heavy chain of cetuximab; a light chain of cetuximab; a heavy chain and a light chain of cetuximab; or the antibody that specifically binds to EGFR or the antigen-binding fragment thereof comprises or consists of two heavy chains and two light chains of cetuximab; or the antibody that specifically binds to EGFR or the antigen-binding fragment thereof comprises complementarity determining regions HCDR1, HCDR2, HCDR3, LCDR1, LCDR2 and LCDR3; wherein the amino acid sequences of the HCDR1, HCDR2, HCDR3, LCDR1, LCDR2 and LCDR3 are the same as those of HCDR1, HCDR2, HCDR3, LCDR1, LCDR2 and LCDR3 of cetuximab; or the HCDR1, HCDR2, HCDR3, LCDR1, LCDR2 and LCDR3 are HCDR1, HCDR2, HCDR3, LCDR1, LCDR2 and LCDR3 of cetuximab; or the antibody that specifically binds to EGFR or the antigen-binding fragment thereof comprises a heavy chain variable region and a light chain variable region, and the amino acid sequence of the heavy chain variable region and the amino acid sequence of the light chain variable region are the same as the amino acid sequence of the heavy chain variable region and the amino acid sequence of the light chain variable region of cetuximab; or the heavy chain variable region and light chain variable region are the heavy chain variable region and light chain variable region of cetuximab; or the antibody that specifically binds to EGFR or the antigen-binding fragment thereof comprises a heavy chain and a light chain, and the amino acid sequence of the heavy chain and the amino acid sequence of the light chain are the same as the amino acid sequence of the heavy chain and the amino acid sequence of the light chain of cetuximab; or the heavy chain and light chain are the heavy chain and the light chain of cetuximab; or the antibody that specifically binds to EGFR has the same heavy chain and the same light chain as cetuximab; or
the antibody that specifically binds to EGFR or the antigen-binding fragment thereof comprises LCDR1, LCDR2, LCDR3, HCDR1, HCDR2 and HCDR3, or comprises a light chain variable region comprising LCDR1, LCDR2 and LCDR3 and a heavy chain variable region comprising HCDR1, HCDR2 and HCDR3; wherein
the HCDR1 comprises an amino acid sequence as set forth in SEQ ID NO: 1, the HCDR2 comprises an amino acid sequence as set forth in SEQ ID NO: 2, and the HCDR3 comprises an amino acid sequence as set forth in SEQ ID NO: 3; and the LCDR1 comprises an amino acid sequence as set forth in SEQ ID NO: 4, the LCDR2 comprises an amino acid sequence as set forth in SEQ ID NO: 5, and the LCDR3 comprises an amino acid sequence as set forth in SEQ ID NO: 6; or
the HCDR1 consists of an amino acid sequence as set forth in SEQ ID NO: 1, the HCDR2 consists of an amino acid sequence as set forth in SEQ ID NO: 2, and the HCDR3 consists of an amino acid sequence as set forth in SEQ ID NO: 3; and the LCDR1 consists of an amino acid sequence as set forth in SEQ ID NO: 4, the LCDR2 consists of an amino acid sequence as set forth in SEQ ID NO: 5, and the LCDR3 consists of an amino acid sequence as set forth in SEQ ID NO: 6; or
the antibody that specifically binds to EGFR or the antigen-binding fragment thereof comprises a heavy chain variable region and a light chain variable region, wherein the heavy chain variable region comprises an amino acid sequence as set forth in SEQ ID NO: 7, and the light chain variable region comprises an amino acid sequence as set forth in SEQ ID NO: 8; or the heavy chain variable region consists of an amino acid sequence as set forth in SEQ ID NO: 7, and the light chain variable region consists of an amino acid sequence as set forth in SEQ ID NO: 8; or
the antibody that specifically binds to EGFR or the antigen-binding fragment thereof comprises a heavy chain and a light chain, wherein the heavy chain comprises an amino acid sequence as set forth in SEQ ID NO: 9, and the light chain comprises an amino acid sequence as set forth in SEQ ID NO: 10; or the heavy chain consists of an amino acid sequence as set forth in SEQ ID NO: 9, and the light chain consists of an amino acid sequence as set forth in SEQ ID NO: 10; or the antibody that specifically binds to EGFR or the antigen-binding fragment thereof comprises two of the heavy chains and two of the light chains; or
the antibody that specifically binds to EGFR is selected from cetuximab, a biosimilar of cetuximab;
the structure of the D is: 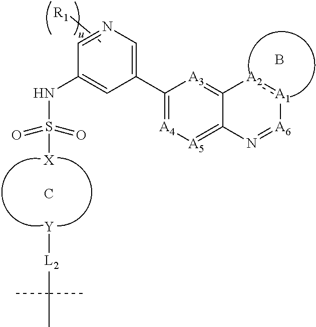
wherein:
the structure of 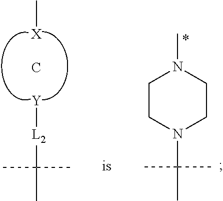
wherein * represents the site to which the parent core structure is linked; - - - represents the site to which the linker L is linked;
R₁ is selected from the group consisting of: —O(C₁₋₆ alkyl), —OC₁₋₆ haloalkyl, or C₃₋₉ cycloalkyl, wherein the C₁₋₆ alkyl is optionally substituted with one or more deuterium;
u, at each occurrence, is independently 0, 1, 2, or 3;
A₃, A₄, A₅, and A₆ are all CH, or A₃ is N, and A₄, A₅, and A₆ are all CH; 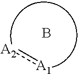
is selected from the following structures: 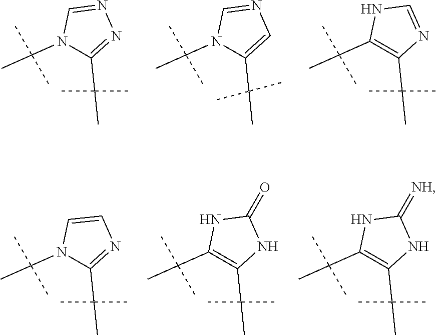
each of which is optionally substituted with 1, 2, or 3 R₃, each R₃ is independently selected from the group consisting of: H, CN, C₁₋₆ alkyl, C₃₋₉ cycloalkyl, and 3- to 12-membered heterocyclyl, wherein the C₁₋₆ alkyl and C₃₋₉ cycloalkyl can each optionally be substituted with one or more groups independently selected from the group consisting of: deuterium, —OH, C₁₋₆ alkylene-OH, halogen, and —C₁₋₆ alkylene-OC₁₋₆ alkyl; and
the structure of -L-D is: 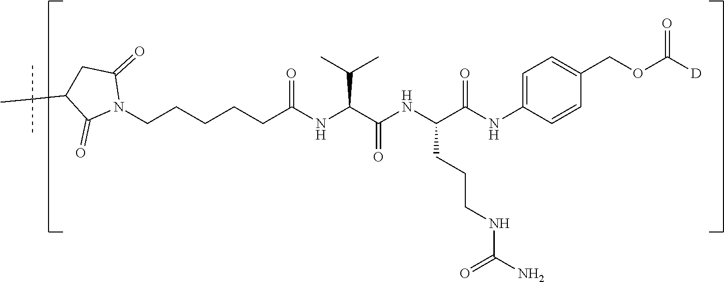
wherein the dashed lines represent the site to which Ab is linked; and
p is an integer from 1 to 20.

57. The antibody-drug conjugate or the pharmaceutically acceptable salt thereof, or the solvate, the racemic mixture, the enantiomer, the diastereomer or the tautomer thereof according to claim 56, wherein 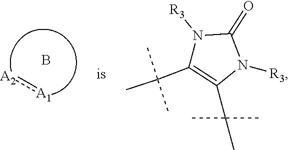
and each R₃ is independently as defined in claim 56.

58. The antibody-drug conjugate or the pharmaceutically acceptable salt thereof, or the solvate, the racemic mixture, the enantiomer, the diastereomer or the tautomer thereof according to claim 56, wherein 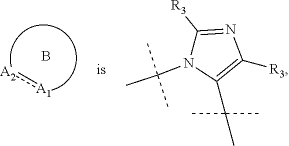
and each R₃ is independently H, —CH₃, or C₃₋₉ cycloalkyl.

59. The antibody-drug conjugate or the pharmaceutically acceptable salt thereof, or the solvate, the racemic mixture, the enantiomer, the diastereomer or the tautomer thereof according to claim 56, wherein R₁ is —O(C₁₋₆ alkyl).

60. The drug conjugate or the pharmaceutically acceptable salt thereof, or the solvate, the racemic mixture, the enantiomer, the diastereomer or the tautomer thereof according to claim 56, wherein each R₃ is independently selected from the group consisting of: hydrogen, —CH₃, —CD₃, —CH(CH₃)₂, —CH₂CF₃, or the following groups:

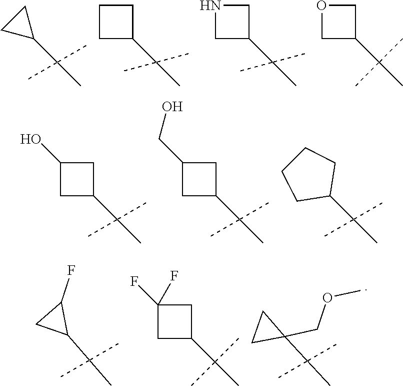

61. The antibody-drug conjugate or the pharmaceutically acceptable salt thereof, or the solvate, the racemic mixture, the enantiomer, the diastereomer or the tautomer thereof according to claim 56, wherein D is selected from the group consisting of: 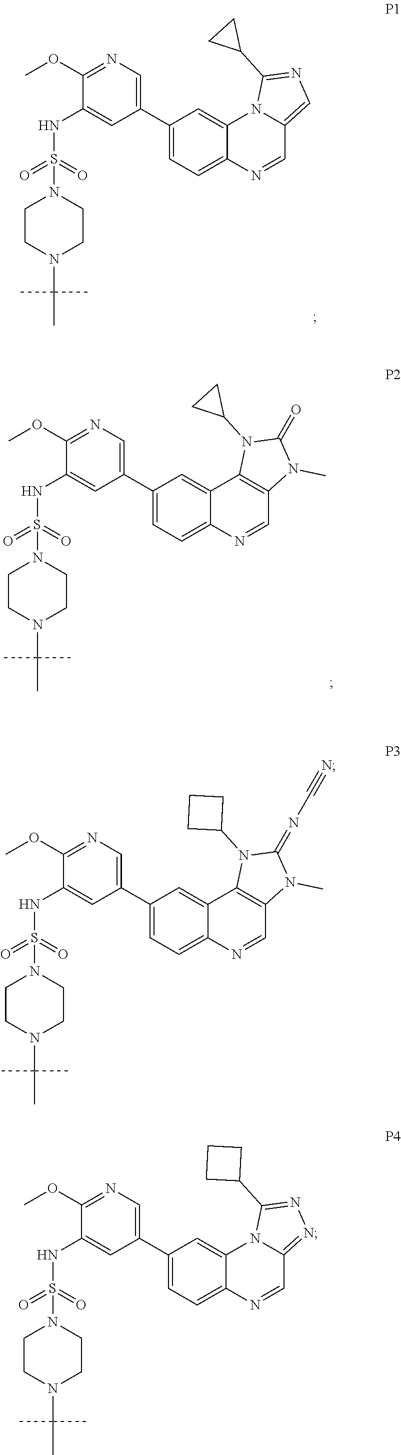 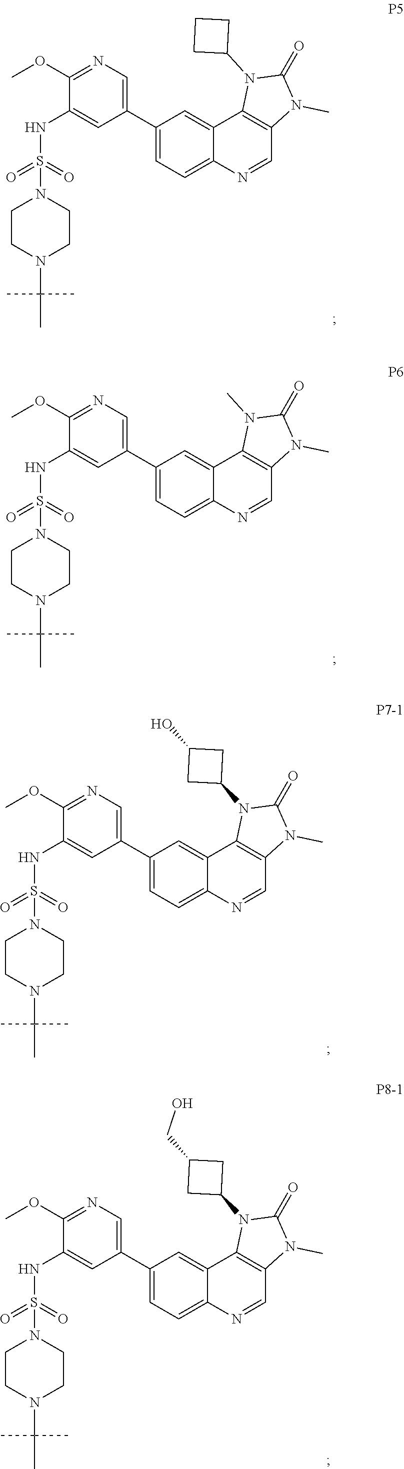 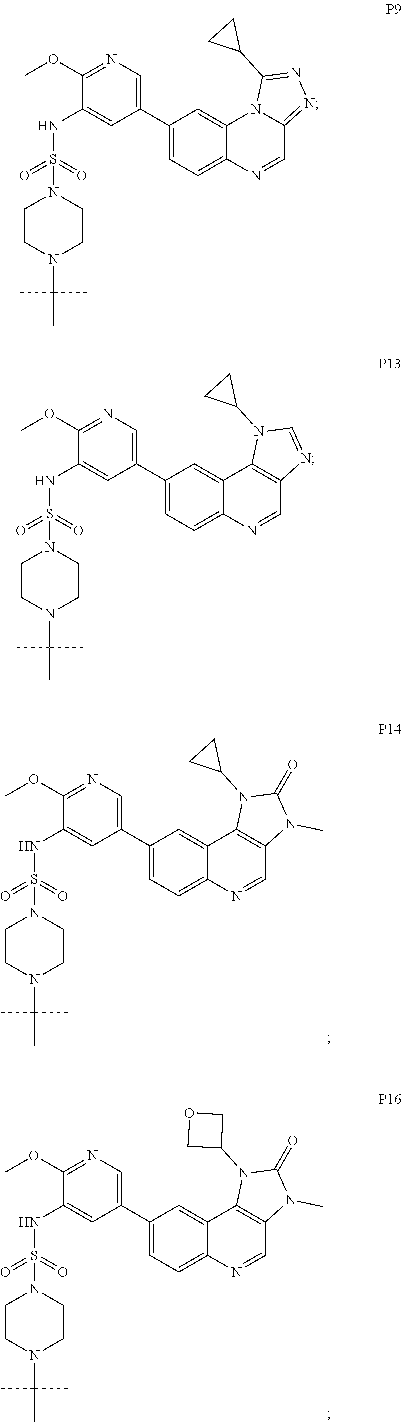 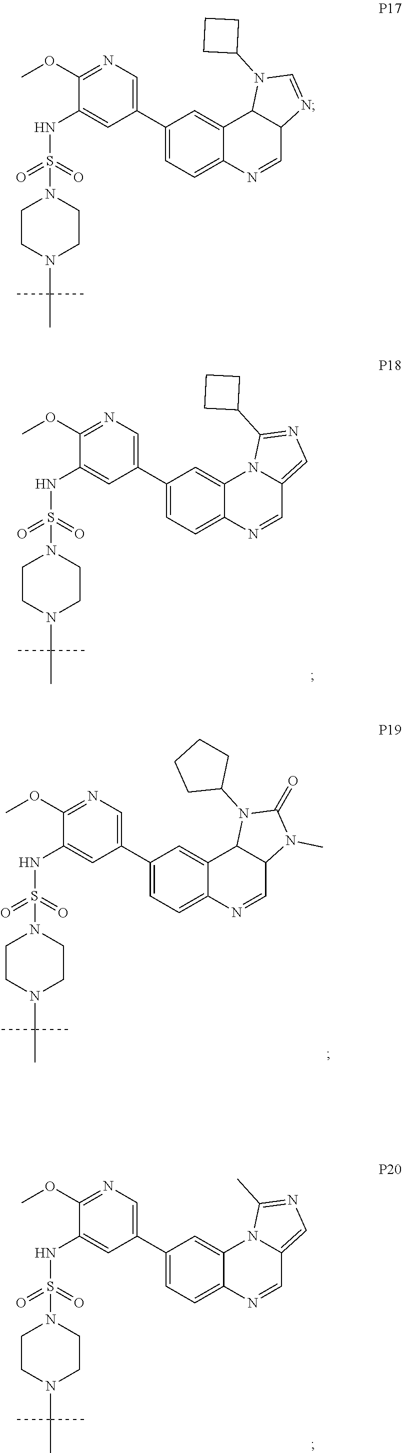 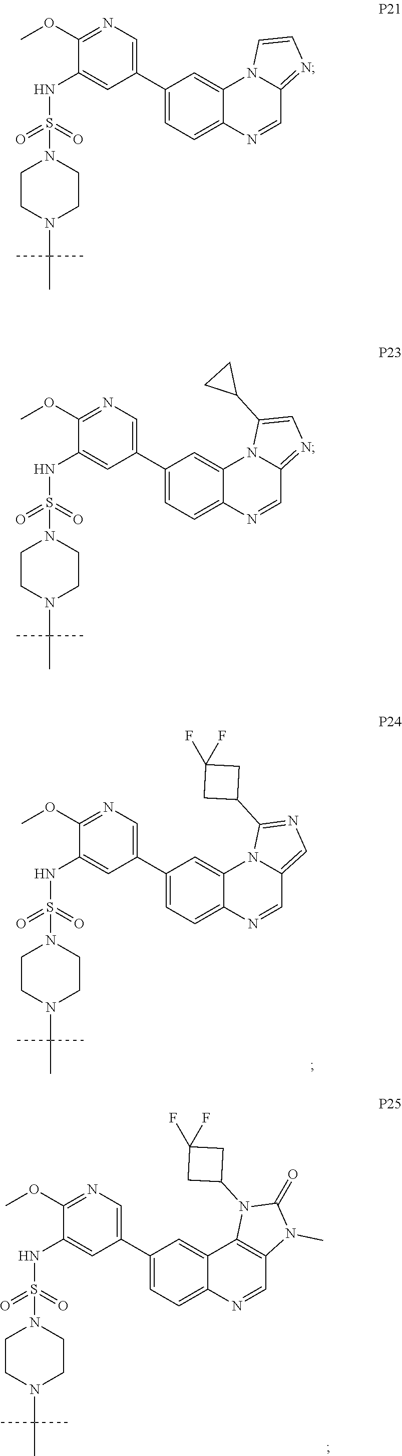 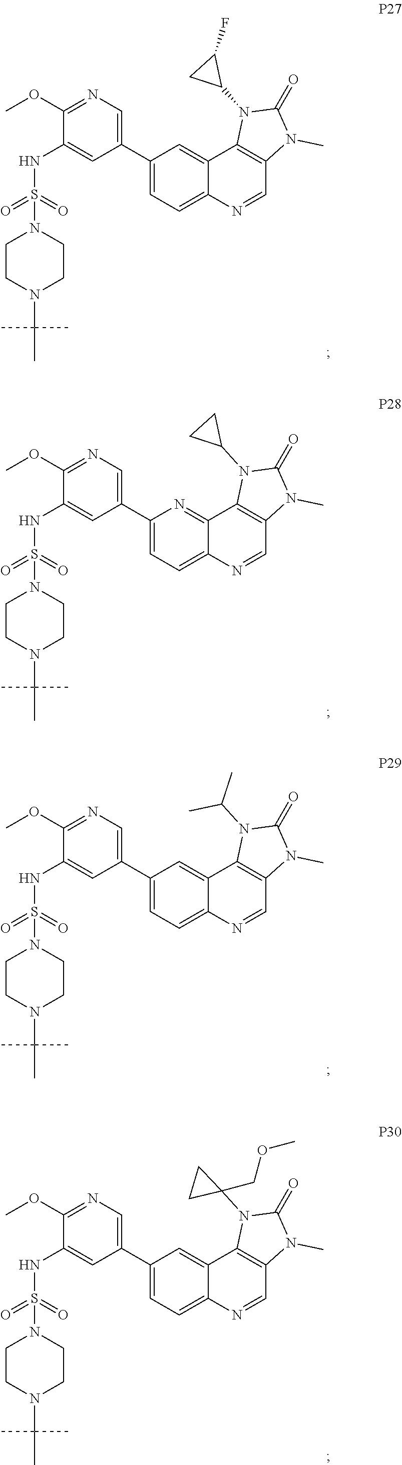 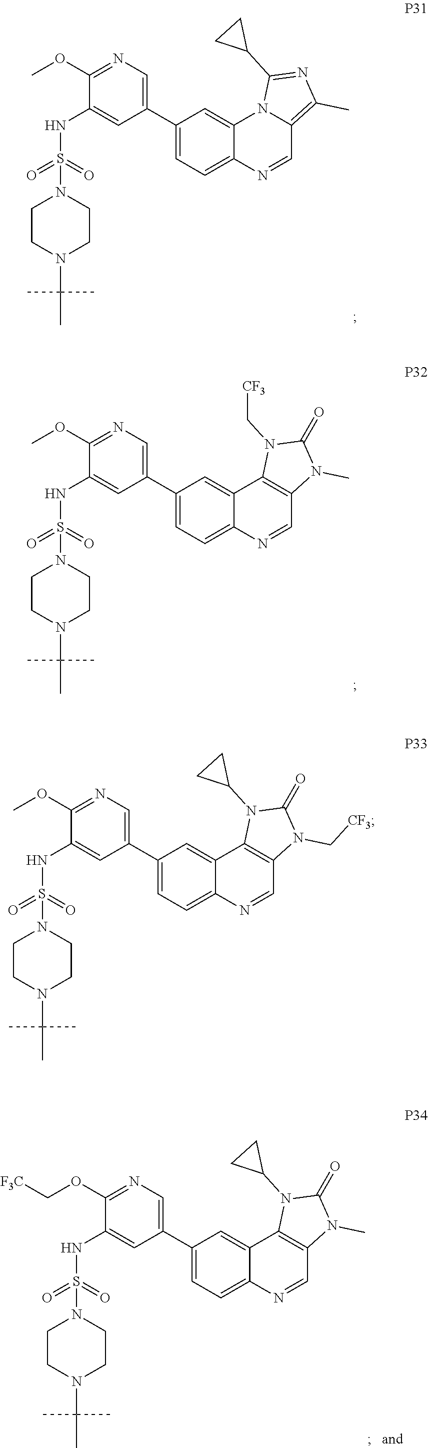 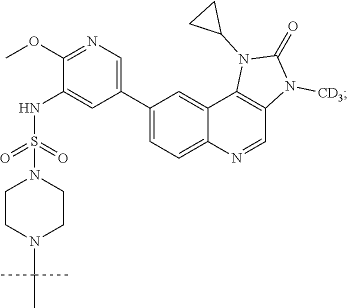
wherein the dashed lines represent the site where D is linked to the linker L.

62. The antibody-drug conjugate or the pharmaceutically acceptable salt thereof, or the solvate, the racemic mixture, the enantiomer, the diastereomer or the tautomer thereof according to claim 56, being selected from the group consisting of: 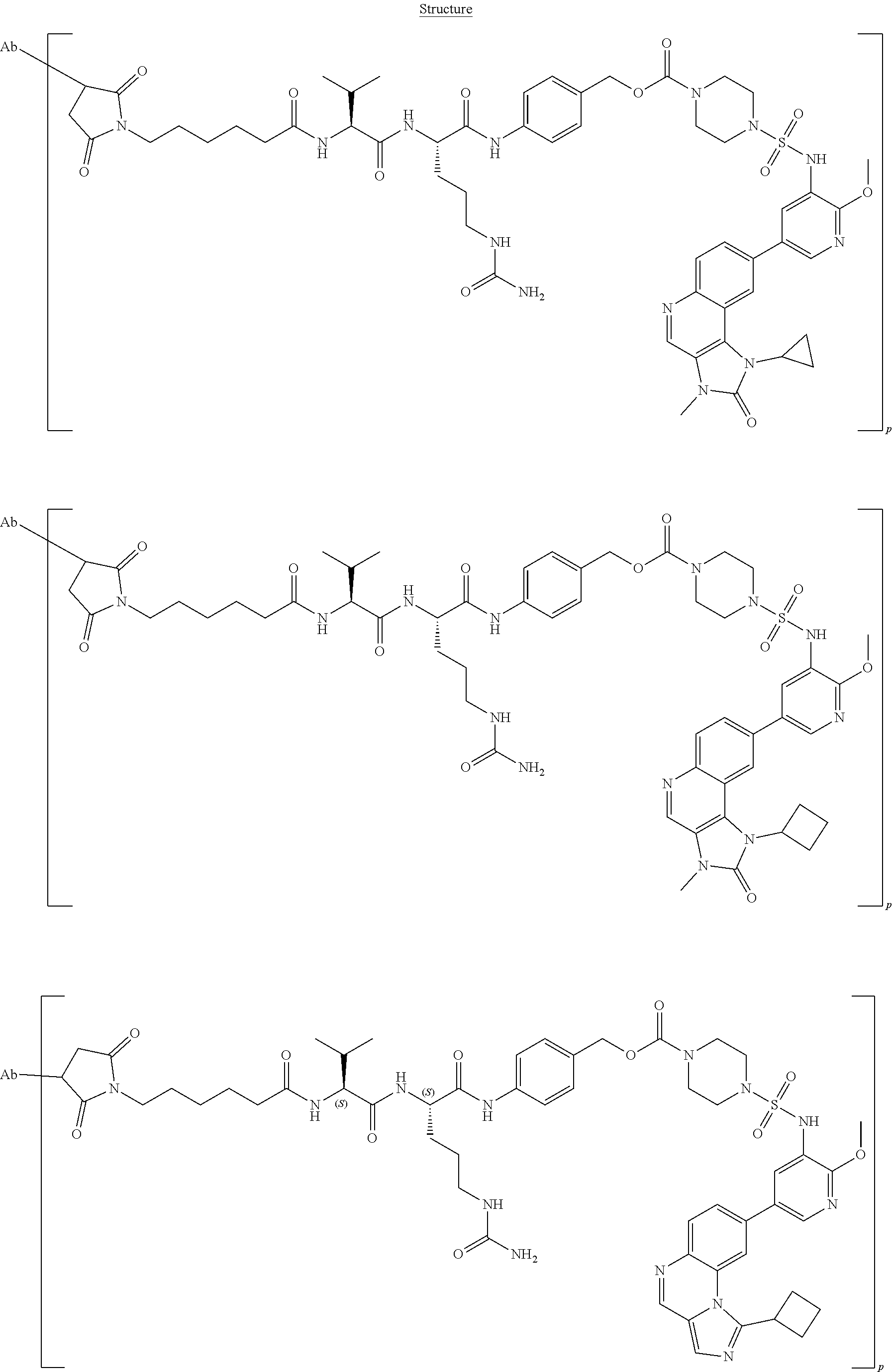 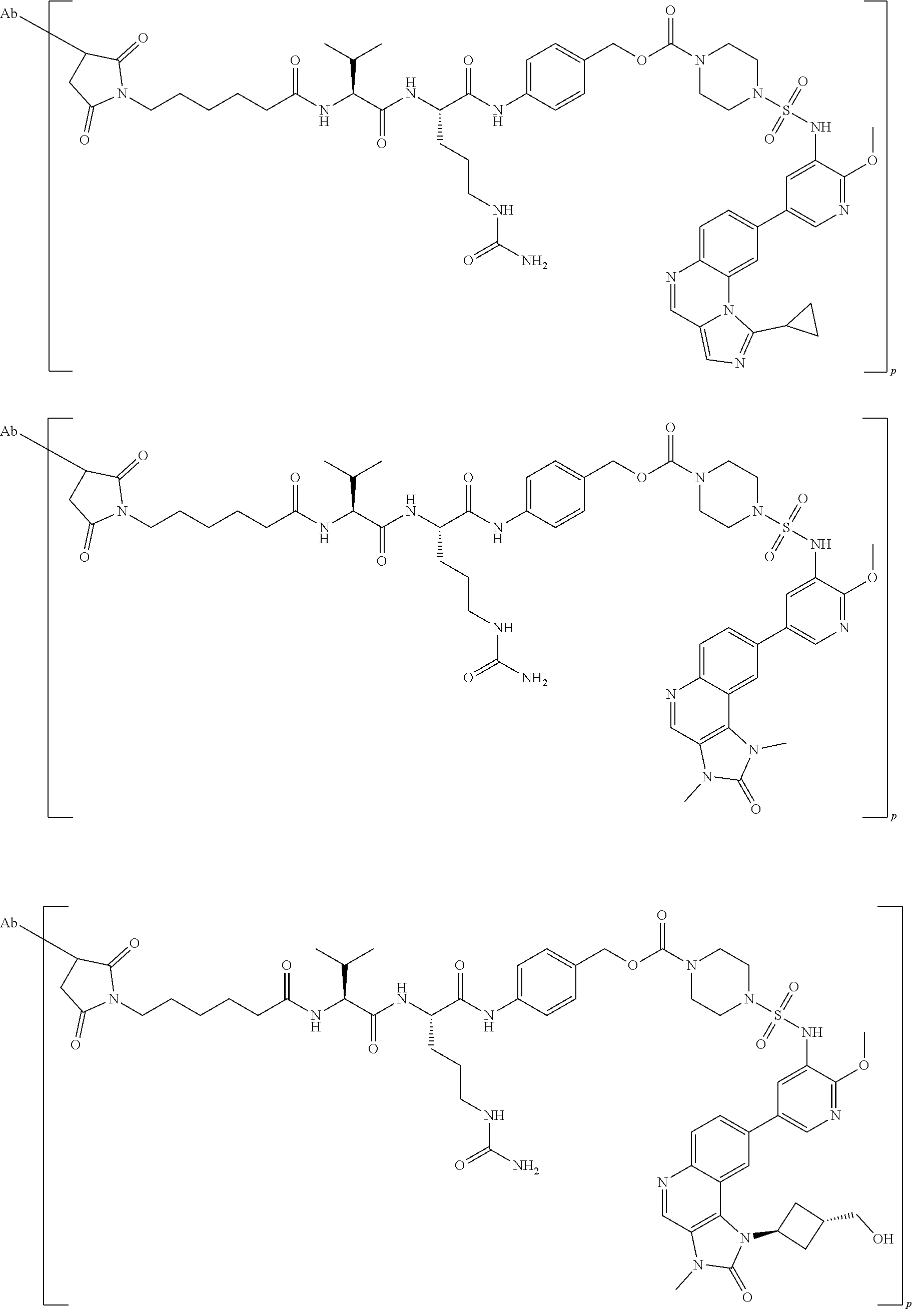 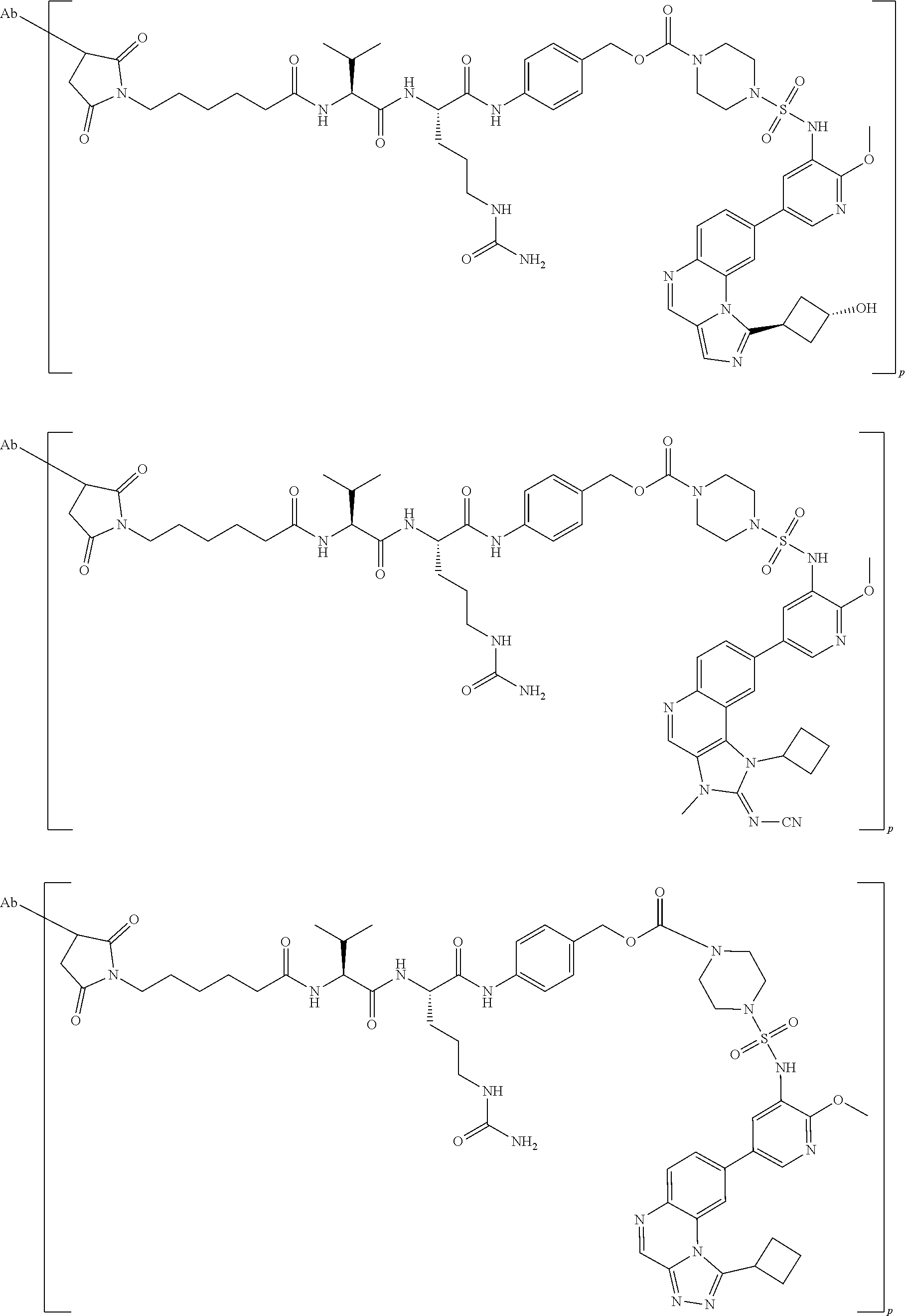 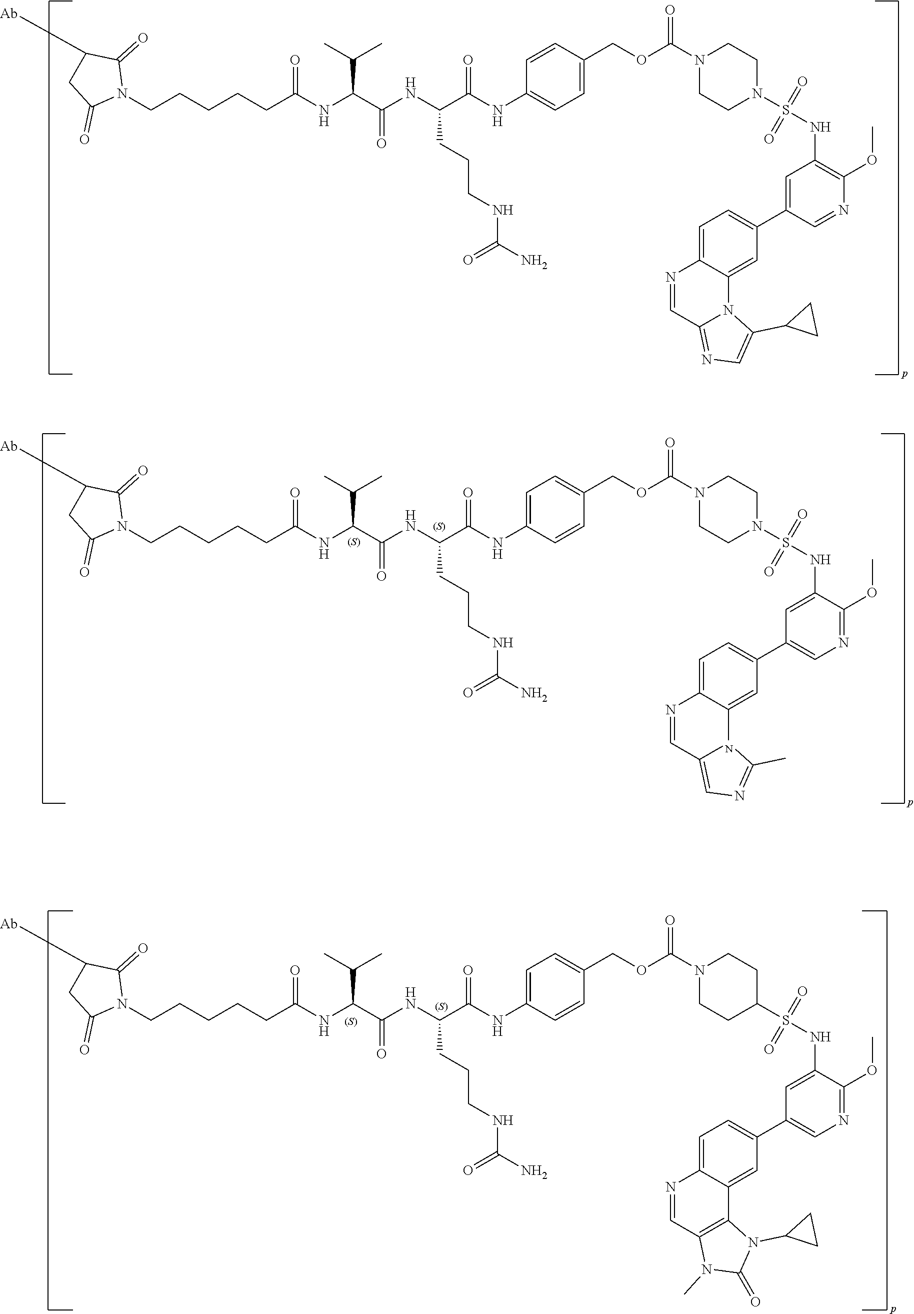 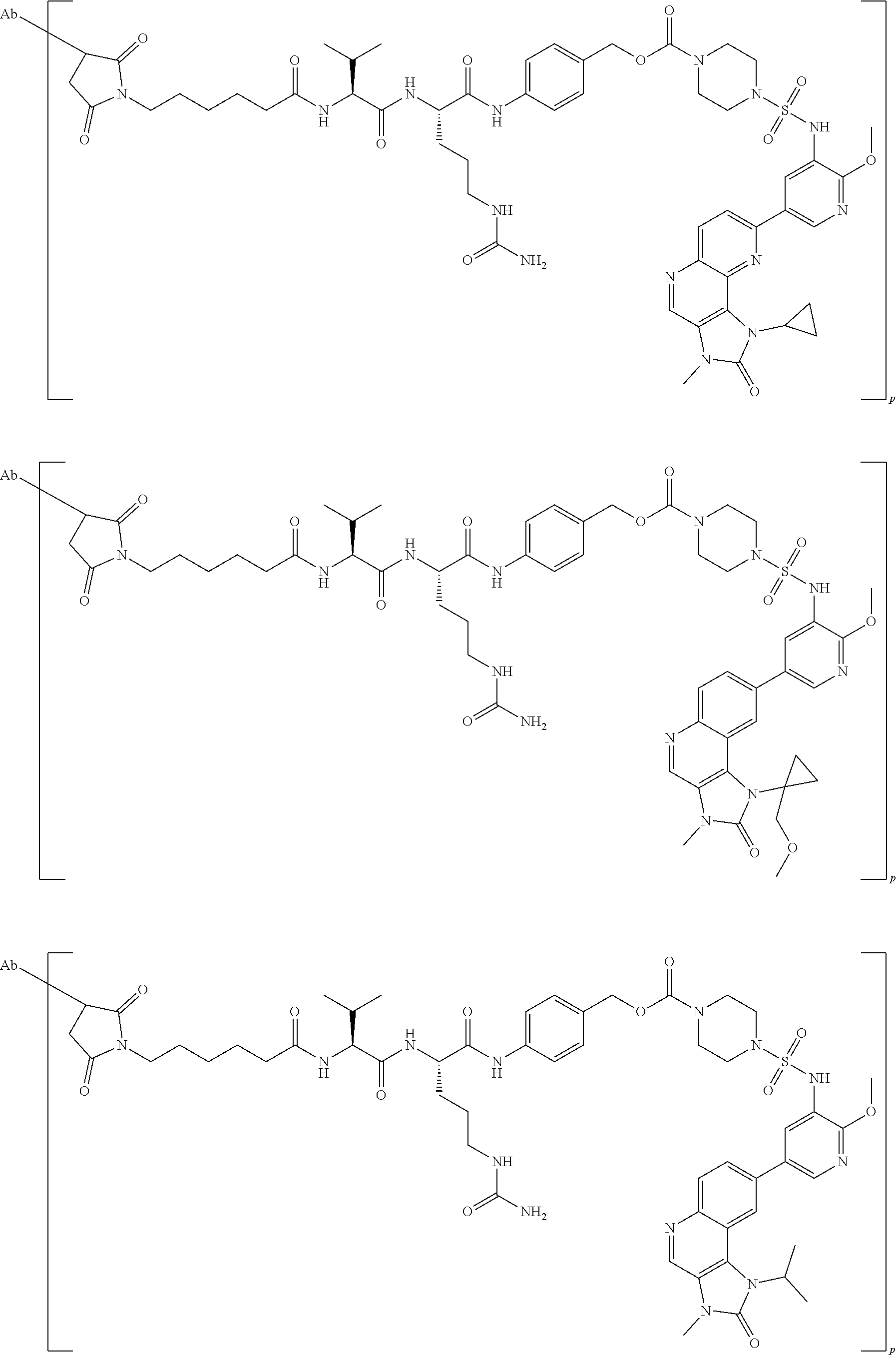 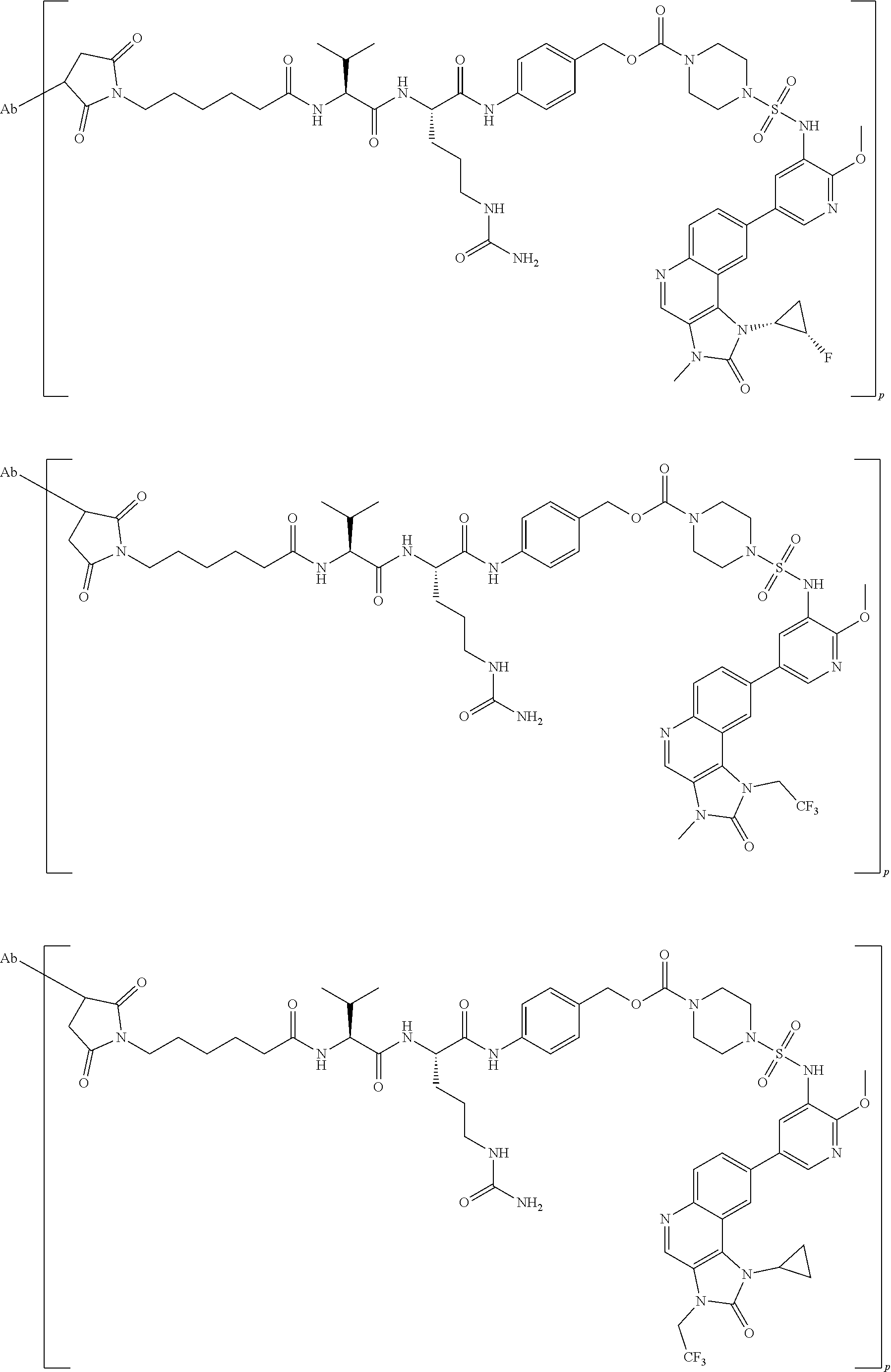 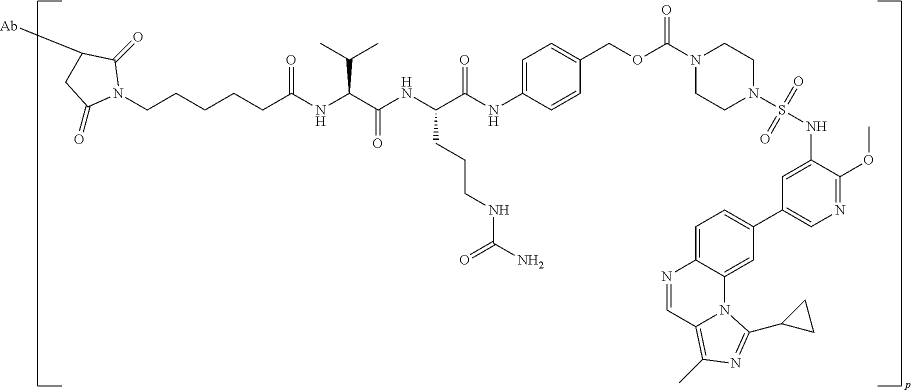
wherein Ab an p are as defined in claim 56.

63. The antibody-drug conjugate or the pharmaceutically acceptable salt thereof, or the solvate, the racemic mixture, the enantiomer, the diastereomer or the tautomer thereof according to claim 62, wherein p is selected from 2, 3, 4, 5, 6, 7, and 8.

64. The antibody-drug conjugate or the pharmaceutically acceptable salt thereof, or the solvate, the racemic mixture, the enantiomer, the diastereomer or the tautomer thereof according to claim 63, wherein Ab is cetuximab.

65. The antibody-drug conjugate or the pharmaceutically acceptable salt thereof, or the solvate, the racemic mixture, the enantiomer, the diastereomer or the tautomer thereof according to claim 56, having an average DAR of 2-8.

66. The antibody-drug conjugate or the pharmaceutically acceptable salt thereof, or the solvate, the racemic mixture, the enantiomer, the diastereomer or the tautomer thereof according to claim 65, having an average DAR of 3-4, 3.8-4.2, 3-5, 4-5.5, 5-7, 6.5-8, or 6-8.

67. The antibody-drug conjugate or the pharmaceutically acceptable salt thereof, or the solvate, the racemic mixture, the enantiomer, the diastereomer or the tautomer thereof according to claim 56, wherein the antibody-drug conjugate is selected from the group consisting of:

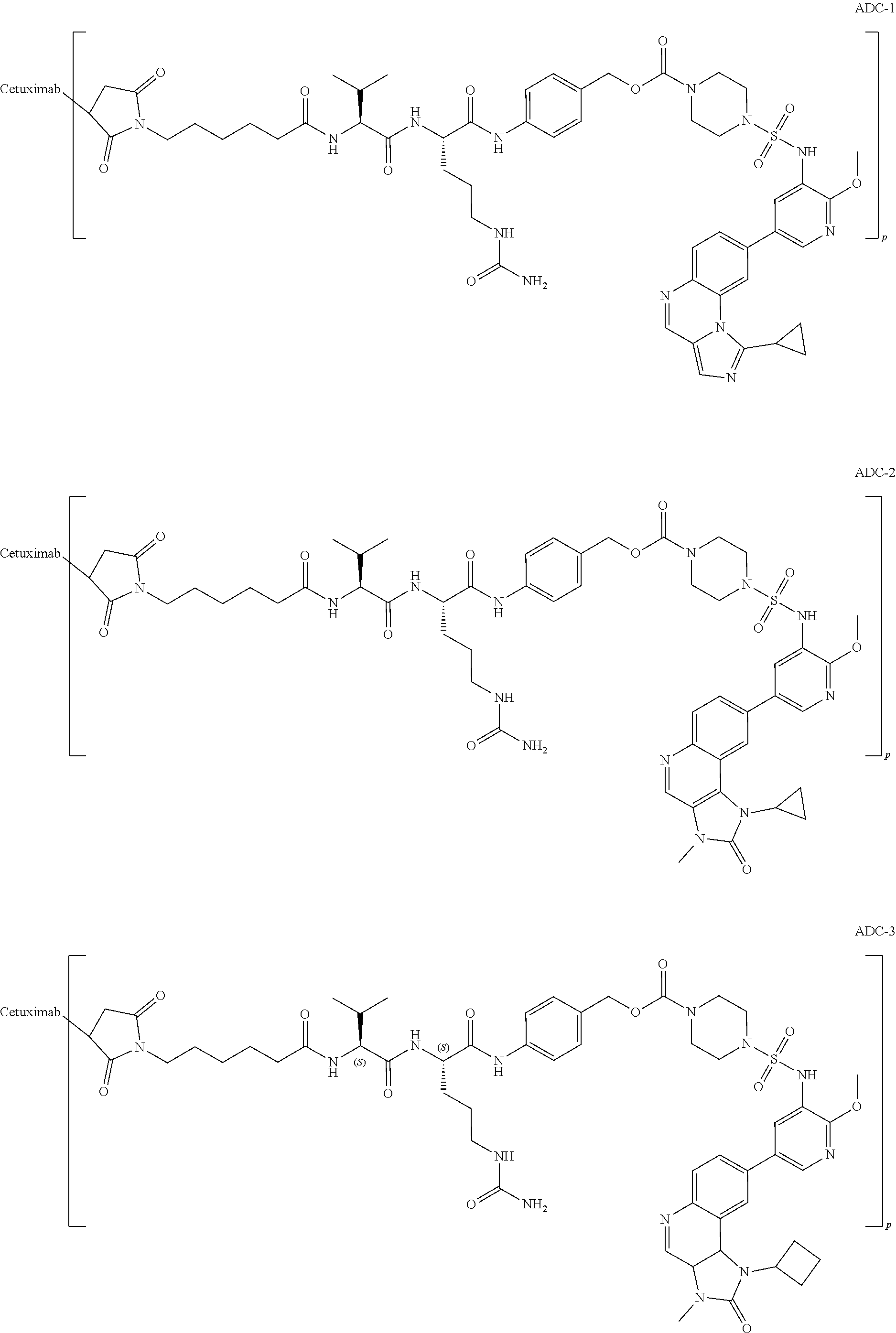

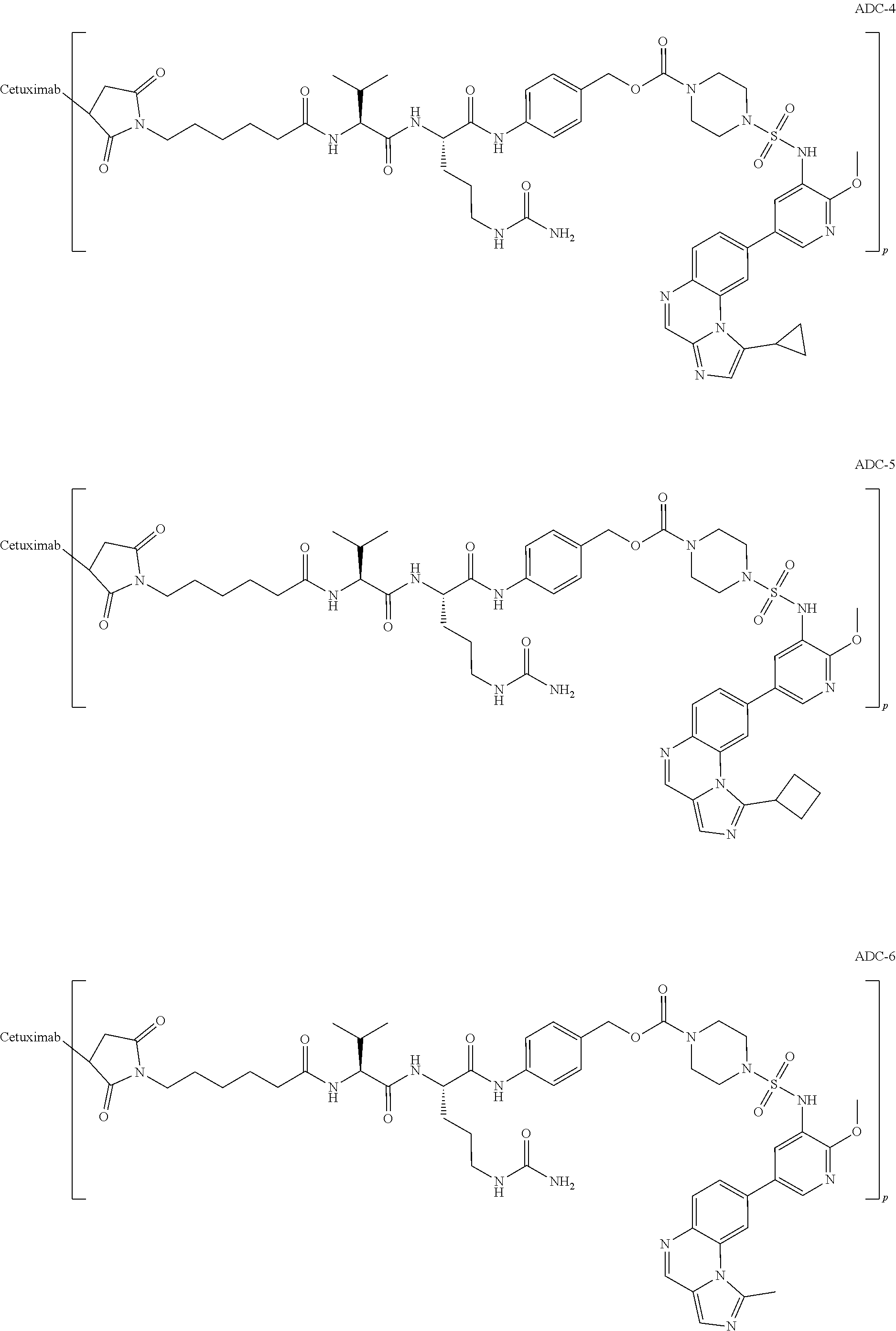

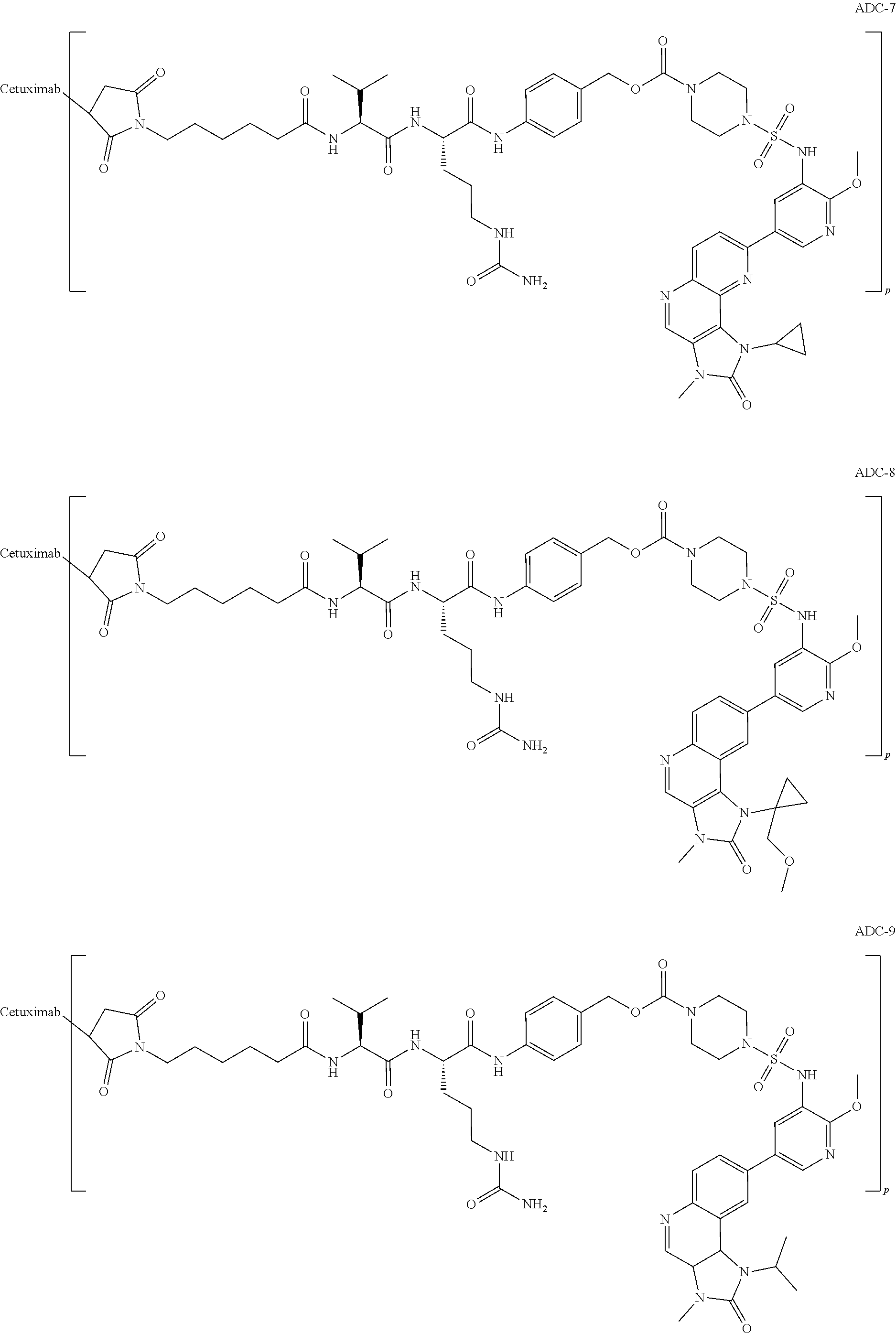

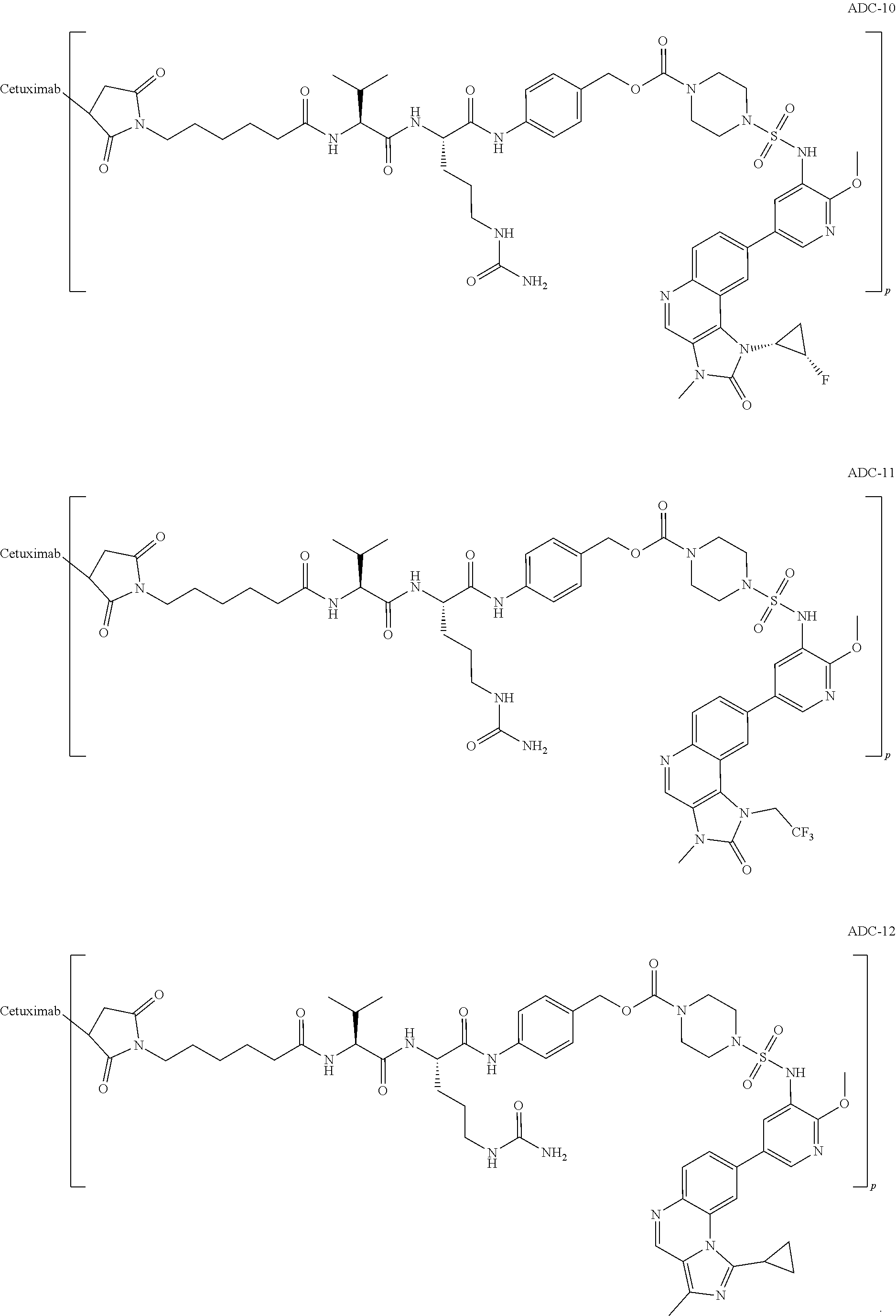

68. A pharmaceutical composition comprising the antibody-drug conjugate or the pharmaceutically acceptable salt thereof, or the solvate, the racemic mixture, the enantiomer, the diastereomer or the tautomer thereof according to claim 56, and a pharmaceutically acceptable carrier.

69. A method of treating a disease or condition mediated at least in part by PI3K and/or PIKK, comprising administering an effective amount of the antibody-drug conjugate or the pharmaceutically acceptable salt thereof according to claim 56 to a subject in need thereof.

70. The method according to claim 69, wherein the disease or condition mediated at least in part by PI3K and/or PIKK is selected from: a cancer or an autoimmune disease.

71. The method according to claim 70, wherein the autoimmune disease is selected from: rheumatoid arthritis, chronic obstructive pulmonary disease, allergic rhinitis, asthma, acquired hemophilia A (AHA), idiopathic thrombocytopenia (ITP), and activated phosphoinositide 3-kinase-delta syndrome (APDS).

72. The method according to claim 70, wherein the cancer is a solid tumor or a hematological malignancy, preferably non-small cell lung cancer or breast cancer.
